(19) United States
(12) Patent Application Publication
(10) Pub. No.: US 2011/0244575 A1
(43) Pub. Date: Oct. 6, 2011

(54) METHODS, SYSTEMS AND COMPOSITIONS FOR INCREASED MICROORGANISM TOLERANCE TO AND PRODUCTION OF 3-HYDROXYPROPIONIC ACID (3-HP)

(76) Inventors: Tanya E.W. Lipscomb, Boulder, CO (US); Michael D. Lynch, Boulder, CO (US); Ryan T. Gill, Denver, CO (US)

(21) Appl. No.: 13/055,138

(22) PCT Filed: Jul. 23, 2009
(86) PCT No.: PCT/US09/51607

§ 371 Date: Apr. 18, 2011

Related U.S. Application Data
(60) Provisional application No. 61/096,937, filed on Sep. 15, 2008.
(60) Provisional application No. 61/088,331, filed on Aug. 12, 2008.
(60) Provisional application No. 61/135,862, filed on Jul. 23, 2008.
(60) Provisional application No. 61/135,861, filed on Jul. 23, 2008.

(51) Int. Cl. C12N 1/00; C12N 15/63

(52) U.S. Cl. 435/471; 435/243

(57) ABSTRACT

The present invention relates to methods, systems and compositions, including genetically modified microorganisms, adapted to exhibit increased tolerance to 3-hydroxypropionic acid (3-HP), particularly through alterations to interrelated metabolic pathways identified herein as the 3-HP toleragenic pathway complex (“3HPTGC”). In various embodiments these organisms are genetically modified so that an increased 3-HP tolerance is achieved. Also, genetic modifications may be made to provide at least one genetic modification to any of one or more 3-HP biosynthesis pathways in microorganisms comprising one or more genetic modifications of the 3HPTGC.

### RELATED APPLICATIONS

This application claims priority to the following U.S. Provisional patent applications: 61/135,862, filed on Jul. 23, 2008; 61/088,331, filed on Aug. 12, 2008; 61/096,937, filed on Sep. 15, 2008; and 61/135,861, filed on Jul. 23, 2008, all of which are hereby incorporated by reference in their entirety.

### STATEMENT REGARDING FEDERALLY SPONSORED DEVELOPMENT

Some embodiments disclosed herein may have been supported in part by grant BES0228584 from the National Science Foundation. The U.S. government may have certain rights to practice such embodiments of the present invention.

### REFERENCE TO A SEQUENCE LISTING

This patent application provides a paper copy of sequence listings that are to be provided on compact disk in appropriate format in a later filing.

### INCORPORATION BY REFERENCE

All references cited herein are hereby incorporated by reference in their entirety.

### TECHNICAL FIELD

The present invention relates to methods, systems and compositions, including genetically modified microorganisms, e.g., recombinant microorganisms, adapted to exhibit increased tolerance to the chemical 3-hydroxypropionic acid (3-HP). Also, genetic modifications may be made to provide one or more 3-HP biosynthesis pathways such as in microorganisms comprising one or more genetic modifications of a complex identified as the 3-HP toleragenic pathway complex.

### BACKGROUND OF THE INVENTION

With increasing acceptance that petroleum hydrocarbon supplies are decreasing and their costs are ultimately increasing, interest has increased for developing and improving industrial microbial systems for production of chemicals and fuels. Such industrial microbial systems could completely or partially replace the use of petroleum hydrocarbons for production of certain chemicals.

One candidate chemical for biosynthesis in industrial microbial systems is 3-hydroxypropionic acid (“3-HP”, CAS No. 503-66-2), which as described herein may be converted to a number of basic building blocks for polymers used in a wide range of industrial and consumer products. Unfortunately, previous efforts to microbially synthesize 3-HP to achieve commercially viable titers have revealed that the microbes being used were inhibited by concentrations of 3-HP far below a determined commercially viable titer.

Metabolically engineering a selected microbe is one way to work toward an economically viable industrial microbial system, such as for production of 3-HP. A great challenge in such directed metabolic engineering is determining which genetic modification(s) to incorporate, increase copy numbers of, and/or otherwise effectuate, and/or which metabolic pathways (or portions thereof) to incorporate, increase copy numbers of, and/or otherwise modify in a particular target microorganism.

Metabolic engineering uses knowledge and techniques from the fields of genomics, proteomics, bioinformatics and metabolic engineering. This knowledge and techniques, combined with general capabilities in molecular genetics and recombinant technologies, present a high level of skill and knowledge as to the metabolic biochemistry of and genetic manipulations in various species of interest.

Despite the high level of knowledge and skill in the art, the identification of genes, enzymes, pathway portions and/or whole metabolic pathways that are related to a particular phenotype of interest remains cumbersome and at times inaccurate. Perspective as to the problem of finding a particular gene or pathway whose modification may provide greater tolerance and production of a product of interest may be further gained with the knowledge that there are at least 4,580 genes (of which 4,389 are identified as protein genes, 191 as RNA genes, and 116 as pseudo genes) and 224 identified metabolic pathways in an E. coli bacterium's genome (source www.biocyc.org, version 12.0 referring to Strain K-12). A review of specific metabolic engineering efforts, which also identifies existing gene identification and modification techniques, is “Engineering primary metabolic pathways of industrial micro-organisms,” Alexander Kern et al., Jl. of Biotechnology 129(2007)6-29, which is incorporated by reference for its listing and descriptions of such techniques.

Recently, however, a substantially more powerful and rapid genetics investigative technique was developed by a group of co-inventors including one or more of Applicants. This investigative tool advances the art by providing an approach to identify, with greater speed and accuracy than other methods, genes that are related to the expression of a particular trait. This technique involves creating multiple broad yet well-defined genetic libraries, introducing the genetic elements of such libraries into a microorganism population, and then exposing cultured cells of that microorganism population to a stressor or other selective pressure, sampling at specified time periods that capture shifts in the respective population toward more adaptive clones, and evaluating the genetic material in those clones. Descriptions of this method are found in U.S. Provisional Application No. 60/611,377 filed Sep. 20, 2004 and U.S. patent application Ser. No. 11/231,018 filed Sep. 20, 2005, published Apr. 20, 2006 as US2006/0084098 and entitled:” Mixed-Library Parallel Gene Mapping, A Quantitative Microarray Technique for Genome Wide Identification of Trait Conferring Genes” (hereinafter, the “SCALES Technique”), and SCALES: multiscale analysis of library enrichment, Lynch, M., Warnecke, T E, Gill, R T, Nature Methods, 2007. 4(87-93) which are incorporated herein by reference in their entirety for the teaching of the technique.

Notwithstanding such methodologies, including the SCALES technique, and in view of the high level of interest and skill in the art, there remains a need for a clearer understanding of how to modify and/or modulate microorganisms to increase 3-HP tolerance and bio-production in industrial microbial bio-production methods and systems.

### SUMMARY OF THE INVENTION

One aspect of the invention relates to a genetically modified microorganism comprising at least one genetic modification effective to increase 3-hydroxypropionic acid (“3-HP”) production, wherein the increased level of 3-HP production is greater than the level of 3-HP production in the wild-type microorganism, and at least one genetic modification of a metabolic complex identified herein as the 3-HP Toleragenic Complex (“3HPTGC”). Under certain conditions, such as culture in minimal media, the 3HPTGC genetic modification(s) allow the genetically modified microorganism to produce 3-HP under specific culture conditions such that 3-HP may accumulate to a relatively higher concentration without the toxic effects observed in unmodified microorganisms. The at least one genetic modification of a 3-HP production pathway may be to improve 3-HP accumulation and/or production of a 3-HP production pathway found in the wild-type microorganism, or may be to provide sufficient enzymatic conversions in a microorganism that normally does not synthesize 3-HP so that 3-HP is thus bio-produced. Methods of making such genetically modified microorganisms also are described and are part of this aspect of the invention.

Another aspect of the invention relates to a genetically modified microorganism comprising at least one genetic modification from two or more of the chorismate, threonine/homocysteine, polyamine synthesis, lysine synthesis, and nucleotide synthesis portions of the 3HPTGC. Non-limiting examples of multiple combinations exemplify the advantages of this aspect of the invention. Additional genetic modifications pertain to other portions of the 3HPTGC. Capability to bio-produce 3-HP may be added to some genetically modified microorganisms by appropriate genetic modification. Methods of identifying genetic modifications to provide to a microorganism to achieve an increased 3-HP tolerance, and microorganisms made by such methods, relate to this aspect of the invention.

Another aspect of the invention relates to a genetically modified microorganism that is able to produce 3-hydroxypropionic acid (“3-HP”), comprising at least one genetic modification to the 3HPTGC that increases enzymatic conversion at one or more enzymatic conversion steps of the 3HPTGC for the microorganism, and wherein the at least one genetic modification increases 3-HP tolerance of the genetically modified microorganism above the 3-HP tolerance of a control microorganism lacking the genetic modification. Methods of making such genetically modified microorganisms also are described and are part of this aspect of the invention.

Another aspect of the invention relates to a genetically modified microorganism comprising various core sets of specific genetic modification(s) of the 3HPTGC. In various embodiments this aspect may additionally comprise at least one genetic modification from one or more or two or more of the chorismate, threonine/homocysteine, polyamine synthesis, lysine synthesis, and nucleotide synthesis portions of the 3HPTGC. Methods of making such genetically modified microorganisms also are described and are part of this aspect of the invention.

Further, the invention includes methods of use of any of the above to improve a microorganism's tolerance to 3-HP, which may be in a microorganism having 3-HP production capability (whether the latter is naturally occurring, enhanced and/or introduced by genetic modification).

Also, another aspect of the invention is directed to providing one or more supplements, which are substrates (i.e., reactants) and/or products of the 3HPTGC (collectively herein “products” noting that substrates of all but the initial conversion steps are also products of the 3HPTGC), to a culture of a microorganism to increase the effective tolerance of that microorganism to 3-HP. This aspect may be combined with other of the above aspects.

Another aspect of the invention regards the genetic modification to introduce a genetic element that encodes a short polypeptide identified herein as IroK. The introduction of genetic elements encoding this short polypeptide has been demonstrated to improve 3-HP tolerance in E. coli under microaerobic conditions. This genetic modification may be combined with other genetic modifications and/or supplement additions of the invention.

Another aspect of the invention regards culture systems that comprise genetically modified microorganisms of the invention and optionally also 3HPTGC-related supplements.

Other aspects of the invention are directed to methods of identifying supplements, methods of identifying genetic modifications, and methods of identifying combinations of supplements and genetic modifications, related to the 3HPTGC that result in increased 3-HP tolerance for a microorganism.

Any of the above aspects may be practiced with a genetically modified microorganism that may comprise genetic deletions and additions in addition to the genetic modifications made to a 3-HP production pathway and/or the 3HPTGC.

### BRIEF DESCRIPTION OF THE DRAWINGS/FIGURES

The invention is explained in the following description in view of the drawings that show:

FIG. 1A, sheets 1-7 is a multi-sheet depiction of portions of metabolic pathways, showing pathway products and enzymes, that together comprise the 3-HP toleragenic complex (3HPTGC) in E. coli. Sheet 1 provides a general schematic depiction of the arrangement of the remaining sheets.

FIG. 1B, sheets 1-7, provides a multi-sheet depiction of the 3HPTGC for Bacillus subtilis. Sheet 1 provides a general schematic depiction of the arrangement of the remaining sheets.

FIG. 1C, sheets 1-7, provides a multi-sheet depiction of the 3HPTGC for Saccharomyces cerevisiae. Sheet 1 provides a general schematic depiction of the arrangement of the remaining sheets.

FIG. 1D, sheets 1-7, provides a multi-sheet depiction of the 3HPTGC for Cupriavidus necator (previously, Ralstonia eutropha). Sheet 1 provides a general schematic depiction of the arrangement of the remaining sheets.

FIG. 2 provides a representation of the glycine cleavage pathway.

FIG. 3 provides, from a prior art reference, a summary of a known 3-HP production pathway from glucose to pyruvate to acetyl-CoA to malonyl-CoA to 3-HP.

FIG. 4AA provides, from a prior art reference, a summary of a known 3-HP production pathway from glucose to phosphoenolpyruvate (PEP) to oxaloacetate (directly or via pyruvate) to aspartate to β-alanine to malonate semialdehyde to 3-HP.

FIG. 4B provides, from a prior art reference, a summary of known 3-HP production pathways including those referred to in FIGS. 2 and 3A.

FIG. 5A provides a schematic diagram of natural mixed fermentation pathways in E. coli.

FIG. 5B provides a schematic diagram of a proposed bio-production pathway modified from FIG. 4A for production of 3-HP.

FIG. 6A-O provides graphic data of control microorganisms responses to 3-HP, and FIG. 6P provides a comparison with one genetic modification of the 3HPTGC.

FIG. 7A depicts a known chemical reaction catalyzed by alpha-ketoglutarate encoded by the kgd gene from M. tuberculosis.

FIG. 7B depicts a new enzymatic function, the decarboxylation of oxaloacetate to malonate semialdehyde, that is to be achieved by modification of the kgd gene.

FIG. 8 shows a proposed selection approach for kgd mutants.

FIG. 9 depicts anticipated selection results based on the proposed selection approach of FIG. 8.

FIG. 10 shows a screening protocol related to the proposed selection approach depicted in FIG. 9.

FIG. 11 provides a comparison regarding the IroK peptide sequence.

FIG. 12 provides a calibration curve for 3-HP conducted with HPLC.

FIG. 13 provides a calibration curve for 3-HP conducted for GC/MS.

Tables are provided as indicated herein and are part of the specification and including the respective examples referring to them.

### DETAILED DESCRIPTION OF EMBODIMENTS OF THE INVENTION

The present invention is directed to methods, systems and compositions related to improved biosynthetic capabilities by metabolically engineered microorganisms to better tolerate and/or produce the compound 3-hydroxypropionic acid (“3-HP”). Various aspects of the present invention relate to 3-HP tolerance-related alterations, which, without being bound to a particular theory are believed to increase forward flux through one or more of a number of interrelated pathways and portions of pathways.

The combination of these pathways and pathway portions into a complex identified herein as the 3-HP toleragenic complex (“3HPTGC”) was conceived as described herein. Alterations may comprise a genetic modification that provides a nucleic acid sequence that encodes for a polypeptide that is believed effective to increase enzymatic conversion at an enzymatic conversion step of the 3HPTGC. Alterations in a culture system, including in a culture system such as an industrial bio-production system, also may comprise an addition of a product of a metabolic conversion step of the 3HPTGC. In various evaluations such alterations were determined to positively correlate with increased 3-HP tolerance.

Other aspects of the present invention are related to approaches regarding production of 3-HP. These respective aspects may be practiced in various combinations, particularly by effecting genetic modifications to a microorganism of interest to enhance tolerance to and optionally also to produce 3-HP in a recombinant microorganism. Such recombinant microorganism may be used in methods to biosynthesize 3-HP, such as in industrial bio-production systems.

To obtain genetic information used for analyses that resulted in certain discoveries related to the present invention, initially 3-HP-related fitness data was obtained by evaluation of fitness of clones from a genomic-library population using the SCALES technique. This technique was cited in the Background section, above, and is described in greater detail in paragraphs below.

Accordingly, the following paragraphs describe a technique employed to acquire genetic data that was analyzed, the analysis resulting in making the discoveries that allowed for the conception and development of the invention. Thereafter the scope of embodiments and other aspects of the invention and the field are discussed, followed by a number of examples that support the scope of the claims of the present invention.

To obtain data that could lead to the discoveries that lead to the conception of aspects of the present invention, an evaluation of 3-HP tolerant clones from a genomic-library population was conducted using the SCALEs technique. These clones were grown in a selective environment imposed by elevated concentrations of 3-HP, shown previously to be a reliable test of 3-HP tolerance.

More particularly, to obtain data potentially useful to identify genetic elements relevant to increased 3-HP tolerance, an initial population of five representative E. coli K12 genomic libraries was produced by methods known to those skilled in the art. The five libraries respectively comprised 500, 1000, 2000, 4000, 8000 base pair (“bp”) inserts of E. coli K12 genetic material. Each of these libraries, essentially comprising the entire E. coli K12 genome, was respectively transformed into MACH1™-T1® E. coli cells and cultured to mid-exponential phase corresponding to microaerobic conditions (OD₆₀₀˜0.2). Batch transfer times were variable and were adjusted as needed to avoid a nutrient limited selection environment (i.e., to avoid the cultures from entering stationary phase). Although not meant to be limiting as to alternative approaches, selection in the presence of 3-HP was carried out over 8 serial transfer batches with a decreasing gradient of 3-HP over 60 hours. More particularly, the 3-HP concentrations were 20 g 3-HP/L for serial batches 1 and 2, 15 g 3-HP/L for serial batches 3 and 4, 10 g 3-HP/L for serial batches 5 and 6, and 5 g 3-HP/L for serial batches 7 and 8. For serial batches 7 and 8 the culture media was replaced as the culture approached stationary phase to avoid nutrient limitations.

Samples were taken during and at the culmination of each batch in the selection, and were subjected to microarray analysis that identified signal strengths. The individual standard laboratory methods for preparing libraries, transformation of cell cultures, and other standard laboratory methods used for the SCALES technique prior to array and data analyses are well-known in the art, such as supported by methods taught in Sambrook and Russell, Molecular Cloning: A Laboratory Manual, Third Edition 2001 (volumes 1-3), Cold Spring Harbor Laboratory Press, Cold Spring Harbor, N.Y. (hereinafter, Sambrook and Russell, 2001). Aspects of individual methods also are discussed in greater detail in Example 1 below and in the SCALES technique patent applications, U.S. Provisional Application No. 60/611,377 filed Sep. 20, 2004 and U.S. patent application Ser. No. 11/231,018 (published as US2006/0084098A1), filed Sep. 20, 2005, both entitled:” Mixed-Library Parallel Gene Mapping Quantitation Microarray Technique for Genome Wide Identification of Trait Conferring Genes” (hereinafter, the “SCALES Technique”), which are incorporated herein by reference for teaching additional details of this technique.

Microarray technology also is well-known in the art (see, e.g. www.affymetrix.com). To obtain data of which clones were more prevalent at different exposure periods to 3-HP, Affymetrix E. Coli Antisense Gene Chip arrays (Affymetrix, Santa Clara, Calif.) were handled and scanned according to the E. Coli expression protocol from Affymetrix producing affymetrix.cel files. A strong microarray signal after a given exposure to 3-HP indicates that the genetic sequence introduced by the plasmid comprising this genetic sequence confers 3-HP tolerance. These clones can be identified by numerous microarray analyses known in the art.

This approach provided data identifying genetic elements conferring 3-HP tolerance for the analysis that led to aspects of the present discoveries and invention(s).

Also, for the purposes of incorporation by reference as applied in the United States, “A genomics approach to improve the analysis and design of strain selections,” T. E. Warnecke et al., Metabolic Engineering 10(2008)154-165, is incorporated by reference herein for its additional specific teachings that demonstrate that SCALEs fitness data correlates with and can be used as a surrogate of increased tolerance to 3-HP. This conclusion is based on the standard use of a receiver operator characteristic curve (ROC) curve. ROC analysis is routinely used in the medical diagnostic field to evaluate the correlation for a diagnostic test to the actual presence or absence of a disease. Currently diagnostic tests used through the world in medical applications that perform well in a ROC analysis are routinely used to identify the absence or presence of a disease. This analysis was adapted to evaluate the sensitivity and specificity of different microbial growth based selections resulting in fitness values as reliable tests for 3-HP tolerance. In particular a growth based selection using serial batch cultures with decreasing levels of 3-HP was identified as a sensitive and specific test for 3-HP tolerance. As a result clones in this selection with a fitness metric greater than a cutoff of 0 are identified as clones conferring tolerance to 3-HP.

As presented in Example 1, Table 1, which is incorporated into this section, lists the genes (introduced by vectors of the libraries) that were shown to have elevated fitness values, shown per above to confer tolerance to 3-HP.

### A. The 3-HP Toleragenic Complex

Analysis of the 3-HP tolerance SCALEs data has led to a more refined understanding of interrelationships among various identified pathways and portions thereof. As to the present application, this analysis led to the discovery of a complex comprising all or part of a number of metabolic pathways. As noted above this complex is named the “3-HP toleragenic complex” (3HPTGC″). It is noted that the 3HPTGC, in its entirety, was deduced from interrelationships between genes having elevated fitness values. Not every enzyme of the 3HPTGC was shown in the SCALES data to have positive fitness values. This may be attributed to certain deficiencies in the commercial arrays used to obtain that SCALES data. Accordingly, some members of the E. coli 3HPTGC not so derived from the SCALES genetic element data were deduced to fill in the 3HPTGC. However, it is noted that most of the enzymes in the 3HPTGC do have positive fitness values, and the overall fitness data in combination with the supplements and genetic modifications data, provided herein, prove the validity of the deduction and the overall significance of the 3HPTGC being related to 3-HP tolerance.

The 3HPTGC is further divided, including for claiming purposes, into an “upper section” comprising the glycolysis pathway, the tricarboxylic acid cycle, the glyoxylate pathway, and a portion of the pentose phosphate pathway, and a “lower section” comprising all or portions of (as is specifically indicated below) the chorismate super-pathway, the carbamoyl-phosphate to carbamate pathway, the threonine/homocysteine super-pathway, the nucleotide synthesis pathway, and the polyamine synthesis pathway.

In various embodiments microorganisms are genetically modified to affect one or more enzymatic activities of the 3HPTGC so that an elevated tolerance to 3-HP may be achieved, such as in industrial systems comprising microbial 3-HP biosynthetic activity. Also, genetic modifications may be made to provide and/or improve one or more 3-HP biosynthesis pathways in microorganisms comprising one or more genetic modifications for the 3-HP toleragenic complex, thus providing for increased 3-HP production. These latter recombinant microorganisms may be referred to as 3-HP-syntha-toleragenic recombinant microorganisms (“3HPSATG” recombinant microorganisms).

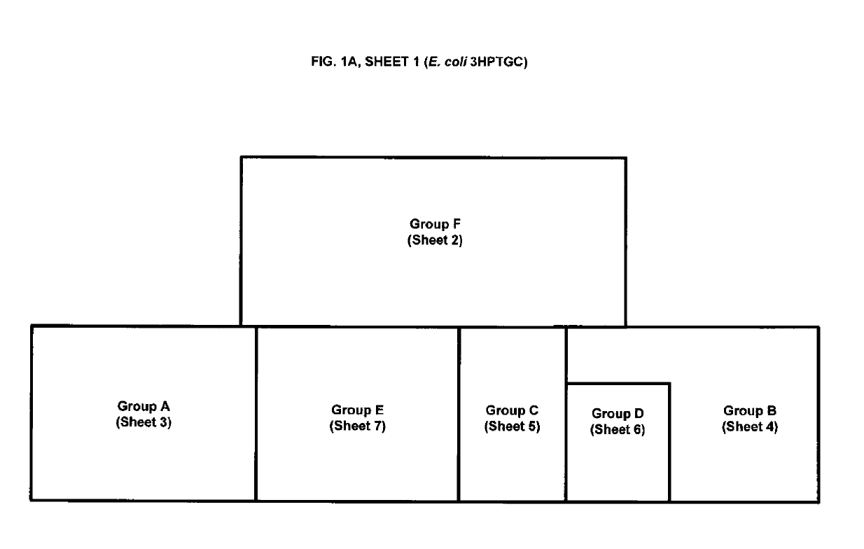

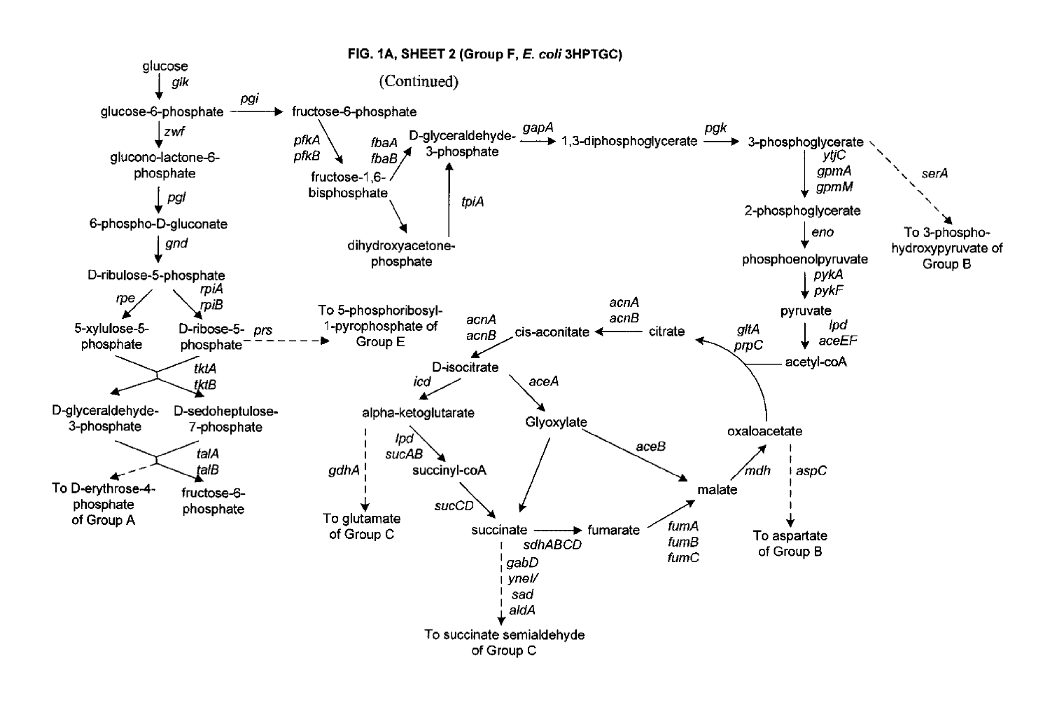

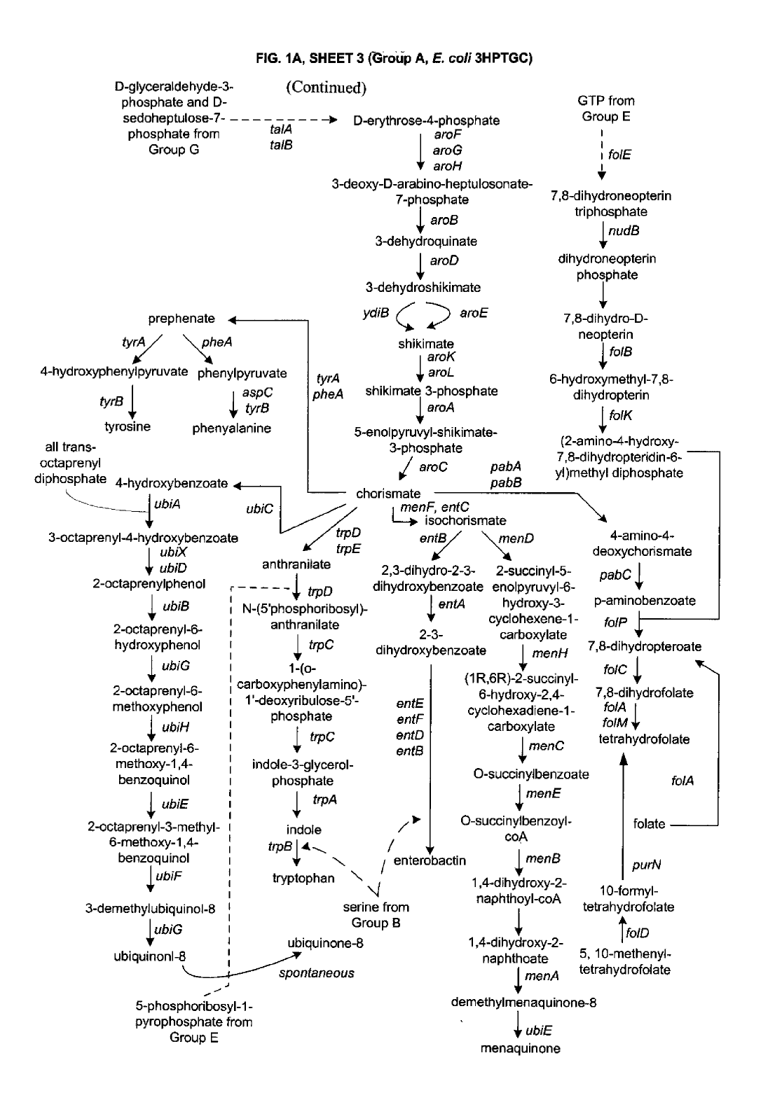

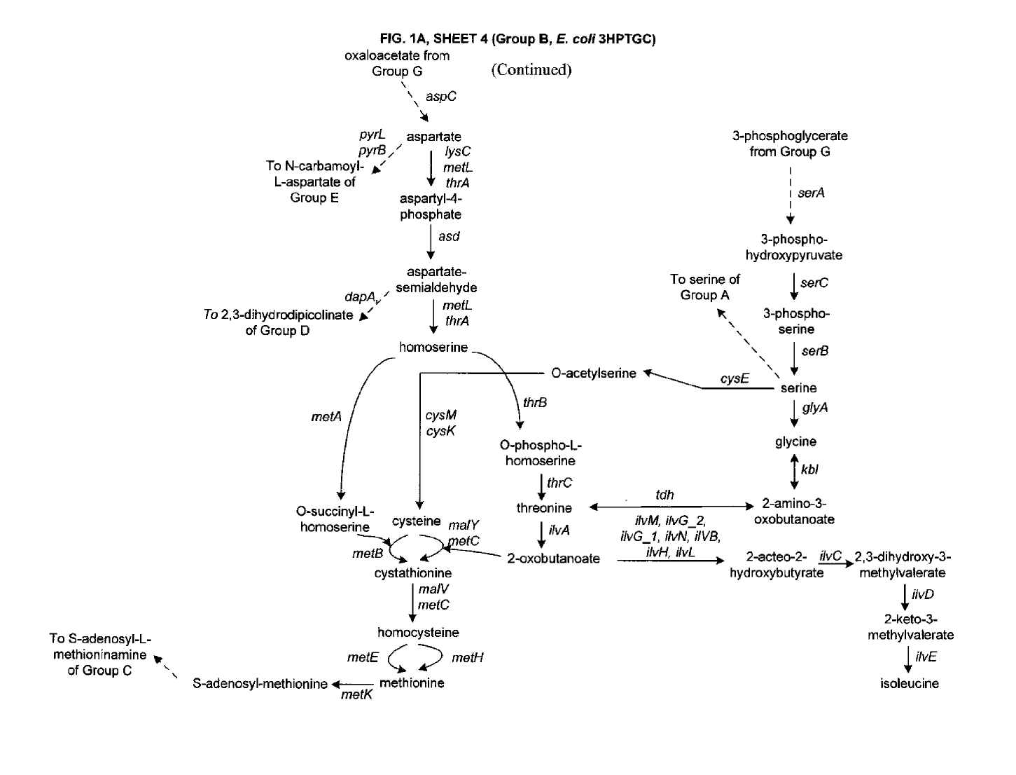

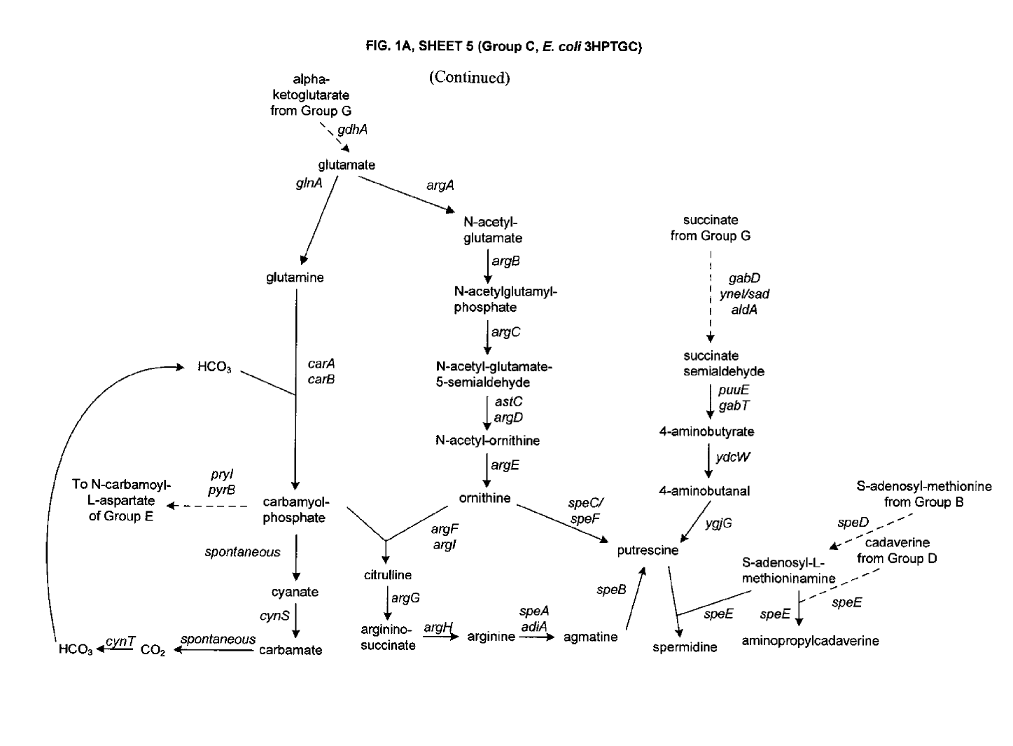

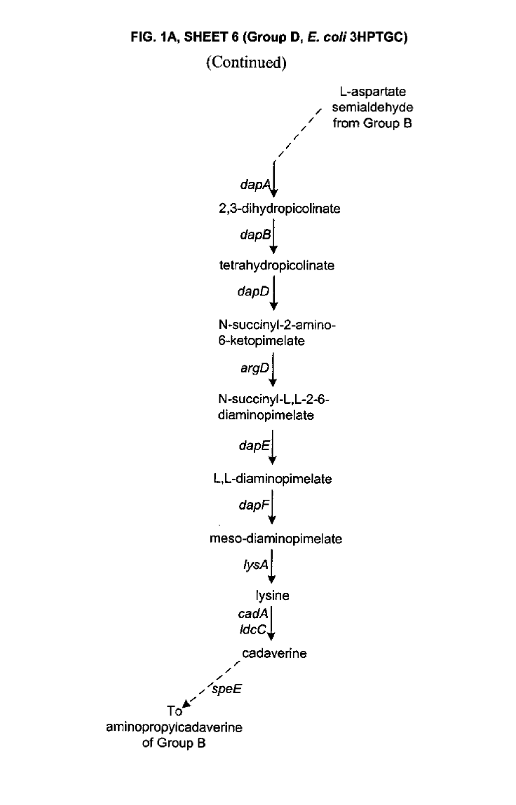

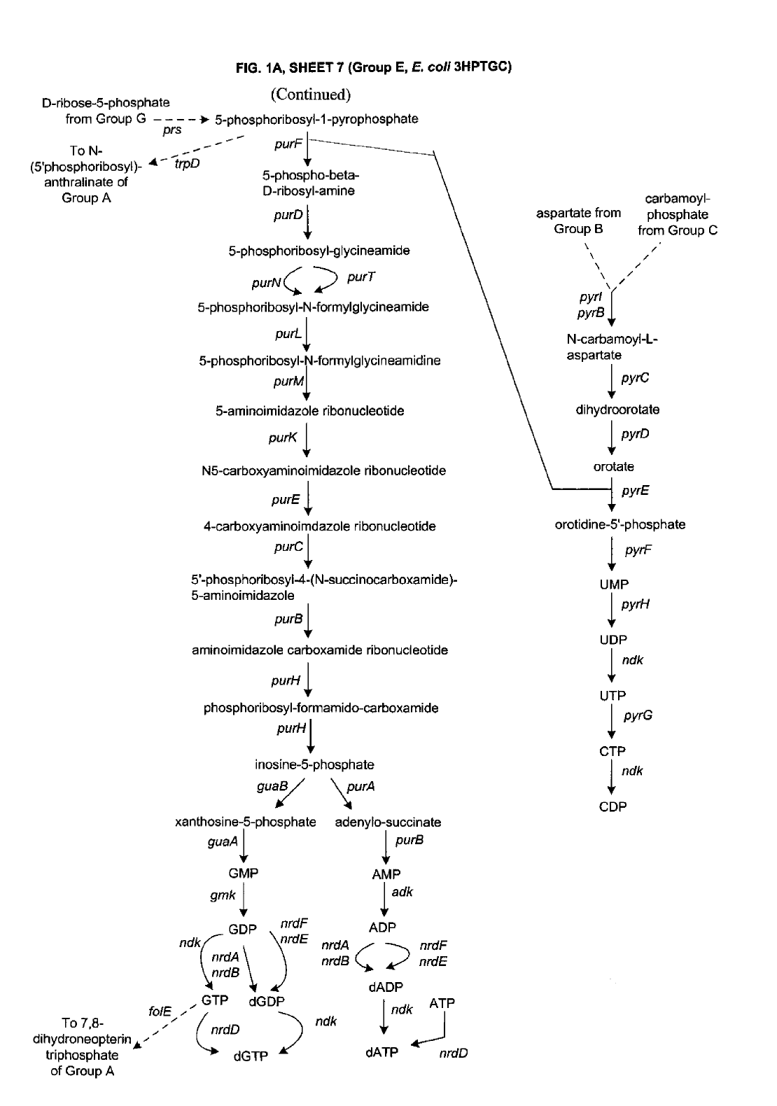

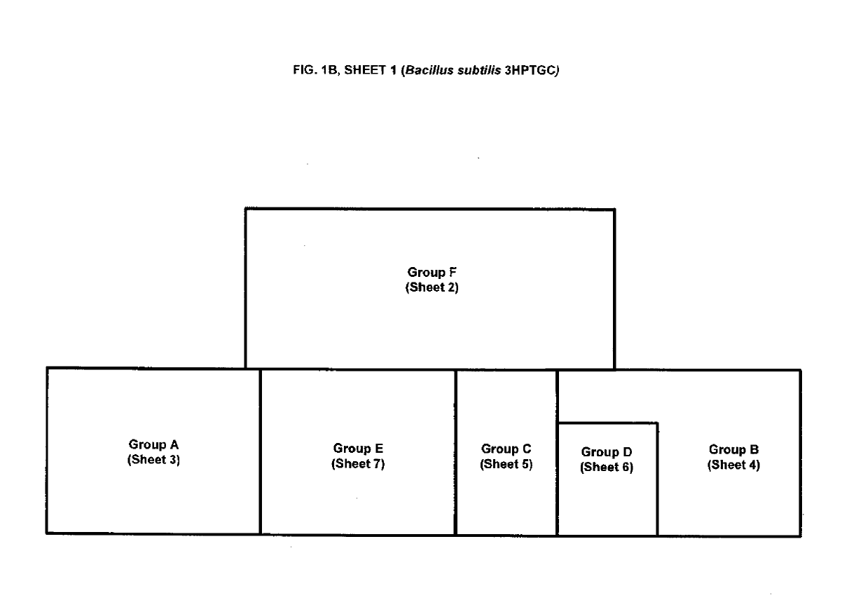

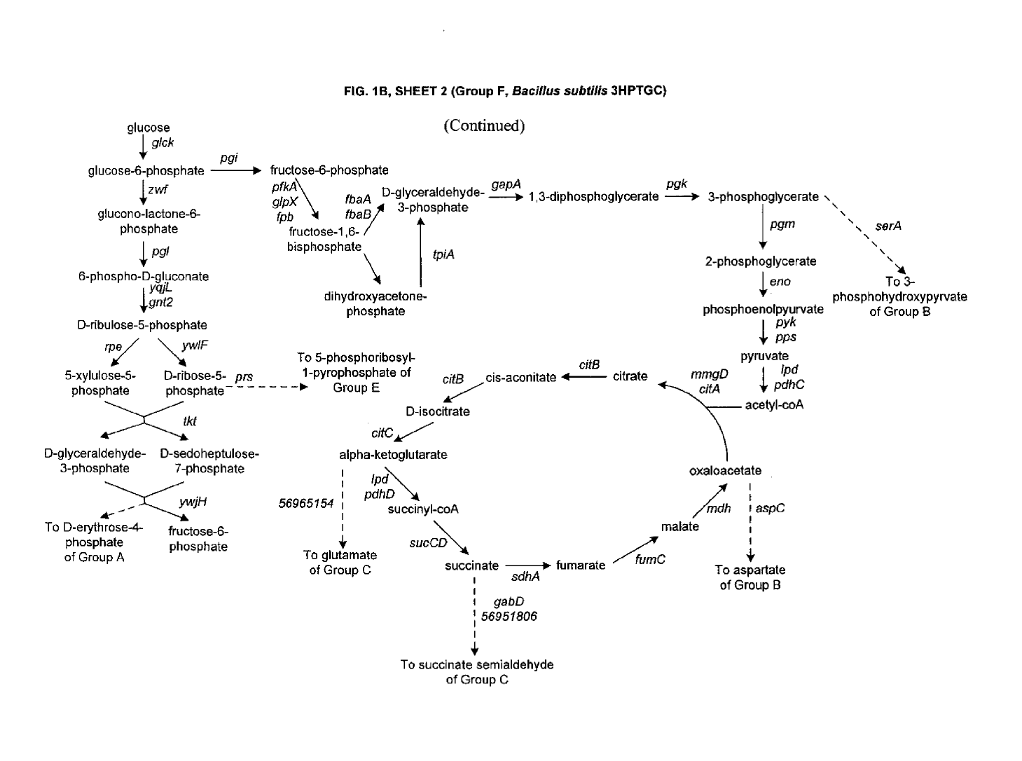

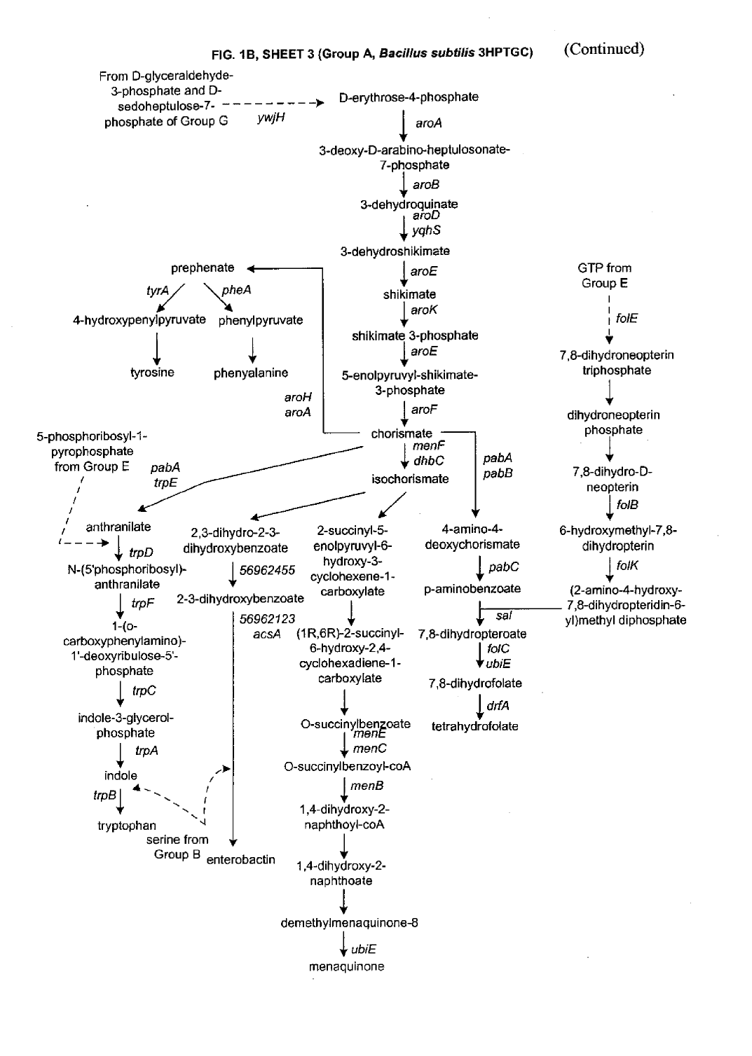

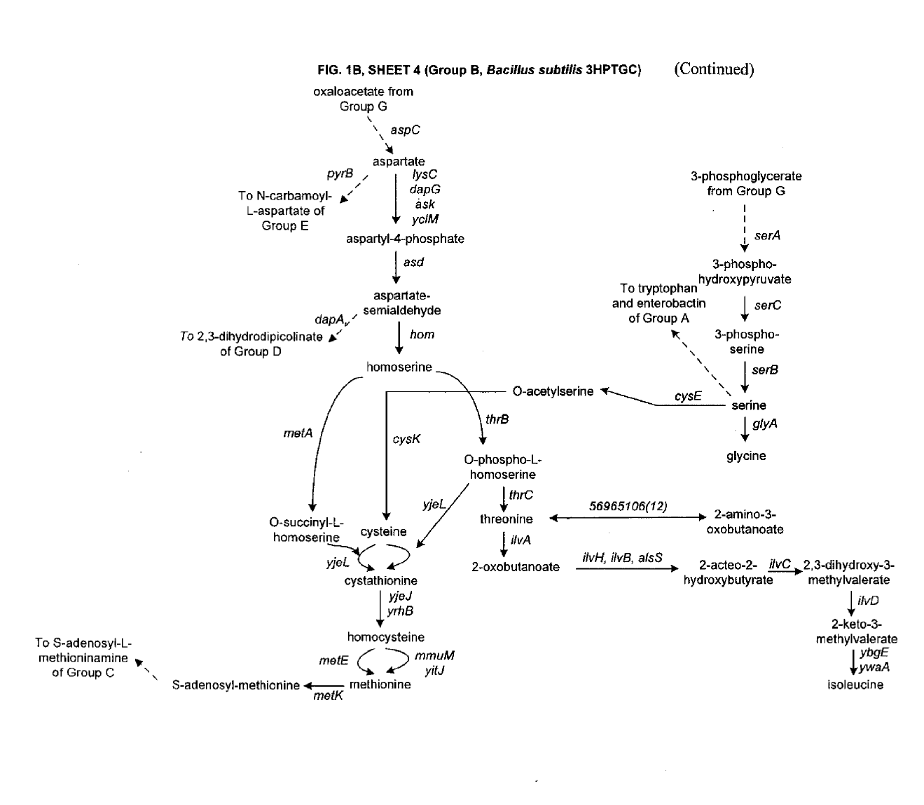

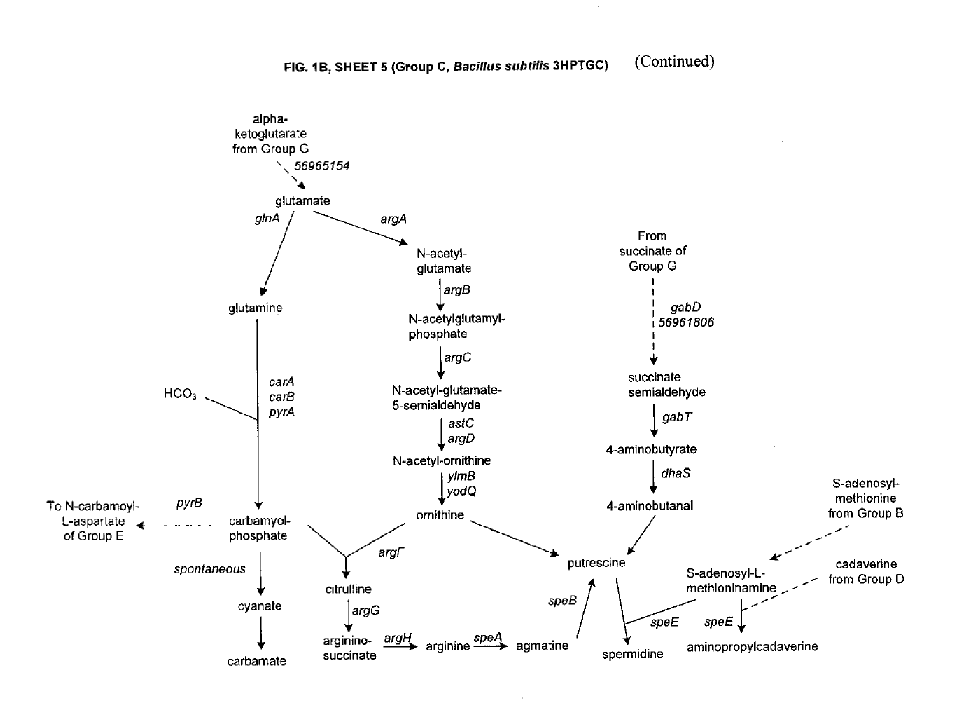

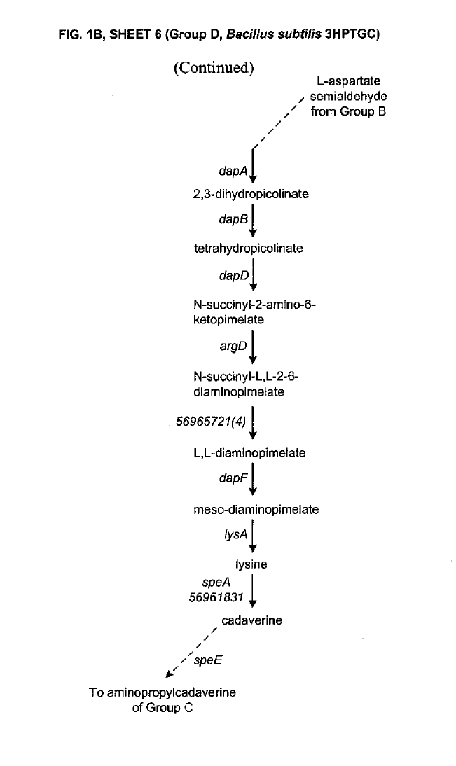

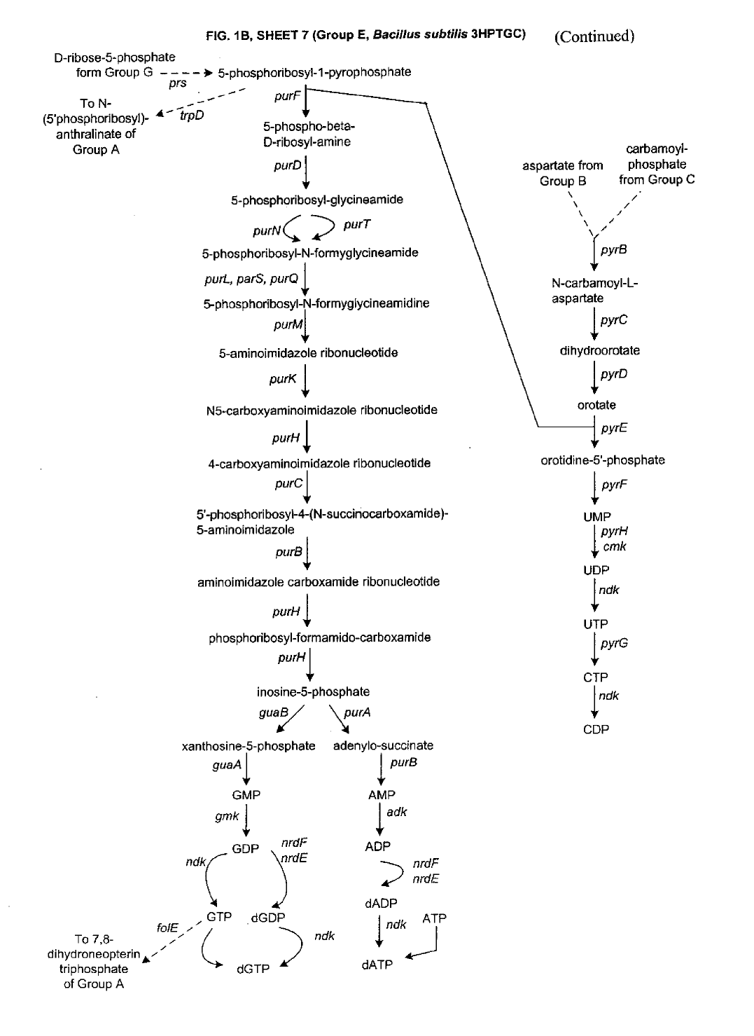

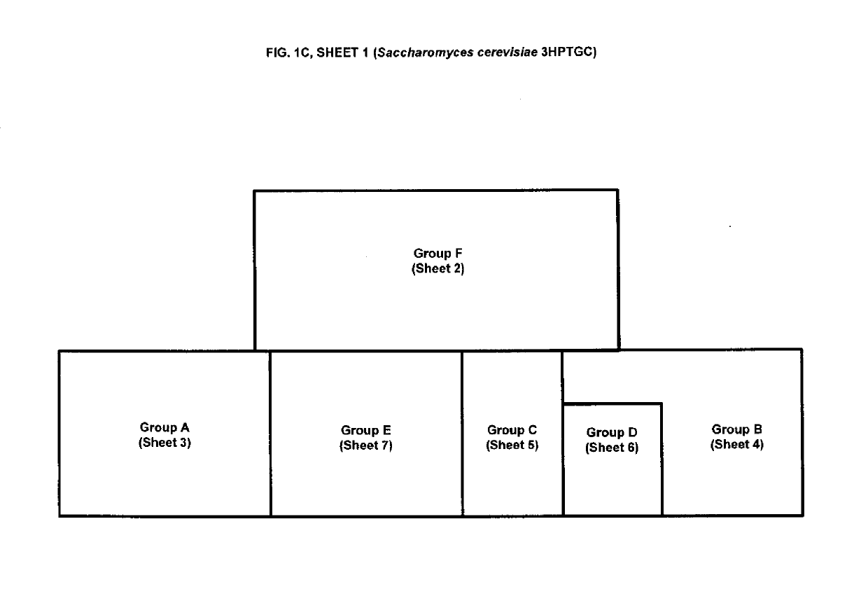

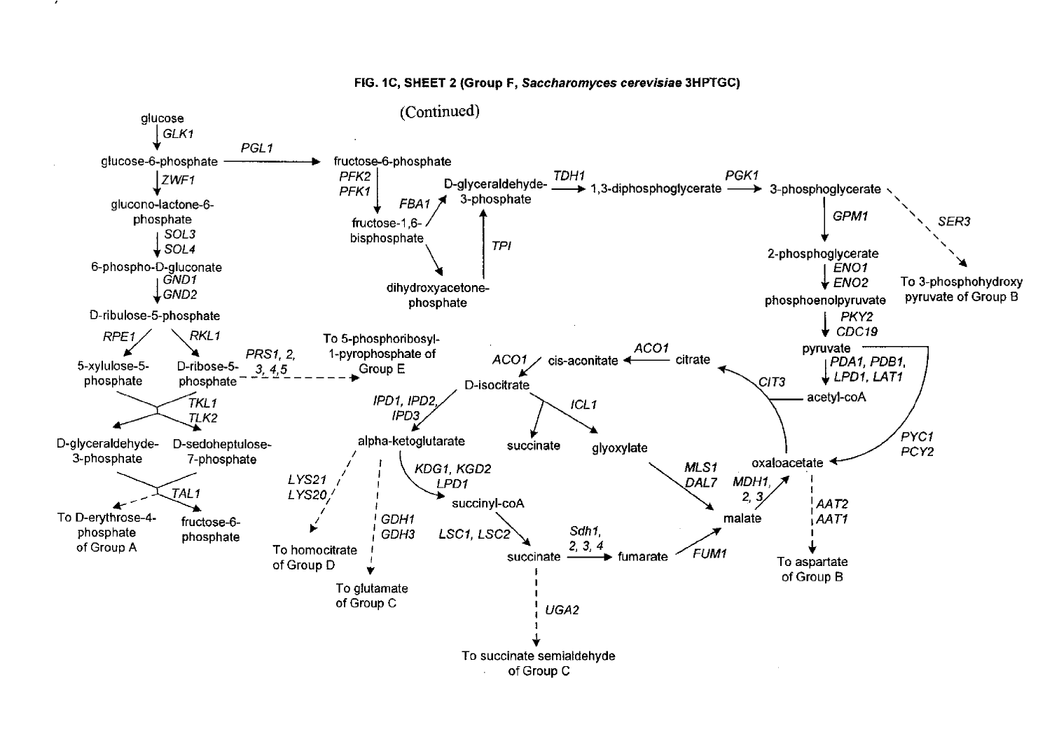

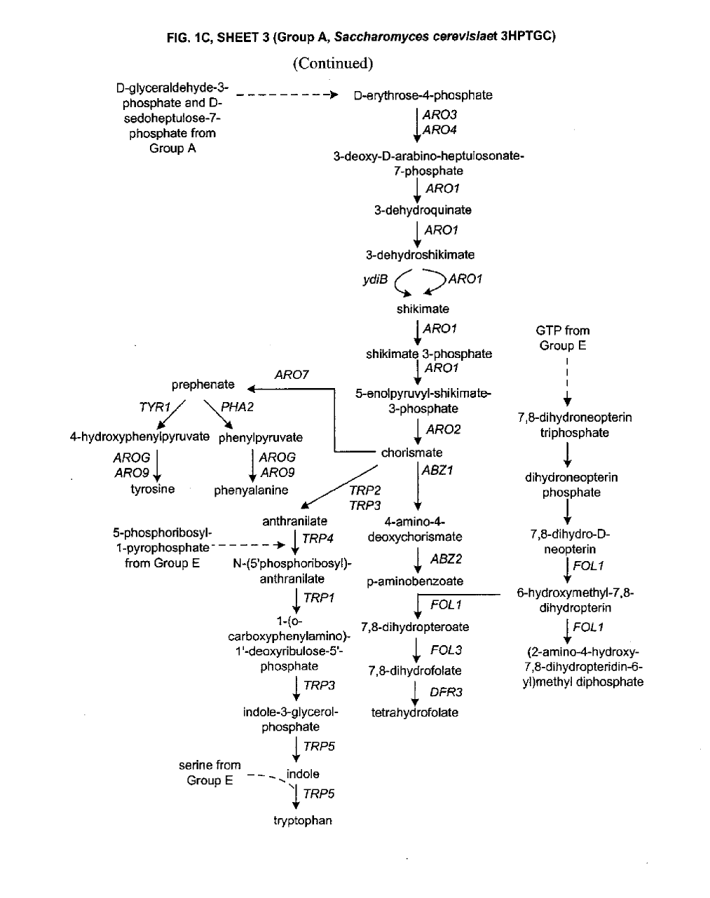

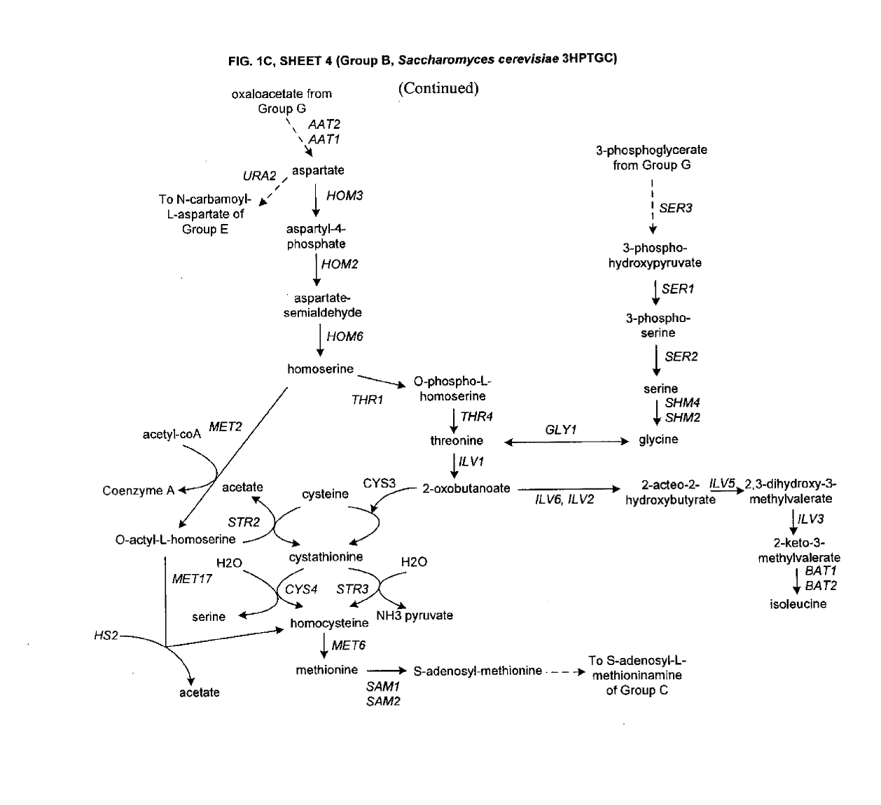

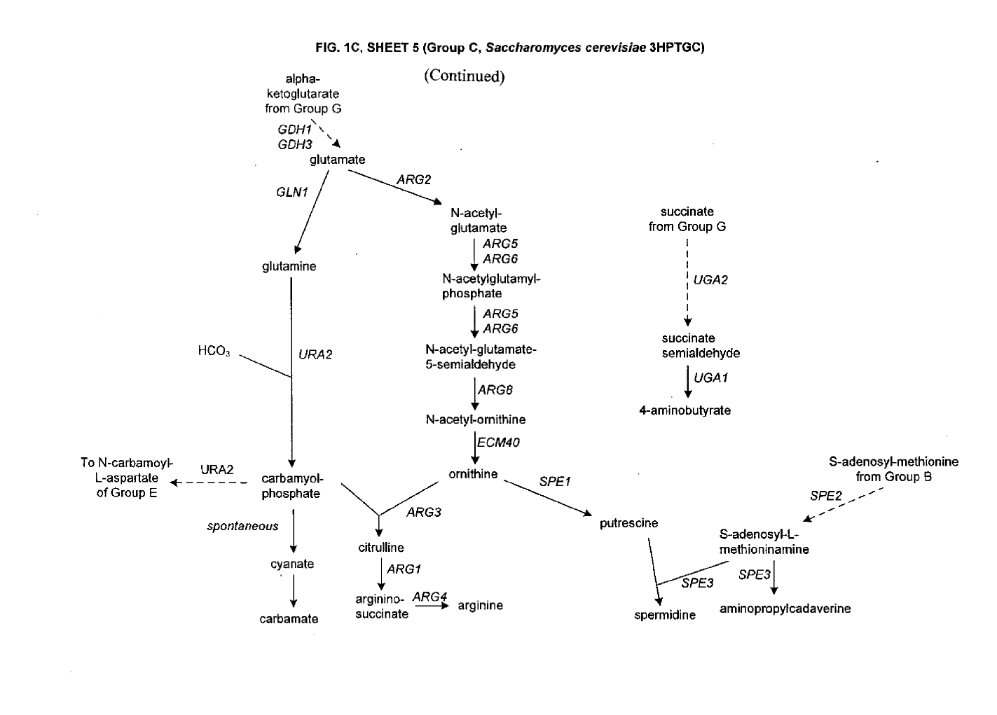

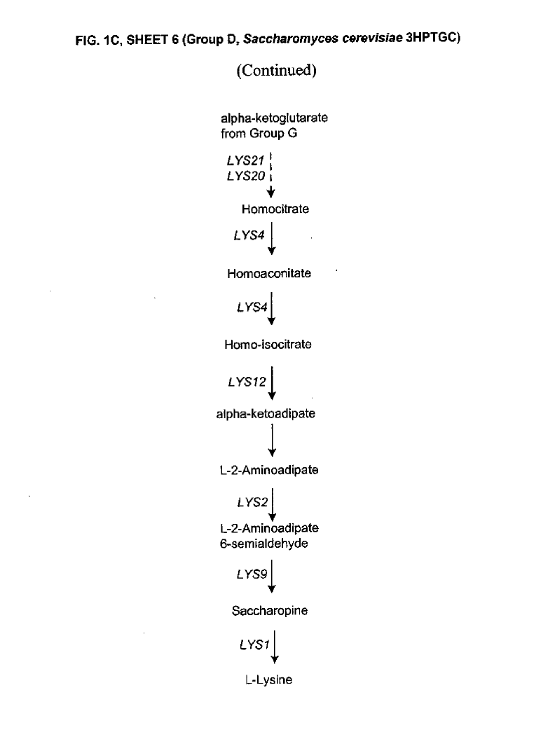

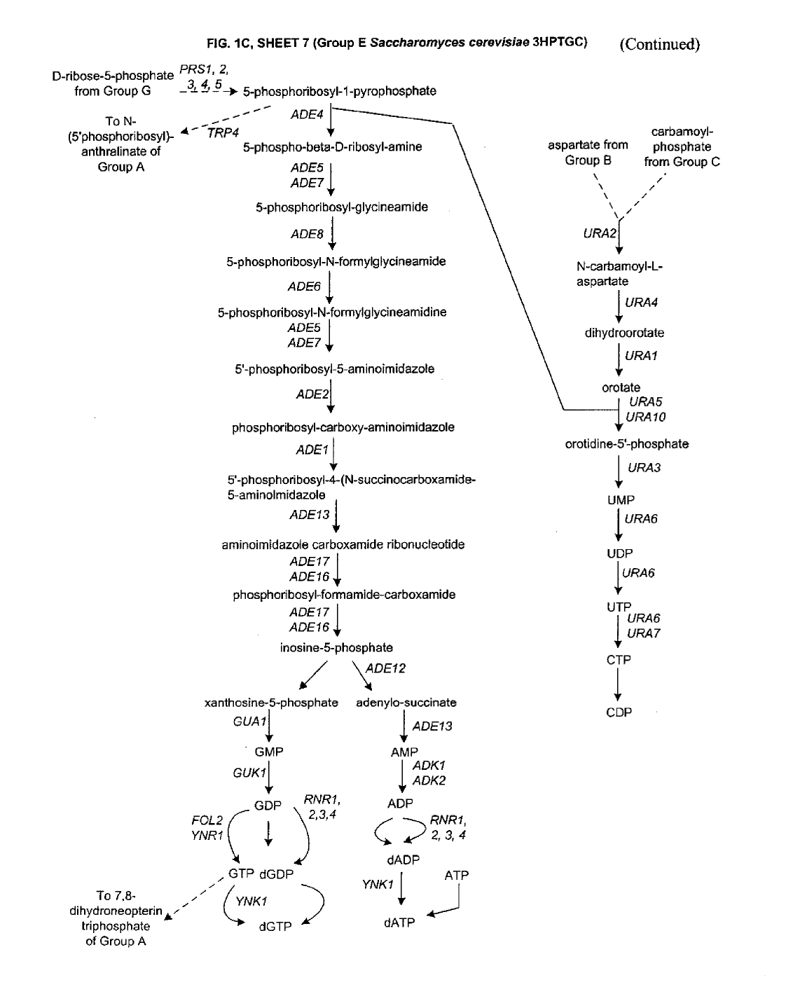

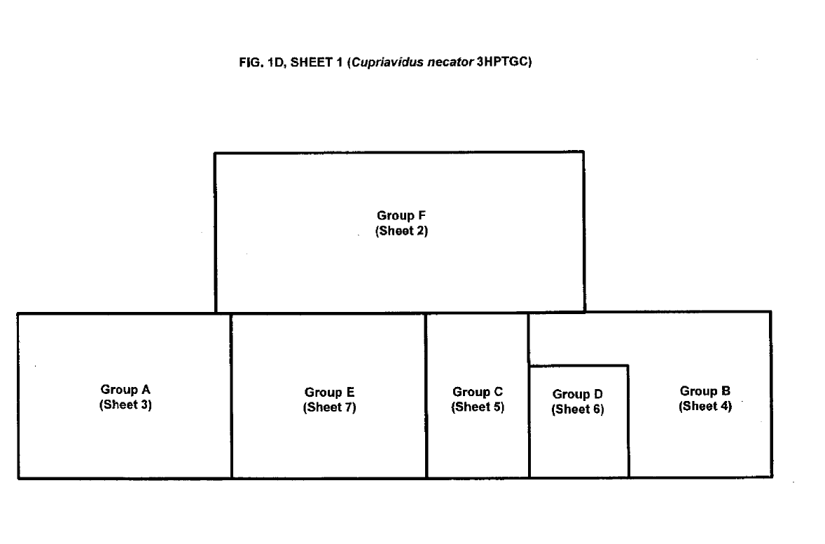

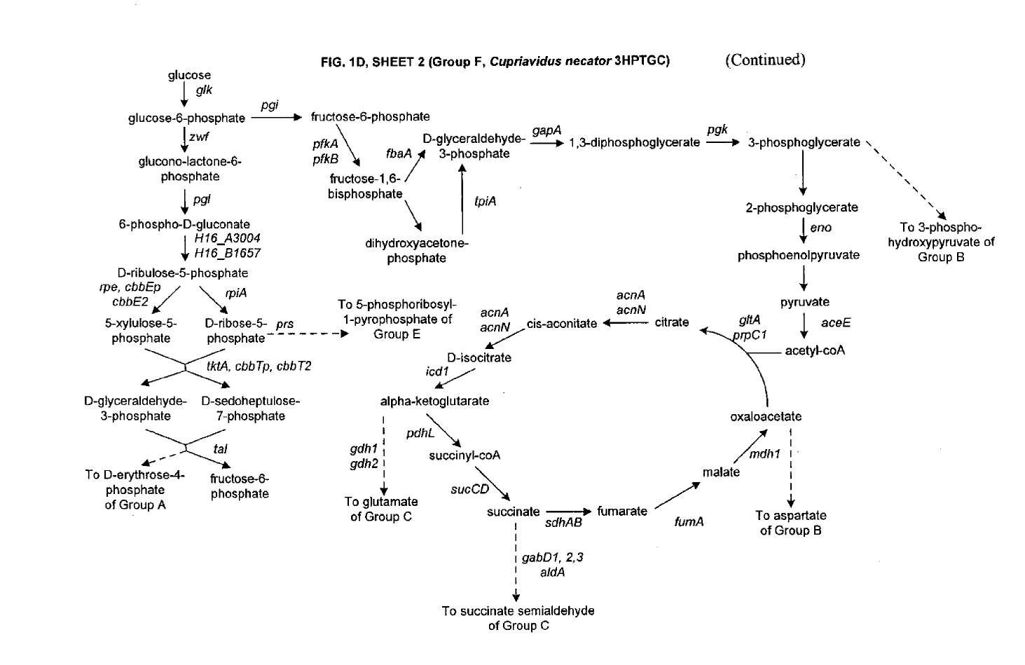

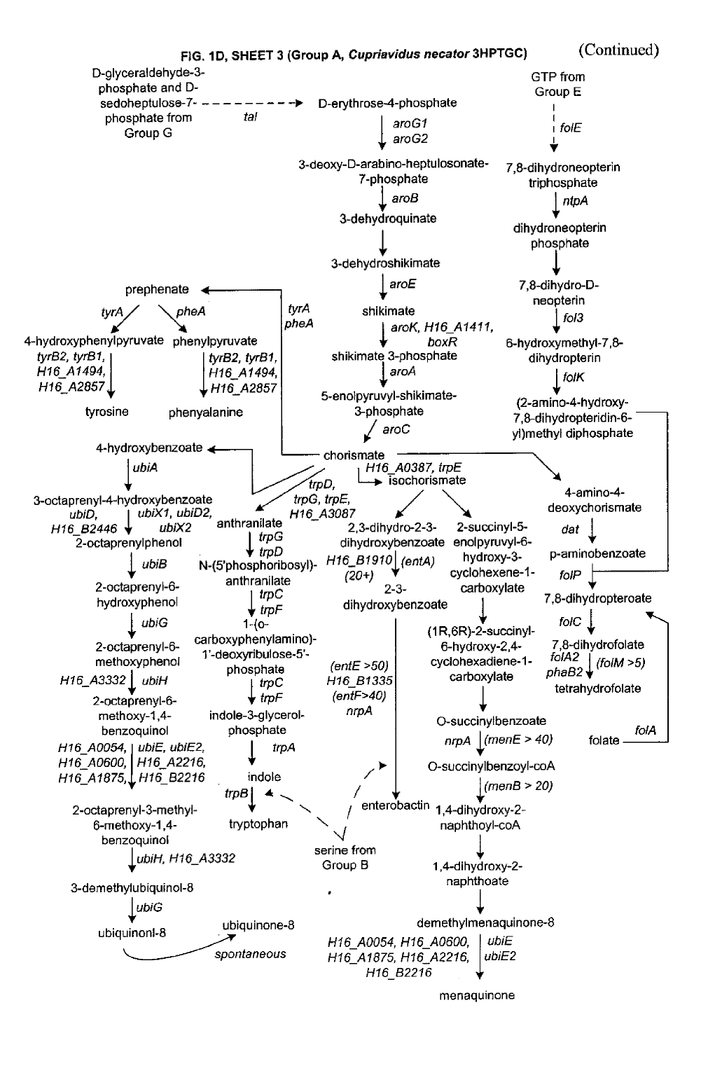

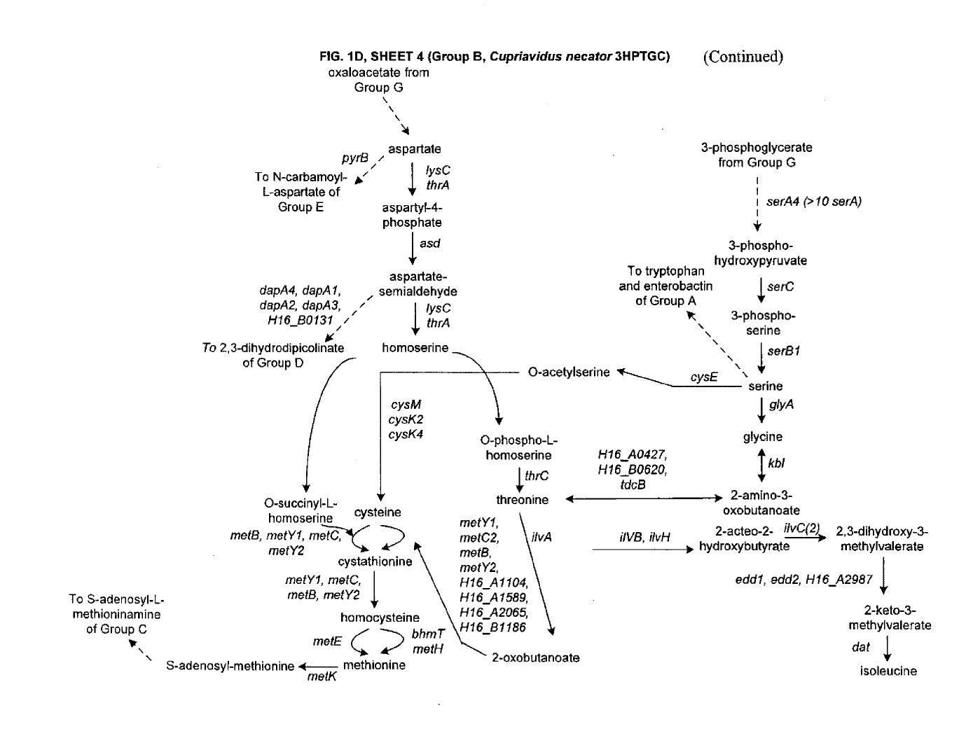

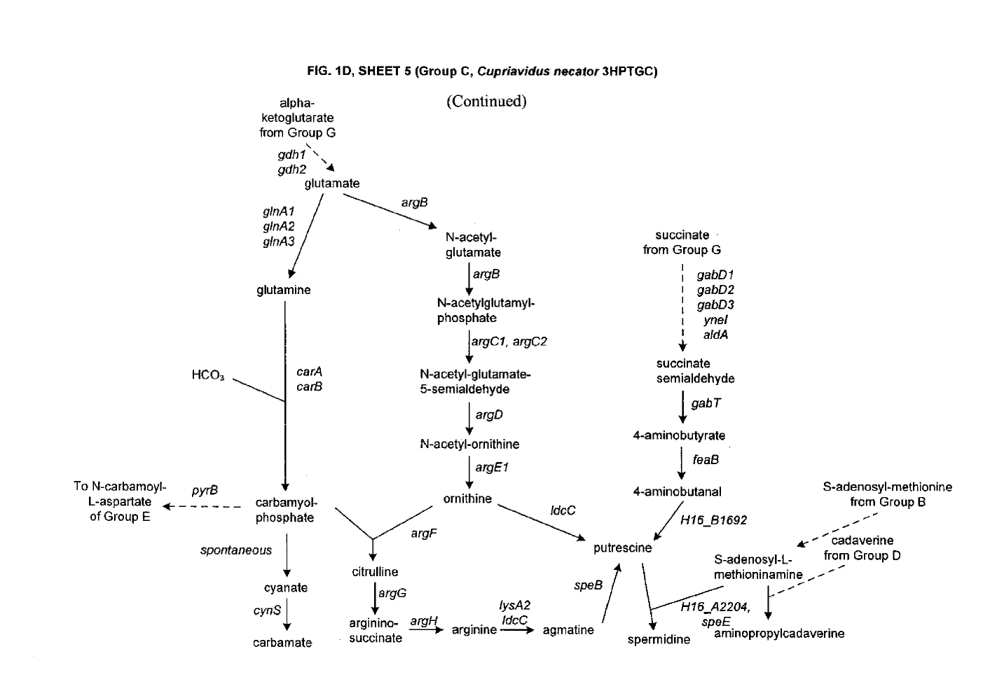

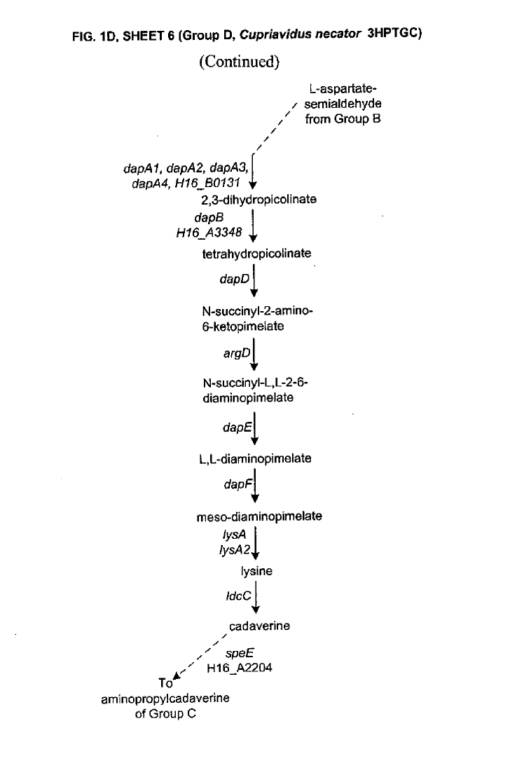

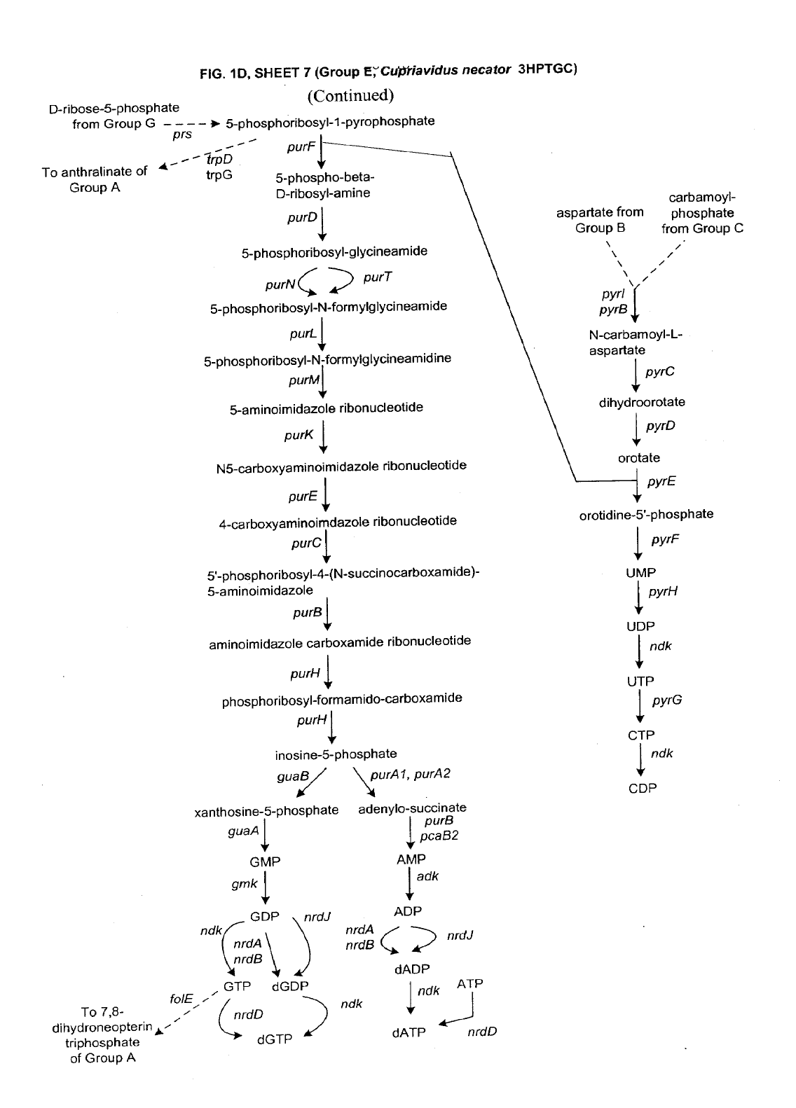

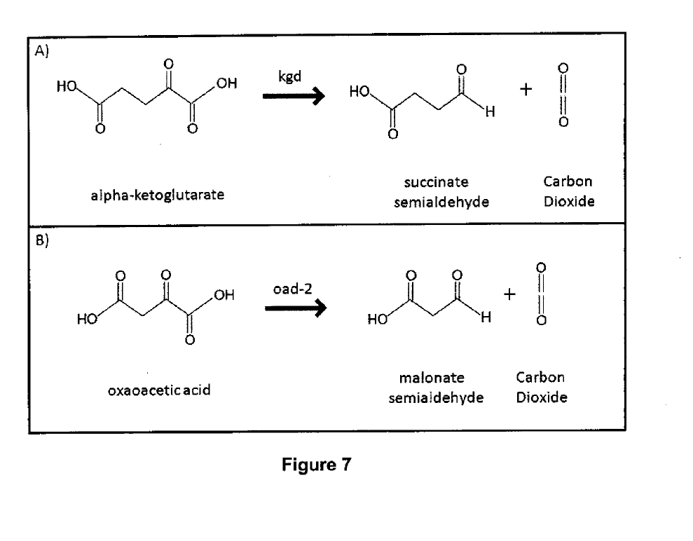

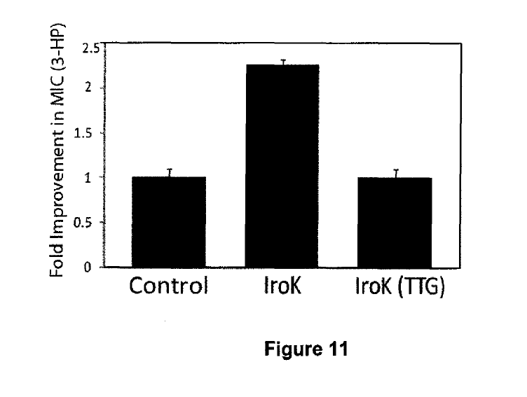

The 3HPTGC for E. coli is disclosed in FIG. 1A, sheets 1-7 (a guide for positioning these sheets to view the entire depicted 3HPTGC is provided in sheet 1 of FIG. 1A). As may be observed in FIG. 11-7, the 3HPTGC comprises all or various indicated portions of the following: the chorismate super-pathway, the carbamoyl-phosphate to carbamate pathway, the threonine/homocysteine super-pathway; a portion of the pentose phosphate pathway; the nucleotide synthesis pathway; the glycolysis/tricarboxylic acid cycle/glyoxylate bypass super-pathway; and the polyamine synthesis pathway. It is noted that the chorismate pathway and the threonine pathway are identified as super-pathways since they respectively encompass a number of smaller known pathways. However, the entire 3HPTGC comprises these as well as other pathways, or portions thereof, that normally are not associated with either the chorismate super-pathway or the threonine/homocysteine super-pathway.

More particularly, FIG. 1A, comprising sheets 1-7, is subdivided into the lower section, which is further subdivided into Groups A-E and the upper section, identified simply as Group F. The lower section groups are identified as follows: Group A, or “chorismate,” comprising the indicated, major portion of the chorismate super-pathway (sheet 3); Group B, or “threonine/homocysteine,” comprising the indicated portion of the threonine/homocysteine pathway (sheet 7); Group C, or “polyamine synthesis,” comprising the indicated portion of the polyamine pathway, which includes arginine synthesis steps and also the carbamoyl-phosphate to carbamate pathway (sheet 5); Group D, or “lysine synthesis,” comprising the indicated portion of the lysine synthesis pathway (sheet 6); Group E, or “nucleotide synthesis,” comprising the indicated portions of nucleotide synthesis pathways (sheet 4). Group F (sheet 2) comprises the upper section of the 3HPTGC and includes the glycolysis pathway, the tricarboxylic acid cycle, and the glyoxylate bypass pathway, and the indicated portions of the pentose phosphate pathway.

It is noted that particular genes are identified at enzymatic conversion steps of the 3HPTGC in FIG. 1A, sheets 1-7. These genes are for E. coli strain K12, substrain MG1655; nucleic acid and corresponding amino acid sequences of these are available at http://www.ncbi.nlm.nih.gov/sites/entrez, and alternatively at www.ecocyc.org. As is known to one skilled in the art, some genes may be found on a chromosome within an operon, under the control of a single promoter, or by other interrelationships. When a nucleic acid sequence herein is referred to as a combination, such as sucCD or cynTS, by this is meant that the nucleic acid sequence comprises, respectively, both sucC and sucD, and both cynT and cynS. Additional control and other genetic elements may also be in such nucleic acid sequences, which may be collectively referred to as “genetic elements” when added in a genetic modification, and which is intended to include a genetic modification that adds a single gene.

However, similarly functioning genes are readily found in different species and strains, encoding enzymes having the same function as shown in FIG. 1A, sheets 1-7, and such genes, and the 3HPTGCs of such other species and strains may be utilized in the practice of the invention. This can be achieved by the following methods, which are not meant to be limiting.

For the set of genes within the 3HPTGC of E. coli, protein sequences were obtained from NCBI. To identify similarly functioning genes in S. cerevisiae, a pathway comparison tool at www.biocyc.org was utilized using the genes identified in the E. coli 3HPTGC. For B. subtilis, this annotated approach was used in part, and enzymes or pathway portions not obtained by that approach were obtained by a homology comparison approach. For the homology approach a local blast (http://www.ncbi.nlm.nih.gov/Tools/) (blastp) comparison using the selected set of E. coli proteins and Bacillus protein sequence (4096 sequences) was performed using different thresholds (http://www.ncbi.nlm.nih.gov/genomes/lproks.cqi). Using the homology information (homology matches having E⁻¹⁰ or less E-value) the remaining genes and enzymes were identified for the 3HPTGC for Bacillus subtilis.

Also, the latter homology approach was used for Cupriavidus necator, Table 2 provides some examples of the homology relationships for genetic elements of C. necator that have a demonstrated homology to E. coli genes that encode enzymes known to catalyze enzymatic conversion steps of the 3HPTGC. This is based on the criterion of the homologous sequences having an E-value less than E⁻¹⁰. Table 2 provides only a few of the many homologies (over 850) obtained by the comparison. Not all of the homologous sequences in C. necator are expected to encode a desired enzyme suitable for an enzymatic conversion step of the 3HPTGC for C. necator. However, through one or more of a combination of selection of genetic elements known to encode desired enzymatic reactions, the most relevant genetic elements are selected for the 3HPTGC for this species.

FIG. 1B, sheets 1-7, shows the 3HPTGC for Bacillus subtilis, FIG. 1C, sheets 1-7, shows the 3HPTGC for the yeast Saccharomyces cerevisiae and FIG. 1D, sheets 1-7, shows the 3HPTGC for Cupriavidus necator. Enzyme names for the latter are shown, along with an indication of the quantity of homologous sequences meeting the criterion of having an E-value less than E⁻¹⁰ when compared against an E. coli enzyme known to catalyze a desired 3HPTGC enzymatic conversion step.

Based on either of the above approaches, and the present existence of or relative ease and low cost of obtaining genomic information of a given microorganism species, one or both of the above approaches may be employed to identify relevant genes and enzymes in a selected microorganism species (for which its genomic sequence is known or has been obtained), evaluate the relative improvements in 3-HP tolerance of selected genetic modifications of such homologously matched and identified genes, and thereby produce a recombinant selected microorganism comprising improved tolerance to 3-HP.

Additionally, it is appreciated that alternative pathways in various microorganisms may yield products of the 3HPTGC, the increased production or presence of which are demonstrated herein to result in increased 3-HP tolerance. For example, in yeast species there are alternative pathways to lysine, a product within Group D. Accordingly, alterations of such alternative pathways are within the scope of the invention for such microorganism species otherwise falling within the scope of the relevant claim(s). Thus, in various embodiments the invention is not limited to the specific pathways depicted in FIGS. 1A-D. That is, various pathways, and enzymes thereof, that yield the products shown in FIGS. 1A-D may be considered within the scope of the invention.

It is noted that when two or more genes are shown for a particular enzymatic conversion step, these may be components of a single multi-enzyme complex, or may represent alternative enzymes that have different control factors that control them, or are induced differently. Also, as is clear to one skilled in the art, only the major reactants (i.e., substrates) and products are shown for the enzymatic conversion steps. This is to minimize details on an already-crowded figure. For example, electron carriers and energy transfer molecules, such as NAD(P)(H) and ADP/ATP, are not shown, and these (and other small-molecule reactants not shown in the 3HPTGC figures) are not considered “products” of the 3HPTGC as that term is used herein. Also, for at least two steps (dihydroneopterin phosphate to 7,8-dihydro-D-neopterin and 1,4-dihydroxy-2-naphthoyl-CoA to 1,4-dihydroxy-2-naphthoate) no enzyme is shown because no enzyme has been known to be identified for this step at the time of filing. Accordingly, in some embodiments the 3HPTGC is understood and/or taken to exclude enzymes, nucleic acid sequences, and the like, for these steps. Also, as discussed below, also included within the scope of the invention are nucleic acid sequence variants encoding identified enzymatic functional variants of any of the enzymes of the 3HPTGC or a related complex or portion thereof as set forth herein, and their use in constructs, methods, and systems claimed herein.

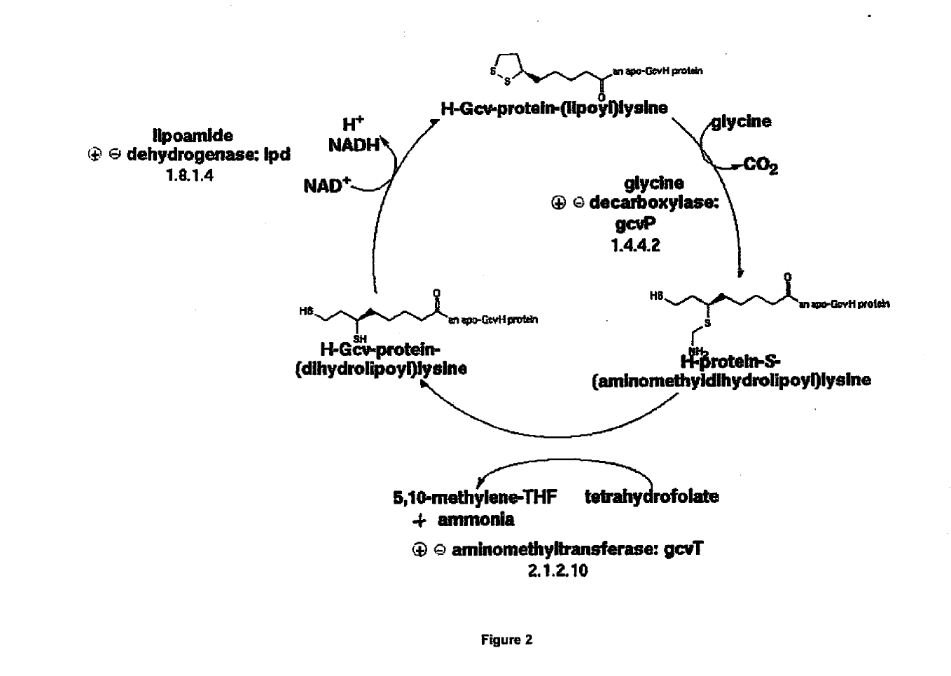

Some fitness data provided in Table 1 is not represented in the figures of the 3HPTGC but nonetheless is considered to support genetic modification(s) and/or supplementation to improve 3-HP tolerance. For example, the relatively elevated fitness scores for gcvH, gcvP and gcvT, related to the glycine cleavage system. These enzymes are involved in the glycine/5,10-methylene-tetrahydrofolate (“5,10mTHF”) conversion pathway, depicted in FIG. 2. In the direction shown in FIG. 2, the three enzymatically catalyzed reactions result in decarboxylation of glycine (a 3HPTGC product, see FIG. 1A, sheet 4), production of 5,10-methylene-THF from tetrahyrdofolate (“THF”), and production of NADH from NAD⁺. The 5,10-methylene-THF product of this complex is a reactant in enzymatically catalyzed reactions that are part of the following: folate polyglutamylation; panthothenate biosynthesis; formylTHF biosynthesis; and de novo biosynthesis of pyrimidine deoxyribonucleotides. Overall, the enzymes, and enzymatic catalytic steps thereof, shown in Table 1 but not represented in FIG. 1, sheets 1-7 are considered part of the invention (as are their functional equivalents for other species).

Actual data and/or prophetic examples directed to alterations of the 3HPTGC are provided below. These examples are intended to demonstrate the breadth of applicability (based on the large number of genomic elements related to the 3HPTGC that demonstrate increased 3-HP tolerance) and some specific approaches to achieve increased tolerance to 3-HP. Approaches may be combined to achieve additive or synergistic improvements in 3-HP tolerance, and may include alterations that are genetic or non-genetic (e.g., relating to system supplementation with particular chemicals, or general alterations to the industrial system). In addition, specific production strategies are disclosed and exemplified.

As described and detailed below, the present invention broadly relates to alterations, using genetic modifications, and/or medium modulations (e.g, additions of enzymatic conversion products or other specific chemicals), to achieve desired results in microbe-based industrial bio-production methods, systems and compositions. As to the tolerance aspects, this invention flows from the discovery of the unexpected importance of the 3HPTPC which comprises certain metabolic pathway portions comprising enzymes whose increased activity (based on increasing copy numbers of nucleic acid sequences that encode there) correlates with increased tolerance of a microorganism to 3-HP.

### B. 3-HP Production

The 3-HP tolerance aspects of the present invention can be used with any microorganism that makes 3-HP, whether that organism makes 3-HP naturally or has been genetically modified by any method to produce 3-HP.

As to the 3-HP production increase aspects of the invention, which may result in elevated titer of 3-HP in industrial bio-production, the genetic modifications comprise introduction of one or more nucleic acid sequences into a microorganism, wherein the one or more nucleic acid sequences encode for and express one or more production pathway enzymes (or enzymatic activities of enzymes of a production pathway). In various embodiments these improvements thereby combine to increase the efficiency and efficacy of, and consequently to lower the costs for, the industrial bio-production production of 3-HP.

Any one or more of a number of 3-HP production pathways may be used in a microorganism such as in combination with genetic modifications directed to improve 3-HP tolerance. In various embodiments genetic modifications are made to provide enzymatic activity for implementation of one or more of such 3-HP production pathways. Several 3-HP production pathways are known in the art. For example, U.S. Pat. No. 6,852,517 teaches a 3-HP production pathway from glycerol as carbon source, and is incorporated by reference for its teachings of that pathway. This reference teaches providing a genetic construct which expresses the dhaB gene from Klebsiella pneumoniae and a gene for an aldehyde dehydrogenase. These are stated to be capable of catalyzing the production of 3-HP from glycerol.

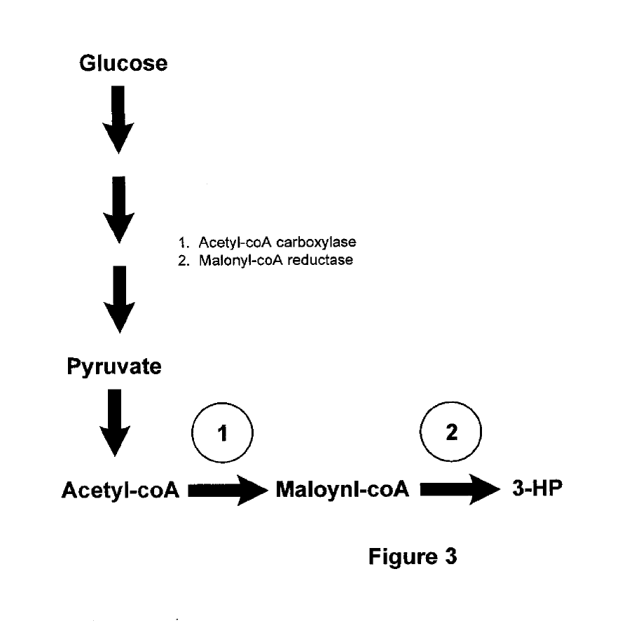

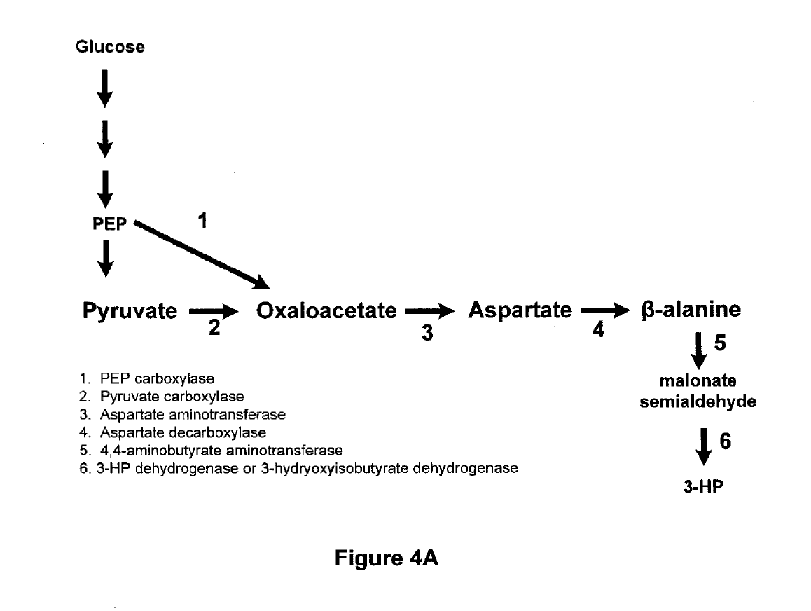

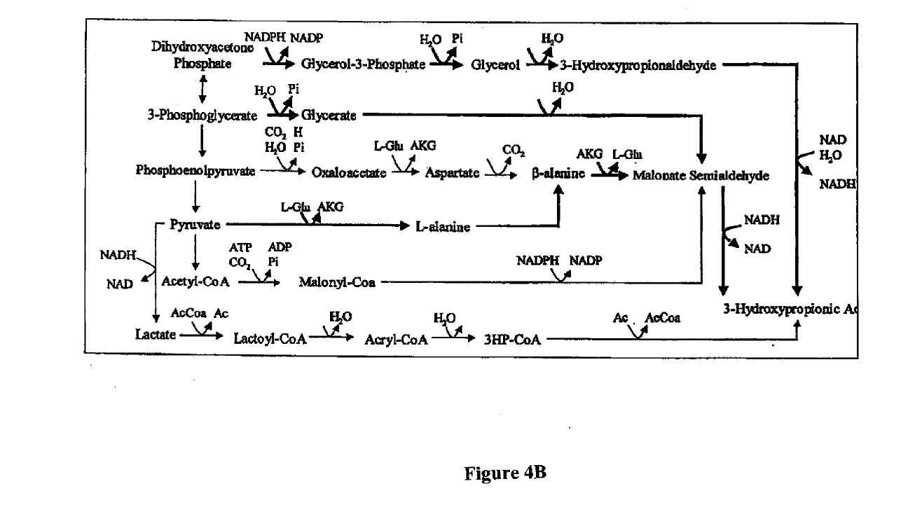

WO2002/042418 (PCT/US01/43607) teaches several 3-HP production pathways. This PCT publication is incorporated by reference for its teachings of such pathways. Also, FIG. 44 of that publication, which summarizes a 3-HP production pathway from glucose to pyruvate to acetyl-CoA to malonyl-CoA to 3-HP, is provided herein as FIG. 3. FIG. 55 of that publication, which summarizes a 3-HP production pathway from glucose to phosphoenolpyruvate (PEP) to oxaloacetate (directly or via pyruvate) to aspartate to β-alanine to malonate semialdehyde to 3-HP, is provided herein as FIG. 4A. Representative enzymes for various conversions are also shown in these figures.

FIG. 4B, from U.S. Patent Publication No. US2008/0199926, published Aug. 21, 2008 and incorporated by reference herein, summarizes the above-described 3-HP production pathways and other known natural pathways. More generally as to developing specific metabolic pathways, of which many may be not found in nature, Hatzimanikatis et al. discuss this in “Exploring the diversity of complex metabolic networks,” Bioinformatics 21(8):1603-1609 (2005). This article is incorporated by reference for its teachings of the complexity of metabolic networks.

Further to the 3-HP production pathway summarized in FIG. 3, Strauss and Fuchs (“Enzymes of a novel autotrophic CO₂ fixation pathway in the phototrophic bacterium Chloroflexus aurantiacus, the 3-hydroxyproprionate cycle,” Eur. J. Bichem. 215, 633-643 (1993)) identified a natural bacterial pathway that produced 3-HP. At that time the authors stated the conversion of malonyl-CoA to malonate semialdehyde was by an NADP-dependant acylating malonate semialdehyde dehydrogenase and conversion of malonate semialdehyde to 3-HP was catalyzed by a 3-hydroxyproprionate dehydrogenase. However, since that time it has become appreciated that, at least for Chloroflexus aurantiacus, a single enzyme may catalyze both steps (M. Hugler et al., “Malonyl-Coenzyme A Reductase from Chloroflexus aurantiacus, a Key Enzyme of the 3-Hydroxypropionate Cycle for Autotrophic CO₂ Fixation,” J. Bacter, 184(9):2404-2410 (2002)).

Accordingly, one production pathway of various embodiments of the present invention comprises malonyl-Co-A reductase enzymatic activity that achieves conversions of malonyl-CoA to malonate semialdehyde to 3-HP. As provided in an example below, introduction into a microorganism of a nucleic acid sequence encoding a polypeptide providing this enzyme (or enzymatic activity) is effective to provide increased 3-HP biosynthesis.

Another 3-HP production pathway is provided in FIG. 5B (FIG. 5A showing the natural mixed fermentation pathways) and explained in this and following paragraphs. This is a 3-HP production pathway that may be used with or independently of other 3-HP production pathways. One possible way to establish this biosynthetic pathway in a recombinant microorganism, one or more nucleic acid sequences encoding anoxaloacetate alpha-decarboxylase (oad-2) enzyme (or respective or related enzyme having such activity) is introduced into a microorganism and expressed. As exemplified in Example 7, which is not meant to be limiting, enzyme evolution techniques are applied to enzymes having a desired catalytic role for a structurally similar substrate, so as to obtain an evolved (e.g., mutated) enzyme (and corresponding nucleic acid sequence(s) encoding it), that exhibits the desired catalytic reaction at a desired rate and specificity in a microorganism.

As noted, the above examples of 3-HP production pathways are not meant to be limiting particularly in view of the various known approaches, standard in the art, to achieve desired metabolic conversions.

Thus, for various embodiments of the invention the genetic manipulations to any pathways of the 3HPTCG and any of the 3-HP bio-production pathways may be described to include various genetic manipulations, including those directed to change regulation of, and therefore ultimate activity of, an enzyme or enzymatic activity of an enzyme identified in any of the respective pathways. Such genetic modifications may be directed to transcriptional, translational, and post-translational modifications that result in a change of enzyme activity and/or selectivity under selected and/or identified culture conditions. Thus, in various embodiments, to function more efficiently, a microorganism may comprise one or more gene deletions. For example, in E. coli, the genes encoding the pyruvate kinase (pfkA and pfkB), lactate dehydrogenase (ldhA), phosphate acetyltransferase (pta), pyruvate oxidase (poxB) and pyruvate-formate lyase (pflB) may be deleted. Such gene deletions are summarized at the bottom of FIG. 5B for a particular embodiment, which is not meant to be limiting. Gene deletions may be accomplished by mutational gene deletion approaches, and/or starting with a mutant strain having reduced or no expression of one or more of these enzymes, and/or other methods known to those skilled in the art.

More generally, and depending on the particular metabolic pathways of a microorganism selected for genetic modification, any subgroup of genetic modifications may be made to decrease cellular production of fermentation product(s) selected from the group consisting of acetate, acetoin, acetone, acrylic, malate, fatty acid ethyl esters, isoprenoids, glycerol, ethylene glycol, ethylene, propylene, butylene, isobutylene, ethyl acetate, vinyl acetate, other acetates, 1,4-butanediol, 2,3-butanediol, butanol, isobutanol, sec-butanol, butyrate, isobutyrate, 2-OH-isobutryate, 3-OH-butyrate, ethanol, isopropanol, D-lactate, L-lactate, pyruvate, itaconate, levulinate, glucarate, glutarate, caprolactam, adipic acid, propanol, isopropanol, fusel alcohols, and 1,2-propanediol, 1,3-propanediol, formate, fumaric acid, propionic acid, succinic acid, valeric acid, and maleic acid. Gene deletions may be made as disclosed generally above, and other approaches may also be used to achieve a desired decreased cellular production of selected fermentation products.

### C. Genetic Modifications and Supplementations, Including Combinations Thereof

For various embodiments of the invention the genetic modifications to any pathways and pathway portions of the 3HPTCG and any of the 3-HP bio-production pathways may be described to include various genetic manipulations, including those directed to change regulation of, and therefore ultimate activity of, an enzyme, or enzymatic activity of an enzyme identified in any of the respective pathways. Such genetic modifications may be directed to transcriptional, translational, and post-translational modifications that result in a change of enzyme activity and/or overall enzymatic conversion rate under selected and/or identified culture conditions, and/or to provision of additional nucleic acid sequences (as provided in some of the Examples) so as to increase copy number and/or mutants of an enzyme of the 3HPTGC. Specific methodologies and approaches to achieve such genetic modification are well known to one skilled in the art, and include, but are not limited to: increasing expression of an endogenous genetic element; decreasing functionality of a repressor gene; introducing a heterologous genetic element; increasing copy number of a nucleic acid sequence encoding a polypeptide catalyzing an enzymatic conversion step of the 3HPTGC; mutating a genetic element to provide a mutated protein to increase specific enzymatic activity; over-expressing; under-expressing; over-expressing a chaperone; knocking out a protease; altering or modifying feedback inhibition; providing an enzyme variant comprising one or more of an impaired binding site for a repressor and/or competitive inhibitor; knocking out a repressor gene; evolution, selection and/or other approaches to improve mRNA stability. Random mutagenesis may be practiced to provide genetic modifications of the 3HPTGC that may fall into any of these or other stated approaches. The genetic modifications further broadly fall into additions (including insertions), deletions (such as by a mutation) and substitutions of one or more nucleic acids in a nucleic acid of interest. In various embodiments a genetic modification results in improved enzymatic specific activity and/or turnover number of an enzyme. Without being limited, changes may be measured by one or more of the following: K_{M}; K_{cat}; and K_{avidity}.

Such genetic modifications overall are directed to increase enzymatic conversion at at least one enzymatic conversion step of the 3HPTGC so as to increase 3-HP tolerance of a microorganism so modified. Also, the enzymatic conversion steps shown in FIGS. 1A-D may be catalyzed by enzymes that are readily identified by one skilled in the art, such as by searching for the enzyme name corresponding to the gene name at a particular enzymatic conversion step in FIGS. 1A-D, and then identifying enzymes, such as in other species, having the same name and function. The latter would be able to convert the respective reactant(s) to the respective product(s) for that enzymatic conversion step. Public database sites, such as www.metacyc.org, www.ecocyc.org, and www.biocyc.org, and www.ncbi.gov, have associated tools to identify such analogous enzymes.

Also, although the MIC analysis is used frequently herein as an endpoint to indicate differences in microorganism growth when placed in various 3-HP concentrations for a specified time, this is by no means considered to be the only suitable metric to determine a difference, such as an improvement, in microorganism tolerance based on aspects of the invention. Without being limiting, other suitable measurement approaches may include growth rate determination, lag time determination, changes in optical density of cultures at specified culture durations, number of doublings of a population in a given time period and, for microorganisms that comprise 3-HP production capability, overall 3-HP production in a culture system in which 3-HP accumulates to a level inhibitory to a control microorganism lacking genetic modifications that increase enzymatic conversion at one or more enzymatic conversion steps of the 3HPTGC. This may result in increased productivities, yields or titers.

It is generally appreciated that a useful metric to assess increases in 3-HP tolerance can be related to a microorganism's or a microorganism culture's ability to grow while exposed to 3-HP over a specified period of time. This can be determined by various quantitative and/or qualitative analyses and endpoints, particularly by comparison to an appropriate control that lacks the 3-HP tolerance-related genetic modification(s) and/or supplements as disclosed and discussed herein. Time periods for such assessments may be, but are not limited to: 12 hours; 24 hours; 48 hours; 72 hours; 96 hours; and periods exceeding 96 hours. Varying exposure concentrations of 3-HP may be assessed to more clearly identify a 3-HP tolerance improvement. The following paragraphs provide non-limiting examples of approaches that may be used to demonstrate differences in a microorganism's ability to grow and/or survive in the presence of 3-HP in its culture system when teachings of the present invention are applied to the microorganism and/or the culture system.

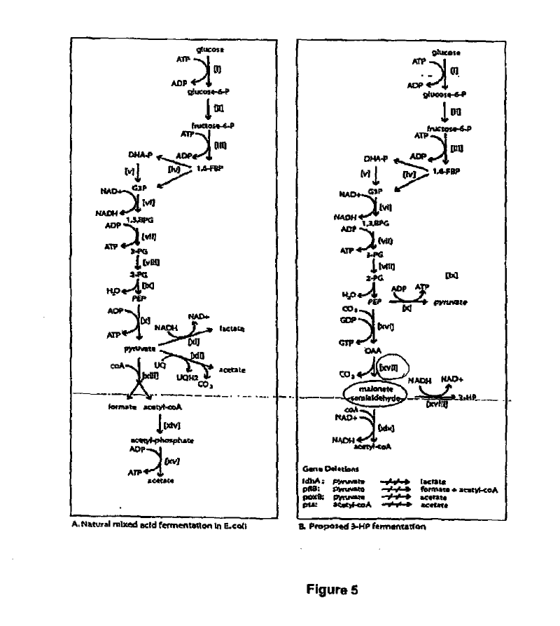

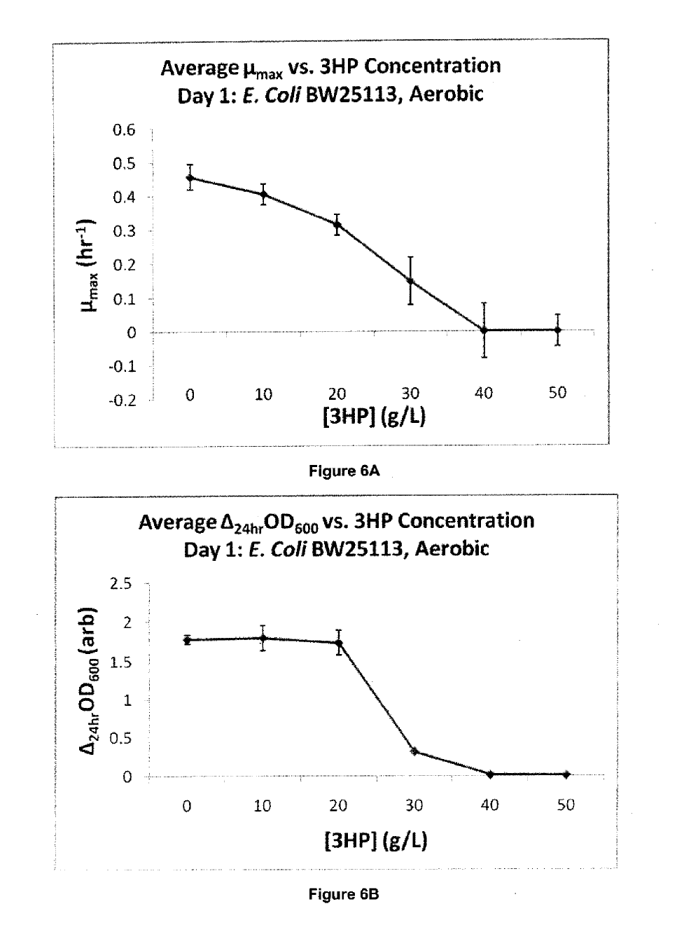

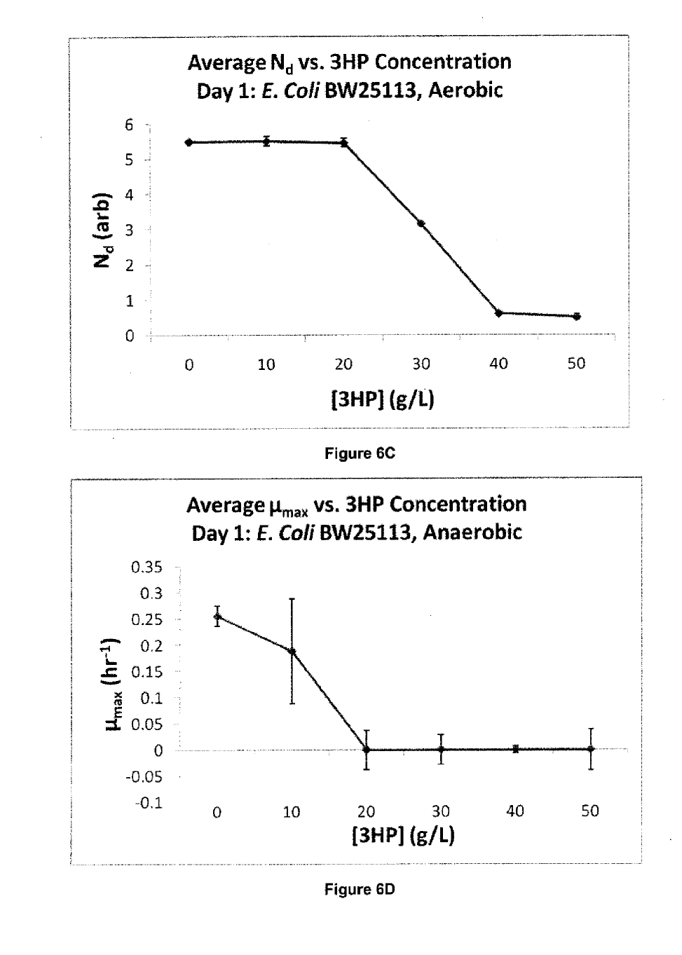

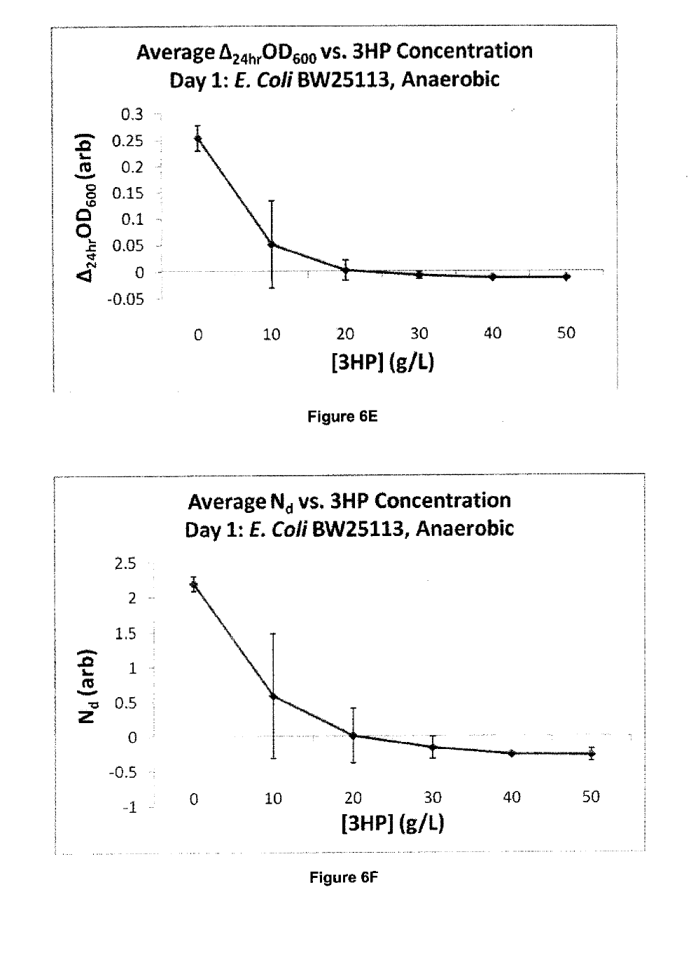

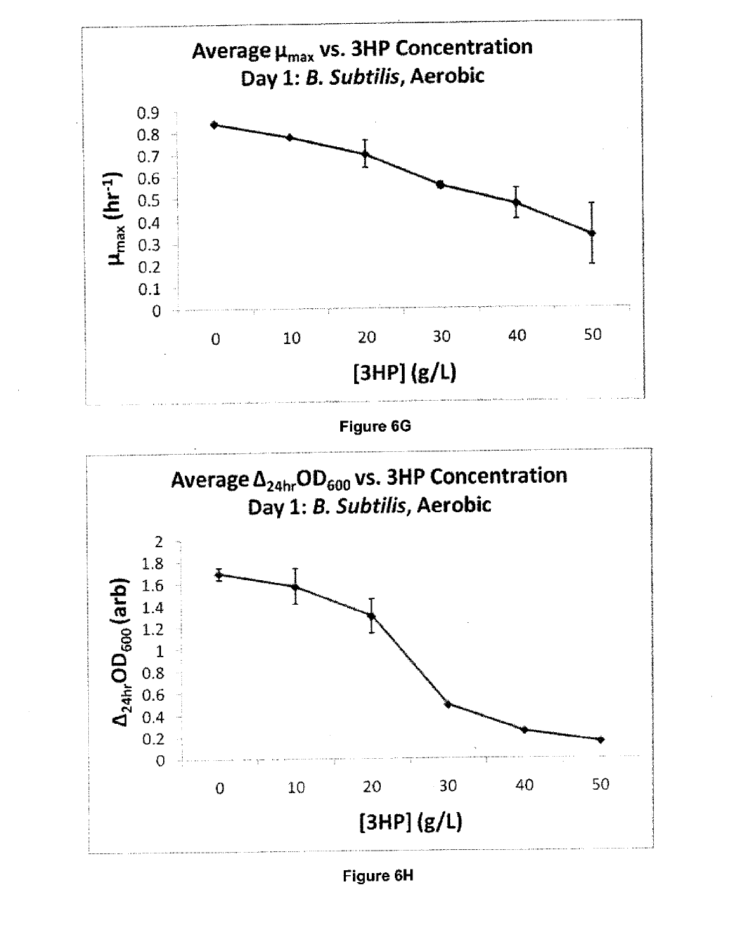

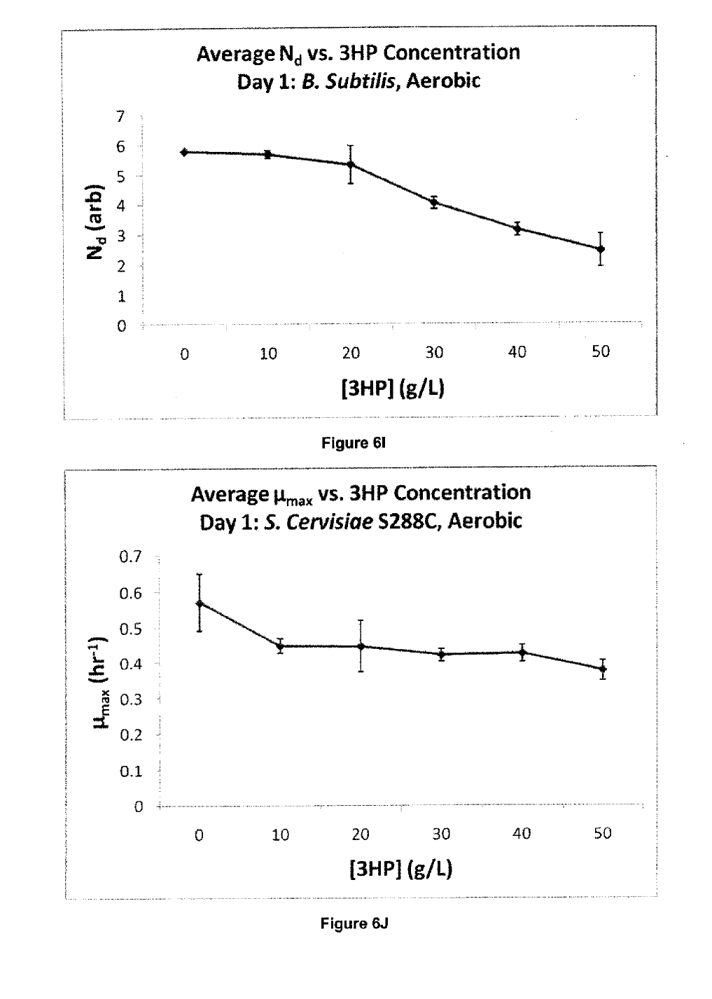

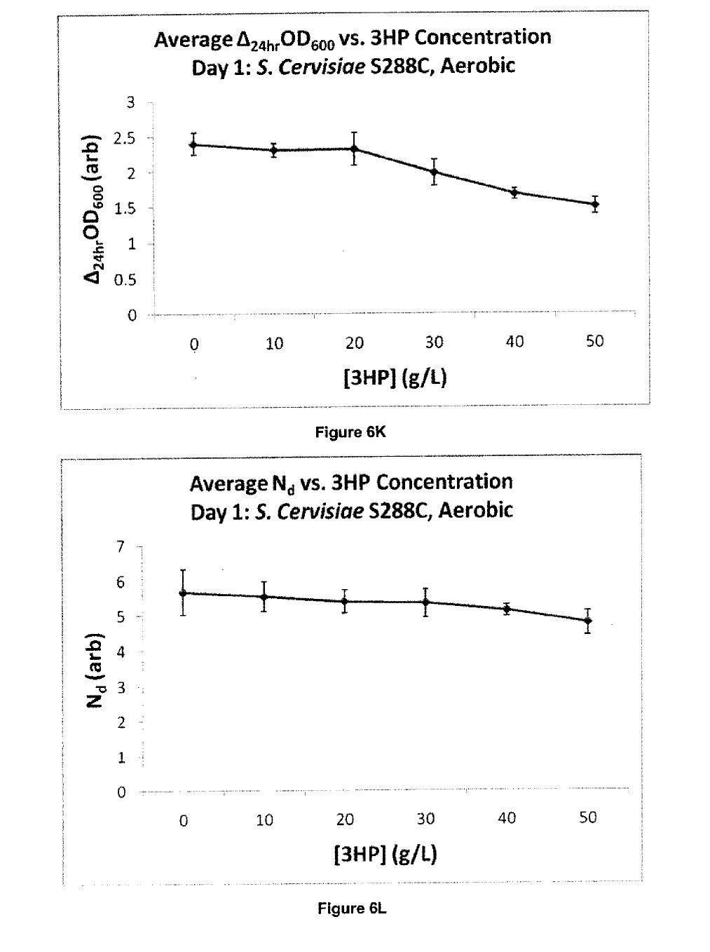

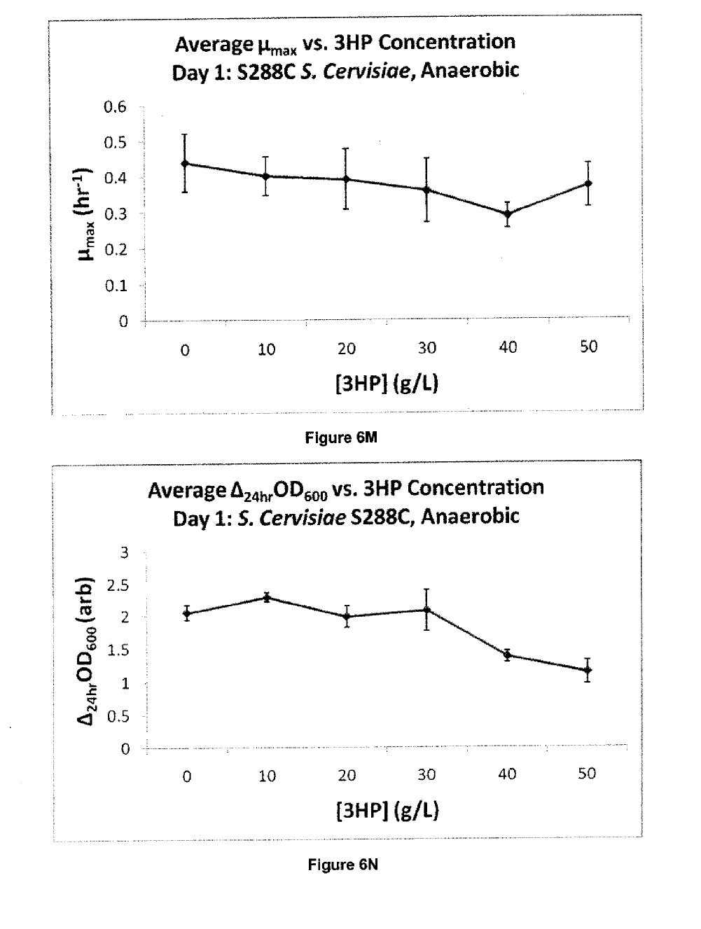

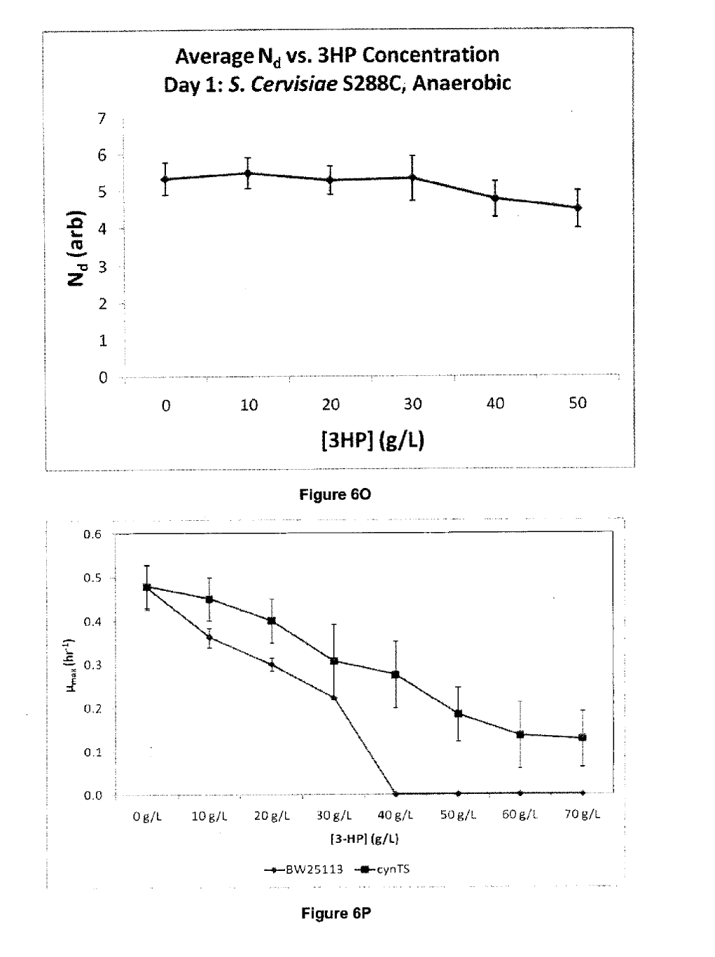

FIGS. 6A-O provide data from various control microorganism responses to different 3-HP concentrations (see Example 10 for the methods used to obtain this data). The data in these figures is shown variously as changes in maximum growth rate (μₘₐₓ), changes in optical density (“OD”), and relative doubling times over a given period, here 24 hours.

Determination of growth rates, lag times and maximum growth rates are commonly used analyses to develop comparative metrics. FIGS. 6A, 6D, 6G, 6J, and 6M demonstrate changes in maximum growth rates over a 24-hour test period for the indicated species under the indicated aerobic or anaerobic test conditions. When representing this data for a range of concentrations of a chemical of interest that is believed toxic and/or inhibitory to growth, this representation is termed a “toleragram” herein. Here, growth toleragrams are generated by measuring the specific growth rates of microorganisms subjected to growth conditions including varying amounts of 3-HP.

Further, FIG. 6P compares the growth toleragrams of a control microorganism culture with a microorganism in which genetic modification was made to increase expression of cynTS (in Group C of the 3HPTGC). The curve for a cynTS genetic modification in E. coli (made by Example 5, below) shows increasing maximum growth rate with increasing 3-HP concentration over a 24-hour evaluation period for each 3-HP concentration. This provides a qualitative visually observable difference. However, the greater area under the curve for the cynTS genetic modification affords a quantitative difference as well, which may be used for comparative purposes with other genetic modifications intended to improve 3-HP tolerance. Evaluation of such curves may lead to more effective identification of genetic modifications and/or supplements, and combinations thereof.

FIGS. 6B, 6E, 6H, 6K, and 6N demonstrate a control microorganism responses to different 3-HP concentrations wherein optical density (“OD,” measured at 600 nanometers) at 24-hours is the metric used. OD600 is a conventional measure of cell density in a microorganism culture. For E. coli under aerobic condition, FIG. 6B demonstrates a dramatic reduction in cell density at 24 hours starting at 30 g/L 3-HP. FIG. 6D shows a relatively sharper and earlier drop for E. coli under anaerobic conditions.

FIGS. 6C, 6F, 6I, 6L, and 6O demonstrate a control microorganism responses to different 3-HP concentrations wherein the number of cell doublings during the 24-hour period are displayed.

The above is intended as a non-limiting description of various ways to assess 3-HP tolerance improvements. Generally, demonstrable improvements in growth and/or survival are viewed as ways to assess an increase in tolerance, such as to 3-HP.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “microorganism” includes a single microorganism as well as a plurality of microorganisms; and the like.

The term “heterologous DNA,” “heterologous nucleic acid sequence,” and the like as used herein refers to a nucleic acid sequence wherein at least one of the following is true: (a) the sequence of nucleic acids is foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature. For example, regarding instance (c), a heterologous nucleic acid sequence that is recombinantly produced will have two or more sequences from unrelated genes arranged to make a new functional nucleic acid. Embodiments of the present invention may result from introduction of an expression vector into a host microorganism, wherein the expression vector contains a nucleic acid sequence coding for an enzyme that is, or is not, normally found in a host microorganism. With reference to the host microorganism's genome prior to the introduction of the heterologous nucleic acid sequence, then, the nucleic acid sequence that codes for the enzyme is heterologous (whether or not the heterologous nucleic acid sequence is introduced into that genome).

Generally, it is within the scope of the invention to provide one or more genetic modifications to increase a recombinant microorganism's tolerance to 3-HP by any one or more of the approaches described herein. Thus, within the scope of any of the above-described alternatives and embodiments thereof are the composition results of respective methods, that is, genetically modified microorganisms that comprise the one or more, two or more, three or more, etc. genetic modifications referred to toward obtaining increased tolerance to 3-HP.

Also, it is within the scope of the invention to provide, in a suitable culture vessel comprising a selected microorganism, one or more supplements that are intermediates or end products (collectively, “products”) of the 3HPTGC. Table 3 recites a non-limiting listing of supplements that may be added in a culture vessel comprising a genetically modified microorganism comprising one or more genetic modifications to the 3HPTGC and/or 3-HP production pathways. For example, not to be limiting, one or more of lysine, methionine, and bicarbonate may be provided. Such supplement additions may be combined with genetic modifications, as described herein, of the selected microorganism.

The examples below provide some examples, not meant to be limiting, of combinations of genetic modifications and supplement additions.

Further as to supplements, as to Group C regarding polyamine synthesis, the results of Example 3, below, demonstrate that 3-HP tolerance of E. coli was increased by adding the polyamines putrescine, spermidine and cadaverine to the media. Minimum inhibitory concentrations (MICs) for E. coli K12 in control and supplemented media were as follows: in M9 minimal media supplemented with putrescine 40 g/L, in M9 minimal media supplemented with spermidine 40 g/L, in M9 minimal media supplemented with cadavarine 30 g/L. Minimum inhibitory concentrations (MICs) for added sodium bicarbonate in M9 minimal media was 30 g/L. The Minimum inhibitory concentrations (MICs) for E. coli K12 in 100 g/L stock solution 3-HP was 20 g/L.

Further, in view of the increase over the control MIC with sodium bicarbonate supplementation, other alteration, such as regulation and/or genetic modification of carbonic anhydrase, such as providing a heterologous nucleic acid sequence to a cell of interest, where that nucleic acid sequence encodes a polypeptide possessing carbonic anhydrase activity are considered of value to increase tolerance to 3-HP (such as in combination with other alterations of the 3HPTGC). Similarly, and as supported by other data provided herein, alterations of the enzymatic activities, such as by genetic modification(s) of enzyme(s) along the 3HPTGC pathway portions that lead to arginine, putrescine, cadaverine and spermidine, are considered of value to increase tolerance to 3-HP (such as in combination with other alterations of the 3HPTGC).

In view of the above, it is appreciated that the results of supplementations evaluations provide evidence of the utility of direct supplementation into a culture media, and also of improving 3-HP tolerance by a genetic modification route, such as is provided in some examples herein. It is appreciated that increasing the concentration of a product of a 3HPTGC enzymatic conversion step, such as by a genetic modification, whether by supplementation and/or genetic modification(s), may be effective to increase the intracellular concentration of one or more 3HPTGC products in a microorganism and/or in the media in which such microorganism is cultured.

Taken together, the fitness data and subsequently obtained data from the examples below, related to genetic modifications and/or supplements pertaining to the 3HPTGC support a concept of a functional relationship between such alterations to increase enzymatic conversion along the pathways of the 3HPTGC and the resulting functional increase in 3-HP tolerance in a microorganism cell or culture system. This is observable for the 3HPTGC as a whole and also within and among its defined groups.

Further, tables 6-9, 11, and 13-17, incorporated into this section, provide non-limiting examples supplements additions, genetic modifications, and combinations of supplements additions and genetic modifications. Additional supplementations, genetic modifications, and combinations thereof, may be made in view of these examples and the described methods of identifying genetic modifications toward achieving an elevated tolerance to 3-HP in a microorganism of interest. Particular combinations may involve only the 3HPTGC lower section, including combinations involving two or more, three or more, or four or more, of the five groups therein (each involving supplement additions and/or genetic modification), any of these in various embodiments also comprising one or more genetic modifications or supplement additions regarding the 3HPTGC upper section.

Based on these results, it is appreciated that in various embodiments of the invention, whether methods or compositions, as a result of genetic modification and/or supplementation of reactants of the 3HPTGC, the alteration(s) directed to the 3HPTGC are effective to increase 3-HP tolerance by at least 5 percent, at least 10 percent, at least 20 percent, at least 30 percent, or at least 50 percent above a 3-HP tolerance of a control microorganism, lacking said at least one 3HPTGC genetic modification.

As is appreciated by the examples, any of the genetically modified microorganisms of the invention may be provided in a culture system and utilized, such as for the production of 3-HP. In some embodiments, one or more supplements (that are products of the 3HPTGC enzymatic conversion steps) are provided to a culture system to further increase overall 3-HP tolerance in such culture system.

Increased tolerance to 3-HP, whether of a microorganism or a culture system, may be assessed by any method or approach known to those skilled in the art, including but not limited to those described herein.

The genetic modification of the 3HPTGC upper portion may involve any of the enzymatic conversion steps. One, non-limiting example regards the tricarboxylic acid cycle. It is known that the presence and activity of the enzyme citrate synthase (E. C. 2.3.3.1 [previously 4.1.3.7]), which catalyzes the first step in that cycle, controls the rate of the overall cycle (i.e., is a rate-limiter). Accordingly, genetic modification of a microorganism, such as to increase copy numbers and/or specific activity, and/or other related characteristics (such as lower effect of a feedback inhibitor or other control molecule), may include a modification of citrase synthase. Ways to effectuate such change for citrate synthase may utilize any number of laboratory techniques, such as are known in the art, including approaches described herein for other enzymatic conversion steps of the 3HPTGC. Further, several commonly known techniques are described in U.S. Pat. Nos. 6,110,714 and 7,247,459, both assigned to Ajinomoto Co., Inc., both of which are herewith incorporated by reference for their respective teachings about amplifying citrate synthase activity (specifically, cols. 3 and 4, and Examples 3 and 4, of U.S. Pat. No. 6,110,714, and cols. 11 and 12 (specifically Examples (1) and (2)) of U.S. Pat. No. 7,247,459).

In various embodiments E. coli strains are provided that comprise selected gene deletions directed to increase enzymatic conversion in the 3HPTGC and accordingly increase microorganism tolerance to 3-HP. For example, the following genes, which are associated with repression of pathways in the indicated 3HPTGC Groups, may be deleted: Group A—tyrR, trpR; Group B—metJ; Group C—purR; Group D—lysR; Group E—nrdR. There are for E. coli and it is known and determinable by one skilled in the art to identify and genetically modify equivalent repressor genes in this and other species.

A disruption of gene function may also be effectuated, in which the normal encoding of a functional enzyme by a nucleic acid sequence has been altered so that the production of the functional enzyme in a microorganism cell has been reduced or eliminated. A disruption may broadly include a gene deletion, and also includes, but is not limited to gene modification (e.g., introduction of stop codons, frame shift mutations, introduction or removal of portions of the gene, introduction of a degradation signal), affecting mRNA transcription levels and/or stability, and altering the promoter or repressor upstream of the gene encoding the polypeptide. In some embodiments, a gene disruption is taken to mean any genetic modification to the DNA, mRNA encoded from the DNA, and the amino acid sequence resulting there from that results in at least a 50 percent reduction of enzyme function of the encoded gene in the microorganism cell.

Further, as to the full scope of the invention and for various embodiments, it is recognized that the above discussion and the examples below are meant to be exemplary and not limiting. Genetic manipulations may be made to achieve a desired alteration in overall enzyme function, such as by reduction of feedback inhibition and other facets of control, including alterations in DNA transcriptional and RNA translational control mechanisms, improved mRNA stability, as well as use of plasmids having an effective copy number and promoters to achieve an effective level of improvement. Such genetic modifications may be chosen and/or selected for to achieve a higher flux rate through certain basic pathways within the 3HPTGC and so may affect general cellular metabolism in fundamental and/or major ways. Accordingly, in certain alternatives genetic modifications are made more selectively, to other parts of the 3HPTGC.

Further, based on analysis of location and properties of committed steps, feedback inhibition, and other factors and constraints, in various embodiments at least one genetic modification is made to increase overall enzymatic conversion for one of the following enzymes of the 3HPTGC: 2-dehydro-3-deoxyphosphoheptonate aldolase (e.g., aroF, aroG, aroH); cyanase (e.g., cynS); carbonic anhydrase (e.g., cynT); cysteine synthase B (e.g., cysM); threonine deaminase (e.g., ilvA); ornithine decarboxylase (e.g., speC, speF); adenosylmethionine decarboxylase (e.g., speD); and spermidine synthase (e.g., speE). Genetic modifications may include increasing copy numbers of the nucleic acid sequences encoding these enzymes, and providing modified nucleic acid sequences that have reduced or eliminated feedback inhibition, control by regulators, increased affinity for substrate, and other modifications. Thus, one aspect of the invention is to genetically modify one or more of these enzymes in a manner to increase enzymatic conversion at one or more 3HPTGC enzymatic conversion steps so as to increase flux and/or otherwise modify reaction flows through the 3HPTGC so that 3-HP tolerance is increased. In addition to Examples 4 and 5 below, which pertain to genetic modifications regarding aroH and cyanase (with carbonic anhydrase), respectively, the following examples are provided. It is noted that in E. coli a second carbonic anhydrase enzyme is known. This is identified variously as Can and yadf.

Also, the invention regards the genetic modification to introduce a genetic element that encodes a short polypeptide identified herein as IroK. The introduction of genetic elements encoding this short polypeptide has been demonstrated to improve 3-HP tolerance in E. coli under microaerobic conditions (such as described herein). In various embodiments this genetic element may be introduced in combination with 3HPTGC-related genetic modifications and/or supplements to further improve 3-HP tolerance

Based on the above, and the examples below and data there from, other aspects of the invention are methods of identifying supplements, methods of identifying genetic modifications, and methods of identifying combinations of supplements and genetic modifications, related to the 3HPTGC that result in increased 3-HP tolerance for a microorganism.

Also, it is appreciated that various embodiments of the invention may comprise genetic modifications of the 3HPTGC, and/or supplements thereof, excluding any one or more designated enzymatic conversion steps, product additions, and/or specific enzymes. For example, an embodiment of the invention may comprise genetic modifications of the 3HPTGC excluding those of Group A, or of Groups A and B, or of a defined one or more members of the 3HPTGC (which may be any subset of the 3HPTGC members).

### D. Discussion of Microorganism Species

The examples below describe specific modifications and evaluations to certain bacterial and yeast microorganisms. The scope of the invention is not meant to be limited to such species, but to be generally applicable to a wide range of suitable microorganisms. As the genomes of various species become known, the present invention easily may be applied to an ever-increasing range of suitable microorganisms. Further, given the relatively low cost of genetic sequencing, the genetic sequence of a species of interest may readily be determined to make application of aspects of the present invention more readily obtainable (based on the ease of application of genetic modifications to an organism having a known genomic sequence).

More particularly, based on the various criteria described herein, suitable microbial hosts for the bio-production of 3-HP that comprise tolerance aspects provided herein generally may include, but are not limited to, any gram negative organisms such as E. coli, Oligotropha carboxidovorans, or Pseudomononas sp.; any gram positive microorganism, for example Bacillus subtilis, Lactobacillus sp. or Lactococcus sp. a yeast, for example Saccharomyces cerevisiae, Pichia pastoris or Pichia stipitis; and other groups or microbial species. More particularly, suitable microbial hosts for the bio-production of 3-HP generally include, but are not limited to, members of the genera Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula and Saccharomyces. Hosts that may be particularly of interest include: Oligotropha carboxidovorans (such as strain OM5), Escherichia coli, Alcaligenes eutrophus (Cupriavidus necator), Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis and Saccharomyces cerevisiae.

Species and other phylogenic identifications, above and elsewhere in this application, are according to the classification known to a person skilled in the art of microbiology.

Tolerance-improving features as described and claimed herein may be provided in a microorganism selected from the above listing, or another suitable microorganism, that also comprises one or more natural, introduced, or enhanced 3-HP bio-production pathways. Thus, in some embodiments the microorganism comprises an endogenous 3-HP production pathway (which may, in some such embodiments, be enhanced), whereas in other embodiments the microorganism does not comprise an endogenous 3-HP production pathway.

A genetically modified microorganism may incorporate genetic modifications based on the teachings of the present application for 3-HP tolerance improvements combined with any of various 3-HP production pathways. Varieties of these genetically modified microorganisms may comprise genetic modifications and/or other system alterations as may be described in other patent applications of one or more of the present inventor(s) and/or subject to assignment to the owner of the present patent application.

More generally, a microorganism used for the present invention may be selected from bacteria, cyanobacteria, filamentous fungi and yeasts. For some embodiments, microbial hosts initially selected for 3-HP toleragenic bio-production should also utilize sugars including glucose at a high rate. Most microbes are capable of utilizing carbohydrates. However, certain environmental microbes cannot utilize carbohydrates to high efficiency, and therefore would not be suitable hosts for such embodiments that are intended for glucose or other carbohydrates as the principal added carbon source.

The ability to genetically modify the host is essential for the production of any recombinant microorganism. The mode of gene transfer technology may be by electroporation, conjugation, transduction or natural transformation. A broad range of host conjugative plasmids and drug resistance markers are available. The cloning vectors are tailored to the host organisms based on the nature of antibiotic resistance markers that can function in that host.

### E. Other Aspects of Scope of the Invention

### Bio-Production Media

Bio-production media, which is used in the present invention with recombinant microorganisms having a biosynthetic pathway for 3-HP, must contain suitable carbon substrates for the intended metabolic pathways. Suitable substrates may include, but are not limited to, monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. Additionally the carbon substrate may also be one-carbon substrates such as carbon dioxide, carbon monoxide, or methanol for which metabolic conversion into key biochemical intermediates has been demonstrated. In addition to one and two carbon substrates methylotrophic organisms are also known to utilize a number of other carbon containing compounds such as methylamine, glucosamine and a variety of amino acids for metabolic activity. For example, methylotrophic yeast are known to utilize the carbon from methylamine to form trehalose or glycerol (Bellion et al., Microb. Growth C1 Compd., [Int. Symp.], 7th (1993), 415-32. Editor(s): Murrell, J. Collin; Kelly, Don P. Publisher: Intercept, Andover, UK). Similarly, various species of Candida will metabolize alanine or oleic acid (Sulter et al., Arch. Microbiol. 153:485-489 (1990)). Hence it is contemplated that the source of carbon utilized in the present invention may encompass a wide variety of carbon containing substrates and will only be limited by the choice of organism.

Although it is contemplated that all of the above mentioned carbon substrates and mixtures thereof are suitable in the present invention as a carbon source, common carbon substrates used as carbon sources are glucose, fructose, and sucrose, as well as mixtures of any of these sugars. Sucrose may be obtained from feedstocks such as sugar cane, sugar beets, cassava, and sweet sorghum. Glucose and dextrose may be obtained through saccharification of starch based feedstocks including grains such as corn, wheat, rye, barley, and oats.

In addition, fermentable sugars may be obtained from cellulosic and lignocellulosic biomass through processes of pretreatment and saccharification, as described, for example, in US patent application publication number US20070031918A1, which is herein incorporated by reference. Biomass refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers and animal manure. Any such biomass may be used in a bio-production method or system to provide a carbon source.

In addition to an appropriate carbon source, such as selected from one of the above-disclosed types, bio-production media must contain suitable minerals, salts, cofactors, buffers and other components, known to those skilled in the art, suitable for the growth of the cultures and promotion of the enzymatic pathway necessary for 3-HP production.

Finally, in various embodiments the carbon source may be selected to exclude acrylic acid, 1,4-butanediol, as well as other downstream products.

### Culture Conditions

Typically cells are grown at a temperature in the range of about 25° C. to about 40° C. in an appropriate medium, as well as up to 70° C. for thermophilic microorganisms. Suitable growth media in the present invention are common commercially prepared media such as Luria Bertani (LB) broth, M9 minimal media, Sabouraud Dextrose (SD) broth, Yeast medium (YM) broth (Ymin) yeast synthetic minimal media and minimal media as described herein, such as M9 minimal media. Other defined or synthetic growth media may also be used, and the appropriate medium for growth of the particular microorganism will be known by one skilled in the art of microbiology or bio-production science. In various embodiments a minimal media may be developed and used that does not comprise, or that has a low level of addition (e.g., less than 0.2, or less than one, or less than 0.05 percent) of one or more of yeast extract and/or a complex derivative of a yeast extract, e.g., peptone, tryptone, etc.

Suitable pH ranges for the bio-production are between pH 3.0 to pH 10.0, where pH 6.0 to pH 8.0 is a typical pH range for the initial condition.

However, the actual culture conditions for a particular embodiment are not meant to be limited by the ranges in this section.

Bio-productions may be performed under aerobic, microaerobic, or anaerobic conditions, with or without agitation.

The amount of 3-HP produced in a bio-production media generally can be determined using a number of methods known in the art, for example, high performance liquid chromatography (HPLC), gas chromatography (GC), or GC/Mass Spectroscopy (MS). Specific HPLC methods for the specific examples are provided herein.

### Bio-Production Reactors and Systems:

Any of the recombinant microorganisms as described and/or referred to above may be introduced into an industrial bio-production system where the microorganisms convert a carbon source into 3-HP in a commercially viable operation. The bio-production system includes the introduction of such a recombinant microorganism into a bioreactor vessel, with a carbon source substrate and bio-production media suitable for growing the recombinant microorganism, and maintaining the bio-production system within a suitable temperature range (and dissolved oxygen concentration range if the reaction is aerobic or microaerobic) for a suitable time to obtain a desired conversion of a portion of the substrate molecules to 3-HP. Industrial bio-production systems and their operation are well-known to those skilled in the arts of chemical engineering and bioprocess engineering. The following paragraphs provide an overview of the methods and aspects of industrial systems that may be used for the bio-production of 3-HP.

In various embodiments, any of a wide range of sugars, including, but not limited to sucrose, glucose, xylose, cellulose or hemicellulose, are provided to a microorganism, such as in an industrial system comprising a reactor vessel in which a defined media (such as a minimal salts media including but not limited to M9 minimal media, potassium sulfate minimal media, yeast synthetic minimal media and many others or variations of these), an inoculum of a microorganism providing one or more of the 3-HP biosynthetic pathway alternatives, and the a carbon source may be combined. The carbon source enters the cell and is cataboliized by well-known and common metabolic pathways to yield common metabolic intermediates, including phosphoenolpyruvate (PEP). (See Molecular Biology of the Cell, 3^{rd} Ed., B. Alberts et al. Garland Publishing, New York, 1994, pp. 42-45, 66-74, incorporated by reference for the teachings of basic metabolic catabolic pathways for sugars; Principles of Biochemistry, 3^{rd} Ed., D. L. Nelson & M. M. Cox, Worth Publishers, New York, 2000, pp 527-658, incorporated by reference for the teachings of major metabolic pathways; and Biochemistry, 4^{th} Ed., L. Stryer, W. H. Freeman and Co., New York, 1995, pp. 463-650, also incorporated by reference for the teachings of major metabolic pathways.). The appropriate intermediates are subsequently converted to 3-HP by one or more of the above-disclosed biosynthetic pathways.

Further to types of industrial bio-production, various embodiments of the present invention may employ a batch type of industrial bioreactor. A classical batch bioreactor system is considered “closed” meaning that the composition of the medium is established at the beginning of a respective bio-production event and not subject to artificial alterations and additions during the time period ending substantially with the end of the bio-production event. Thus, at the beginning of the bio-production event the medium is inoculated with the desired organism or organisms, and bio-production is permitted to occur without adding anything to the system. Typically, however, a “batch” type of bio-production event is batch with respect to the addition of carbon source and attempts are often made at controlling factors such as pH and oxygen concentration. In batch systems the metabolite and biomass compositions of the system change constantly up to the time the bio-production event is stopped. Within batch cultures cells moderate through a static lag phase to a high growth log phase and finally to a stationary phase where growth rate is diminished or halted. If untreated, cells in the stationary phase will eventually die. Cells in log phase generally are responsible for the bulk of production of a desired end product or intermediate.

A variation on the standard batch system is the Fed-Batch system. Fed-Batch bio-production processes are also suitable in the present invention and comprise a typical batch system with the exception that the nutrients including the substrate is added in increments as the bio-production progresses. Fed-Batch systems are useful when catabolite repression is apt to inhibit the metabolism of the cells and where it is desirable to have limited amounts of substrate in the media. Measurement of the actual nutrient concentration in Fed-Batch systems may be measured directly, such as by sample analysis at different times, or estimated on the basis of the changes of measurable factors such as pH, dissolved oxygen and the partial pressure of waste gases such as CO₂. Batch and Fed-Batch approaches are common and well known in the art and examples may be found in Thomas D. Brock in Biotechnology: A Textbook of Industrial Microbiology, Second Edition (1989) Sinauer Associates, Inc., Sunderland, Mass., Deshpande, Mukund V., Appl. Biochem. Biotechnol., 36:227, (1992), and Biochemical Engineering Fundamentals, 2^{nd} Ed. J. E. Bailey and D. F. Ollis, McGraw Hill, New York, 1986, herein incorporated by reference for general instruction on bio-production, which as used herein may be aerobic, microaerobic, or anaerobic.

Although the present invention may be performed in batch mode, as provided in Example 8, or in fed-batch mode, it is contemplated that the method would be adaptable to continuous bio-production methods. Continuous bio-production is considered an “open” system where a defined bio-production medium is added continuously to a bioreactor and an equal amount of conditioned media is removed simultaneously for processing. Continuous bio-production generally maintains the cultures within a controlled density range where cells are primarily in log phase growth. Two types of continuous bioreactor operation include: 1) Chemostat—where fresh media is fed to the vessel while simultaneously removing an equal rate of the vessel contents. The limitation of this approach is that cells are lost and high cell density generally is not achievable. In fact, typically one can obtain much higher cell density with a fed-batch process. 2) Perfusion culture, which is similar to the chemostat approach except that the stream that is removed from the vessel is subjected to a separation technique which recycles viable cells back to the vessel. This type of continuous bioreactor operation has been shown to yield significantly higher cell densities than fed-batch and can be operated continuously. Continuous bio-production is particularly advantageous for industrial operations because it has less down time associated with draining, cleaning and preparing the equipment for the next bio-production event. Furthermore, it is typically more economical to continuously operate downstream unit operations, such as distillation, than to run them in batch mode.

Continuous bio-production allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Methods of modulating nutrients and growth factors for continuous bio-production processes as well as techniques for maximizing the rate of product formation are well known in the art of industrial microbiology and a variety of methods are detailed by Brock, supra.

It is contemplated that embodiments of the present invention may be practiced using either batch, fed-batch or continuous processes and that any known mode of bio-production would be suitable. Additionally, it is contemplated that cells may be immobilized on an inert scaffold as whole cell catalysts and subjected to suitable bio-production conditions for 3-HP production.

The following published resources are incorporated by reference herein for their respective teachings to indicate the level of skill in these relevant arts, and as needed to support a disclosure that teaches how to make and use methods of industrial bio-production of 3-HP from sugar sources, and also industrial systems that may be used to achieve such conversion with any of the recombinant microorganisms of the present invention (Biochemical Engineering Fundamentals, 2^{nd} Ed. J. E. Bailey and D. F. Ollis, McGraw Hill, New York, 1986, entire book for purposes indicated and Chapter 9, pages 533-657 in particular for biological reactor design; Unit Operations of Chemical Engineering, 5^{th} Ed., W. L. McCabe et al., McGraw Hill, New York 1993, entire book for purposes indicated, and particularly for process and separation technologies analyses; Equilibrium Staged Separations, P. C. Wankat, Prentice Hall, Englewood Cliffs, N.J. USA, 1988, entire book for separation technologies teachings).

Also, the scope of the present invention is not meant to be limited to the exact sequences provided herein. It is appreciated that a range of modifications to nucleic acid and to amino acid sequences may be made and still provide a desired functionality, such as a desired enzymatic activity and specificity. The following discussion is provided describe ranges of variation that may be practiced and still remain within the scope of the present invention.

It has long been recognized in the art that some amino acids in amino acid sequences can be varied without significant effect on the structure or function of proteins. Variants included can constitute deletions, insertions, inversions, repeats, and type substitutions so long as the indicated enzyme activity is not significantly adversely affected. Guidance concerning which amino acid changes are likely to be phenotypically silent can be found, inter alia, in Bowie, J. U., et Al., “Deciphering the Message in Protein Sequences: Tolerance to Amino Acid Substitutions,” Science 247:1306-1310 (1990). This reference is incorporated by reference for such teachings, which are, however, also generally known to those skilled in the art.

In various embodiments polypeptides obtained by the expression of the polynucleotide molecules of the present invention may have at least approximately 50%, 60%, 70%, 80%, 90%, 95%, 96%, 97%, 98%, 99% or 100% identity to one or more amino acid sequences encoded by the genes and/or nucleic acid sequences described herein for the 3-HP tolerance-related and biosynthesis pathways. A truncated respective polypeptide has at least about 90% of the full length of a polypeptide encoded by a nucleic acid sequence encoding the respective native enzyme, and more particularly at least 95% of the full length of a polypeptide encoded by a nucleic acid sequence encoding the respective native enzyme. By a polypeptide having an amino acid sequence at least, for example, 95% “identical” to a reference amino acid sequence of a polypeptide is intended that the amino acid sequence of the claimed polypeptide is identical to the reference sequence except that the claimed polypeptide sequence can include up to five amino acid alterations per each 100 amino acids of the reference amino acid of the polypeptide. In other words, to obtain a polypeptide having an amino acid sequence at least 95% identical to a reference amino acid sequence, up to 5% of the amino acid residues in the reference sequence can be deleted or substituted with another amino acid, or a number of amino acids up to 5% of the total amino acid residues in the reference sequence can be inserted into the reference sequence. These alterations of the reference sequence can occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 50%, 60%, 70%, 80%, 85%, 90%, 92%, 95%, 96%, 97%, 98% or 99% identical to any reference amino acid sequence of any polypeptide described herein (which may correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set such that the percentage of identity is calculated over the full length of the reference amino acid sequence and that gaps in homology of up to 5% of the total number of amino acid residues in the reference sequence are allowed.

For example, in a specific embodiment the identity between a reference sequence (query sequence, i.e., a sequence of the present invention) and a subject sequence, also referred to as a global sequence alignment, may be determined using the FASTDB computer program based on the algorithm of Brutlag et al. (Comp. App. Biosci. 6:237-245 (1990)). Preferred parameters for a particular embodiment in which identity is narrowly construed, used in a FASTDB amino acid alignment, are: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. According to this embodiment, if the subject sequence is shorter than the query sequence due to N- or C-terminal deletions, not because of internal deletions, a manual correction is made to the results to take into consideration the fact that the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are lateral to the N- and C-terminal of the subject sequence, which are not matched/aligned with a corresponding subject residue, as a percent of the total bases of the query sequence. A determination of whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of this embodiment. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence are considered for this manual correction. For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for.

Also as used herein, the term “homology” refers to the optimal alignment of sequences (either nucleotides or amino acids), which may be conducted by computerized implementations of algorithms. “Homology”, with regard to polynucleotides, for example, may be determined by analysis with BLASTN version 2.0 using the default parameters. “Homology”, with respect to polypeptides (i.e., amino acids), may be determined using a program, such as BLASTP version 2.2.2 with the default parameters, which aligns the polypeptides or fragments being compared and determines the extent of amino acid identity or similarity between them. It will be appreciated that amino acid “homology” includes conservative substitutions, i.e. those that substitute a given amino acid in a polypeptide by another amino acid of similar characteristics. Typically seen as conservative substitutions are the following replacements: replacements of an aliphatic amino acid such as Ala, Val, Leu and Ile with another aliphatic amino acid; replacement of a Ser with a Thr or vice versa; replacement of an acidic residue such as Asp or Glu with another acidic residue; replacement of a residue bearing an amide group, such as Asn or Gln, with another residue bearing an amide group; exchange of a basic residue such as Lys or Arg with another basic residue; and replacement of an aromatic residue such as Phe or Tyr with another aromatic residue. A polypeptide sequence (i.e., amino acid sequence) or a polynucleotide sequence comprising at least 50% homology to another amino acid sequence or another nucleotide sequence respectively has a homology of 50% or greater than 50%, e.g., 60%, 70%, 80%, 90% or 100%.

The above descriptions and methods for sequence identity and homology are intended to be exemplary and it is recognized that these concepts are well-understood in the art. Further, it is appreciated that nucleic acid sequences may be varied and still encode an enzyme or other polypeptide exhibiting a desired functionality, and such variations are within the scope of the present invention. Nucleic acid sequences that encode polypeptides that provide the indicated functions for 3-HP increased tolerance or production are considered within the scope of the present invention. These may be further defined by the stringency of hybridization, described below, but this is not meant to be limiting when a function of an encoded polypeptide matches a specified 3-HP tolerance-related or biosynthesis pathway enzyme activity.

Further to nucleic acid sequences, “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1M, more usually less than about 500 mM and less than about 200 mM. Hybridization temperatures can be as low as 5° C., but are typically greater than 22° C., more typically greater than about 30° C., and often are in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tₘ for the specific sequence at a defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook and Russell and Anderson “Nucleic Acid Hybridization” 1^{st} Ed., BIOS Scientific Publishers Limited (1999), which are hereby incorporated by reference for hybridization protocols. “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

Based on the above, it is appreciated that various non-limiting aspects of the invention may include, but are not limited to:

A genetically modified (recombinant) microorganism comprising a nucleic acid sequence that encodes a polypeptide with at least 85% amino acid sequence identity to any of the enzymes of any of 3-HP tolerance-related or biosynthetic pathways, wherein the polypeptide has enzymatic activity and specificity effective to perform the enzymatic reaction of the respective 3-HP tolerance-related or biosynthetic pathway enzyme, and the recombinant microorganism exhibits greater 3-HP tolerance and/or 3-HP bio-production than an appropriate control microorganism lacking such nucleic acid sequence.

A genetically modified (recombinant) microorganism comprising a nucleic acid sequence that encodes a polypeptide with at least 90% amino acid sequence identity to any of the enzymes of any of 3-HP tolerance-related or biosynthetic pathways, wherein the polypeptide has enzymatic activity and specificity effective to perform the enzymatic reaction of the respective 3-HP tolerance-related or biosynthetic pathway enzyme, and the recombinant microorganism exhibits greater 3-HP tolerance and/or 3-HP bio-production than an appropriate control microorganism lacking such nucleic acid sequence.

A genetically modified (recombinant) microorganism comprising a nucleic acid sequence that encodes a polypeptide with at least 95% amino acid sequence identity to any of the enzymes of any of 3-HP tolerance-related or biosynthetic pathways, wherein the polypeptide has enzymatic activity and specificity effective to perform the enzymatic reaction of the respective 3-HP tolerance-related or biosynthetic pathway enzyme, and the recombinant microorganism exhibits greater 3-HP tolerance and/or 3-HP bio-production than an appropriate control microorganism lacking such nucleic acid sequence. In some embodiments, the at least one polypeptide has at least 99% or 100% sequence identity to at least one of the enzymes of a 3-HPTGC pathway and/or a 3-HP biosynthetic pathway.

In one aspect of the invention the identity values in the preceding paragraphs are determined using the parameter set described above for the FASTDB software program. It is recognized that identity may be determined alternatively with other recognized parameter sets, and that different software programs (e.g., Bestfit vs. BLASTp) are expected to provide different results. Thus, identity can be determined in various ways. Further, for all specifically recited sequences herein it is understood that conservatively modified variants thereof are intended to be included within the invention.

In some embodiments, the invention contemplates a genetically modified (e.g., recombinant) microorganism comprising a heterologous nucleic acid sequence that encodes a polypeptide that is an identified enzymatic functional variant of any of the enzymes of any of 3-HP tolerance-related pathways, or pathway portions (i.e., of the 3HPTGC), wherein the polypeptide has enzymatic activity and specificity effective to perform the enzymatic reaction of the respective 3-HP tolerance-related enzyme, so that the recombinant microorganism exhibits greater 3-HP tolerance than an appropriate control microorganism lacking such nucleic acid sequence. Relevant methods of the invention also are intended to be directed to identified enzymatic functional variants and the nucleic acid sequences that encode them.

The term “identified enzymatic functional variant” means a polypeptide that is determined to possess an enzymatic activity and specificity of an enzyme of interest but which has an amino acid sequence different from such enzyme of interest. A corresponding “variant nucleic acid sequence” may be constructed that is determined to encode such an identified enzymatic functional variant. For a particular purpose, such as increased tolerance to 3-HP via genetic modification to increase enzymatic conversion at one or more of the enzymatic conversion steps of the 3HPTGC in a microorganism, one or more genetic modifications may be made to provide one or more heterologous nucleic acid sequence(s) that encode one or more identified 3HPTGC enzymatic functional variant(s). That is, each such nucleic acid sequence encodes a polypeptide that is not exactly the known polypeptide of an enzyme of the 3HPTGC, but which nonetheless is shown to exhibit enzymatic activity of such enzyme. Such nucleic acid sequence, and the polypeptide it encodes, may not fall within a specified limit of homology or identity yet by its provision in a cell nonetheless provide for a desired enzymatic activity and specificity. The ability to obtain such variant nucleic acid sequences and identified enzymatic functional variants is supported by recent advances in the states of the art in bioinformatics and protein engineering and design, including advances in computational, predictive and high-throughput methodologies.

It is understood that the steps described herein and also exemplified in the non-limiting examples below comprise steps to make a genetic modification, and steps to identify a genetic modification and/or supplement, and combination thereof, to improve 3-HP tolerance in a microorganism and/or in a microorganism culture. Also, the genetic modifications so obtained and/or identified comprise means to make a microorganism exhibiting an increased tolerance to 3-HP.

Having so described the present invention and provided examples below, and in view of the above paragraphs, it is appreciated that various non-limiting aspects of the present invention may include, but are not limited to, the following embodiments.

In some embodiments, the invention contemplates a recombinant microorganism comprising at least one genetic modification effective to increase 3-hydroxypropionic acid (“3-HP”) production, wherein the increased level of 3-HP production is greater than the level of 3-HP production in the wild-type microorganism, and at least one genetic modification of the 3-HP Toleragenic Complex (“3HPTGC”). In some embodiments, the wild-type microorganism produces 3-HP. In some embodiments, the wild-type microorganism does not produce 3-HP. In some embodiments, the recombinant microorganism comprises at least one vector, such as at least one plasmid, wherein the at least one vector comprises at least one heterologous nucleic acid molecule.

In some embodiments of the invention, the at least one genetic modification of the 3HPTGC is effective to increase the 3-HP tolerance of the recombinant microorganism above the 3-HP tolerance of a control microorganism, wherein the control microorganism lacks the at least one 3HPTGC genetic modification. In some embodiments, the 3-HP tolerance of the recombinant microorganism is increased above the 3-HP tolerance of a control microorganism by about 5%, 10%, or 20%. In some embodiments, the 3-HP tolerance of the recombinant microorganism is increased above the 3-HP tolerance of a control microorganism by about 30%, 40%, 50%, 60%, 80%, or 100%.

Also, in various embodiments, the at least one genetic modification of the 3HPTGC encodes at least one polypeptide exhibiting at least one enzymatic conversion of at least one enzyme of the 3HPTGC, wherein the recombinant microorganism exhibits an increased 3-HP tolerance at least about 5, 10, 20, 30, 40, 50, 60, or 100 percent greater, or more, than the 3-HP tolerance of a control microorganism lacking the at least one genetic modification of the 3HPTGC, Any evaluations for such tolerance improvements may be based on a Minimum Inhibitory Concentration evaluation in a minimal media.

In some embodiments, the microorganism further comprises at least one additional genetic modification encoding at least one polypeptide exhibiting at least one enzymatic conversion of at least one enzyme of a second Group different from the genetic modification of a first Group of the 3HPTGC, wherein the recombinant microorganism exhibits an increased 3-HP tolerance at least about 5, 10, 20, 30, 40, 50, 60, or 100 percent greater, or more, than the 3-HP tolerance of a control microorganism lacking all said genetic modifications of the 3HPTGC. In the various embodiments, the at least one additional genetic modification further comprises a genetic modification from each of two or more, or three or more, of the Groups A-F.

For example, the genetic modifications may comprise at least one genetic modification of Group A and at least one genetic modification of Group B, at least one genetic modification of Group A and at least one genetic modification of Group C, at least one genetic modification of Group A and at least one genetic modification of Group D, at least one genetic modification of Group A and at least one genetic modification of Group E, at least one genetic modification of Group B and at least one genetic modification of Group C, at least one genetic modification of Group B and at least one genetic modification of Group D, at least one genetic modification of Group B and at least one genetic modification of Group E, at least one genetic modification of Group C and at least one genetic modification of Group D, at least one genetic modification of Group C and at least one genetic modification of Group E, or at least one genetic modification of Group D and at least one genetic modification of Group E. Any such combinations may be further practiced with Group F genetic modifications.

In some embodiments, the recombinant microorganism comprises one or more gene disruptions of 3HPTGC repressor genes selected from tyrR, trpR, metJ, argR, purR, lysR and nrdR.

In some embodiments, the recombinant microorganism is a gram-negative bacterium. In some embodiments, the recombinant microorganism is selected from the genera Zymomonas, Escherichia, Pseudomonas, Alcaligenes, and Klebsiella, In some embodiments, the recombinant microorganism is selected from the species Escherichia coli, Cupriavidus necator, Oligotropha carboxidovorans, and Pseudomonas putida. In some embodiments, the recombinant microorganism is an E. coli strain.

In some embodiments, the recombinant microorganism is a gram-positive bacterium. In some embodiments, the recombinant microorganism is selected from the genera Clostridium, Salmonella, Rhodococcus, Bacillus, Lactobacillus, Enterococcus, Paenibacillus, Arthrobacter, Corynebacterium, and Brevibacterium. In some embodiments, the recombinant microorganism is selected from the species Bacillus licheniformis, Paenibacillus macerans, Rhodococcus erythropolis, Lactobacillus plantarum, Enterococcus faecium, Enterococcus gallinarium, Enterococcus faecalis, and Bacillus subtilis. In some embodiments, the recombinant microorganism is a B. subtilis strain.

In some embodiments, the recombinant microorganism is a yeast. In some embodiments, the recombinant microorganism is selected from the genera Pichia, Candida, Hansenula and Saccharomyces. In some embodiments, the recombinant microorganism is Saccharomyces cerevisiae.

In some embodiments, the at least one genetic modification of the 3HPTGC comprises means to increase expression of SEQ ID NO: 129 (Irok peptide). In some embodiments, the recombinant microorganism is an E. coli strain. In some embodiments, the recombinant microorganism is a Cupriavidus necator strain.

In some embodiments, the at least one genetic modification encodes at least one polypeptide with at least 85% amino acid sequence identity to at least one of the enzymes of a 3-HPTGC pathway, a 3-HP biosynthetic pathway, and/or SEQ ID NO: 129 (Irok).

Some embodiments of the invention contemplate a culture system. In some embodiments, the culture system comprises a genetically modified microorganism as described herein and a culture media. Such genetically modified microorganism may comprise a single genetic modification of the 3HPTGC, or any of the combinations described herein, and may additionally comprise one or more genetic modifications of a 3-HP production pathway. In some embodiments, the culture media comprises at least about 1 g/L, at least about 5 g/L, at least about 10 g/L, at least about 15 g/L, or at least about 20 g/L of 3-HP. In some embodiments, the culture system comprises a 3HPTGC supplement at a respective concentration such as that shown in Table 3.

In some embodiments the invention contemplates a method of making a genetically modified microorganism comprising providing at least one genetic modification to increase the enzymatic conversion of the genetically modified microorganism over the enzymatic conversion of a control microorganism, wherein the control microorganism lacks the at least one genetic modification, at an enzymatic conversion step of the 3-hydroxypropionic acid Toleragenic Complex (“3HPTGC”), wherein the genetically modified microorganism synthesizes 3-HP. In some embodiments, the control microorganism synthesizes 3-HP. In some embodiments, the at least one genetic modification increases the 3-HP tolerance of the genetically modified microorganism above the 3-HP tolerance of the control microorganism. In some embodiments, the 3-HP tolerance of the genetically modified microorganism is at least about 5 percent, at least about 10 percent, at least about 20 percent, at least about 30 percent, at least about 40 percent, at least about 50 percent, or at least about 100 percent above the 3-HP tolerance of the control microorganism. In some embodiments, the 3-HP tolerance of the genetically modified microorganism is from about 50 to about 300 percent above the 3-HP tolerance of the control microorganism, based on a Minimum Inhibitory Concentration evaluation in a minimal media. In some embodiments, the genetically modified microorganism further comprises one or more gene disruptions of 3HPTGC repressor genes selected from tyrR, trpR, metJ, argR, purR, lysR and nrdR. In some embodiments, the control microorganism does not synthesize 3-HP. In some embodiments, providing at least one genetic modification comprises providing at least one vector. In some embodiments, the at least one vector comprises at least one plasmid. In some embodiments, providing at least one genetic modification comprises providing at least one nucleic acid molecule. In some embodiments, the at least one nucleic acid molecule is heterologous. In some embodiments, the at least one nucleic acid molecule encodes SEQ ID NO: 129 (Irok).

In some embodiments the invention provides a method of making a genetically modified microorganism comprising:

a. selecting a microorganism comprising the steps of:
i. providing a microorganism species or strain, wherein the microorganism species or strain of interest has a genomic sequence;
ii. identifying the genomic sequence of the microorganism;
iii. identifying homologies between the genomic sequence of the microorganism and the 3-hydroxypropionic acid toleragenic complex (3HPTGC) of FIGS. 1A-D,

b. genetically modifying the microorganism selected in step a. by introducing into the microorganism at least one selected genetic modification, wherein the at least one selected genetic modification increases the conversion at one or more enzymatic conversion steps that are functionally equivalent to one or more 3HPTGC enzymatic conversion steps of FIGS. 1A-D; wherein increasing the conversion at one or more enzymatic conversion steps increases the 3-HP tolerance of the microorganism over the 3-HP tolerance of a control microorganism lacking the at least one selected genetic modification;

c. evaluating the at least one selected genetic modification introduced in step b. to identify a product microorganism, wherein the product microorganism has 3-HP tolerance that is greater than the 3-HP tolerance of the control microorganism;

d. selecting the at least one selected genetic modification evaluated in step b.; and

e. making the genetically modified microorganism by introducing into a cell or a plurality of cells the at least one genetic modification of the product microorganism of step c. to generate a genetically modified microorganism, wherein the genetically modified microorganism has 3-HP tolerance that is at least about 5 percent greater than the 3-HP tolerance of the control microorganism

In some embodiments, the invention contemplates a method of improving 3-hydroxypropionic acid (3-HP) tolerance comprising:

a. introducing at least one genetic modification into a selected microorganism that synthesizes 3-HP wherein the at least one genetic modification increases enzymatic conversion at at least one enzymatic conversion step of a portion of the 3HPTGC, wherein the portion of the 3HPTGC is threonine/homocysteine, polyamine synthesis, lysine synthesis, or nucleotide synthesis (or any other selected portion of the 3HPTGC); and

b. exposing the selected microorganism to a medium comprising at least about 1, 5, 10, 20, 25, 30, 40 or 50 g/L 3-HP,

wherein the selected microorganism exhibits 3-HP tolerance at least about 5, 10, 20, 30, 40, 50, or 100 percent better than the 3-HP tolerance of a control microorganism lacking the at least one genetic modification of step a. Thus, in some embodiments, the selected microorganism exhibits 3-HP tolerance at least about 5 percent, at least about 10 percent, at least about 20 percent, at least about 30 percent, at least about 40 percent, at least about 50 percent, or at least about 100 percent above greater than the 3-HP tolerance of a control microorganism lacking the at least one genetic modification of step a.

In some embodiments, genetic modifications are made to increase enzymatic conversion at an enzymatic conversion step identified to have an elevated fitness score in Table 1 and/or evaluated in the Examples below. Enzymes that catalyze such reactions are numerous and include cyanase and carbonic anhydrase.

In some embodiments, the invention contemplates a recombinant microorganism comprising:

a. at least one genetic modification increasing enzymatic conversion of one or both of cyanase and carbonic anhydrase; and

b. at least one additional genetic modification of a portion of the 3-HP Toleragenic Complex (“3HPTGC”), wherein the portion of the 3HPTGC is the chorismate, threonine/homocysteine, lysine synthesis, or nucleotide synthesis portion of the 3HPTGC. In some embodiments, the microorganism further comprises at least one further genetic modification of the polyamine portion of the 3HPTGC.

Also, for some embodiments the genetic modification of the 3HPTGC is not from Group A, or not from Groups A and B.

Also, it is appreciated that various embodiments of the invention may be directed to amino acid sequences of enzymes that catalyze the enzymatic conversion steps of the 3HPTGC for any species. More particularly, the amino acid sequences of the 3HPTGC for FIGS. 1A-D are readily obtainable from one or more of commonly used bioinformatics databases (e.g., www.ncbi.gov; www.metacyc.org) by entering a respective gene for an enzymatic conversion step therein.

The practice of the present invention will employ, unless otherwise indicated, conventional techniques of the biosynthetic industry and the like, which are within the skill of the art. Such techniques are fully explained in the literature and exemplary methods are provided below.

Also, while steps of the example involve use of plasmids, other vectors known in the art may be used instead. These include cosmids, viruses (e.g., bacteriophage, animal viruses, plant viruses), and artificial chromosomes (e.g., yeast artificial chromosomes (YAC) and bacteria artificial chromosomes (BAC)).

Before the specific examples of the invention are described in detail, it is to be understood that, unless otherwise indicated, the present invention is not limited to particular sequences, expression vectors, enzymes, host microorganisms, compositions, processes or systems, or combinations of these, as such may vary. It is also to be understood that the terminology used herein is for purposes of describing particular embodiments only, and is not intended to be limiting.

Also, and more generally, in accordance with disclosures, discussions, examples and embodiments herein, there may be employed conventional molecular biology, cellular biology, microbiology, and recombinant DNA techniques within the skill of the art. Such techniques are explained fully in the literature. (See, e.g., Sambrook and Russell, Molecular Cloning: A Laboratory Manual, Third Edition 2001 (volumes 1-3), Cold Spring Harbor Laboratory Press, Cold Spring Harbor, N.Y.; Animal Cell Culture, R. I. Freshney, ed., 1986). These published resources are incorporated by reference herein for their respective teachings of standard laboratory methods found therein. Further, all patents, patent applications, patent publications, and other publications referenced herein (collectively, “published resource(s)”) are hereby incorporated by reference in this application. Such incorporation, at a minimum, is for the specific teaching and/or other purpose that may be noted when citing the reference herein. If a specific teaching and/or other purpose is not so noted, then the published resource is specifically incorporated for the teaching(s) indicated by one or more of the title, abstract, and/or summary of the reference. If no such specifically identified teaching and/or other purpose may be so relevant, then the published resource is incorporated in order to more fully describe the state of the art to which the present invention pertains, and/or to provide such teachings as are generally known to those skilled in the art, as may be applicable. However, it is specifically stated that a citation of a published resource herein shall not be construed as an admission that such is prior art to the present invention. Also, in the event that one or more of the incorporated published resources differs from or contradicts this application, including but not limited to defined terms, term usage, described techniques, or the like, this application controls.

While various embodiments of the present invention have been shown and described herein, it is emphasized that such embodiments are provided by way of example only. Numerous variations, changes and substitutions may be made without departing from the invention herein in its various embodiments. Specifically, and for whatever reason, for any grouping of compounds, nucleic acid sequences, polypeptides including specific proteins including functional enzymes, metabolic pathway enzymes or intermediates, elements, or other compositions, or concentrations stated or otherwise presented herein in a list, table, or other grouping (such as metabolic pathway enzymes shown in a figure), unless clearly stated otherwise, it is intended that each such grouping provides the basis for and serves to identify various subset embodiments, the subset embodiments in their broadest scope comprising every subset of such grouping by exclusion of one or more members (or subsets) of the respective stated grouping. For example, a claimable subset of the enzymes or enzymatic conversion steps of FIG. 1A, sheets 1-7, and its equivalents in other species, may exclude the enzymes of the tricarboxylic acid pathway or the entire upper section. Moreover, when any range is described herein, unless clearly stated otherwise, that range includes all values therein and all sub-ranges therein. Accordingly, it is intended that the invention be limited only by the spirit and scope of appended claims, and of later claims, and of either such claims as they may be amended during prosecution of this or a later application claiming priority hereto.

### EXAMPLES SECTION

Most of the following examples disclose specific methods for providing an cell with heterologous nucleic acid sequences that encode for enzymes or other polypeptides that confer increased tolerance to 3-HP. Where there is a method to achieve a certain result that is commonly practiced in two or more specific examples (or for other reasons), that method may be provided in a separate Common Methods section that follows the examples. Each such common method is incorporated by reference into the respective specific example that so refers to it. Also, where supplier information is not complete in a particular example, additional manufacturer information may be found in a separate Summary of Suppliers section that may also include product code, catalog number, or other information. This information is intended to be incorporated in respective specific examples that refer to such supplier and/or product.

In the following examples, efforts have been made to ensure accuracy with respect to numbers used (e.g., amounts, temperatures, etc.), but some experimental error and deviation should be accounted for. Unless indicated otherwise, temperature is in degrees Celsius and pressure is at or near atmospheric pressure at approximately 5340 feet (1628 meters) above sea level. It is noted that work done at external analytical and synthetic facilities was not conducted at or near atmospheric pressure at approximately 5340 feet (1628 meters) above sea level. All reagents, unless otherwise indicated, were obtained commercially. Species and other phylogenic identifications provided in the examples and the Common Methods Section are according to the classification known to a person skilled in the art of microbiology.

The meaning of abbreviations is as follows: “C” means Celsius or degrees Celsius, as is clear from its usage, “s” means second(s), “min” means minute(s), “h,” “hr,” or “hrs” means hour(s), “psi” means pounds per square inch, “nm” means nanometers, “d” means day(s), “μL” or “uL” or “ul” means microliter(s), “mL” means milliliter(s), “L” means liter(s), “mm” means millimeter(s), “nm” means nanometers, “mM” means millimolar, “μM” or “uM” means micromolar, “M” means molar, “mmol” means millimole(s), “μmol” or “uMol” means micromole(s)“, “g” means gram(s), “μg” or “ug” means microgram(s) and “ng” means nanogram(s), “PCR” means polymerase chain reaction, “OD” means optical density, “OD₆₀₀” means the optical density measured at a wavelength of 600 nm, “kDa” means kilodaltons, “g” means the gravitation constant, “bp” means base pair(s), “kbp” means kilobase pair(s), “% w/v” means weight/volume percent, % v/v” means volume/volume percent, “IPTG” means isopropyl-μ-D-thiogalactopyranoiside, “RBS” means ribosome binding site, “rpm” means revolutions per minute, “HPLC” means high performance liquid chromatography, and “GC” means gas chromatography. As disclosed above, “3-HP” means 3-hydroxypropionic acid and “3HPTGC” means the 3-HP toleragenic complex. Also, 10̂5 and the like are taken to mean 10⁵ and the like.

### Example 1

### Increased Copy of Genetic Elements in the 3HPTGC Confer Tolerance to 3-HP

Data from a SCALEs evaluation of library clone fitness related to 3-HP exposure, using the SCALEs technique, affords clear evidence of the relevance as to 3-HP tolerance of a number of genes and enzymes. From this data, and in view of fitness data from other portions of the 3HPTGC, a broad view may be obtained that appropriate modifications of any of the genes or enzymes of the 3HPTGC and/or provision of nucleic acid sequences that provide an enzyme activity of such enzymes (without necessarily encoding the entire enzyme) may result in an altered enzymatic activity that leads to increased 3-HP tolerance.

The method used to measure 3-HP tolerance conferred by genes in the 3HPTGC is summarized as follows. The methods disclosed immediately below describe aspects of the SCALES methodology, which also was described above in somewhat less detail overall.

Bacteria, Plasmids, and Library Construction

Wild-type Escherichia coli K12 (ATCC #29425) was used for the preparation of genomic DNA. Six samples of purified genomic DNA were digested with two blunt cutters AluI and RsaI (Invitrogen, Carlsbad, Calif. USA) for different respective times—10, 20, 30, 40, 50 and 60 minutes at 37 C, and then were heat inactivated at 70C for 15 minutes. Restriction digestions were mixed and the fragmented DNA was separated based on size using agarose gel electrophoresis. Respective DNA fragments of 0.5, 1, 2, 4 and greater than 8 kb sizes were excised from the gel and purified with a gel extraction kit (Quagen) according to manufacturer's instructions. Genomic libraries were constructed by ligation of the respective purified fragmented DNA with the pSMART-LCKAN vector (Lucigen, Middleton, Wis. USA) according to manufacturer's instructions. Each ligation product was then electroporated into E. Cloni 10 G Supreme Electrocompetent Cells (Lucigen) and plated on LB+kanamycin. Colonies were harvested and plasmid DNA was extracted using Quiagen HiSpeed Plasmid Midi Kit according to manufacturer's instructions. Purified plasmid DNA of each library was introduced into Escherichia coli strain Mach1-T1® (Invitrogen, Carlsbad, Calif. USA) by electroporation. These cultures, representing each library—0.5, 1.0, 2.0, 4.0 and >8.0 kb of genomic DNA, were combined and incubated at 37 C to a desired density, to an OD₆₀₀ of approximately 0.50. This combined library culture mixture was used for selections below. (See section below and also see Lynch, M., Warencke, T E, Gill, R T, SCALEs: multiscale analysis of library enrichment. Nature Methods, 2007. 4(87-93); Warnecke, T. E., Lynch, M. D., Karimpour-Fard, A., Sandoval, N., Gill, R. T., A genomics approach to improve the analysis and design of strain selections. Metabolic Engineering, 2008 10(154-156)). Mach1-T1® containing pSMART-LCKAN empty vector were used for all control studies. Growth curves were done in MOPS Minimal Medium (See Neidhardt, F., Culture medium for enterobacteria. J Bacteriol, 1974. 119: p. 736-747.). Antibiotic concentration was 20 ug kanamycin/mL.

3-HP Preparation

3-HP was obtained from TCI America (Portland, Oreg.). Significant acrylic acid and 2-oxydipropionic contamination was observed via HPLC analysis. Samples were subsequently treated by diethyl ether extraction to remove acrylic acid and a portion of the 2-oxydipropionic contaminants. Samples were then neutralized with 10 M NaOH to a final pH of 7.0. Considerable insoluble matter was observed at neutral pH at concentrations in excess of approximately 35 g/L. Neutralized samples were centrifuged at 4000 rpm for 30 minutes at 4° C. The soluble 3-HP fraction was isolated from the thus-centrifuged insoluble matter and further analyzed by HPLC for a final quantification of concentration and purity of the working stock solution. The working stock solution was used for the selection and MIC evaluations in this example.

Selections

As noted above, five representative genomic libraries were created from E. coli K12 genomic DNA with defined insert sizes of 0.5, 1, 2, 4, and 8 kb, each library was transformed into MACH1™-T1® E. coli, cultured and then mixed. The mixture was aliquoted into two 15 mL screw cap tubes with a final concentration of 20 g/L 3-HP (TCI America) neutralized to pH 7 with 10 M NaOH. The cell density of the selection cultures was monitored as they approached a final OD₆₀₀ of 0.3-0.4. The original selection cultures were subsequently used to inoclulate another round of 15 mL MOPS minimal media+kanamycin+3-HP as part of a repeated batch selection strategy. Overall, a selection was carried out over 8 serial transfer batches with a decreasing gradient of 3-HP over 60 hours. More particularly, the 3-HP concentrations were 20 g 3-HP/L for serial batches 1 and 2, 15 g 3-HP/L for serial batches 3 and 4, 10 g 3-HP/L for serial batches 5 and 6, and 5 g 3-HP/L for serial batches 7 and 8. For serial batches 7 and 8 the culture media was replaced as the culture approached stationary phase to avoid nutrient limitations (Also see Warnecke, T. E., Lynch, M. D., Karimpour-Fard, A., Sandoval, N., Gill, R. T., A genomics approach to improve the analysis and design of strain selections. Metabolic Engineering, 2008 10(154-156), incorporated by reference herein). Batch transfer times were adjusted as needed to avoid a nutrient limited selection environment. Samples were taken at the culmination of each batch. Repeated batch cultures containing 3-HP were monitored and inoculated over a 60 hour period to enhance the concentration of clones exhibiting increased growth in the presence of 3-HP. Samples were taken by plating 1 mL of the selected population onto selective plates (LB with kanamycin) with each batch. Plasmid DNA was extracted from each sample and hybridized to Affymetrix E. Coli Antisense GeneChip® arrays (Affymetrix, Santa Clara, Calif.) according to previous work (See Lynch, M., Warencke, T E, Gill, R T, SCALEs: multiscale analysis of library enrichment. Nature Methods, 2007. 4(87-93)) and manufacturer's instructions.

Data Analysis

Data analysis was completed by utilizing SCALEs-appropriate software as described herein and also in Lynch, M., Warencke, T E, Gill, R T, SCALEs: multiscale analysis of library enrichment. Nature Methods, 2007. 4(87-93)). Fitness contributions from specific genomic elements were calculated from the enrichment of each region as a fraction of the selected population, as was previously described (Lynch, M., Warencke, T E, Gill, R T, SCALEs: multiscale analysis of library enrichment. Nature Methods, 2007. 4(87-93)). Briefly, plasmid DNA from samples taken at the culmination of each batch in the selection were hybridized to Affymetrix E. Coli Antisense GeneChip® arrays per above and data obtained from this was further analyzed. For each array, signal values corresponding to individual probe sets were extracted from the Affymetrix data file and partitioned into probe sets based on similar affinity values (Naef, F. and Magnasco, M. O., 2003, Solving the riddle of the bright mismatches: labeling and effective binding in oligonucelotide arrays. Phys. Rev. E 68, 011906). Background signal for each probe was subtracted according to conventional Affymetriz algorithms (MAS 5.0). Non-specific noise was determined as the intercept of the robust regression of the difference of the perfect match and mismatch signal against the perfect match signal. Probe signals were then mapped to genomic position as the tukey bi-weight of the nearest 25 probe signals and were de-noised by applying a medium filter with a 1000 bp window length. Gaps between probes were filled in by linear interpolation. This continuous signal was decomposed using an N-sieve based analysis and reconstructed on a minimum scale of 500 bp as described in detail by Lynch et al (2007). Signals were further normalized by the total repressor of primer (ROP) signal, which is on the library vector backbone and represents the signal corresponding to the total plasmid concentration added to the chip.

The analysis decomposed the microarray signals into corresponding library clones and calculated relative enrichment of specific regions over time. In this way, genome-wide fitness (ln(Xᵢ/Xᵢ₀)) was measured based on region specific enrichment patterns for the selection in the presence of 3-HP. Genetic elements and their corresponding fitness were then segregated by metabolic pathway based on their EcoCyc classifications (ecocyc.org). This fitness matrix was used to calculate both pathway fitness (W) and frequency of enrichment found in the selected population.

$${W}_{pathway} = {\sum }_{1}^{n} {W}_{i}$$

$$frequency = \frac{number of genes from metabolic pathway}{total genes in pathway}$$

Pathway redundancies were identified by an initial rank ordering of pathway fitness, followed by a specific assignment for genetic elements associated with multiple pathways to the primary pathway identified in the first rank, and subsequent removal of the gene-specific fitness values from the secondary pathways.

Similarly genes in a given genetic element were assigned fitness independent of neighboring genes in a genetic element as follows: The fitness of any gene was calculated as the sum of the fitness of all clones that contained that gene. This was followed by an initial rank ordering of gene fitness, followed by a specific assignment for genetic elements associated with multiple genes to the dominant gene identified in genetic element with the highest rank, with the subsequent removal of the fitness values from the non dominant genes in a genetic element.

Data was further analyzed by construction of receiver operator characteristics (“ROC”) according to traditional signal detection theory (T. Fawcett, “An introduction to ROC analysis,” Pattern Recog. Let. (2006)27:861-874). Data was categorized according to four standard classes—true positive, false positive, true negative, and false negative, using the fitness values for respective genetic elements per above and specific growth rates measured in the presence of 20 g/L 3-HP, using standard methods of analysis and cutoff values for fitness of 0.1, 1.0, 10 and 20 were chosen in an effort to optimize the range of true and false positive rates. A data point representing a genetic element of a clone was denoted a true positive if the reported fitness was greater than the cutoff value and the separately measured growth rate was significantly increased when compared with the negative control. A false positive had reported fitness that was greater than the cutoff value but a growth rate not significantly greater than that of the negative control. A clone was designated a true negative only if the corresponding fitness was less than the cutoff value and it yielded significantly reduced growth rates, i.e., not significantly greater than that of the negative control, and a false negative refers to a clone having a reduced fitness score but demonstrating an increased growth rate, i.e., significantly greater than that of the negative control.

An ROC curve is constructed by plotting the true positive rate (sensitivity) versus the false positive rate (1-specificity) (See T. E. Warnecke et al. Met. Engineering 10 (2008):154-165). Accordingly, it may be stated with confidence that clones (and their respective genetic elements) identified with increased fitness confer tolerance to 3-HP over the control.

Results

FIG. 1A, sheets 1-7, graphically shows the genes identified in the 3HPTGC for E. coli. In addition Table 1 gives cumulative fitness values as calculated above for the genes in the 3HPTGC.

As discussed above, 3-HP Toleragenic Complexes also were developed for the gram-positive bacterium Bacillus subtilis, for the yeast Saccharomyces cerevisiae, and for the bacterium Cupriavidus necator. These are depicted, respectively, in FIGS. 1B-D, sheets 1-7

### Example 2

### Additions of 3HPTGC Products, Part 1

Based on the above examples, and conceptualization of the 3HPTGC, it is possible to increase the 3-HP tolerance of a microorganism by adding limiting enzymatic conversion products (i.e., product(s) of an enzymatic conversion step) of the 3HPTGC. This example demonstrates the addition of some such products to increase 3-HP tolerance in E. coli.

Bacteria, Plasmids, and Media

Wild-type Escherichia coli K12 (ATCC #29425) was used for the preparation of genomic DNA. Mach1-T1® was obtained from Invitrogen (Carlsbad, Calif. USA).

3-HP Preparation

3-HP was obtained from TCI America (Portland, Oreg.). Significant acrylic acid and 2-oxydipropionic contamination was observed via HPLC analysis. Samples were subsequently treated by diethyl ether extraction to remove acrylic acid and a portion of the 2-oxydipropionic contaminants. Samples were then neutralized with 10 M NaOH to a final pH of 7.0. Considerable 3-HP polymerization was observed at neutral pH at concentrations in excess of approximately 35 g/L. Neutralized samples were centrifuged at 4000 rpm for 30 minutes at 4° C. The soluble 3-HP fraction was isolated from the solid polymer product and further analyzed by HPLC for a final quantification of concentration and purity of the working stock solution. The working stock solution was used for the selection, growth rates and MIC evaluations in this example.

Minimum Inhibitory Concentrations

The minimum inhibitory concentration (MIC) using commercially obtained 3-HP (TCI America, Portland, Oreg. USA, see 3-HP preparation above) was determined microaerobically in a 96 well-plate format. Overnight cultures of strains were grown in 5 ml LB (with antibiotic where appropriate). A 1 v/v % was used to inoculate a 15 ml conical tube filled to the top with MOPS minimal media and capped. After the cells reached mid exponential phase, the culture was diluted to an OD₆₀₀ of 0.200. The cells were further diluted 1:20 and a 10 ul aliquot was used to inoculate each well (˜10⁴ cells per well). The plate was arranged to measure the growth of variable strains or growth conditions in increasing 3-HP concentrations, 0-70 g/L, in 5 g/L increments, as well as either media supplemented with optimal supplement concentrations which were determined to be: 2.4 mM tyrosine (Sigma), 3.3 mM phenylalanine (Sigma), 1 mM tryptophan (Sigma), 0.2 mM p-hydroxybenzoic acid hydrazide (MP Biomedicals), 0.2 mM p-aminobenzoic acid (MP Biomedicals), 0.2 mM 2,3-dihydroxybenzoic acid (MP Biomedicals), 0.4 mM shikimic acid (Sigma), 2 mM pyridoxine hydrochloride (Sigma), 35 uM homoserine (Acros), 45 uM homocysteine thiolactone hydrochloride (MP Biomedicals), 0.5 mM oxobutanoate (Fluka), 5 mM threonine (Sigma).The minimum inhibitory 3-HP concentration (i.e., the lowest concentration at which there is no visible growth) and the maximum 3-HP concentration corresponding to visible cell growth (OD˜0.1) were recorded after 24 hours (between 24 and 25 hours, although data (not shown) indicated no substantial change in results when the time period was extended).

Results

3-HP tolerance of E. coli Mach1-T1® was increased by adding the supplements to the media. The supplementation described above resulted in the following MIC increases: 40% (tyrosine), 33% (phenylalanine), 33% (tryptophan), 33% (p-hydroxybenzoic acid hydrazide), 7% (p-aminobenzoic acid), 33% (2,3-didyroxybenzoic acid), 0% (pyridoxine hydrochloride), 33% (homoserine), 60% (homocysteine thiolactone hydrochloride), 7% (oxobutanoate), and 3% (threonine).

### Example 3

### Additions of 3HPTGC Products, Part 2 (using New Source of 3-HP)

Based on the above examples, and conceptualization of the 3HPTGC, it is possible to increase the 3-HP tolerance of a microorganism by adding limiting enzymatic conversion products (at least some of which alternatively may be termed “intermediates”) of the 3HPTGC. This example demonstrates the addition of putrescine, spermidine, cadaverine and sodium bicarbonateto increase 3-HP tolerance in E. coli. The concept of ‘limiting’ as used in this context refers to a hypothesized limitation that if overcome may demonstrate increased 3-HP tolerance by a subject microorganism or system. As a non-exclusive approach, such hypothesized limitation may be confirmed experimentally, as by a demonstration of increased tolerance to 3-HP upon addition of a particular enzymatic conversion product or other compound.

Bacteria, Plasmids, and Media

Wild-type Escherichia coli K12 (ATCC #29425) was used for the preparation of genomic DNA. M9 minimal and EZ rich media are described in Subsection II of the Common Methods Section.

3-HP Preparation

3-HP was obtained from Beta-propiolactone as described below in Subsection III of the Common Method Section.

Minimum Inhibitory Concentrations

The minimum inhibitory concentration (MIC) of 3-HP for E. coli (see 3-HP preparation above) was determined aerobically in a 96 well-plate format. Overnight cultures of strains were grown in 5 ml LB (with antibiotic where appropriate) at 37° C. in a shaking incubator. A 1 v/v% was used to inoculate 10 mL of M9 minimal media. After the cells reached mid-exponential phase, the culture was diluted to an OD₆₀₀ of 0.200. The cells were further diluted 1:20 and a 10 ul aliquot was used to inoculate each well (˜10⁴ cells per well). The plate was arranged to measure the growth of variable strains or growth conditions in increasing 3-HP concentrations, 0-100 g/L, in 10 g/L increments, in M9 minimal media, supplemented with putrescine (0.1 g/L, MP Biomedicals, Santa Ana, Calif. USA), cadaverine (0.1 g/L, MP Biomedicals) or spermidine (0.1 g/L, Sigma-Aldrich, St. Louis, Mo., USA) or sodium bicarbonate (20 mM, Fisher Scientific, Pittsburgh, Pa. USA) (values in parentheses indicate final concentrations in media). The minimum inhibitory 3-HP concentration (i.e., the lowest concentration at which there is no visible growth) and the maximum 3-HP concentration corresponding to visible cell growth (OD˜0.1) were recorded after 24 hours (between 24 and 25 hours, although data (not shown) indicated no substantial change in results when the time period was extended). The MIC endpoint is the lowest concentration of compound at which there was no visible growth.

Results

3-HP tolerance of E. coli was increased by adding the polyamines putrescine, spermidine and cadaverine to the media. Minimum inhibitory concentrations (MICs) for E. coli K12 in control and supplemented media were as follows: in M9 minimal media supplemented with putrescine 40 g/L, in M9 minimal media supplemented with spermidine 40 g/L, in M9 minimal media supplemented with cadavarine 30 g/L. Minimum inhibitory concentrations (MICs) for added sodium bicarbonate in M9 minimal media was 30 g/L. The Minimum inhibitory concentrations (MICs) for E. coli K12 in 100 g/L stock solution 3-HP was 20 g/L.

In view of the increase over the control MIC with sodium bicarbonate supplementation, other alteration, such as regulation and/or genetic modification of carbonic anhydrase (not presently shown in FIG. 1A1-7, but related directly to HCO₃⁻), such as providing a heterologous nucleic acid sequence to a cell of interest, where that nucleic acid sequence encodes a polypeptide possessing carbonic anhydrase activity are considered of value to increase tolerance to 3-HP (such as in combination with other alterations of the 3HPTGC). Similarly, and as supported by other data provided herein, alterations of the enzymatic activities, such as by genetic modification(s) of enzyme(s) along the 3HPTGC pathway portions that lead to arginine, putrescine, cadaverine and spermidine, are considered of value to increase tolerance to 3-HP (such as in combination with other alterations of the 3HPTGC).

### Example 4

### Genetic Modification of aroH for Increased 3-HP Tolerance

Based on the identification of the tyrA-aroF operon as a genetic element conferring tolerance to 3-HP at increased copy, this enzymatic activity was further examined. The wild type aroF gene is inhibited by increasing concentrations of end products tyrosine and phenylalanine. However, to bypass this inherent feedback inhibition control, a feedback resistant mutant of the aroH gene was obtained and introduced into a cell as follows.

Clone Construction

PCR was used to amplify the E. coli K12 genomic DNA corresponding to the aroF-tyrA region with primers designed to include the upstream aroFp promoter and the rho-independent transcriptional terminators. Ligation of the purified, fragmented DNA with the pSMART-kanamycin vectors was performed with the CloneSMART kit (Lucigen, Middleton, Wis. USA) according to manufacturer's instructions. The ligation product was then transformed into chemically competent Mach1-T1® E. coli cells (Invitrogen, Carlsbad, Calif. USA), plated on LB+kanamycin, and incubated at 37° C. for 24 hours. To confirm the insertion of positive transformants, plasmids were isolated from clones using a Qiaprep Spin MiniPrep Kit from Qiagen (Valencia, Calif.) and sequenced (Macrogen, South Korea).

Plasmids containing the wild-type aroH gene (CB202) and a mutant version exhibiting resistance to tryptophan feedback inhibition (CB447) via a single amino acid change (G149D) were obtained from Ray et al (Ray, J. M., C. Yanofsky, and R. Baurele, Mutational analysis of the catalytic and feedback sites of the tryptophan-sensitive 3-deoxy-D-arabino-heptulosante-7-phosphate synthase of Escherichia coli. J Bacteriol, 1988. 170(12):p. 5500-6.). These plasmids were constructed with the pKK223-3 backbone vector containing the ptac promoter and rrNBT1 transcriptional terminator. The aroH inset DNA was amplified according to traditional PCR methodology with primers designed to include both the promoter and terminator. Purified PCR products were ligated with the pBT-1 plasmid and transformed into electrocompetent Mach1-T1® (Lynch, M. D. and R. T. Gill, A series of broad host range vectors for stable genomic library construction. Biotechnology and Bioengineering, 2006. 94(1): p. 151-158). The resulting plasmid sequence is given in (SEQ ID NO:001). Optimal induction levels were determined by minimum inhibitory concentration assays to be 0.001 mM IPTG.

MIC Comparisons

MIC evaluations were conducted as described for Example 1. A Mach1-T1® cell culture comprising the aroH mutant was compared with a control cell culture, both in MOPS minimal media.

Results

As measured by fold increase in MIC, the cells comprising the aroH mutant exhibited a MIC 1.4 times greater than the control MIC. This represents a 40 percent improvement.

Accordingly, this example demonstrates one of many possible genetic modification approaches to increasing 3-HP tolerance in a selected cell, based on knowledge of the importance of the 3HPTGC in 3-HP tolerance.

### Example 5

### Genetic Modification via Cyanase Introduction for Increased 3-HP Tolerance

A plasmid clone containing the cynTS genes from E. coli K12 was obtained from selections described in Example 1. This plasmid called pSMART-LC-Kan-cynTS was isolated and purified according to standard methods. (Sequencing of the plasmid revealed a final sequence (SEQ ID NO:002)). Purified plasmid was retransformed into E. coli K12 by standard techniques and MIC measured as described above in Example 3.

3-HP Tolerance Improvement by the Plasmid Containing the cynTS Genes.

Minimum inhibitory concentrations (MICs) of 3-HP for E. coli K12 and E. coli K12+pSMART-LC-Kan-cynTS in M9 minimal media were 30 g/L, and 50 g/L respectively. Thus, an over sixty percent improvement in the MIC, signifying an increase in 3-HP tolerance, was observed in this example which comprised only one genetic modification of the 3HPTGC in the E. coli host cell.

Accordingly, this example again demonstrates one of many possible genetic modification approaches to increasing 3-HP tolerance in a selected cell, based on knowledge of the importance of the 3HPTGC in 3-HP tolerance and appropriate use of that knowledge.

### Example 6

### Genetic Modification/Introduction of Malonyl-CoA Reductase for 3-HP Production in E. coli DF40

The nucleotide sequence for the malonyl-coA reductase gene from Chloroflexus aurantiacus was codon optimized for E. coli according to a service from DNA 2.0 (Menlo Park, Calif. USA), a commercial DNA gene synthesis provider. This gene sequence incorporated an EcoRI restriction site before the start codon and was followed by a HindIII restriction site. In addition a Shine Delgarno sequence (i.e., a ribosomal binding site) was placed in front of the start codon preceded by an EcoRI restriction site. This gene construct was synthesized by DNA 2.0 and provided in a pJ206 vector backbone. Plasmid DNA pJ206 containing the synthesized mcr gene was subjected to enzymatic restriction digestion with the enzymes EcoRI and HindIII obtained from New England BioLabs (Ipswich, Mass. USA) according to manufacturer's instructions. The digestion mixture was separated by agarose gel electrophoresis, and visualized under UV transillumination as described in Subsection II of the Common Methods Section. An agarose gel slice containing a DNA piece corresponding to the mcr gene was cut from the gel and the DNA recovered with a standard gel extraction protocol and components from Qiagen (Valencia, Calif. USA) according to manufacturer's instructions. An E. coli cloning strain bearing pKK223-aroH was obtained as a kind a gift from the laboratory of Prof. Ryan T. Gill from the University of Colorado at Boulder. Cultures of this strain bearing the plasmid were grown by standard methodologies and plasmid DNA was prepared by a commercial miniprep column from Qiagen (Valencia, Calif. USA) according to manufacturer's instructions. Plasmid DNA was digested with the restriction endonucleases EcoRI and HindIII obtained from New England Biolabs (Ipswich, Mass. USA) according to manufacturer's instructions. This digestion served to separate the aroH reading frame from the pKK223 backbone. The digestion mixture was separated by agarose gel electrophoresis, and visualized under UV transillumination as described in Subsection II of the Common Methods Section. An agarose gel slice containing a DNA piece corresponding to the backbone of the pKK223 plasmid was cut from the gel and the DNA recovered with a standard gel extraction protocol and components from Qiagen according to manufacturer's instructions.

Pieces of purified DNA corresponding to the mcr gene and pK223 vector backbone were ligated and the ligation product was transformed and electroporated according to manufacturer's instructions. The sequence of the resulting vector termed pKK223-mcr (SEQ ID NO:003) was confirmed by routine sequencing performed by the commercial service provided by Macrogen(USA). pKK223-mcr confers resistance to beta-lactamase and contains the kgd gene of m. tuberculosis under control of a ptac promoter inducible in E. coli hosts by IPTG.

The expression clone pKK223-mcr and pKK223 control were transformed into both E. coli K12 and E. coli DF40 via standard methodologies. (Sambrook and Russell, 2001).

3-HP production of E. coli DF40+pKK223-MCR was demonstrated at 10 mL scale in M9 minimal media. Cultures of E. coli DF40, E. coli DF40+pKK223, and E. coli DF40+pKK223-MCR were started from freezer stocks by standard practice (Sambrook and Russell, 2001) into 10 mL of LB media plus 100 ug/mL ampicillin where indicated and grown to stationary phase overnight at 37 degrees shaking at 225 rpm overnight. In the morning, these cells from these cultures were pelleted by centrifugation and resuspended in 10 mL of M9 minimal media plus 5% (w/v) glucose. This suspension was used to inoculate 5% (v/v) fresh 10 ml cultures [5% (v/v)] in M9 minimal media plus 5% (w/v) glucose plus 100 ug/mL ampicillin where indicated. These cultures were grown in at least triplicate, with 1 mM IPTG added. To monitor growth of these cultures, Optical density measurements (absorbance at 600 nm, 1 cm pathlength), which correlate to cell numbers, were taken at time=0 and every 2 hrs after inoculation for a total of 12 hours. After 12 hours, cells were pelleted by centrifugation and the supernatant collected for analysis of 3-HP production as described under “Analysis of cultures for 3-HP production” in the Common Methods section.

Results

Preliminary final titers of 3-HP in these 10 mL cultures was calculated after HPLC analysis and 3.19+/−1.041 mM 3-HP. It is acknowledged that there is likely co-production of malonate semialdehyde or possibly another aldehyde that is indistinguishable from 3-HP by our current HPLC analysis.

### Example 7

### Development of a Nucleic Acid Sequence Encoding a Protein Sequence Comprising Oxaloacetate Alpha—Decarboxylase Activity (Partial Prophetic)

Several 2-keto acid decarboxylases with a broad substrate range have been previously characterized (Pohl, M., Sprenger, G. A., Muller, M., A new perspective on thiamine catalysis. Current Opinion in Biotechnology, 15(4), 335-342 (2004)). Of particular interest is an enzyme from M. tuberculosis, alpha-ketoglutarate decarboxylase, which has been purified and characterized (Tian, J., Bryk, R. Itoh, M., Suematsu, M., and Carl Nathan, C. Variant tricarboxylic acid cycle in Mycobacterium tuberculosis: Identification of alpha-ketoglutarate decarboxylase. PNAS. Jul. 26, 2005 vol. 102(30): 10670-10677; Stephanopoulos, G., Challenges in engineering microbes for biofuels production. Science, 2007. 315(5813):801-804). The reaction carried out by this enzyme is depicted in FIG. 7B (FIG. 7A showing the predominant known chemical reaction by the enzyme encoded by the native kgd gene). The native kgd gene has previously been cloned, expressed and purified from E. coli without technical difficulty or toxic effects to the host strain (Tian, J., Bryk, R. Itoh, M., Suematsu, M., and Carl Nathan, C. Variant tricarboxylic acid cycle in Mycobacterium tuberculosis: Identification of alpha-ketoglutarate decarboxylase. PNAS. Jul. 26, 2005 vol. 102(30):10670-10677; Stephanopoulos, G., Challenges in engineering microbes for biofuels production. Science, 2007. 315(5813):801-804). This enzyme has also been chosen as it is unlikely to associated with the alpha-ketoglutarate dehydrogenase. Of additional interest is that a convenient colorimetric method has been developed to assay this enzymatic activity. The kgd enzyme is evolved as provided herein to have a measurable enzymatic function depicted in FIG. 7B, the decarboxylation of oxaloacetate to malonate semialdehyde. The technical work to achieve this relies largely upon traditional selection and screening of mutants of the alpha-keto-glutarate decarboxylase that have the desired oxaloacetate alpha-decarboxylase activity.

As a first step a mutant library is constructed of the kgd gene that will be used for selections or screening. The protein sequence for the alpha-ketoglutarate decarboxylase from M. tuberculosis was codon optimized for E. coli according to a service from DNA 2.0 (Menlo Park, Calif. USA), a commercial DNA gene synthesis provider. The nucleic acid sequence was synthesized with an eight amino acid N-terminal tag to enable affinity based protein purification. This gene sequence incorporated an NcoI restriction site overlapping the gene start codon and was followed by a HindIII restriction site. In addition a Shine Delgarno sequence (i.e., a ribosomal binding site) was placed in front of the start codon preceded by an EcoRI restriction site. This gene construct was synthesized by DNA 2.0 and provided in a pJ206 vector backbone.

A circular plasmid based cloning vector termed pKK223-kgd for expression of the alpha-ketoglutarate decarboxylase in E. coli was constructed as follows. Plasmid DNA pJ206 containing the gene synthesized kgd gene was subjected to enzymatic restriction digestion with the enzymes EcoRI and HindIII obtained from New England BioLabs (Ipswich, Mass. USA) according to manufacturer's instructions. The digestion mixture was separated by agarose gel electrophoresis, and visualized under UV transillumination as described in Subsection II of the Common Methods Section. An agarose gel slice containing a DNA piece corresponding to the kgd gene was cut from the gel and the DNA recovered with a standard gel extraction protocol and components from Qiagen according to manufacturer's instructions. An E. coli cloning strain bearing pKK223-aroH was obtained as a kind a gift from the laboratory of Prof. Ryan T. Gill from the University of Colorado at Boulder. Cultures of this strain bearing the plasmid were grown by standard methodologies and plasmid DNA was prepared by a commercial miniprep column from Qiagen (Valencia, Calif. USA) according to manufacturer's instructions. Plasmid DNA was digested with the restriction endonucleases EcoRI and HindIII obtained from New England BioLabs (Ipswich, Mass. USA) according to manufacturer's instructions. This digestion served to separate the aroH reading frame from the pKK223 backbone. The digestion mixture was separated by agarose gel electrophoresis, and visualized under UV transillumination as described in Subsection II of the Common Methods Section. An agarose gel slice containing a DNA piece corresponding to the backbone of the pKK223 plasmid was cut from the gel and the DNA recovered with a standard gel extraction protocol and components from Qiagen (Valencia, Calif. USA) according to manufacturer's instructions.

Pieces of purified DNA corresponding to the kgd gene and pKK223 vector backbone were ligated and the ligation product was transformed via electroporation according to manufacturer's instructions. The sequence of the resulting vector termed pKK223-kgd (SEQ ID NO:004) was confirmed by routine sequencing performed by the commercial service provided by Macrogen (Rockville, Md. USA). pKK223-kgd confers resistance to beta-lactamase and contains the kgd gene of M. tuberculosis under control of a ptac promoter inducible in E. coli hosts by IPTG.

Plasmid pKK223-kgd was propagated and purified DNA prepared by standard methodologies. Plasmids were introduced into XL1-Red chemically competent cells (Stratagene, LaJolla, Calif.) in accordance with the manufacturer's instructions, plated onto LB+100 micrograms/mL ampicillin, and incubated at 37° C. for >24 hours. Dilution cultures with 1/1000 of the original transformation volume were plated on LB+100 micrograms/mL ampicillin in triplicate. Greater than 1000 colonies were obtained, corresponding to approximately 10⁷ mutant cells per transformation. Colonies were harvested by gently scraping the plates into TB media. The cultures were immediately resuspended by vortexing, and aliquoted into 1 mL freezer stock cultures with a final glycerol concentration of 15% (v/v) (Sambrook and Russell, 2001). The remainder of the culture was pelleted by centrifugation for 15 minutes at 3000 rpm. Plasmid DNA was extracted according to the manufacturer's instructions using a HiSpeed Plasmid Midi Kit (Qiagen, Valencia, Calif.). Purified plasmid DNA from each mutant library was introduced into E. coli 10GF′ (Lucigen, Middleton, Wis. USA) by electroporation. 1/1000 volume of this transformation was plated on LB+kanamycin in triplicate to determine transformation efficiency and adequate transformant numbers (>10̂6).

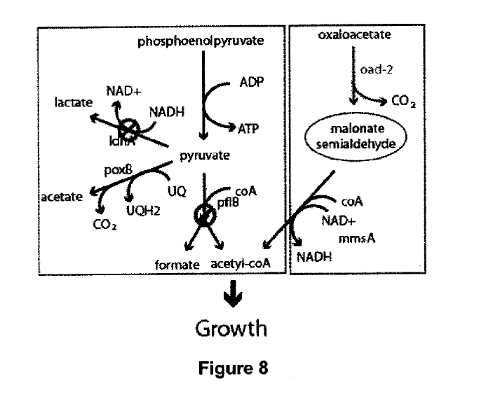

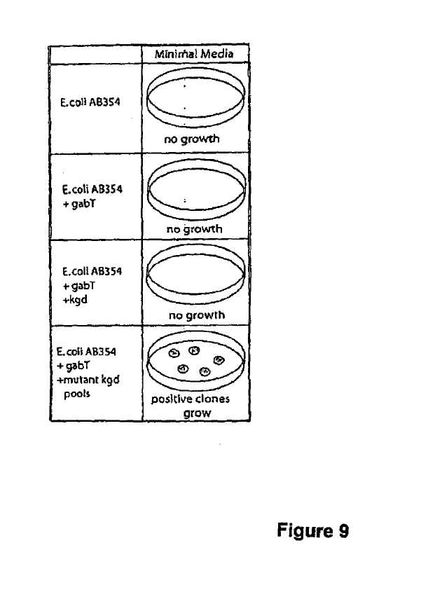

The selection based approach described herein allows for the rapid identification of a kgd mutant with oxaloacetate alpha-decarboxylase activity. An available strain of E. coli, strain AB354, is used as a host for the selection (Bunch, P. K., F. Mat-Jan, N. Lee, and D. P. Clark. 1997. The IdhA gene encoding the fermentative lactate dehydrogenase of Escherichia coli. Microbiology 143:187-195). This auxotrophic E. coli strain has a mutation in panD, encoding aspartate decarboxylase. The product of this reaction, beta-alanine is an essential intermediate in the synthesis of pantothenate, a precursor to coenzyme A. The block in coenzyme A synthesis confers an inability of this E. coli strain to grow on minimal media without supplementation (Cronoan, J. E., Little, K. J., Jackowski, S.; Genetic and Biochemical Analyses of Pantothenate Biosynthesis in Escherichia coli and Salmonella typhimurium. J. of Bacteriology, 149(3), 916-922 (1982); Cronan, J. E., Beta-Alanine Synthesis in Escherichia coli J. of Bacteriology, 141(3), 1291-1297 (1980)) (See FIG. 8). The expression of gabT from R. norvegicus confers beta-alanine aminotransferase activity to E. coli (Tunnicliff, G.; Ngo, T. T.; Rojo-Ortega, J. M.; Barbeau, A.; The inhibition by substrate analogues of gamma-aminobutyrate aminotransferase from mitochondria of different subcellular fractions of rat brain Can. J. Biochem. 55, 479-484 (1977)). This enzyme can utilize malonate semialdehyde as a substrate to produce beta-alanine. A strain of E. coli AB354 expressing gabT (E. coli AB354+gabT) in addition to a mutant kgd gene having oxaloacetate alpha-decarboxylase activity is capable of producing the metabolite beta-alanine and have a restored ability to grown on minimal media. Expected results of the selection are depicted in FIG. 9.

Similar to the kgd gene, a codon and expression optimized R. norvegicus gabT gene is obtained via gene synthesis from the commercial provider DNA 2.0 (Menlo Park, Calif. USA). It is subsequently cloned into an expression plasmid.

The mutant library of kgd genes is introduced into E. coli strain AB354 expressing the gabT gene. This population will then be grown on minimal media plates. Individual mutants expressing the desired oxaloacetate alpha-decarboxylase activity are expected to show a restored ability to form colonies under these conditions. These clones are isolated and the mutant proteins they express subsequently are selected for oxaloacetate alpha-decarboxylase activity.

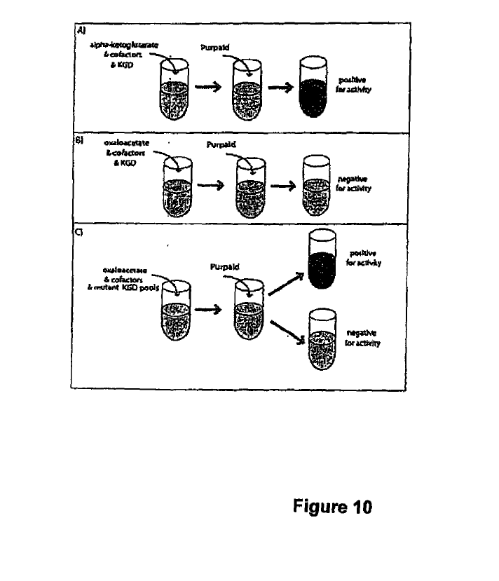

With the successful construction selection of a mutant kgd library for oxaloacetate alpha-decarboxylase activity, it will be necessary to confirm that these mutants have the desired enzymatic activity. Thus, mutants positive for oxaloacetate alpha-decarboxylase activity are confirmed for alpha-decarboxylase activity. To accomplish this, a colorimetric screening approach is taken from current standard methodologies. This approach is illustrated in FIG. 10. This approach necessitates the expression and purification of the mutant enzymes and reaction with the purified enzyme, its cofactor (thiamin pyrophosphate) and the appropriate substrate. Protein expression and purification is performed with standard methodologies.

### Example 8

### One-Liter Scale Bio-Production of 3-HP using E. coli DF40+pKK223+MCR

Using E. coli strain DF40+pKK223+MCR that was produced in accordance with Example 6 above, a batch culture of approximately 1 liter working volume was conducted to assess microbial bio-production of 3-HP.

E. coli DF40+pKK223+MCR was inoculated from freezer stocks by standard practice (Sambrook and Russell, 2001) into a 50 mL baffled flask of LB media plus 200 μg/mL ampicillin where indicated and grown to stationary phase overnight at 37° C. with shaking at 225 rpm. In the morning, this culture was used to inoculate (5% v/v) a 1-L bioreactor vessel comprising M9 minimal media plus 5% (w/v) glucose plus 200 μg/mL ampicillin, plus 1 mM IPTG, where indicated. The bioreactor vessel was maintained at pH 6.75 by addition of 10 M NaOH or 1 M HCl, as appropriate. The dissolved oxygen content of the bioreactor vessel was maintained at 80% of saturation by continuous sparging of air at a rate of 5 L/min and by continuous adjustment of the agitation rate of the bioreactor vessel between 100 and 1000 rpm. These bio-production evaluations were conducted in at least triplicate. To monitor growth of these cultures, optical density measurements (absorbance at 600 nm, 1 cm path length), which correlates to cell number, were taken at the time of inoculation and every 2 hrs after inoculation for the first 12 hours. On day 2 of the bio-production event, samples for optical density and other measurements were collected every 3 hours. For each sample collected, cells were pelleted by centrifugation and the supernatant was collected for analysis of 3-HP production as described per “Analysis of cultures for 3-HP production” in the Common Methods section, below. Preliminary final titer of 3-HP in this 1-liter bio-production volume was calculated based on HPLC analysis to be 0.7 g/L 3-HP. It is acknowledged that there is likely co-production of malonate semialdehyde, or possibly another aldehyde, or possibly degradation products of malonate semialdehyde or other aldehydes, that are indistinguishable from 3-HP by this HPLC analysis.

### Example 9

### Tolerance Plus Bio-Production Pathway

Using methods known to those skilled in the art, including those provided in the Common Methods Section, below, and also using specific methods from the other examples herein as to making and incorporating nucleic acid sequences to provide increased 3-HP tolerance and to provide 3-HP bio-production, genetic modifications are made to a selected microorganism to provide heterologous nucleic acid sequences that increase both 3-HP tolerance and 3-HP production above levels found in the non-modified microorganism. A plasmid or other vector or a DNA sequence (for direct incorporation) is constructed that comprises one or more nucleic acid sequences that encode for enzyme(s) or other polypeptide(s) that, when combined into and expressed in the selected microorganism, increase(s) tolerance to 3-HP by modifying one or more aspects of the 3HPTGC. That or a different plasmid or other vector or a DNA sequence (for direct incorporation) is constructed to comprise one or more nucleic acid sequences that encode for enzyme(s) or other polypeptide(s) that, when expressed in the selected microorganism, provide for (or increase) 3-HP bio-production.

In the case of plasmids, the plasmid(s) is/are contacted with the selected microorganism under suitable conditions to promote transformation, and transformed microorganisms are selected for and identified. In the case of other vectors or the DNA sequence(s), these are introduced to the selected microorganism by methods well-known to those skilled in the art. Selection for transformed recombinant microorganisms likewise may be conducted according to methods well-known to those skilled in the art.

A first particular resultant recombinant microorganism comprises enhanced 3-HP tolerance and bio-production capabilities compared to the control, non-tolerance-modified microorganism, in which 3-HP tolerance is at least 20 percent greater than tolerance of the non-tolerance-modified control and 3-HP bio-production is at least 20 percent greater than 3-HP bio-production of the non-tolerance-modified control. 3-HP tolerance is assessed by a 24-hour Minimum Inhibitory Concentration (MIC) evaluation based on the MIC protocol provided in the Common Methods Section. 3-HP bio-production is based on a batch culture comparison lasting for at least 24 hours past lag phase, and final 3-HP titers are determined using the HPLC methods provided in the Common Methods Section.

It is appreciated that iterative improvements using the strategies and methods provided herein, and based on the discoveries of the interrelationships of the pathways and pathway portions of the 3HPTGC, may lead to even greater 3-HP tolerance and more elevated 3-HP titers at the conclusion of a 3-HP bio-production event.

Accordingly, it is within the scope of the present invention to produce, and to utilize in bio-production methods and systems, including industrial bio-production systems for production of 3-HP, a recombinant microorganism genetically engineered to modify one or more aspects of the 3HPTGC effective to increase tolerance to 3-HP (and, in some embodiments, also 3-HP bio-production) by at least 20 percent over control microorganism lacking the one or more tolerance-altering modifications.

### Example 10

### Demonstration of Suitable Metrics for Comparison of Tolerance Improvements

Growth rate data was determined for the following species under the specified conditions, aerobic and anaerobic, across a range of 3-HP concentrations in the cell cultures. This demonstrates methods that may be used to assess differences between a control and a treatment microorganism. These or other methods may be used to demonstrate tolerance differences for various embodiments of the present invention.

As shown in the accompanying figures, FIGS. 6A-O, the data may be evaluated and presented in a number of ways: a “toleragram” (showing growth rates at different 3-HP concentrations); change in optical density over the evaluation period; and number of cell doublings over the evaluation period.

These are provided to indicate non-limiting methodologies and approaches to assessing changes in tolerance, including microorganism and culture system tolerance, in addition to the use of MIC evaluations.

The following methods were used to generate the data in the noted figures. Example 17 provides a direct comparison of one genetic modification of the 3HPTC with a control using a growth rate-based toleragram over a 24-hour period.

### E. coli Aerobic

Overnight cultures of wild-type E. coli BW25113 were grown in triplicate in 5 mL standard LB medium. 100 uL of overnight cultures were used to inoculate triplicate 5 mL samples of M9 minimal medium+3HP, containing 47.7 mM Na₂HPO₄, 22 mM KH₂PO₄, 8.6 mM NaCl, 18.7 mM NH₄Cl, 2 mM MgSO₄, 0.1 mM CaCl₂, and 0.4% glucose, with 3HP concentrations ranging from 0-50 g/L. Starting OD₆₀₀ ranged from 0.02-0.08. Cultures were incubated at 37 C for about 24 hours, and OD₆₀₀ was recorded every 1-2 hours for the first 8 hours with a final OD₆₀₀ recorded at about 24 hours. Maximum specific growth rates (μₘₐₓ) were calculated by determining the optimal fit of exponential trend lines with OD data for the evaluation period. Specific changes in OD₆₀₀ over approximately 24 hours (Δ_{24 hr}OD₆₀₀) were calculated as the difference in t=24 hr and t=0 optical density, Δ_{24 hr}OD₆₀₀=(ODₜ₌₂₄)−(ODₜ₌₀). Specific number of doublings (N_{d}) were calculated by solving for N in the equation 2^{N}=(ODₜ₌₂₄)/(ODₜ₌₀).

E. coli Anaerobic

Overnight cultures of wild-type E. coli BW25113 were grown in triplicate in 5 mL standard LB medium. 100 uL of overnight cultures were used to inoculate triplicate 5 mL samples of M9 minimal medium+3HP, containing 47.7 mM Na₂HPO₄, 22 mM KH₂PO₄, 8.6 mM NaCl, 18.7 mM NH₄Cl, 2 mM MgSO₄, 0.1 mM CaCl₂, and 0.4% glucose, with 3HP concentrations ranging from 0-50 g/L. Starting OD₆₀₀ ranged from 0.02-0.08. Cultures were sparged with CO₂ for 10 seconds, sealed, and incubated at 37 C for about 24 hours. OD₆₀₀ was recorded every 1-2 hours during the first 8 hours with a final OD₆₀₀ recorded at about 24 hours. For each data point the sample was opened, sampled, re-sparged with CO₂, and sealed once again. Maximum specific growth rates (μₘₐₓ) were calculated by determining the optimal fit of exponential trend lines with OD data for the evaluation period. Specific changes in OD₆₀₀ over approximately 24 hours (Δ_{24 hr}OD₆₀₀) were calculated as the difference in t=24 hr and t=0 optical density, Δ_{24 hr}OD₆₀₀=(ODₜ=24)−(ODₜ₌₀). Specific number of doublings (N_{d}) were calculated by solving for N in the equation 2^{N}=(ODₜ₌₂₄)/(ODₜ₌₀).

Bacillus Subtilis Aerobic

Overnight cultures of wild-type B. Subtilis were grown in triplicate in 5 mL standard LB medium. 100 uL of overnight cultures were used to inoculate triplicate 5 mL samples of M9 minimal medium+3HP+glutamate supplementation, containing 47.7 mM Na₂HPO₄, 22 mM KH₂PO₄, 8.6 mM NaCl, 18.7 mM NH₄Cl, 2 mM MgSO₄, 0.1 mM CaCl₂, 0.4% glucose, and 10 mM glutamate, with 3HP concentrations ranging from 0-50 g/L. Starting OD₆₀₀ ranged from 0.02-0.08. Cultures were incubated at 37 C for about 24 hours, and OD₆₀₀ was recorded every 1-2 hours for the first 8 hours with a final OD₆₀₀ recorded at about 24 hours. Maximum specific growth rates (μₘₐₓ) were calculated by determining the optimal fit of exponential trend lines with OD data for the evaluation period. Specific changes in OD₆₀₀ over approximately 24 hours (Δ_{24 hr}OD₆₀₀) were calculated as the difference in t=24 hr and t=0 optical density, Δ_{24 hr}OD₆₀₀=(ODₜ₌₂₄)−(ODₜ₌₀). Specific number of doublings (N_{d}) were calculated by solving for N in the equation 2^{N}=(ODₜ₌₂₄)/(ODₜ₌₀).

S. cervisiae Aerobic

Overnight cultures of S. cervisiae were grown in triplicate in 5 mL standard YPD medium containing 10 g/L yeast extract, 20 g/L peptone, and 2% glucose. 100 uL of overnight cultures were used to inoculate triplicate 5 mL samples of SD minimal medium (without vitamins)+3HP, containing 37.8 mM (NH₄)₂SO₄, 8.1 uM H₃BO₃, 0.25 uM CuSO₄, 0.6 uM KI, 1.25 uM FeCl₃, 2.65 uM MnSO₄, 1 uM Na₂MoO₄, 2.5 uM ZnSO₄, 6.25 mM KH₂PO₄, 0.86 mM K₂HPO₄, 4.15 mM MgSO₄, 1.71 mM NaCl, 0.90 mM CaCl₂, and 2% glucose, with 3HP concentrations ranging from 0-50 g/L. Starting OD₆₀₀ ranged from 0.03-0.08. Cultures were sparged with CO₂ for 10 seconds, sealed, and incubated at 30C for about 24 hours. OD₆₀₀ was recorded every 1-2 hours for the first 8-12 hours with a final OD₆₀₀ recorded at about 24 hours. Maximum specific growth rates (μₘₐₓ) were calculated by determining the optimal fit of exponential trend lines with OD data for the evaluation period. Specific changes in OD₆₀₀ over approximately 24 hours (Δ_{24 hr}OD₆₀₀) were calculated as the difference in t=24 hr and t=0 optical density, Δ_{24 hr}OD₆₀₀=(ODₜ₌₂₄)−(ODₜ₌₀). Specific number of doublings (N_{d}) were calculated by solving for N in the equation 2^{N}=(ODₜ₌₂₄)/(ODₜ₌₀).

S. cervisiae Anaerobic

Overnight cultures of S. cervisiae were grown in triplicate in 5 mL standard YPD medium containing 10 g/L yeast extract, 20 g/L peptone, and 2% glucose. 100 uL of overnight cultures were used to inoculate triplicate 5 mL samples of SD minimal medium (without vitamins)+3HP, containing 37.8 mM (NH₄)₂SO₄, 8.1 uM H₃BO₃, 0.25 uM CuSO₄, 0.6 uM KI, 1.25 uM FeCl₃, 2.65 uM MnSO₄, 1 uM Na₂MoO₄, 2.5 uM ZnSO₄, 6.25 mM KH₂PO₄, 0.86 mM K₂HPO₄, 4.15 mM MgSO₄, 1.71 mM NaCl, 0.90 mM CaCl₂, and 2% glucose, with 3HP concentrations ranging from 0-50 g/L. Starting OD₆₀₀ ranged from 0.03-0.08. Cultures were sparged with CO₂ for 10 seconds, sealed, and incubated at 30 C for about 24 hours. OD₆₀₀ was recorded every 1-2 hours for the first 8-12 hours with a final OD₆₀₀ recorded at about 24 hours. For each data point the sample was opened, sampled, re-sparged with CO₂, and sealed once again. Maximum specific growth rates (μₘₐₓ) were calculated by determining the optimal fit of exponential trend lines with OD data for the evaluation period. Specific changes in OD₆₀₀ over approximately 24 hours (Δ_{24 hr}OD₆₀₀) were calculated as the difference in t=24 hr and t=0 optical density, Δ_{24 hr}OD₆₀₀=(ODₜ₌₂₄)−(ODₜ₌₀). Specific number of doublings (N_{d}) were calculated by solving for N in the equation 2^{N}=(ODₜ₌₂₄)/(ODₜ₌₀).

### Example 11

### Genetic Modification by Introduction of Genes Identified as able to Increase Microorganism Tolerance to 3-HP

### Background

Genetic elements containing one to several genes have been identified by the SCALES 3-HP tolerance data as important to 3-HP tolerance. In order to develop an optimal combination of these elements suitable to imparting greater tolerance on an organism, a number of these genetic elements have been cloned into a series of compatible plasmids containing different origins of replication and selection markers. As such, combinations of these compatible plasmids can be transformed into cell lines in order to assess a combinatorial affect on 3-HP tolerance. The parent plasmid vectors containing the different origins of replication and selection markers are identified in Table 4A, which provides SEQ ID numbers (SEQ ID NOs:005-012 and 183-186) for each such parent plasmid vectors. These plasmids were used to construct the plasmids describes below, and these plasmids, without insert, were also used for constructing control cell lines for tolerance MIC testing.

### Method A: Plasmid Design and Construction of Toleragenic Genetic Elements by Gene Synthesis

A single plasmid comprising a number of identified genetic elements was constructed in a manner that a plurality of other plasmids could easily be constructed (some of which were constructed as described below). These operons, including a constitutive E. coli promoter, ribosome binding sites, and open region frames of these genetic elements, were combined in the single plasmid, which was produced by the gene synthesis services of DNA2.0 (Menlo Park, Calif. USA), a commercial DNA gene synthesis provider. Each of the open reading frames for producing proteins was codon optimized according to the services of DNA2.0. Additionally, restriction sites were incorporated between each operon and gene to generate plasmids capable of expressing all combinations of these proteins through a series of restriction digests and self ligation. Other features of this constructs include an rrnB terminator sequence after the final operons and mosaic ends containing AfeI restriction sites flanking each end of the coding region for use with a EZ::TN™ Transposon system obtained from EPICENTRE (Madison, Wis.) for future genomic incorporation of these elements into strains. This constructed plasmid was provided in a pJ61 vector backbone. The sequence of the resulting vector, termed pJ61:25135, is provided as SEQ ID NO:012 (see Table 4A).

By the method described herein various nucleic acid sequences encoding enzymes that catalyze enzymatic conversion steps of the 3HPTGC were introduced into the pJ61:25135 plasmid. As shown in Table 4B, the pJ61:25135 plasmid (in Table 4A) was variously modified to contain gene optimized sequences for CynS and CynT expressed under a modified Ptrc promoter located between PmlI and SfoI restriction sites, AroG expressed under a PtpiA promoter located between SfoI and SmaI restriction sites (SEQ ID NO:013), SpeD, SpeE,and SpeF expressed under a modified Ptrc promoter located between SmaI and ZraI restriction sites (SEQ ID NO:014), ThrA expressed under a PtalA promoter located between ZraI and HpaI restriction sites (SEQ ID NO:015), Asd expressed under a PrpiA promoter located between HpaI and PmeI restriction sites (SEQ ID NO:016), CysM expressed under a Ppgk promoter located between PmeI and ScaI restriction sites (SEQ ID NO:017), IroK expressed under a PtpiA promoter located between ScaI and NaeI restriction sites, and IlvA expressed under a PtalA promoter located between NaeI and EcoICRI restriction sites (SEQ ID NO:018). Each of these restriction sites is unique within the pJ61:25135 plasmid.

To create a set of plasmids containing each of these single operons, a series of restrictions and self-ligations are performed. As such, any operons can be isolated by removal of the DNA sequences between its flanking restriction sites and the EcoICRI and PmlI sites flanking the entire protein coding region of the plasmid. For example, the plasmid comprising the operon comprising the AroG polypeptide, expressed under a PtpiA promoter and located between SfoI and SmaI restriction sites, was created by first digesting the pJ61:25135 plasmid with PmlI and SfoI obtained from New England BioLabs (Ipswich, Mass. USA) according to manufacturer's instructions. The resulting DNA was then self-ligated with T4 DNA ligase obtained from New England BioLabs (Ipswich, Mass. USA) according to manufacturer's instructions, and transformed into E. coli K12. Individual colonies from this E. coli K12 transformation were grown in liquid culture and plasmids from individual colonies were isolated using a Qiagen Miniprep kit (Valencia, Calif. USA) according to manufacturer's instructions, The isolated plasmids were screened by restriction digests with AfeI, and correct plasmids were carried on the next round of restriction and self ligation. In the second round, these plasmids were subjected to restriction with SmaI and EcoICRI obtained from New England BioLabs (Ipswich, Mass. USA) and Promega Corporation (Madison, Wis.), respectively, according to manufacturer's instructions. The resulting DNA was then self-ligated with T4 DNA ligase obtained from New England BioLabs (Ipswich, Mass. USA) according to manufacturer's instructions, and transformed into E. coli K12. Individual colonies from this E. coli K12 transformation were grown in liquid culture and plasmids from individual colonies were isolated using a Qiagen Miniprep kit (Valencia, Calif. USA) according to manufacturer's instructions, The isolated plasmids were screened by restriction digests with AfeI, and verified by sequencing.

In a similar manner using the corresponding restriction sites listed above the following plasmids were created: pJ61-IlvA expressed under a PtalA promoter located between NaeI and EcoICRI restriction sites; pJ61-CysM expressed under a Ppgk promoter located between PmeI and ScaI restriction sites; pJ61-Asd expressed under a PrpiA promoter located between HpaI and PmeI restriction sites; pJ61-ThrA expressed under a PtalA promoter located between ZraI and HpaI restriction sites; pJ61-SpeDEF expressed under a Ptrc promoter located between SmaI and ZraI restriction sites; pJ61-AroG expressed under a PtpiA promoter located between SfoI and SmaI restriction sites; and pJ61-CynTS expressed under a Ptrc promoter located between PmlI and SfoI restriction sites. Likewise, any combination of these operons can be obtained via a similar restriction and self-ligation scheme.

These sequence-verified plasmids were transformed into BW25113 E. coli cells as tested for tolerance to 3-HP. In addition, these plasmids can be restricted with AfeI and the purified piece containing the individual operons with mosaic ends can be incorporated into the genome of a cell line using the EZ::TN™ Transposon system obtained from EPICENTRE (Madison, Wis.) using the manufactures instructions. Likewise, these operons can be moved to any variety of plasmids from providing additional control of expression or for propagation in a variety of strains or organisms.

Method B: Plasmid Containing Identified Elements Received from Other Labs

After development of the map of the 3HPTGC, a literature review identified previous work on several of the identified genes. Requests were made to the laboratories that made these reports for plasmids containing either the wild-type or mutated genes for the elements identified in the 3HPTGC. The so-obtained gene and the proteins they encode are identified by sequence numbers in Table 4B under the Method B section thereof.

Plasmids containing the wild-type aroH gene and aroH mutants were kindly provided as a gift from the Bauerle laboratory at the University of Virginia. These mutants were described in Ray J M, Yanofsky C, Bauerle R., J Bacteriol. 1988 December; 170(12):5500-6. Mutational analysis of the catalytic and feedback sites of the tryptophan-sensitive 3-deoxy-D-arabino-heptulosonate-7-phosphate synthase of Escherichia coli. Along with a pKK223 plasmid containing the wild-type gene, three additional pKK223 plasmids were provided containing mutated genes coding for a glycine to cysteine mutation at position 149, a glycine to aspartic acid mutation at position 149, and a proline to leucine mutation at position 18.

A plasmid containing a mutant metE gene was kindly provided as a gift from the Matthews laboratory at the University of Michigan. This mutant was described in Hondorp E R, Matthews R G. J Bacteriol. 2009 May; 191(10):3407-10. Epub 2009 Mar. 13. Oxidation of cysteine 645 of cobalamin- independent methionine synthase causes a methionine limitation in Escherichia coli. This pKK233 plasmid carries a metE gene coding for a mutation of a cysteine to an alanine at position 645.

The sequences for the encoded proteins for these genes are provided as SEQ ID NOs: 022 to 026.

Method C: Tolerance Plasmids Construction in a pSMART-LC-Kan Vector

Several of the genetic elements that were assessed for their affects on 3-HP tolerance were constructed in a pSMART-LC-kan vector (SEQ ID NO:027) obtained from Lucigen Corporation (Middleton Wis., USA). This vector provides a low copy replication origin and kanamycin selection. All of these plasmids were created in a similar method and the introduced genetic elements and the proteins they encode are identified by sequence numbers in Table 4B under the method C section therein. Each row in Table 4B, under method C, contains the respective sequence information for the protein contained within the cloned plasmid, the primers used in any polymerase chain reactions, and the sequence of the polymerase chain reaction product used to create the new plasmid.

In each case, an identical procedure was used to create the final plasmid. The primers listed were used to amplify the correct insert using pfx DNA polymerase from Invitrogen Corporation (Carlsbad, Calif. USA) and genomic E. coli K12 DNA as template using the manufacturer's instructions. The 5′ termini or the amplified DNA product were phophorylated using T4 polynucleotide kinase for New England Biolabs (Ipswich, Mass. USA) using the manufacturer's instructions. The resulting product of this reaction was separated by agarose gel electrophoresis, and a band of the expected size was isolated by dissecting it from the gel and gel extracting the DNA using a gel extraction kit provided by Qiagen Corporation (Valencia, Calif. USA). The extracted phophorylated DNA was then blunt-end ligated into the pSMART-LC-Kan vector and transformed into 10 G E. coli cells using the manufacturer's instructions. Transformed cells were allowed to recover in rich media and then were plated on to LB agar plated containing kanamycin for proper selection. After colony growth, single colonies were grown in LB media and plasmid DNA was extracted using miniprep kits obtained from Qiagen Corporation (Valencia, Calif. USA). The isolated plasmid DNA was checked by restriction digest and sequenced verified before use in other experiments.

Method D: Tolerance Plasmids Construction in a pSMART-HC-Amp Vector

Several of the genetic elements that were assessed for their affects on 3-HP tolerance were constructed in a pSMART-HC-AMP vector obtained from Lucigen Corporation (Middleton Wis., USA). This vector provides a high copy replication origin and ampicillin selection. All of these plasmids were created in a similar method and are identified as method D in Table 4B. Each row in Table 4B contains the sequence information for the protein contained within the cloned plasmid, the primers used in any polymerase chain reactions, and the sequence of the polymerase chain reaction product used to create the new plasmid.

In each case, an identical procedure was used to create the final plasmid. The primers listed were used to amplify the correct insert using KOD DNA polymerase from EMD Chemical Corporation (Gibbstown, N.J. USA) and the pKK223 plasmids for each corresponding gene or genetic elements created with method B of Table 4B as template using the manufacturer's instructions. The 5′ termini or the amplified DNA product were phophorylated using T4 polynucleotide kinase for New England Biolabs (Ipswich, Mass. USA) using the manufacturer's instructions. The resulting product of this reaction was separated by agarose gel electrophoresis, and a band of the expected size was isolated by dissecting it from the gel and gel extracting the DNA using a gel extraction kit provided by Qiagen Corporation (Valencia, Calif. USA). The extracted phophorylated DNA was then blunt-end ligated into the pSMART-HC-AMP vector and transformed into 10 G E. coli cells using the manufacturer's instructions. Transformed cells were allowed to recover in rich media and then were plated on to LB agar plated containing ampicillin for proper selection. After colony growth, single colonies were grown in LB media and plasmid DNA was extracted using miniprep kits obtained from Qiagen Corporation (Valencia, Calif. USA). The isolated plasmid DNA was checked by restriction digest and sequenced verified before use in other experiments.

Method E: Additional Tolerance Plasmids Construction in a pSMART-HC-Amp Vector

Several of the genetic elements that were assessed for their affects on 3-HP tolerance were constructed in a pSMART-HC-AMP vector obtained from Lucigen Corporation (Middleton Wis., USA). This vector provides a high copy replication origin and ampicillin selection. All of these plasmids were created in a similar method and are identified as method E in Table 4B. Each row in Table 4B contains the sequence information for the protein contained within the cloned plasmid, the primers used in any polymerase chain reactions, and the sequence of the polymerase chain reaction product used to create the new plasmid.

In each case, an identical procedure was used to create the final plasmid. The primers listed were used to amplify the correct insert using KOD DNA polymerase from EMD Chemical Corporation (Gibbstown, N.J. USA) and genomic E. coli K12 DNA as template using the manufacturer's instructions. Since the 5′ termini of the primers were already phophorylated, no other treatment was needed to the amplified product. The resulting product of this reaction was separated by agarose gel electrophoresis, and a band of the expected size was isolated by dissecting it from the gel and gel extracting the DNA using a gel extraction kit provided by Qiagen Corporation (Valencia, Calif. USA). The extracted phophorylated DNA was then blunt-end ligated into the pSMART-HC-Amp vector and transformed into 10 G E. coli cells using the manufacturer's instructions. Transformed cells were allowed to recover in rich media and then were plated on to LB agar plated containing ampicillin for proper selection. After colony growth, single colonies were grown in LB media and plasmid DNA was extracted using miniprep kits obtained from Qiagen Corporation (Valencia, Calif. USA). The isolated plasmid DNA was checked by restriction digest and sequenced verified before use in other experiments.

Method F: Tolerance Plasmids Construction in a pACYC177 (Kan Only) Vector

Several of the genetic elements that were assessed for their affects on 3-HP tolerance were constructed in a pACYC177 (Kan only) vector. This backbone was created by amplifying a portion of the pACYC177 plasmid using the primer CPM0075 (5′-CGCGGTATCATTGCAGCAC-3′) (SEQ ID NO:123) and primer CPM0018 (5′-GCATCGGCTCTTCCGCGTCAAGTCAGCGTAA-3′) (SEQ ID NO:124) using KOD polymerase from EMD Chemical Corporation (Gibbstown, N.J. USA). The resulting product of this reaction was separated by agarose gel electrophoresis, and a band of the expected size was isolated by dissecting it from the gel and gel extracting the DNA using a gel extraction kit provided by Qiagen Corporation (Valencia, Calif. USA). This DNA was designated pACYC177 (Kan only) and was kept for ligation to the products created below. This pACYC177 (Kan only) backbone DNA provides low copy replication origin and kanamycin selection. All of these plasmids were created in a similar method and are identified as method F in Table 4B. Each row in Table 4B contains the sequence information for the protein contained within the cloned plasmid, the primers used in any polymerase chain reactions, and the sequence of the polymerase chain reaction product used to create the new plasmid.

In each case, an identical procedure was used to create the final plasmid. The primers listed were used to amplify the correct insert using KOD DNA polymerase from EMD Chemical Corporation (Gibbstown, N.J. USA) using the manufacturer's instructions with either the pKK223 plasmids for each corresponding gene (or genetic element) created with method B of Table 4B or with genomic E. coli DNA as template. The 5′ termini or the amplified DNA product were phophorylated using T4 polynucleotide kinase for New England Biolabs (Ipswich, Mass. USA) using the manufacturer's instructions. The resulting product of this reaction was separated by agarose gel electrophoresis, and a band of the expected size was isolated by dissecting it from the gel and gel extracting the DNA using a gel extraction kit provided by Qiagen Corporation (Valencia, Calif. USA). The extracted phophorylated DNA was then blunt-end ligated to the pACYC177 (Kan only) backbone DNA described above and transformed into 10 G E. coli cells using the manufacturer's instructions. Transformed cells were allowed to recover in rich media and then were plated on to LB agar plated containing kanamycin for proper selection. After colony growth, single colonies were grown in LB media and plasmid DNA was extracted using miniprep kits obtained from Qiagen Corporation (Valencia, Calif. USA). The isolated plasmid DNA was checked by restriction digest and sequenced verified before use in other experiments.

Method G: Tolerance Plasmids Construction in a pBT-3 Vector

Several of the genetic elements that were assessed for their affects on 3-HP tolerance were constructed in a pBT-3 vector. This backbone was created by amplifying a portion of the pBT-3 plasmid using the primer PBT-FOR (5′-AACGAATTCAAGCTTGATATC-3′) (SEQ ID NO:125) and primer PBT-REV (5′-GAATTCGTTGACGAATTCTCTAG-3′) (SEQ ID NO:126) using KOD polymerase from EMD Chemical Corporation (Gibbstown, N.J. USA). The resulting product of this reaction was separated by agarose gel electrophoresis, and a band of the expected size was isolated by dissecting it from the gel and gel extracting the DNA using a gel extraction kit provided by Qiagen Corporation (Valencia, Calif. USA). This DNA was designated pBT-3 backbone and was kept for ligation to the products created below. This pBT-3 backbone DNA provides low copy replication origin and chloramphenicol selection. All of these plasmids were created in a similar method and are identified as method G in Table 4B. Each row in Table 4B contains the sequence information for the protein contained within the cloned plasmid, the primers used in any polymerase chain reactions, and the sequence of the polymerase chain reaction product used to create the new plasmid.

In each case, an identical procedure was used to create the final plasmid. The primers listed were used to amplify the correct insert using KOD DNA polymerase from EMD Chemical Corporation (Gibbstown, N.J. USA) using the manufacturer's instructions with either the pKK223 plasmids for each corresponding gene (or genetic element) created with method B of Table 4B or with genomic E. coli DNA as template. The 5′ termini or the amplified DNA product were phophorylated using T4 polynucleotide kinase for New England Biolabs (Ipswich, Mass. USA) using the manufacturer's instructions. The resulting product of this reaction was separated by agarose gel electrophoresis, and a band of the expected size was isolated by dissecting it from the gel and gel extracting the DNA using a gel extraction kit provided by Qiagen Corporation (Valencia, Calif. USA). The extracted phophorylated DNA was then blunt-end ligated to the pBT-3 backbone DNA described above and transformed into 10 G E. coli cells using the manufacturer's instructions. Transformed cells were allowed to recover in rich media and then were plated on to LB agar plated containing chloramphenicol for proper selection. After colony growth, single colonies were grown in LB media and plasmid DNA was extracted using miniprep kits obtained from Qiagen Corporation (Valencia, Calif. USA). The isolated plasmid DNA was checked by restriction digest and sequenced verified before use in other experiments.

### Example 12

### Evaluation of a Novel Peptide Related to 3-HP Tolerance

A novel 21 amino acid peptide, termed IroK, has been discovered that increases 3-HP tolerance.

Methods:

IroK Expression Studies

Primers including the entire IroK polypeptide region and RBS flanked by EcorI and HindIII restriction sites were obtained for expression studies (Operon, Huntsville, Ala.):

(SEQ ID NO: 127)
(5′-AATTCGTGGAAGAAAGGGGAGATGAAGCCGGCATTACGCGATTTCA
TCGCCATTGTGCAGGAACGTTTGGCAAGCGTAACGGCATAA-3′,
(SEQ ID NO: 128)
5′-AGCTTTATGCCGTTACGCTTGCCAAACGTTCCTGCACAATGGCGATG
AAATCGCGTAATGCCGGCTTCATCTCCCCTTTCTTCCACG-3′)

Primers including the IroK peptide region and RBS with a mutated start site (ATG to TTG) were used for the translational analysis:

(SEQ ID NO: 187)
(5′-AATTCGTGGAAGAAAGGGGAGTTGAAGCCGGCATTACGCGATTTCA
TCGCCATTGTGCAGGAACGTTTGGCAAGCGTAACGGCATAA-3′,
(SEQ ID NO: 188)
5′-AGCTTTATGCCGTTACGCTTGCCAAACGTTCCTGCACAATGGCGATG
AAATCGCGTAATGCCGGCTTCAACTCCCCTTTCTTCCACG-3′)

The two oligonucleotides were added in a 1:1 ratio and annealed according to standard methodology in a thermal cycler. Ligation of the annealed primer product with the pKK223-3 expression vector (SEQ ID NO:008, Pharmacia, Piscataway, N.J.) was performed with T4 Ligase (Invitrogen, Carlsbad, Calif.) and incubated at 25° C. overnight. The ligation product was then electroporated into competent MACH1™-T1®, plated on LB+ampicillan, and incubated at 37° C. for 24 hours. Plasmids were isolated and confirmed by purification and subsequent restriction digest and sequencing (Macrogen, Rockville, Md.). MICs were then determined corresponding to 1 mM IPTG induction.

Minimum Inhibitory Concentrations (MIC)

The minimum inhibitory concentration (MIC) was determined microaerobically in a 96 well-plate format. Overnight cultures of strains were grown in 5 mL LB (with antibiotic where appropriate). A 1% (v/v) inoculum was introduced into a 15 ml culture of MOPS minimal media. After the cells reached mid-exponential phase, the culture was diluted to an OD₆₀₀ of 0.200. The cells were further diluted 1:20 and a 10 μL aliquot was used to inoculate each well of a 96 well plate (˜10⁴ cells per well). The plate was arranged to measure the growth of variable strains or growth conditions in increasing 3-HP concentrations, 0 to 70 g/L, in 5 g/L increments. The minimum inhibitory 3-HP concentration and maximum 3-HP concentration corresponding to visible cell growth (OD˜0.1) was recorded after 24 hours.

Results:

To explore the effects of IroK, a peptide comprised of 21 amino acids (MKPALRDFIAIVQERLASVTA, SEQ ID NO:129), the sequence encoding for it along with the native predicted RBS was incorporated into an inducible expression vector (pKK223-3). FIG. 11 shows increased expression of the short 87 bp sequence which is sufficient to enhance tolerance to 3-HP (>2 fold increase in MIC). Additionally, the tolerance mechanism appears to be specific to 3-HP growth inhibition, as MICs remained unchanged for several other organic acids of similar molecular makeup including lactic, acrylic, and acetic acids (data not shown). In an effort to dissect the mode of tolerance conferred, a nearly identical sequence was incorporated into the same vector with a single mutation in the translational start site (ATG to TTG), resulting in a decreased MIC equivalent to that of wild-type E. coli (FIG. 11). This result implies that the mechanism of tolerance is specific to the expression of the translated polypeptide rather than mapped to the DNA or RNA level.

A nucleic acid sequence encoding the IroK peptide, or suitable variants of it, may be provided to a microorganism, that may comprise one or more genetic modifications of the 3HPTGC to further increase 3-HP tolerance, and that also may have 3-HP production capability.

### Example 13

### Genetic Modification/Introduction of Malonyl-CoA Reductase for 3-HP production in E. coli DF40

The nucleotide sequence for the malonyl-coA reductase gene from Chloroflexus aurantiacus was codon optimized for E. coli according to a service from DNA 2.0 (Menlo Park, Calif. USA), a commercial DNA gene synthesis provider. This gene sequence incorporated an EcoRI restriction site before the start codon and was followed by a HindIII restriction site. In addition a Shine Delgarno sequence (i.e., a ribosomal binding site) was placed in front of the start codon preceded by an EcoRI restriction site. This gene construct was synthesized by DNA 2.0 and provided in a pJ206 vector backbone. Plasmid DNA pJ206 containing the synthesized mcr gene was subjected to enzymatic restriction digestion with the enzymes EcoRI and HindIII obtained from New England BioLabs (Ipswich, Mass. USA) according to manufacturer's instructions. The digestion mixture was separated by agarose gel electrophoresis, and visualized under UV transillumination as described in Subsection II of the Common Methods Section. An agarose gel slice containing a DNA piece corresponding to the mcr gene was cut from the gel and the DNA recovered with a standard gel extraction protocol and components from Qiagen (Valencia, Calif. USA) according to manufacturer's instructions. An E. coli cloning strain bearing pKK223-aroH was obtained as a kind a gift from the laboratory of Prof. Ryan T. Gill from the University of Colorado at Boulder. Cultures of this strain bearing the plasmid were grown by standard methodologies and plasmid DNA was prepared by a commercial miniprep column from Qiagen (Valencia, Calif. USA) according to manufacturer's instructions. Plasmid DNA was digested with the restriction endonucleases EcoRI and HindIII obtained from New England Biolabs (Ipswich, Mass. USA) according to manufacturer's instructions. This digestion served to separate the aroH reading frame from the pKK223 backbone. The digestion mixture was separated by agarose gel electrophoresis, and visualized under UV transillumination as described in Subsection II of the Common Methods Section. An agarose gel slice containing a DNA piece corresponding to the backbone of the pKK223 plasmid was cut from the gel and the DNA recovered with a standard gel extraction protocol and components from Qiagen according to manufacturer's instructions.

Pieces of purified DNA corresponding to the mcr gene and pK223 vector backbone were ligated and the ligation product was transformed and electroporated according to manufacturer's instructions. The sequence of the resulting vector termed pKK223-mcr (SEQ ID NO:189) was confirmed by routine sequencing performed by the commercial service provided by Macrogen (USA). pKK223-mcr confers resistance to beta-lactamase and contains mcr gene under control of a Ptac promoter inducible in E. coli hosts by IPTG.

The expression clone pKK223-mcr and pKK223 control were transformed into both E. coli K12 and E. coli DF40 via standard methodologies. (Sambrook and Russell, 2001).

### Example 14

### Construction of E. coli Gene Deletion Strains

The following strains were obtained from the Keio collection: JW1650 (ΔpurR), JW2807 (ΔlysR), JW1316 (ΔtyrR), JW4356 (ΔtrpR), JW3909 (ΔmetJ), JW0403 (ΔnrdR). The Keio collection was obtained from Open Biosystems (Huntsville, Ala. USA 35806). Individual clones may purchased from the Yale Genetic Stock Center (New Haven, Conn. USA 06520). These strains each contain a kanamycin marker in place of the deleted gene. For more information concerning the Keio Collection and the curing of the kanamycin cassette please refer to: Baba, T et al (2006). Construction of Escherichia coli K12 in-frame, single-gene knockout mutants: the Keio collection. Molecular Systems Biology doi:10.1038/msb4100050 and Datsenko K A and B L Wanner (2000). One-step inactivation of chromosomal genes in Escherichia coli K-12 using PCR products. PNAS 97, 6640-6645. These strains were made electro-competent by standard methodologies. Each strain was then transformed via standard electroporation methods with the plasmid pCP20, which was a kind gift from Dr. Ryan Gill (University of Colorado, Boulder, Colo. USA). Transformations were plated on Luria Broth agar plates containing 20 μg/mL chloramphenicol and 100 μg/mL ampicillin and incubated for 36 hours at 30 degrees Celsius. Clones were isolated from these transformation and grown overnight in 10 mL of M9 media lacking any antibiotics. Colonies were isolated from these cultures by streaking onto Luria Broth agar plates lacking any antibiotics. Colonies were confirmed to have lost the kanamycin marker as well as the plasmid pCP20 by confirming no growth on Luria broth agar plates containing the antibiotics, kanamycin (20 μg/mL), chloramphenicol (20 μg/mL) and ampicillin (100 μg/mL). Isolated clones were confirmed by colony PCR to have lost the kanamycin cassette. PCRs were carried out using EconoTaq PLUS GREEN 2X master PCR mix, Obtained from Lucigen, (Catalog #30033) (Middleton, Wis. USA). PCRs were carried out using a 96 well gradient ROBOcycler (Stratagene, La Jolla, Calif. USA 92037) with the following cycles: 1) 10 min at 95 degrees Celsius, 2) 30 of the following cycles, a) 1 min at 95 degrees Celsius, b) 1 min at 52 degrees Celsius, b) 2 min at 72 degrees Celsius, followed by 3) 1 cycle of 10 minutes at 72 degrees Celsius. The Primers used for the PCRs to confirm the removal of the kanamycin cassette for each of the clones are given in Table 5. Primers were purchased from Integrated DNA Technologies (Coralville, Iowa USA). The resulting cured strains, called BX_{—}00341.0, BX_{—}00342.0, BX_{—}00345.0, BX_{—}00346.0, BX_{—}00348.0 and BX_{—}00349.0, correspond to JW1316 (ΔtyrR), JW4356 (ΔtrpR), JW3909 (ΔmetJ), JW1650 (ΔpurR), JW2807 (ΔlysR) and JW0403 (ΔnrdR) respectively.

### Example 15

### E. coli Strain Construction

According to the respective combinations indicated in Tables 6 and 7, the plasmids of Table 4B were introduced into the respective base strains. All plasmids were introduced at the same time via electroporation using standard methods. Transformed cells were grown on the appropriate media with antibiotic supplementation and colonies were selected based on their appropriate growth on the selective media.

### Example 16

### Evaluation of 3HPTGC-Related Supplements on Wild-Type E. coli

The effects of supplementation on 3HP tolerance was determined by MIC evaluations using the methods described in the Common Methods Section. Supplements tested are listed in table 3. Results of the MIC evaluations are provided in Table 8 for aerobic condition and Table 9 for anaerobic condition. This data, which includes single- and multiple-supplement additions, demonstrates improvement in 3-HP tolerance in these culture systems based on 24-hour MIC evaluations.

### Example 17

### Evaluation of 3HPTGC-Related Genetically Modified E. coli

The effects of genetic modifications on 3HP tolerance was determined by MIC evaluations using the methods described in the Common Methods Section. Genetic modifications tested in E. coli and the MIC results thereof are listed in Table 6 for aerobic condition and Table 7 for anaerobic condition. This data, which includes single and multiple genetic modifications, demonstrates improvement in 3-HP tolerance in these culture systems based on 24-hour MIC evaluations.

### Example 18

### Toleragram Comparison with CynTS Genetic Modification

Twenty-four hour duration toleragram evaluations were conducted to compare a control (wild-type) E. coli (strain BW25113) with a genetically modified E. coli (strain BW25113) comprising a genetic modification to introduce cynTS. This introduction was made by the method of Example 5

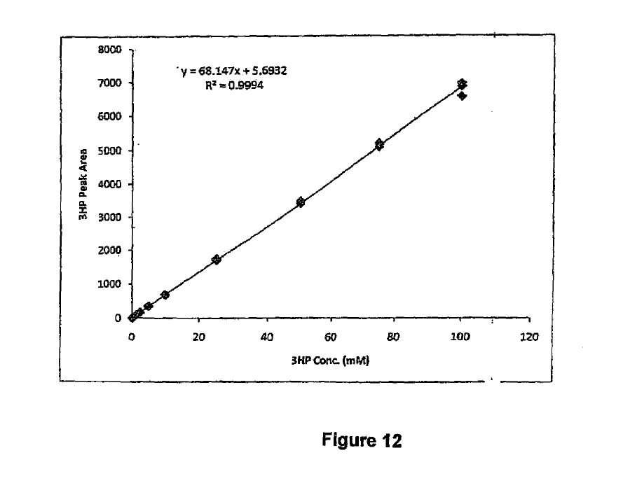

Results are provided in FIG. 12, which show the control strain also tested under indicated additional conditions.

Based on the area under the curve, the cynTS treatment is demonstrated to exhibit greater tolerance to 3-HP, at various elevated 3-HP concentrations, versus the control.

### Example 19

### Genetic Modification/Introduction of Tolerance Pieces into Bacillus subtilus

For creation of a 3-HP production tolerance pieces into Bacillus subtilus several genes from the E. coli toleragenic complex were cloned into a Bacillus shuttle vector, pWH1520 (SEQ ID NO:010) obtained from Boca Scientific (Boca Raton, Fla. USA). This shuttle vector carries an inducible Pxyl xylose-inducible promoter, as well as an ampicillin resistance cassette for propagation in E. coli and a tetracycline resistance cassette for propagation in Bacillus subtilus. Cloning strategies for these genes are shown in Table 10.

### Method A

Tolerance genes cloned for testing in B. subtilus designated a cloning method A in Table 10 were created in a similar manner. The cloning method described here places the gene under the xylose-inducible promoter. Each gene was amplified by polymerase chain reaction using their corresponding Primers A and Primer B listed in each row of the table. Primer A of each set contains homology to the start of the gene and a SpeI restriction site. Primer B contains homology for the region downstream of the stop codon of the gene and a BamHI restriction site. The polymerase chain reaction product was purified using a PCR purification kit obtained from Qiagen Corporation (Valencia, Calif. USA) according to manufacturer's instructions. Next, the purified product was digested with SpeI and BamHI obtained from New England BioLabs (Ipswich, Mass. USA) according to manufacturer's instructions. The digestion mixture was separated by agarose gel electrophoresis, and visualized under UV transillumination as described in Subsection II of the Common Methods Section. An agarose gel slice containing a DNA piece corresponding to the digested and purified tolerance gene was cut from the gel and the DNA recovered with a standard gel extraction protocol and components from Qiagen (Valencia, Calif. USA) according to manufacturer's instructions.

This pWH1520 shuttle vector DNA was isolated using a standard miniprep DNA purification kit from Qiagen (Valencia, Calif. USA) according to manufacturer's instructions. The resulting DNA was restriction digested with SpeI and SphI obtained from New England BioLabs (Ipswich, Mass. USA) according to manufacturer's instructions. The digestion mixture was separated by agarose gel electrophoresis, and visualized under UV transillumination as described in Subsection II of the Common Methods Section. An agarose gel slice containing a DNA piece corresponding to digested pWH1520 backbone product was cut from the gel and the DNA recovered with a standard gel extraction protocol and components from Qiagen (Valencia, Calif. USA) according to manufacturer's instructions.

Both the digested and purified tolerance gene and pWH1520 DNA products were ligated together using T4 ligase obtained from New England BioLabs (Ipswich, Mass. USA) according to manufacturer's instructions. The ligation mixture was then transformed into chemically competent 10 G E. coli cells obtained from Lucigen Corporation (Middleton Wis., USA) according to the manufacturer's instructions and plated LB plates augmented with ampicillin for selection. Several of the resulting colonies were cultured and their DNA was isolated using a standard miniprep DNA purification kit from Qiagen (Valencia, Calif. USA) according to manufacturer's instructions. The recovered DNA was checked by restriction digest followed by agarose gel electrophoresis. DNA samples showing the correct banding pattern were further verified by DNA sequencing.

### Example 20

### Genetic Modification/Introduction of Malonyl-CoA Reductase for 3-HP Production in Bacillus subtilus

For creation of a 3-HP production pathway in Bacillus Subtilus the codon optimized nucleotide sequence for the malonyl-coA reductase gene from Chloroflexus aurantiacus that was constructed by the gene synthesis service from DNA 2.0 (Menlo Park, Calif. USA), a commercial DNA gene synthesis provider, was added to a Bacillus Subtilus shuttle vector. This shuttle vector, pHT08 (SEQ ID NO:011), was obtained from Boca Scientific (Boca Raton, Fla. USA) and carries an inducible Pgrac IPTG-inducible promoter.

This mcr gene sequence was prepared for insertion into the pHT08 shuttle vector by polymerase chain reaction amplification with primer 1 (5′GGAAGGATCCATGTCCGGTACGGGTCG-3′) (SEQ ID NO:148), which contains homology to the start site of the mcr gene and a BamHI restriction site, and primer 2 (5′-Phos-GGGATTAGACGGTAATCGCACGACCG-3′) (SEQ ID NO:149), which contains the stop codon of the mcr gene and a phosphorylated 5′ terminus for blunt ligation cloning. The polymerase chain reaction product was purified using a PCR purification kit obtained from Qiagen Corporation (Valencia, Calif. USA) according to manufacturer's instructions. Next, the purified product was digested with BamHI obtained from New England BioLabs (Ipswich, Mass. USA) according to manufacturer's instructions. The digestion mixture was separated by agarose gel electrophoresis, and visualized under UV transillumination as described in Subsection II of the Common Methods Section. An agarose gel slice containing a DNA piece corresponding to the mcr gene was cut from the gel and the DNA recovered with a standard gel extraction protocol and components from Qiagen (Valencia, Calif. USA) according to manufacturer's instructions.

This pHT08 shuttle vector DNA was isolated using a standard miniprep DNA purification kit from Qiagen (Valencia, Calif. USA) according to manufacturer's instructions. The resulting DNA was restriction digested with BamHI and SmaI obtained from New England BioLabs (Ipswich, Mass. USA) according to manufacturer's instructions. The digestion mixture was separated by agarose gel electrophoresis, and visualized under UV transillumination as described in Subsection II of the Common Methods Section. An agarose gel slice containing a DNA piece corresponding to digested pHT08 backbone product was cut from the gel and the DNA recovered with a standard gel extraction protocol and components from Qiagen (Valencia, Calif. USA) according to manufacturer's instructions.

Both the digested and purified mcr and pHT08 products were ligated together using T4 ligase obtained from New England BioLabs (Ipswich, Mass. USA) according to manufacturer's instructions. The ligation mixture was then transformed into chemically competent 10 G E. coli cells obtained from Lucigen Corporation (Middleton Wis., USA) according to the manufacturer's instructions and plated LB plates augmented with ampicillin for selection. Several of the resulting colonies were cultured and their DNA was isolated using a standard miniprep DNA purification kit from Qiagen (Valencia, Calif. USA) according to manufacturer's instructions. The recovered DNA was checked by restriction digest followed by agarose gel electrophoresis. DNA samples showing the correct banding pattern were further verified by DNA sequencing. The sequence verified DNA was designated as pHT08-mcr, and was then transformed into chemically competent Bacillus subtilus cells using directions obtained from Boca Scientific (Boca Raton, Fla. USA). Bacillus subtilus cells carrying the pHT08-mcr plasmid were selected for on LB plates augmented with chloramphenicol.

Bacillus subtilus cells carrying the pHT08-mcr, were grown overnight in 5 ml of LB media supplemented with 20 ug/mL chloramphenicol, shaking at 225 rpm and incubated at 37 degrees Celsius. These cultures were used to inoculate 1% v/v, 75 mL of M9 minimal media supplemented with 1.47 g/L glutamate, 0.021 g/L tryptophan, 20 ug/mL chloramphenicol and 1 mM IPTG. These cultures were then grown for 18 hours in a 250 mL baffled erylenmeyer flask at 25 rpm, incubated at 37 degrees Celsius. After 18 hours, cells were pelleted and supernatants subjected to GC_MS detection of 3-HP (described in Common Methods Section IIIb)). Trace amounts of 3-HP were detected with qualifier ions.

### Example 21

### Bacillus subtilus Strain Construction

Plasmids for tolerance genetic elements in pWH1520 and the production plasmid, pHT08-mcr, were transformed in to two Bacillus subtilus strains. The Bacillus subtilus subspecies subtilis 168 strain was obtained as a kind a gift from the laboratory of Prof. Ryan T. Gill from the University of Colorado at Boulder. Transformations were performed using a modified protocol developed from Anagnostopoulos and Spizizen (Requirements for transformation in Bacillus subtilis. J. Bacteriol. 81:741-746 (1961)) as provided with the instructions for the pHT08 shuttle vector by Boca Scientific (Boca Raton, Fla. USA).

### Example 22

### Evaluation of 3HPTGC-Related Supplements on Wild-Type B. subtilis

The effects of supplementation on 3HP tolerance was determined by MIC evaluations using the methods described in the Common Methods Section. Supplements tested are listed in table 3. Results of the MIC evaluations under anaerobic condition are provided in Table 11.

### Example 23

### Evaluation of 3HPTGC-Related Genetically Modified B. subtilis without and with 3HPTGC-Related Supplements

The effects of supplementation and/or genetic modifications on 3HP tolerance in B. subtilis was determined by MIC evaluations using the methods described in the Common Methods Section. Supplements tested are listed in table 3. Genetic modifications tested and the MIC results under aerobic condition for B. subtilis are provided in Table 11. This data, which includes single genetic modifications and single and multiple supplement additions, demonstrates improvement in 3-HP tolerance in this culture system based changes in OD.

### Example 24

### Yeast Aerobic Pathway for 3HP Production (Prophetic)

The following construct (SEQ ID NO:150) containing: 200 bp 5′ homology to ACC1, His3 gene for selection, Adh1 yeast promoter, BamHI and SpeI sites for cloning of MCR, cyc1 terminator, Tef1 promoter from yeast and the first 200 bp of homology to the yeast ACC1 open reading frame will be constructed using gene synthesis (DNA 2.0). The MCR open reading frame (SEQ ID NO:151)will be cloned into the BamHI and SpeI sites, this will allow for constitutive transcription by the adh1 promoter. Following the cloning of MCR into the construct the genetic element (SEQ ID NO:152) will be isolated from the plasmid by restriction digestion and transformed transformed into relevant yeast strains. The genetic element will knockout the native promoter of yeast ACC1 and replace it with MCR expressed from the adh1 promoter and the Tef1 promoter will now drive yeast ACC1 expression. The integration will be selected for by growth in the absence of histidine. Positive colonies will be confirmed by PCR. Expression of MCR and increased expression of ACC1 will be confirmed by RT-PCR.

An alternative approach that could be utilized to express MCR in yeast is expression of MCR from a plasmid. The genetic element containing MCR under the control of the ADH1 promoter (SEQ ID 4) could be cloned into a yeast vector such as pRS421 (SEQ ID NO:153) using standard molecular biology techniques creating a plasmid containing MCR (SEQ ID NO:154). A plasmid based MCR could then be transformed into different yeast strains.

### Example 25

### Cloning of Saccharomyces cerevisiae Genetic Elements for Increased Tolerance to 3HP

Yeast genes were identified by homology and pathway comparison using biocyc.org, outlined in FIG. 1D, sheets 1-7. Genetic elements were amplified by PCR using the primers in Table 12. Yeast genetic elements were amplified to contain native promoters and 3′ untranslated region, PCR product sequences table 12. PCR products were isolated by gel electrophoresis and gel purification using Qiagen gel extraction (Valencia, Calif. USA, Cat. No. 28706) following the manufactures instructions. Gel purified yeast genetic elements were then cloned into pYes2.1-topo vector (SEQ ID NO:183, Invitrogen Corp, Carlsbad, Calif., USA) following manufacture instructions. Colonies were screened by PCR and then sequenced by Genewiz.

### Example 26

### Sub-Cloning Yeast Genetic Elements into E. coli/Yeast Shuttle Vectors pRS423 and pRS425

Genetic elements were excised from pYes2.1 by restriction digestion with restriction enzymes PvuII and XbaI. Restriction fragments containing yeast genetic elements were isolated by gel electrophoresis and gel purification using Qiagen gel extraction (Valencia, Calif. USA, Cat. No. 28706) following manufactures instructions. Backbone vectors pRS423 and pRS425 were digested with SmaI and SpeI restriction enzymes and gel purified. Yeast genetic elements were ligated into pRS423 and pRS425 (SEQ ID NO:184 and 185). All plasmids were checked using PCR analysis and sequencing.

### Example 27

### Yeast Strain Construction

Yeast strains were constructed using standard yeast transformation and selected for by complementation of auxotrophic markers. All strains are S288C background. For general yeast transformation methods, see Gietz, R. D. and R. A. Woods. (2002) TRANSFORMATION OF YEAST BY THE Liac/SS CARRIER DNA/PEG METHOD. Methods in Enzymology 350: 87-96.

### Example 28

### Evaluation of Supplements and/or Genetic Modifications on 3HP Tolerance in Yeast

The effects of supplementation and/or genetic modifications on 3HP tolerance was determined by MIC evaluations using the methods described in this Example. Supplements tested are listed in Tables 13 and 14 for aerobic and anaerobic conditions, respectively. Genetic modifications tested in yeast are listed in Tables 15 and 16 for aerobic and anaerobic conditions, respectively. Results of the MIC evaluations are provided in Tables 13-16. This data, which includes single and multiple supplement additions and genetic modifications, demonstrates improvement in 3-HP tolerance in these culture systems based on the MIC evaluations described below.

Method for Yeast aerobic Minimum Inhibitory Concentration Evaluation

The minimum inhibitory concentration (MIC) was determined aerobically in a 96 well-plate format. Plates were setup such that each individual well, when brought to a final volume of 100 uL following inoculation, had the following component levels (corresponding to synthetic minimal glucose medium (SD) standard media without vitamins): 20 g/L dextrose, 5 g/L ammonium sulfate, 850 mg/L potassium phosphate monobasic, 150 mg/L potassium phosphate dibasic, 500 mg/L magnesium sulfate, 100 mg/L sodium chloride, 100 mg/L calcium chloride, 500 μg/L boric acid, 40 μg/L copper sulfate, 100 μg/L potassium iodide, 200 μg/L ferric chloride, 400 μg/L manganese sulfate, 200 μg/L sodium molybdate, and 400 μg/L zinc sulfate. Media supplements were added according to levels reported in Table 3, where specified. Overnight cultures of strains were grown in triplicate in 5 mL SD media with vitamins (Methods in Enzymology vol. 350, page 17 (2002)). A 1% (v/v) inoculum was introduced into a 5 ml culture of SD minimal media without vitamins. After the cells reached mid-exponential phase, the culture was diluted to an OD₆₀₀ of 0.200. The cells were further diluted 1:5 and a 10 μL aliquot was used to inoculate each well of a 96 well plate (˜10⁴ cells per well) to total volume of 100 uL. The plate was arranged to measure the growth of variable strains or growth conditions in increasing 3-HP concentrations, 0 to 60 g/L, in 5 g/L increments. Plates were incubated for 72 hours at 30C. The minimum inhibitory 3-HP concentration and maximum 3-HP concentration corresponding to visible cell growth (OD˜0.1) was recorded after 72 hours. For cases when MIC>60 g/L, assessments were performed in plates with extended 3-HP concentrations (0-100 g/L, in 5 g/L increments).

Method for Yeast Anaerobic Minimum Inhibitory Concentration Evaluation

The minimum inhibitory concentration (MIC) was determined anaerobically in a 96 well-plate format. Plates were setup such that each individual well, when brought to a final volume of 100 uL following inoculation, had the following component levels (corresponding to synthetic minimal glucose medium (SD) standard media without vitamins): 20 g/L dextrose, 5 g/L ammonium sulfate, 850 mg/L potassium phosphate monobasic, 150 mg/L potassium phosphate dibasic, 500 mg/L magnesium sulfate, 100 mg/L sodium chloride, 100 mg/L calcium chloride, 500 g/L boric acid, 40 g/L copper sulfate, 100 g/L potassium iodide, 200 g/L ferric chloride, 400 g/L manganese sulfate, 200 g/L sodium molybdate, and 400 g/L zinc sulfate. Media supplements were added according to levels reported in Table X, where specified. Overnight cultures of strains were grown in triplicate in 5 mL SD media with vitamins (Methods in Enzymology vol. 350, page 17 (2002)). A 1% (v/v) inoculum was introduced into a 5 ml culture of SD minimal media without vitamins. After the cells reached mid-exponential phase, the culture was diluted to an OD₆₀₀ of 0.200. The cells were further diluted 1:5 and a 10 μL aliquot was used to inoculate each well of a 96 well plate (˜10⁴ cells per well) to total volume of 100 uL. The plate was arranged to measure the growth of variable strains or growth conditions in increasing 3-HP concentrations, 0 to 60 g/L, in 5 g/L increments. Plates were incubated for 72 hours at 30C. The minimum inhibitory 3-HP concentration and maximum 3-HP concentration corresponding to visible cell growth (OD˜0.1) was recorded after 72 hours. For cases when MIC>60 g/L, assessments were performed in plates with extended 3-HP concentrations (0-100 g/L, in 5 g/L increments). Plates were sealed in biobag anaerobic chambers that contained gas generators for anaerobic conditions and incubated for 72 hours at 30C. The minimum inhibitory 3-HP concentration and maximum 3-HP concentration corresponding to visible cell growth (OD˜0.1) was recorded after 72 hours. For cases when MIC>60 g/L, assessments were performed in plates with extended 3-HP concentrations (0-100 g/L, in 5 g/L increments).

### Example 29

### Evaluation of 3HPTGC-Related Supplements in Cupriavidus necator

The effects of supplementation on 3HP tolerance in C. necator was determined by MIC evaluations using the methods described in the Common Methods Section. Supplements tested are listed in table 3.

MIC results under aerobic condition for C. necator are provided in Table 17. This data, which includes single and multiple supplement additions, demonstrates improvement in 3-HP tolerance in these culture systems based on the MIC evaluations.

### Example 30

### Additional Example of 3HPTGC Tolerance-Directed Genetic Modification(s) in Combination with 3-HP Production Genetic Modification(s)

In addition to Example 9, which provides a general example to combine tolerance and 3-HP production genetic modifications to obtain a desired genetically modified microorganism suitable for use to produce 3-HP, and in view of the examples following Example 9, and considering additional disclosure herein, and methods known to those skilled in the art (e.g., Sambrook and Russell, 2001, incorporated into this example for its methods of genetic modifications), this example 28 provides a microorganism species genetically modified to comprise one or more genetic modifications of the 3HPTGC to provide an increase tolerance to 3-HP (which may be assessed by any metric such as those discussed herein) and one or more genetic modifications to increase 3-HP production (such as of a 3-HP production pathway such as those disclosed herein).

The so-genetically modified microorganism may be evaluated both for tolerance to and production of 3-HP under varying conditions including oxygen content of the culture system and nutrient composition of the media.

In various aspects of this example, multiple sets of genetic modifications are made and are compared to identify one or more genetically modified microorganisms that comprise desired attributes and/or metrics for increased 3-HP tolerance and production.

### Example 31

### Introduction of Genetic Modification Encoding the Irok Sequence Combined with 3HPTGC Genetic Modifications

Example 12 describes Irok, a peptide comprised of 21 amino acids, and its 3-HP tolerance improving effect when a plasmid encoding it is introduced into an E. coli strain and evaluated under microaerobic conditions.

Considering the disclosure herein regarding the 3HPTGC, and methods known to those skilled in the art (e.g., Sambrook and Russell, 2001, incorporated into this example for its methods of genetic modifications), a microorganism species is genetically modified to comprise a nucleic acid sequence that encodes the IroK peptide sequence and one or more genetic modifications of the 3HPTGC, collectively to provide an increase tolerance to 3-HP. Such increase in 3-HP tolerance may be assessed by any metric such as those discussed herein.

Thus, based on the above results various genetic modification combinations that include representation from two or more of the Groups A-E may be evaluated, and employed, in a microorganism to achieve a desired elevated tolerance to 3-HP. Tables 6, 7, 11, 15 and 16 show results of particular genetic modification combinations that include combinations from these groups. Also, additional genetic modifications may be provided from Group F. As described elsewhere herein, any such combination may be combined with other genetic modifications that may include one or more of: 3-HP bio-production pathways to provide and/or increase 3-HP synthesis and accumulation by the recombinant microorganism, and deletions or other modifications to direct more metabolic resources (e.g., carbon and energy) into 3-HP bio-production.

In view of the above disclosure, the following pertain to exemplary methods of modifying specific species of host organisms that span a broad range of microorganisms of commercial value. These examples further support that the use of E. coli, although convenient for many reasons, is not meant to be limiting. The following are non-limiting general prophetic examples directed to practicing the present invention in other microorganism species.

### General Prophetic Example 32

Improvement of 3-HP Tolerance in Rhodococcus erythropolis

A series of E. coli-Rhodococcus shuttle vectors are available for expression in R. erythropolis, including, but not limited to, pRhBR17 and pDA71 (Kostichka et al., Appl. Microbiol. Biotechnol. 62:61-68(2003)). Additionally, a series of promoters are available for heterologous gene expression in R. erythropolis (see for example Nakashima et al., Appl. Environ. Microbiol. 70:5557-5568 (2004), and Tao et al., Appl. Microbiol. Biotechnol. 2005, DOI 10.1007/s00253-005-0064). Targeted gene disruption of chromosomal genes in R. erythropolis may be created using the method described by Tao et al., supra, and Brans et al. (Appl. Environ. Microbiol. 66: 2029-2036 (2000)). These published resources are incorporated by reference for their respective indicated teachings and compositions.

The nucleic acid sequences required for providing an increase in 3-HP tolerance, as described above, optionally with nucleic acid sequences to provide and/or improve a 3-HP biosynthesis pathway, are cloned initially in pDA71 or pRhBR71 and transformed into E. coli. The vectors are then transformed into R. erythropolis by electroporation, as described by Kostichka et al., supra. The recombinants are grown in synthetic medium containing glucose and the tolerance to and/or bio-production of 3-HP are followed using methods known in the art or described herein.

### General Prophetic Example 33

Improvement of 3-HP Tolerance in B. licheniformis

Most of the plasmids and shuttle vectors that replicate in B. subtilis are used to transform B. licheniformis by either protoplast transformation or electroporation. The nucleic acid sequences required for improvement of 3-HP tolerance, and/or for 3-HP biosynthesis are isolated from various sources, codon optimized as appropriate, and cloned in plasmids pBE20 or pBE60 derivatives (Nagarajan et al., Gene 114:121-126 (1992)). Methods to transform B. licheniformis are known in the art (for example see Fleming et al. Appl. Environ. Microbiol., 61(11):3775-3780 (1995)). These published resources are incorporated by reference for their respective indicated teachings and compositions.

The plasmids constructed for expression in B. subtilis are transformed into B. licheniformis to produce a recombinant microorganism that then demonstrates improved 3-HP tolerance, and, optionally, 3-HP bio-production.

### General Prophetic Example 34

Improvement of 3-HP Tolerance in Paenibacillus macerans

Plasmids are constructed as described above for expression in B. subtilis and used to transform Paenibacillus macerans by protoplast transformation to produce a recombinant microorganism that demonstrates improved 3-HP tolerance, and, optionally, 3-HP bio-production.

### General Prophetic Example 35

Expression of 3-HP Tolerance in Alcaligenes (Ralstonia) Eutrophus (currently referred to as Cupriavidus necator).

Methods for gene expression and creation of mutations in Alcaligenes eutrophus are known in the art (see for example Taghavi et al., Appl. Environ. Microbiol., 60(10):3585-3591 (1994)). This published resource is incorporated by reference for its indicated teachings and compositions. Any of the nucleic acid sequences identified to improve 3-HP tolerance, and/or for 3-HP biosynthesis are isolated from various sources, codon optimized as appropriate, and cloned in any of the broad host range vectors described above, and electroporated to generate recombinant microorganisms that demonstrate improved 3-HP tolerance, and, optionally, 3-HP bio-production. The poly(hydroxybutyrate) pathway in Alcaligenes has been described in detail, a variety of genetic techniques to modify the Alcaligenes eutrophus genome is known, and those tools can be applied for engineering a 3-HP toleragenic or, optionally, a 3-HP-gena-toleragenic recombinant microorganism.

### General Prophetic Example 36

### Improvement of 3-HP Tolerance in Pseudomonas putida

Methods for gene expression in Pseudomonas putida are known in the art (see for example Ben-Bassat et al., U.S. Pat. No. 6,586,229, which is incorporated herein by reference for these teachings). Any of the nucleic acid sequences identified to improve 3-HP tolerance, and/or for 3-HP biosynthesis are isolated from various sources, codon optimized as appropriate, and cloned in any of the broad host range vectors described above, and electroporated to generate recombinant microorganisms that demonstrate improved 3-HP tolerance, and, optionally, 3-HP biosynthetic production. For example, these nucleic acid sequences are inserted into pUCP18 and this ligated DNA are electroporated into electrocompetent Pseudomonas putida KT2440 cells to generate recombinant P. pudita microorganisms that exhibit increased 3-HP tolerance and optionally also comprise 3-HP biosynthesis pathways comprised at least in part of introduced nucleic acid sequences.

### General Prophetic Example 37

Improvement of 3-HP Tolerance in Lactobacillus plantarum

The Lactobacillus genus belongs to the Lactobacillales family and many plasmids and vectors used in the transformation of Bacillus subtilis and Streptococcus are used for lactobacillus. Non-limiting examples of suitable vectors include pAM.beta.1 and derivatives thereof (Renault et al., Gene 183:175-182 (1996); and O'Sullivan et al., Gene 137:227-231 (1993)); pMBB1 and pHW800, a derivative of pMBB1 (Wyckoff et al. Appl. Environ. Microbiol 62:1481-1486 (1996)); pMG1, a conjugative plasmid (Tanimoto et al., J. Bacteriol. 184:5800-5804 (2002)); pNZ9520 (Kleerebezem et al., Appl. Environ. Microbiol. 63:4581-4584 (1997)); pAM401 (Fujimoto et al., Appl. Environ. Microbiol. 67:1262-1267 (2001)); and pAT392 (Arthur et al., Antimicrob. Agents Chemother. 38:1899-1903 (1994)). Several plasmids from Lactobacillus plantarum have also been reported (e.g., van Kranenburg R, Golic N, Bongers R, Leer R J, de Vos W M, Siezen R J, Kleerebezem M. Appl. Environ. Microbiol. 2005 March; 71(3): 1223-1230).

### General Prophetic Example 38

Improvement of 3-HP Tolerance in Enterococcus faecium, Enterococcus gallinarium, and Enterococcus faecalis

The Enterococcus genus belongs to the Lactobacillales family and many plasmids and vectors used in the transformation of Lactobacillus, Bacillus subtilis, and Streptococcus are used for Enterococcus. Non-limiting examples of suitable vectors include pAM.beta.1 and derivatives thereof (Renault et al., Gene 183:175-182 (1996); and O'Sullivan et al., Gene 137:227-231 (1993)); pMBB1 and pHW800, a derivative of pMBB1 (Wyckoff et al. Appl. Environ. Microbiol. 62:1481-1486 (1996)); pMG1, a conjugative plasmid (Tanimoto et al., J. Bacterial. 184:5800-5804 (2002)); pNZ9520 (Kleerebezem et al., Appl. Environ. Microbiol. 63:4581-4584 (1997)); pAM401 (Fujimoto et al., Appl. Environ. Microbiol. 67:1262-1267 (2001)); and pAT392 (Arthur et al., Antimicrob. Agents Chemother. 38:1899-1903 (1994)). Expression vectors for E. faecalis using the nisA gene from Lactococcus may also be used (Eichenbaum et al., Appl. Environ. Microbiol. 64:2763-2769 (1998). Additionally, vectors for gene replacement in the E. faecium chromosome are used (Nallaapareddy et al., Appl. Environ. Microbiol. 72:334-345 (2006)).

For each of the General Prophetic Examples 32-38, the following 3-HP bio-production comparison may be incorporated thereto: Using analytical methods for 3-HP such as are described in Subsection III of Common Methods Section, below, 3-HP is obtained in a measurable quantity at the conclusion of a respective bio-production event conducted with the respective recombinant microorganism (see types of bio-production events, below, incorporated by reference into each respective General Prophetic Example). That measurable quantity is substantially greater than a quantity of 3-HP produced in a control bio-production event using a suitable respective control microorganism lacking the functional 3-HP pathway so provided in the respective General Prophetic Example. Tolerance improvements also may be assessed by any recognized comparative measurement technique, such as by using a MIC protocol provided in the Common Methods Section.

### Common Methods Section

All methods in this Section are provided for incorporation into the above methods where so referenced therein and/or below.

Subsection I. Bacterial Growth Methods: Bacterial Growth Culture Methods, and Associated Materials and Conditions, are Disclosed for Respective Species, that may be Utilized as Needed, as Follows:

Acinetobacter calcoaceticus (DSMZ #1139) is obtained from the German Collection of Microorganisms and Cell Cultures (Braunschweig, Germany) as a vacuum dried culture. Cultures are then resuspended in Brain Heart Infusion (BHI) Broth (RPI Corp, Mt. Prospect, Ill., USA). Serial dilutions of the resuspended A. calcoaceticus culture are made into BHI and are allowed to grow for aerobically for 48 hours at 37° C. at 250 rpm until saturated.

Bacillus subtilis is a gift from the Gill lab (University of Colorado at Boulder) and is obtained as an actively growing culture. Serial dilutions of the actively growing B. subtilis culture are made into Luria Broth (RPI Corp, Mt. Prospect, Ill., USA) and are allowed to grow for aerobically for 24 hours at 37° C. at 250 rpm until saturated.

Chlorobium limicola (DSMZ #245) is obtained from the German Collection of Microorganisms and Cell Cultures (Braunschweig, Germany) as a vacuum dried culture. Cultures are then resuspended using Pfennig's Medium I and II (#28 and 29) as described per DSMZ instructions. C. limicola is grown at 25° C. under constant vortexing.

Citrobacter braakii (DSMZ #30040) is obtained from the German Collection of Microorganisms and Cell Cultures (Braunschweig, Germany) as a vacuum dried culture. Cultures are then resuspended in Brain Heart Infusion (BHI) Broth (RPI Corp, Mt. Prospect, Ill., USA). Serial dilutions of the resuspended C. braakii culture are made into BHI and are allowed to grow for aerobically for 48 hours at 30° C. at 250 rpm until saturated.

Clostridium acetobutylicum (DSMZ #792) is obtained from the German Collection of Microorganisms and Cell Cultures (Braunschweig, Germany) as a vacuum dried culture. Cultures are then resuspended in Clostridium acetobutylicum medium (#411) as described per DSMZ instructions. C. acetobutylicum is grown anaerobically at 37° C. at 250 rpm until saturated.

Clostridium aminobutyricum (DSMZ #2634) is obtained from the German Collection of Microorganisms and Cell Cultures (Braunschweig, Germany) as a vacuum dried culture. Cultures are then resuspended in Clostridium aminobutyricum medium (#286) as described per DSMZ instructions. C. aminobutyricum is grown anaerobically at 37° C. at 250 rpm until saturated.

Clostridium kluyveri (DSMZ #555) is obtained from the German Collection of Microorganisms and Cell Cultures (Braunschweig, Germany) as an actively growing culture. Serial dilutions of C. kluyveri culture are made into Clostridium kluyveri medium (#286) as described per DSMZ instructions. C. kluyveri is grown anaerobically at 37° C. at 250 rpm until saturated.

Cupriavidus metallidurans (DMSZ #2839) is obtained from the German Collection of Microorganisms and Cell Cultures (Braunschweig, Germany) as a vacuum dried culture. Cultures are then resuspended in Brain Heart Infusion (BHI) Broth (RPI Corp, Mt. Prospect, Ill., USA). Serial dilutions of the resuspended C. metallidurans culture are made into BHI and are allowed to grow for aerobically for 48 hours at 30° C. at 250 rpm until saturated.

Cupriavidus necator (DSMZ #428) is obtained from the German Collection of Microorganisms and Cell Cultures (Braunschweig, Germany) as a vacuum dried culture. Cultures are then resuspended in Brain Heart Infusion (BHI) Broth (RPI Corp, Mt. Prospect, Ill., USA). Serial dilutions of the resuspended C. necator culture are made into BHI and are allowed to grow for aerobically for 48 hours at 30° C. at 250 rpm until saturated. As noted elsewhere, previous names for this species are Alcaligenes eutrophus and Ralstonia eutrophus.

Desulfovibrio fructosovorans (DSMZ #3604) is obtained from the German Collection of Microorganisms and Cell Cultures (Braunschweig, Germany) as a vacuum dried culture. Cultures are then resuspended in Desulfovibrio fructosovorans medium (#63) as described per DSMZ instructions. D. fructosovorans is grown anaerobically at 37° C. at 250 rpm until saturated.

Escherichia coli Crooks (DSMZ #1576) is obtained from the German Collection of Microorganisms and Cell Cultures (Braunschweig, Germany) as a vacuum dried culture. Cultures are then resuspended in Brain Heart Infusion (BHI) Broth (RPI Corp, Mt. Prospect, Ill., USA). Serial dilutions of the resuspended E. coli Crooks culture are made into BHI and are allowed to grow for aerobically for 48 hours at 37° C. at 250 rpm until saturated.

Escherichia coli K12 is a gift from the Gill lab (University of Colorado at Boulder) and is obtained as an actively growing culture. Serial dilutions of the actively growing E. coli K12 culture are made into Luria Broth (RPI Corp, Mt. Prospect, Ill., USA) and are allowed to grow for aerobically for 24 hours at 37° C. at 250 rpm until saturated.

Halobacterium salinarum (DSMZ #1576) is obtained from the German Collection of Microorganisms and Cell Cultures (Braunschweig, Germany) as a vacuum dried culture. Cultures are then resuspended in Halobacterium medium (#97) as described per DSMZ instructions. H. salinarum is grown aerobically at 37° C. at 250 rpm until saturated.

Lactobacillus delbrueckii (#4335) is obtained from WYEAST USA (Odell, Oreg., USA) as an actively growing culture. Serial dilutions of the actively growing L. delbrueckii culture are made into Brain Heart Infusion (BHI) broth (RPI Corp, Mt. Prospect, Ill., USA) and are allowed to grow for aerobically for 24 hours at 30° C. at 250 rpm until saturated.

Metallosphaera sedula (DSMZ #5348) is obtained from the German Collection of Microorganisms and Cell Cultures (Braunschweig, Germany) as an actively growing culture. Serial dilutions of M. sedula culture are made into Metallosphaera medium (#485) as described per DSMZ instructions. M. sedula is grown aerobically at 65° C. at 250 rpm until saturated.

Propionibacterium freudenreichii subsp. shermanii (DSMZ #4902) is obtained from the German Collection of Microorganisms and Cell Cultures (Braunschweig, Germany) as a vacuum dried culture. Cultures are then resuspended in PYG-medium (#104) as described per DSMZ instructions. P. freudenreichii subsp. shermanii is grown anaerobically at 30° C. at 250 rpm until saturated.

Pseudomonas putida is a gift from the Gill lab (University of Colorado at Boulder) and is obtained as an actively growing culture. Serial dilutions of the actively growing P. putida culture are made into Luria Broth (RPI Corp, Mt. Prospect, Ill., USA) and are allowed to grow for aerobically for 24 hours at 37° C. at 250 rpm until saturated.

Streptococcus mutans (DSMZ #6178) is obtained from the German Collection of Microorganisms and Cell Cultures (Braunschweig, Germany) as a vacuum dried culture. Cultures are then resuspended in Luria Broth (RPI Corp, Mt. Prospect, Ill., USA). S. mutans is grown aerobically at 37° C. at 250 rpm until saturated.

Subsection II: Gel Preparation, DNA Separation, Extraction, Ligation, and Transformation Methods:

Molecular biology grade agarose (RPI Corp, Mt. Prospect, Ill., USA) is added to 1× TAE to make a 1% Agarose: TAE solution. To obtain 50× TAE add the following to 900 mL of distilled water: add the following to 900 ml distilled H₂O: 242 g Tris base (RPI Corp, Mt. Prospect, Ill., USA), 57.1 ml Glacial Acetic Acid (Sigma-Aldrich, St. Louis, Mo., USA) and 18.6 g EDTA (Fisher Scientific, Pittsburgh, Pa. USA) and adjust volume to 1 L with additional distilled water. To obtain 1× TAE, add 20 mL of 50× TAE to 980 mL of distilled water. The agarose-TAE solution is then heated until boiling occurred and the agarose is fully dissolved. The solution is allowed to cool to 50° C. before 10 mg/mL ethidium bromide (Acros Organics, Morris Plains, N.J., USA) is added at a concentration of 5 ul per 100 mL of 1% agarose solution. Once the ethidium bromide is added, the solution is briefly mixed and poured into a gel casting tray with the appropriate number of combs (Idea Scientific Co., Minneapolis, Minn., USA) per sample analysis. DNA samples are then mixed accordingly with 5× TAE loading buffer. 5× TAE loading buffer consists of 5× TAE (diluted from 50× TAE as described above), 20% glycerol (Acros Organics, Morris Plains, N.J., USA), 0.125% Bromophenol Blue (Alfa Aesar, Ward Hill, Mass., USA), and adjust volume to 50 mL with distilled water. Loaded gels are then run in gel rigs (Idea Scientific Co., Minneapolis, Minn., USA) filled with 1× TAE at a constant voltage of 125 volts for 25-30 minutes. At this point, the gels are removed from the gel boxes with voltage and visualized under a UV transilluminator (FOTODYNE Inc., Hartland, Wis., USA).

The DNA isolated through gel extraction is then extracted using the QIAquick Gel Extraction Kit following manufacturer's instructions (Qiagen (Valencia Calif. USA)). Similar methods are known to those skilled in the art.

The thus-extracted DNA then may be ligated into pSMART (Lucigen Corp, Middleton, Wis., USA), StrataClone (Stratagene, La Jolla, Calif., USA) or pCR2.1-TOPO TA (Invitrogen Corp, Carlsbad, Calif., USA) according to manufacturer's instructions. These methods are described in the next subsection of Common Methods.

Ligation Methods:

For Ligations into pSMART Vectors:

Gel extracted DNA is blunted using PCRTerminator (Lucigen Corp, Middleton, Wis., USA) according to manufacturer's instructions. Then 500 ng of DNA is added to 2.5 uL 4× CloneSmart vector premix, 1 ul CloneSmart DNA ligase (Lucigen Corp, Middleton, Wis., USA) and distilled water is added for a total volume of 10 ul. The reaction is then allowed to sit at room temperature for 30 minutes and then heat inactivated at 70° C. for 15 minutes and then placed on ice. E. coli 10 G Chemically Competent cells (Lucigen Corp, Middleton, Wis., USA) are thawed for 20 minutes on ice. 40 ul of chemically competent cells are placed into a microcentrifuge tube and 1 ul of heat inactivated CloneSmart Ligation is added to the tube. The whole reaction is stirred briefly with a pipette tip. The ligation and cells are incubated on ice for 30 minutes and then the cells are heat shocked for 45 seconds at 42° C. and then put back onto ice for 2 minutes. 960 ul of room temperature Recovery media (Lucigen Corp, Middleton, Wis., USA) and places into microcentrifuge tubes. Shake tubes at 250 rpm for 1 hour at 37° C. Plate 100 ul of transformed cells on Luria Broth plates (RPI Corp, Mt. Prospect, Ill., USA) plus appropriate antibiotics depending on the pSMART vector used. Incubate plates overnight at 37° C.

For Ligations into StrataClone:

Gel extracted DNA is blunted using PCRTerminator (Lucigen Corp, Middleton, Wis., USA) according to manufacturer's instructions. Then 2 ul of DNA is added to 3 ul StrataClone Blunt Cloning buffer and 1 ul StrataClone Blunt vector mix amp/kan (Stratagene, La Jolla, Calif., USA) for a total of 6 ul Mix the reaction by gently pipeting up at down and incubate the reaction at room temperature for 30 minutes then place onto ice. Thaw a tube of StrataClone chemically competent cells (Stratagene, La Jolla, Calif., USA) on ice for 20 minutes. Add 1 ul of the cloning reaction to the tube of chemically competent cells and gently mix with a pipette tip and incubate on ice for 20 minutes. Heat shock the transformation at 42° C. for 45 seconds then put on ice for 2 minutes. Add 250 ul pre-warmed Luria Broth (RPI Corp, Mt. Prospect, Ill., USA) and shake at 250 rpm for 37° C. for 2 hour. Plate 100 ul of the transformation mixture onto Luria Broth plates (RPI Corp, Mt. Prospect, Ill., USA) plus appropriate antibiotics. Incubate plates overnight at 37° C.

For Ligations into pCR2.1-TOPO TA:

Add 1 ul TOPO vector, 1 ul Salt Solution (Invitrogen Corp, Carlsbad, Calif., USA) and 3 ul gel extracted DNA into a microcentrifuge tube. Allow the tube to incubate at room temperature for 30 minutes then place the reaction on ice. Thaw one tube of TOP1OF′ chemically competent cells (Invitrogen Corp, Carlsbad, Calif., USA) per reaction. Add 1 l of reaction mixture into the thawed TOP10F′ cells and mix gently by swirling the cells with a pipette tip and incubate on ice for 20 minutes. Heat shock the transformation at 42° C. for 45 seconds then put on ice for 2 minutes. Add 250 ul pre-warmed SOC media (Invitrogen Corp, Carlsbad, Calif., USA) and shake at 250 rpm for 37° C. for 1 hour. Plate 100 ul of the transformation mixture onto Luria Broth plates (RPI Corp, Mt. Prospect, Ill., USA) plus appropriate antibiotics. Incubate plates overnight at 37° C.

General Transformation and Related Culture Methodologies:

Chemically competent transformation protocols are carried out according to the manufacturer's instructions or according to the literature contained in Molecular Cloning (Sambrook and Russell, 2001). Generally, plasmid DNA or ligation products are chilled on ice for 5 to 30 min. in solution with chemically competent cells. Chemically competent cells are a widely used product in the field of biotechnology and are available from multiple vendors, such as those indicated above in this Subsection. Following the chilling period cells generally are heat-shocked for 30 seconds at 42° C. without shaking, re-chilled and combined with 250 microliters of rich media, such as S. O. C. Cells are then incubated at 37° C. while shaking at 250 rpm for 1 hour. Finally, the cells are screened for successful transformations by plating on media containing the appropriate antibiotics.

Alternatively, selected cells may be transformed by electroporation methods such as are known to those skilled in the art.

The choice of an E. coli host strain for plasmid transformation is determined by considering factors such as plasmid stability, plasmid compatibility, plasmid screening methods and protein expression. Strain backgrounds can be changed by simply purifying plasmid DNA as described above and transforming the plasmid into a desired or otherwise appropriate E. coli host strain such as determined by experimental necessities, such as any commonly used cloning strain (e.g., DH5α, Top10F′, E. coli 10 G, etc.).

To make 1 L M9 Minimal Media:

M9 minimal media was made by combining 5× M9 salts, 1M MgSO_{4,} 20% glucose, 1M CaCl₂ and sterile deionized water. The 5× M9 salts are made by dissolving the following salts in deionized water to a final volume of 1 L: 64 g Na₂HPO₄.7H₂O, 15 g KH₂PO₄, 2.5 g NaCl, 5.0 g NH₄Cl. The salt solution was divided into 200 mL aliquots and sterilized by autoclaving for 15 minutes at 15 psi on the liquid cycle. A 1M solution of MgSO₄ and 1M CaCl₂ were made separately, then sterilized by autoclaving. The glucose was filter sterilized by passing it thought a 0.22 μm filter. All of the components are combined as follows to make 1 L of M9: 750 mL sterile water, 200 mL 5× M9 salts, 2 mL of 1M MgSO_{4,} 20 mL 20% glucose, 0.1 mL CaCl₂, Q. S. to a final volume of 1 L.

To Make EZ Rich Media:

All media components were obtained from TEKnova (Hollister Calif. USA) and combined in the following volumes. 100 mL 10× MOPS mixture, 10 mL 0.132M K₂ HPO_{4,} 100 mL 10×ACGU, 200 mL 5× Supplement EZ, 10 mL 20% glucose, 580 mL sterile water.

Subsection IIIa. 3-HP Preparation

A 3-HP stock solution was prepared as follows and used in examples other than Example 1. A vial of β-propriolactone (Sigma-Aldrich, St. Louis, Mo., USA) was opened under a fume hood and the entire bottle contents was transferred to a new container sequentially using a 25-mL glass pipette. The vial was rinsed with 50 mL of HPLC grade water and this rinse was poured into the new container. Two additional rinses were performed and added to the new container. Additional HPLC grade water was added to the new container to reach a ratio of 50 mL water per 5 mL δ-propriolactone. The new container was capped tightly and allowed to remain in the fume hood at room temperature for 72 hours. After 72 hours the contents were transferred to centrifuge tubes and centrifuged for 10 minutes at 4,000 rpm. Then the solution was filtered to remove particulates and, as needed, concentrated by use of a rotary evaporator at room temperature. Assay for concentration was conducted per below, and dilution to make a standard concentration stock solution was made as needed.

It is noted that there appear to be small lot variations in the toxicity of 3-HP solutions. Without being bound to a particular theory, it is believed the variation can be correlated with a low level of contamination by acrylic acid, which is more toxic than 3-HP, and also, to a lesser extent, to presence of a polymer of δ-propriolactone. HPLC results show the presence of the acrylic peak, which, as noted, is a minor contaminant varying in concentration from batch to batch.

Subsection IIIb. HPLC and GC/MS Analytical Methods for 3-HP Detection

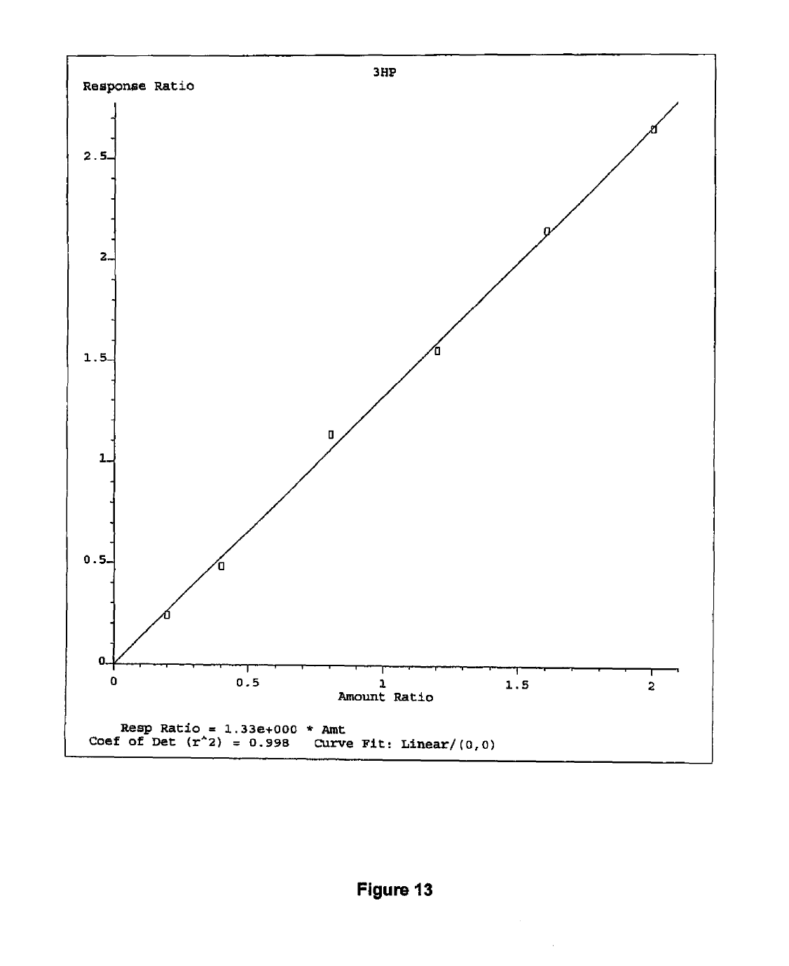

For HPLC analysis of 3-HP, the Waters chromatography system (Milford, Mass.) consisted of the following: 600S Controller, 616 Pump, 717 Plus Autosampler, 486 Tunable UV Detector, and an in-line mobile phase Degasser. In addition, an Eppendorf external column heater is used and the data are collected using an SRI (Torrance, Calif.) analog-to-digital converter linked to a standard desk top computer. Data are analyzed using the SRI Peak Simple software. A Coregel 64H ion exclusion column (Transgenomic, Inc., San Jose, Calif.) is employed. The column resin is a sulfonated polystyrene divinyl benzene with a particle size of 10 μm and column dimensions are 300×7.8 mm. The mobile phase consisted of sulfuric acid (Fisher Scientific, Pittsburgh, Pa. USA) diluted with deionized (18 MΩcm) water to a concentration of 0.02 N and vacuum filtered through a 0.2 μm nylon filter. The flow rate of the mobile phase is 0.6 mL/min. The UV detector is operated at a wavelength of 210 nm and the column is heated to 60° C. The same equipment and method as described herein is used for 3-HP analyses for relevant prophetic examples. Calibration curves using this HPLC method with a 3-HP standard (TCI America, Portland, Oreg.) is provided in FIG. 13.

The following method is used for GC-MS analysis of 3-HP. Soluble monomeric 3-HP is quantified using GC-MS after a single extraction of the fermentation media with ethyl acetate. The GC-MS system consists of a Hewlett Packard model 5890 GC and Hewlett Packard model 5972 MS. The column is Supelco SPB-1 (60 m×0.32 mm×0.25μm film thickness). The capillary coating is a non-polar methylsilicone. The carrier gas is helium at a flow rate of 1 mL/min. 3-HP is separated from other components in the ethyl acetate extract, using a temperature gradient regime starting with 40° C. for 1 minute, then 10° C./minute to 235° C., and then 50° C./minute to 300° C. Tropic acid (1 mg/mL) is used as the internal standard. 3-HP is quantified using a 3HP standard curve at the beginning of the run and the data are analyzed using HP Chemstation. A calibration curve, automatically generated with use of a standard, is provided as FIG. 14.

Subsection IVa. Minimum Inhibitory Concentration Evaluation (MIC) General Protocols (for Evaluations Other than in Examples 1-4)

E. coli Aerobic

The minimum inhibitory concentration (MIC) was determined aerobically in a 96 well-plate format. Plates were setup such that each individual well, when brought to a final volume of 100 uL following inoculation, had the following component levels (corresponding to standard M9 media): 47.7 mM Na₂HPO₄, 22 mM KH₂PO₄, 8.6 mM NaCl, 18.7 mM NH₄Cl, 2 mM MgSO₄, 0.1 mM CaCl₂, and 0.4% glucose. Media supplements were added according to levels reported in Table 3, where specified. Overnight cultures of strains were grown in triplicate in 5 mL LB (with antibiotic where appropriate). A 1% (v/v) inoculum was introduced into a 5 ml culture of M9 minimal media. After the cells reached mid-exponential phase, the culture was diluted to an OD₆₀₀ of about 0.200 (i.e., 0.195-0.205. The cells were further diluted 1:50 and a 10 μL aliquot was used to inoculate each well of a 96 well plate (˜10⁴ cells per well) to total volume of 100 uL. The plate was arranged to measure the growth of variable strains or growth conditions in increasing 3-HP concentrations, 0 to 60 g/L, in 5 g/L increments. Plates were incubated for 24 hours at 37 C. The minimum inhibitory 3-HP concentration and maximum 3-HP concentration corresponding to visible cell growth (OD˜0.1) was recorded after 24 hours. For cases when MIC>60 g/L, assessments were performed in plates with extended 3-HP concentrations (0-100 g/L, in 5 g/L increments).

E. coli Anaerobic

The minimum inhibitory concentration (MIC) was determined anerobically in a 96 well-plate format. Plates were setup such that each individual well, when brought to a final volume of 100 uL following inoculation, had the following component levels (corresponding to standard M9 media): 47.7 mM Na₂HPO₄, 22 mM KH₂PO₄, 8.6 mM NaCl, 18.7 mM NH₄Cl, 2 mM MgSO₄, 0.1 mM CaCl₂, and 0.4% glucose. Media supplements were added according to levels reported in Table 3, where specified. Overnight cultures of strains were grown in triplicate in 5 mL LB (with antibiotic where appropriate). A 1% (v/v) inoculum was introduced into a 5 ml culture of M9 minimal media. After the cells reached mid-exponential phase, the culture was diluted to an OD₆₀₀ of about 0.200 (i.e., 0.195-0.205. The cells were further diluted 1:50 and a 10 μL aliquot was used to inoculate each well of a 96 well plate (˜10⁴ cells per well) to total volume of 100 uL. The plate was arranged to measure the growth of variable strains or growth conditions in increasing 3-HP concentrations, 0 to 60 g/L, in 5 g/L increments. Plates were sealed in biobag anaerobic chambers that contained gas generators for anaerobic conditions and incubated for 24 hours at 37 C. The minimum inhibitory 3-HP concentration and maximum 3-HP concentration corresponding to visible cell growth (OD˜0.1) was recorded after 24 hours. For cases when MIC>60 g/L, assessments were performed in plates with extended 3-HP concentrations (0-100 g/L, in 5 g/L increments).

B. subtilis Aerobic

The minimum inhibitory concentration (MIC) was determined aerobically in a 96 well-plate format. Plates were setup such that each individual well, when brought to a final volume of 100 uL following inoculation, had the following component levels (corresponding to standard M9 media+supplemental glutamate): 47.7 mM Na₂HPO₄, 22 mM KH₂PO₄, 8.6 mM NaCl, 18.7 mM NH₄Cl, 2 mM MgSO₄, 0.1 mM CaCl₂, 10 mM glutamate and 0.4% glucose. Media supplements were added according to levels reported in Table 3, where specified. Overnight cultures of strains were grown in triplicate in 5 mL LB (with antibiotic where appropriate). A 1% (v/v) inoculum was introduced into a 5 ml culture of M9 minimal media+glutamate. After the cells reached mid-exponential phase, the culture was diluted to an OD₆₀₀ of about 0.200 (i.e., 0.195-0.205. The cells were further diluted 1:50 and a 10 μL aliquot was used to inoculate each well of a 96 well plate (˜10⁴ cells per well) to total volume of 100 uL. The plate was arranged to measure the growth of variable strains or growth conditions in increasing 3-HP concentrations, 0 to 60 g/L, in 5 g/L increments. Plates were incubated for 24 hours at 37 C. The minimum inhibitory 3-HP concentration and maximum 3-HP concentration corresponding to visible cell growth (OD˜0.1) was recorded after 24 hours. For cases when MIC>60 g/L, assessments were performed in plates with extended 3-HP concentrations (0-100 g/L, in 5 g/L increments).

C. necator (R. eutropha) Aerobic

The minimum inhibitory concentration (MIC) was determined aerobically in a 96 well-plate format. Plates were setup such that each individual well, when brought to a final volume of 100 uL following inoculation, had the following component levels (corresponding to FGN media): 21.5 mM K₂HPO₄, 8.5 mM KH₂PO₄, 18 mM NH₄Cl, 12 mM NaCl, 7.3 uM ZnCl, 0.15 uM MnCl₂, 4.85 uM H₃BO₃, 0.21 uM CoCl₂, 0.41 uM CuCl₂, 0.50 uM NiCl₂, 0.12 uM Na₂MoO₄, 0.19 uM CrCl₃, 0.06 mM CaCl₂, 0.5 mM MgSO₄, 0.06 mM FeSO₄, 0.2% glycerol, 0.2% fructose. Media supplements were added according to levels reported in Table 3, where specified. Overnight cultures of strains were grown in triplicate in 5 mL LB (with antibiotic where appropriate). A 1% (v/v) inoculum was introduced into a 5 ml culture of FGN media. After the cells reached mid-exponential phase, the culture was diluted to an OD₆₀₀ of about 0.200 (i.e., 0.195-0.205. The cells were further diluted 1:50 and a 10 μL aliquot was used to inoculate each well of a 96 well plate (˜10⁴ cells per well) to total volume of 100 uL. The plate was arranged to measure the growth of variable strains or growth conditions in increasing 3-HP concentrations, 0 to 60 g/L, in 5 g/L increments. Plates were incubated for 24 hours at 30 C. The minimum inhibitory 3-HP concentration and maximum 3-HP concentration corresponding to visible cell growth (OD˜0.1) was recorded after 24 hours. For cases when MIC>60 g/L, assessments were performed in plates with extended 3-HP concentrations (0-100 g/L, in 5 g/L increments).

For the above MIC evaluations, the final results are expressed in chemical agent concentrations determined by analysis of the stock solution by HPLC (i.e., see Subsection IIIb).

### Summary of Suppliers Section

This section is provided for a summary of suppliers, and may be amended to incorporate additional supplier information in subsequent filings. The names and city addresses of major suppliers are provided in the methods above. In addition, as to Qiagen products, the DNeasy® Blood and Tissue Kit, Cat. No. 69506, is used in the methods for genomic DNA preparation; the QIAprep® Spin (“mini prep”), Cat. No. 27106, is used for plasmid DNA purification, and the QIAquick® Gel Extraction Kit, Cat. No. 28706, is used for gel extractions as described above.

TABLE 1
SCALES Fitness Data

| | Cumulative |
|---|---|
| Gene | Fitness |
| aceE | 11.2 |
| aceF | 8.39 |
| ackA | 2.36 |
| acnA | 3.58 |
| acnB | 3.18 |
| adhE | 3.68 |
| adiA | 1.95 |
| adk | 2.18 |
| aldA | 1.83 |
| argA | 3.94 |
| argB | 8.94 |
| argC | 4.02 |
| argD | 2.87 |
| argE | 2.15 |
| argF | 2.04 |
| argG | 2.62 |
| argH | 8.06 |
| argI | 4.06 |
| aroA | 2.31 |
| aroB | 8.68 |
| aroC | 1.95 |
| aroD | 1.93 |
| aroE | 8.44 |
| aroF | 6.24 |
| aroG | 2.26 |
| aroH | 1.61 |
| aroK | 4 |
| aroL | 1.63 |
| asd | 2.96 |
| aspC | 2.82 |
| astC | 2.29 |
| carA | 0.89 |
| carB | 1.17 |
| cynS | 4.83 |
| cysE | 1.19 |
| cysK | 2.41 |
| pabC | 1.75 |
| pfkA | 1.78 |
| pflB | 2.83 |
| purB | 3.65 |
| purC | 1.78 |
| purD | 1.32 |
| purE | 1.82 |
| purF | 2.04 |
| purH | 1.66 |
| purK | 2.65 |
| purL | 4.83 |
| purM | 3.13 |
| purN | 2.94 |
| purT | 3.73 |
| puuE | 1.53 |
| pyrB | 6.36 |
| pyrC | 14.48 |
| pyrD | 2.26 |
| pyrE | 1.03 |
| pyrF | 1.38 |
| pyrG | 2.23 |
| pyrH | 1.78 |
| pyrI | 0.83 |
| rpe | 2.06 |
| cysM | 26.63 |
| eno | 6.98 |
| entA | 1.58 |
| entB | 0.93 |
| entC | 1.26 |
| entD | 1 |
| entE | 1.03 |
| entF | 1.03 |
| fbaA | 2.87 |
| fbaB | 2.28 |
| folA | 15.07 |
| folB | 0.57 |
| folC | 1.72 |
| folD | 8.54 |
| folE | 1.08 |
| folK | 1.73 |
| folP | 2.45 |
| fumA | 3.84 |
| fumB | 2.51 |
| fumC | 1.86 |
| gabD | 1.83 |
| gabT | 1.41 |
| gapA | 3.03 |
| gcvH | 5.9 |
| gcvP | 7.91 |
| gcvT | 1.78 |
| gdhA | 2.84 |
| gldA | 2.08 |
| glk | 1.17 |
| glnA | 1.34 |
| gltA | 6.37 |
| glyA | 5.06 |
| gmk | 1.86 |
| gnd | 1.69 |
| gpmA | 2.01 |
| guaA | 3.65 |
| guaB | 2.63 |
| ilvA | 12.21 |
| ilvB | 2.7 |
| rpiA | 1.85 |
| sdaA | 1.62 |
| sdaB | 1.22 |
| serA | 3.11 |
| serB | 2.46 |
| serC | 2.15 |
| speA | 2.09 |
| speB | 1.66 |
| speC | 1.52 |
| speD | 3.43 |
| talA | 1.24 |
| talB | 4.78 |
| tdcB | 1.87 |
| tdcD | 1.64 |
| tdcE | 1.16 |
| tdh | 1.38 |
| tktA | 1.89 |
| tktB | 1.21 |
| trpA | 2.45 |
| trpB | 1.93 |
| ilvC | 2.61 |
| ilvD | 1.6 |
| ilvE | 0.94 |
| ilvH | 1.18 |
| ilvI | 1.77 |
| ilvM | 1.02 |
| ilvN | 1.53 |
| kbl | 3.11 |
| itaE | 1.14 |
| lysC | 1.97 |
| malY | 2.58 |
| menA | 3.2 |
| menB | 0.86 |
| menC | 0.92 |
| menD | 2.33 |
| menE | 3.06 |
| menF | 3.09 |
| metA | 1.56 |
| metB | 1.83 |
| metC | 6.08 |
| metE | 2.46 |
| metH | 2.44 |
| metK | 3.35 |
| metL | 2.97 |
| mhpF | 1.44 |
| ndk | 1.66 |
| nrdA | 2.01 |
| nrdB | 1.81 |
| nrdD | 2.79 |
| nrdE | 1.91 |
| nrdF | 1.25 |
| pabA | 2.33 |
| pabB | 1.92 |
| thrA | 2.79 |
| thrB | 0.96 |
| thrC | 1.51 |
| pheA | 6.7 |
| pta | 2.7 |
| purA | 5.1 |
| trpC | 1.56 |
| trpD | 2.48 |
| trpE | 2.85 |
| tynA | 2.36 |
| tyrA | 9.1 |
| tyrB | 1.49 |
| ubiA | 1.51 |
| ubiB | 2.09 |
| ubiC | 2.4 |
| ubiD | 0.91 |
| ubiE | 1.02 |
| ubiF | 1.78 |
| ubiG | 3.17 |
| ubiH | 5.35 |
| ubiX | 1.72 |
| ydcW | 0.89 |
| ydiB | 0.87 |
| ygjG | 2.51 |
| ynel/sad | 4.18 |

TABLE 2
Homology Relationships for Genetic Elements of C. necator

| E. coli | | | C. necator | | |
|---|---|---|---|---|---|
| Gene | | E. coli enzyme | Gene | C. necator | |
| Symbol | E. coli enzyme product | substrate | Symbol | E-value | C. necator Gene Product |
| acee | pyruvate | acetyl-coA | aceE | 0 | pyruvate dehydrogenase subunit E1 |
| acee | pyruvate | acetyl-coA | aceE | 0 | pyruvate dehydrogenase subunit E1 |
| acee | pyruvate | acetyl-coA | aceE | 0 | 2-oxoacid dehydrogenase subunit E1 |
| acef | gi\|16128108\|ref\|NP_414657.1\| | pyruvate | pdhB | 2.00E−102 | dihydrolipoamide acetyltransferase |
| acef | gi\|16128108\|ref\|NP_414657.1\| | pyruvate | pdhB | 2.00E−25 | dihydrolipoamide acetyltransferase |
| acef | pyruvate | acetyl-coA | pdhB | 2.00E−22 | dihydrolipoamide acetyltransferase |
| acef | pyruvate | acetyl-coA | pdhB | 1.00E−10 | dihydrolipoamide acetyltransferase |
| acef | pyruvate | acetyl-coA | pdhL | 6.00E−11 | dihydrolipoamide dehydrogenase (E3) |
| | | | | | component ofpyruvate dehydrogenase |
| acef | pyruvate | acetyl-coA | pdhL | 2.00E−09 | dihydrolipoamide dehydrogenase (E3) |
| | | | | | component ofpyruvate dehydrogenase |
| acef | pyruvate | acetyl-coA | pdhL | 8.00E−08 | dihydrolipoamide dehydrogenase (E3) |
| | | | | | component ofpyruvate dehydrogenase |
| acef | pyruvate | acetyl-coA | odhB | 9.00E−36 | dihydrolipoamide acetyltransferase |
| acef | pyruvate | acetyl-coA | bkdB | 1.00E−30 | branched-chain alpha-keto acid |
| | | | | | dehydrogenase subunit E2 |
| acef | pyruvate | acetyl-coA | bkdB | 1.00E−07 | branched-chain alpha-keto acid |
| | | | | | dehydrogenase subunit E2 |
| acef | pyruvate | acetyl-coA | bkdB | 2.00E−07 | branched-chain alpha-keto acid |
| | | | | | dehydrogenase subunit E2 |
| acna | gi\|16129237\|ref\|NP_415792.1\| | citrate | leuC1 | 2.00E−19 | isopropylmalate isomerase large subunit |
| acna | gi\|16129237\|ref\|NP_415792.1\| | citrate | leuC2 | 7.00E−22 | isopropylmalate isomerase large subunit |
| acna | gi\|16129237\|ref\|NP_415792.1\| | citrate | acnM | 0 | aconitate hydratase |
| acna | gi\|16129237\|ref\|NP_415792.1\| | citrate | leuC3 | 6.00E−20 | isopropylmalate isomerase large subunit |
| acna | citrate | cis-aconitate | acnA | 0 | aconitate hydratase |
| acna | citrate | cis-aconitate | leuC4 | 6.00E−14 | 3-isopropylmalate dehydratase large |
| | | | | | subunit |
| acna | citrate | cis-aconitate | leuC5 | 1.00E−12 | isopropylmalate isomerase large subunit |

. . . (intervening data removed to shorten table)

| | | | | | |
|---|---|---|---|---|---|
| ytjc | gi\|16132212\|ref\|NP_418812.1\| | 3-phosphoglycerate | pgam2 | 3.00E−25 | phosphoglycerate mutase 2 protein |
| ytjc | 3-phosphoglycerate | 2-phosphoglycerate | pgam2 | 3.00E−25 | phosphoglycerate mutase 2 protein |
| zwf | gi\|16129805\|ref\|NP_416366.1\| | glucose-6-phosphate | zwf1 | 2.00E−132 | glucose-6-phosphate 1-dehydrogenase |
| zwf | glucose-6-phosphate | glucono-lactone-6- | zwf2 | 7.00E−126 | glucose-6-phosphate 1-dehydrogenase |
| | | phosphate | | | |
| zwf | glucose-6-phosphate | glucono-lactone-6- | zwf3 | 8.00E−130 | glucose-6-phosphate 1-dehydrogenase |
| | | phosphate | | | |

TABLE 3
Supplement

| | | TGC | Concentration, | |
|---|---|---|---|---|
| Supplement | Source | Group | g/L | Note |
| Tyrosine | Sigma, St. Louis, MO | A | 0.036 | dissolve in 0.01 |
| | | | | KOH, pH final to 7 |
| Phenylalanine | Sigma, St. Louis, MO | A | 0.0664 | |
| Tryptophan | Sigma, St. Louis, MO | A | 0.0208 | |
| Shikimate | Sigma, St. Louis, MO | A | 0.1 | |
| p-aminobenzoate | MP Biomedicals, | A | 0.069 | |
| | Aurora, OH | | | |
| Dihydroxybenzoate | Sigma, St. Louis, MO | A | 0.077 | |
| Tetrahydrofolate | Sigma, St. Louis, MO | A | 0.015 | 10% DMSO |
| Homocysteine | MP Biomedicals, | B | 0.008 | |
| | Aurora, OH | | | |
| Isoleucine | Sigma, St. Louis, MO | B | 0.0052 | |
| Serine | Sigma, St. Louis, MO | B | 1.05 | |
| Glycine | Fisher Scientific, Fair | B | 0.06 | |
| | Lawn, NJ | | | |
| Methionine | Sigma, St. Louis, MO | B | 0.03 | |
| Threonine | Sigma, St. Louis, MO | B | 0.0476 | |
| 2-oxobutyrate | Fluka Biochemika, | B | 0.051 | |
| | Hungary | | | |
| Homoserine | Acros Organics, NJ | B | 0.008 | |
| Aspartate | Sigma, St. Louis, MO | B | 0.0684 | |
| Putrescine | MP Biomedicals, Salon, | C | 0.9 | |
| | OH | | | |
| Cadaverine | MP Biomedicals, Salon, | C | 0.6 | |
| | OH | | | |
| Spermidine | MP Biomedicals, Salon, | C | 0.5 | |
| | OH | | | |
| Ornithine | Sigma, St. Louis, MO | C | 0.2 | |
| Citrulline | Sigma, St. Louis, MO | C | 0.2 | |
| Bicarbonate | Fisher Scientific, Fair | C | 1 | |
| | Lawn, NJ | | | |
| Glutamine | Sigma, St. Louis, MO | C | 0.09 | dissolve in 1M HCl, |
| | | | | pH final to 7 |
| Lysine | Sigma, St. Louis, MO | D | 0.0732 | |
| Uracil | Sigma, St. Louis, MO | E | 0.224 | |
| Citrate | Fisher Scientific, Fair | F | 2 | |
| | Lawn, NJ | | | |
| Chorismate Group | See above | A | See respective | |
| Mix (includes all | | | concentrations | |
| Group A supplements | | | above | |
| listed above) | | | | |
| Homocysteine Group | See above | B | See respective | |
| Mix (includes all | | | concentrations | |
| Group B supplements | | | above | |
| listed above) | | | | |
| Polyamine Group Mix | See above | C | See respective | |
| (includes all Group C | | | concentrations | |
| supplements listed | | | above | |
| above) | | | | |

TABLE 4A
Vectors

| Vector | Sequence ID NOs. |
|---|---|
| pSMART-HC-Amp | SEQ ID NO: 005 |
| pSMART-LC-Kan | SEQ ID NO: 006 |
| pBT-3 | SEQ ID NO: 007 |
| pKK223-3 | SEQ ID NO: 008 |
| pACYC177 (kan only) | SEQ ID NO: 009 |
| pWH1520 | SEQ ID NO: 010 |
| pHT08 | SEQ ID NO: 011 |
| pJ61:25125 | SEQ ID NO: 012 |
| pYes2.1-topo | SEQ ID NO: 183 |
| pRS423 | SEQ ID NO: 184 |
| pRS425 | SEQ ID NO: 185 |
| pJ251 | SEQ ID NO: 186 |

TABLE 4B
E. coli Tolerance Plasmid Construction

| | | | | | PCR Sequence or | |
|---|---|---|---|---|---|---|
| Gene(s) or | | Cloning | | | Codon Optimized | |
| Region Name | Vector | Method | Primer A | Primer B | Sequence (Region) | Plasmid Name |
| aroG | pJ61 | A | N/A | N/A | SEQ ID NO: 013 | pJ61-aroG |
| speFED | pJ61 | A | N/A | N/A | SEQ ID NO: 014 | pJ61-speFED |
| thrA | pJ61 | A | N/A | N/A | SEQ ID NO: 015 | pJ61-thrA |
| asd | pJ61 | A | N/A | N/A | SEQ ID NO: 016 | pJ61-asd |
| cysM | pJ61 | A | N/A | N/A | SEQ ID NO: 017 | pJ61-cysM |
| ilvA | pJ61 | A | N/A | N/A | SEQ ID NO: 018 | pJ61-ilvA |
| aroH | pKK223 | B | N/A | N/A | (See SEQ ID NO: 001) | pKK223-aroH |
| aroH G149C | pKK223 | B | N/A | N/A | N/A | pKK223-aroH*445 |
| aroH G149D | pKK223 | B | N/A | N/A | N/A | pKK223-aroH*447 |
| aroH P18L | pKK223 | B | N/A | N/A | N/A | pKK223-aroH*457 |
| metE C645A | pKK223 | B | N/A | N/A | N/A | pKK223-metE C645A |
| thrA | pKK223 | B | N/A | N/A | SEQ ID NO: 019 | pKK223-thrA |
| cynTS | pSMART-LC-Kan | B | N/A | N/A | SEQ ID NO: 020 (and | pSmart-LC-Kan-cynTS |
| | | | | | See SEQ ID NO: 002) | |
| folA C1 | pSMART-LC-KAN | C | SEQ ID NO: 021 | SEQ ID NO: 022 | SEQ ID NO: 023 | pSmart-LC-Kan-folA-C1 |
| folA ORF | pSMART-LC-KAN | C | SEQ ID NO: 024 | SEQ ID NO: 025 | SEQ ID NO: 026 | pSmart-LC-Kan-folA-ORF |
| folD | pSMART-LC-KAN | C | SEQ ID NO: 027 | SEQ ID NO: 028 | SEQ ID NO: 029 | pSmart-LC-Kan-folD |
| aroKB C1 | pSMART-LC-KAN | C | SEQ ID NO: 030 | SEQ ID NO: 031 | SEQ ID NO: 032 | pSmart-LC-Kan-aroKB C1 |
| pheA C1 | pSMART-LC-KAN | C | SEQ ID NO: 033 | SEQ ID NO: 034 | SEQ ID NO: 035 | pSmart-LC-Kan-pheA C1 |
| pheA C2 | pSMART-LC-KAN | C | SEQ ID NO: 036 | SEQ ID NO: 037 | SEQ ID NO: 038 | pSmart-LC-Kan-pheA C2 |
| menA C1 | pSMART-LC-KAN | C | SEQ ID NO: 039 | SEQ ID NO: 040 | SEQ ID NO: 041 | pSmart-LC-Kan-menA C1 |
| menA ORF | pSMART-LC-KAN | C | SEQ ID NO: 042 | SEQ ID NO: 043 | SEQ ID NO: 044 | pSmart-LC-Kan-menA ORF |
| serA | pSMART-LC-KAN | C | SEQ ID NO: 045 | SEQ ID NO: 046 | SEQ ID NO: 047 | pSmart-LC-Kan-serA |
| glyA C1 | pSMART-LC-KAN | C | SEQ ID NO: 048 | SEQ ID NO: 049 | SEQ ID NO: 050 | pSmart-LC-Kan-glyA C1 |
| glyA ORF | pSMART-LC-KAN | C | SEQ ID NO: 051 | SEQ ID NO: 052 | SEQ ID NO: 053 | pSmart-LC-Kan-glyA ORF |
| metC C1 | pSMART-LC-KAN | C | SEQ ID NO: 054 | SEQ ID NO: 055 | SEQ ID NO: 056 | pSMART-LC-KAN-metC C1 |
| tyrA | pSMART-LC-KAN | C | SEQ ID NO: 057 | SEQ ID NO: 058 | SEQ ID NO: 059 | pSmart-LC-Kan-tyrA |
| tyrA-aroF | pSMART-LC-KAN | C | SEQ ID NO: 060 | SEQ ID NO: 061 | SEQ ID NO: 062 | pSmart-LC-Kan-tyrA-aroF |
| aroE | pSMART-LC-KAN | C | SEQ ID NO: 063 | SEQ ID NO: 064 | SEQ ID NO: 065 | pSmart-LC-Kan-aroE |
| ilvA | pSMART-LC-KAN | C | SEQ ID NO: 066 | SEQ ID NO: 067 | SEQ ID NO: 068 | pSmart-LC-KAN-ilvA C1 |
| ilvA | pSMART-LC-KAN | C | SEQ ID NO: 069 | SEQ ID NO: 070 | SEQ ID NO: 071 | pSmart-LC-KAN-ilvA |
| | | | | | | operon |
| cysM | pSMART-LC-KAN | C | SEQ ID NO: 072 | SEQ ID NO: 073 | SEQ ID NO: 074 | pSmart-LC-Kan-cysM |
| cynTS | pSMART-HC-AMP | D | SEQ ID NO: 075 | SEQ ID NO: 076 | SEQ ID NO: 077 | pSmart-HC-Amp-cynTS |
| metC | pSMART-HC-Amp | D | SEQ ID NO: 078 | SEQ ID NO: 079 | SEQ ID NO: 080 | pSmart-HC-Amp-metC |
| dapA | pSMART-HC-Amp | E | SEQ ID NO: 081* | SEQ ID | SEQ ID NO: 083 | pSmart-HC-Amp-dapA |
| | | | | NO: 082* | | |
| cadA | pSMART-HC-Amp | E | SEQ ID NO: 084* | SEQ ID | SEQ ID NO: 086 | pSmart-HC-Amp-cadA |
| | | | | NO: 085* | | |
| prs | pSMART-HC-Amp | E | SEQ ID NO: 087* | SEQ ID | SEQ ID NO: 089 | pSmart-HC-Amp-prs |
| | | | | NO: 088* | | |
| nrdAB | pSMART-HC-Amp | E | SEQ ID NO: 090* | SEQ ID | SEQ ID NO: 092 | pSmart-HC-Amp-nrdAB |
| | | | | NO: 091* | | |
| nrdLEF | pSMART-HC-Amp | E | SEQ ID NO: 093* | SEQ ID | SEQ ID NO: 095 | pSmart-HC-Amp-nrdLEF |
| | | | | NO: 094* | | |
| lysA | pSMART-HC-Amp | E | SEQ ID NO: 096* | SEQ ID | SEQ ID NO: 098 | pSMART-HC-Amp-lysA |
| | | | | NO: 097* | | |
| cyntTS | pACYC177 (kan only) | F | SEQ ID NO: 099 | SEQ ID NO: 100 | SEQ ID NO: 101 | pACYC177-cynTS |
| aroH G149C | pACYC177 (kan only) | F | SEQ ID NO: 102 | SEQ ID NO: 103 | SEQ ID NO: 104 | pACYC177-aroH* |
| speB | pACYC177 (kan only) | F | SEQ ID NO: 105 | SEQ ID NO: 106 | SEQ ID NO: 107 | pACYC177-speB |
| metE C645A | pACYC177 (kan only) | F | SEQ ID NO: 108 | SEQ ID NO: 109 | SEQ ID NO: 110 | pACYC177-metE* |
| metC | pACYC177 (kan only) | F | SEQ ID NO: 111 | SEQ ID NO: 112 | SEQ ID NO: 113 | pACYC177-metC |
| cyntTS | pBT-3 | G | SEQ ID NO: 114 | SEQ ID NO: 115 | SEQ ID NO: 116 | pBT-3-cynTS |
| aroH G149C | pBT-3 | G | SEQ ID NO: 117 | SEQ ID NO: 118 | SEQ ID NO: 119 | pBT-3-aroH* |
| speB | pBT-3 | G | SEQ ID NO: 120 | SEQ ID NO: 121 | SEQ ID NO: 122 | pBT-3-speB |

*5′phosphorylated

TABLE 5
Deletion Constructs

| Keio Clone | | | |
|---|---|---|---|
| Number | Gene Deletion | Forward Primer | Reverse Primer |
| JW1650 | purR | SEQ ID: 130 | SEQ ID: 131 |
| JW2807 | lysR | SEQ ID: 132 | SEQ ID: 133 |
| JW1316 | tyrR | SEQ ID: 134 | SEQ ID: 135 |
| JW4356 | trpR | SEQ ID: 136 | SEQ ID: 137 |
| JW3909 | metJ | SEQ ID: 138 | SEQ ID: 139 |
| JW0403 | nrdR | SEQ ID: 140 | SEQ ID: 141 |

TABLE 6
E. coli Genetic Modification Results under Aerobic Conditions

| | | | | | | | | | % |
|---|---|---|---|---|---|---|---|---|---|
| | | | Chromosomal | Vector based | | MIC Assay | | MIC | Increase |
| Strain | | | Genetic | Genetic | Tolerance | Result | | Assay | Over |
| Name | Media (M9+) | Parent | Modifications | Modifications | Group | (g/L 3-HP) | P-value | Number | Control |
| BX_00138.0 | Kan (20 μg/mL) | BW25113 | wild type | pSmart-LC-Kan | None | 25 | <0.1 | ≧3 | — |
| BX_00300.0 | Kan 20 μg/mL | BW25113 | wild type | pSmart-LC-Kan- | A | 35 | <0.1 | ≧3 | 40 |
| | | | | tyrA-aroF | | | | | |
| BX_00301.0 | Kan 20 μg/mL | BW25113 | wild type | pSmart-LC-Kan- | A | 35 | <0.1 | ≧3 | 40 |
| | | | | folA-C1 | | | | | |
| BX_00302.0 | Kan 20 μg/mL | BW25113 | wild type | pSmart-LC-Kan- | A | 30 | <0.1 | ≧3 | 20 |
| | | | | folA-ORF | | | | | |
| BX_00304.0 | Kan 20 μg/mL | BW25113 | wild type | pSmart-LC-Kan- | A | 35 | <0.1 | ≧3 | 40 |
| | | | | menA-ORF | | | | | |
| BX_00305.0 | Kan 20 μg/mL | BW25113 | wild type | pSmart-LC-Kan- | A | 35 | <0.1 | ≧3 | 40 |
| | | | | pheA-C1 | | | | | |
| BX_00307.0 | Kan 20 μg/mL | BW25113 | wild type | pSmart-LC-Kan- | A | 35 | <0.1 | ≧3 | 40 |
| | | | | tyrA-C1 | | | | | |
| BX_00309.0 | Kan 20 μg/mL | BW25113 | wild type | pSmart-LC-Kan- | C | 35 | <0.1 | ≧3 | 40 |
| | | | | cynTS | | | | | |
| BX_00310.0 | Kan 20 μg/mL | BW25113 | wild type | pSmart-LC-Kan-glyA | B | 35 | <0.1 | ≧3 | 40 |
| BX_00312.0 | Kan 20 μg/mL | BW25113 | wild type | pSmart-LC-Kan- | B | 35 | <0.1 | ≧3 | 40 |
| | | | | serA | | | | | |
| BX_00313.0 | Kan 20 μg/mL | BW25113 | wild type | pSmart-LC-Kan-folD | A | 30 | <0.1 | ≧3 | 20 |
| BX_00314.0 | Kan 20 μg/mL | BW25113 | wild type | pSmart-LC-Kan- | A | 35 | <0.1 | ≧3 | 40 |
| | | | | aroE | | | | | |
| BX_00315.0 | Kan 20 μg/mL | BW25113 | wild type | pSmart-LC-Kan- | A | 35 | <0.1 | ≧3 | 40 |
| | | | | aroKB C1 | | | | | |
| BX_00317.0 | Kan 20 μg/mL | BW25113 | wild type | pSmart-LC-Kan-ilvA | B | 35 | <0.1 | ≧3 | 40 |
| | | | | operon | | | | | |
| BX_00318.0 | Kan 20 μg/mL | BW25113 | wild type | pSmart-LC-Kan- | B | 35 | <0.1 | ≧3 | 40 |
| | | | | cysM | | | | | |
| BX_00352.0 | Amp 100 μg/mL | BW25113 | wild type | pSmart-LC-Kan- | B | 35 | <0.1 | ≧3 | 40 |
| | | | | metC C1 | | | | | |
| BX_00387.0 | Kan (20 μg/mL) | BW25113 | ΔlysR::frt | pSmart-LC-Kan- | A | 35 | <0.1 | ≧3 | 40 |
| | | | | menA-ORF | | | | | |
| BX_00002.0 | Amp (100 μg/mL) | BW25113 | wild type | pKK223-mcs1 | None | 20 | <0.1 | ≧3 | — |
| BX_00319.0 | Amp 100 μg/mL + | BW25113 | wild type | pK223-aroH | A | 30 | <0.1 | ≧3 | 50 |
| | 1 mM IPTG | | | | | | | | |
| BX_00320.0 | Amp 100 μg/mL + | BW25113 | wild type | pK223-metE C645A | B | 35 | <0.1 | ≧3 | 75 |
| | 1 mM IPTG | | | | | | | | |
| BX_00321.0 | Amp 100 μg/mL + | BW25113 | wild type | pK223-ct-his-thrA | B | 35 | <0.1 | ≧3 | 75 |
| | 1 mM IPTG | | | | | | | | |
| BX_00357.0 | Amp 100 μg/mL + | BW25113 | wild type | pKK223-aroH*445 | A | 30 | <0.1 | ≧3 | 50 |
| | 1 mM IPTG | | | | | | | | |
| BX_00358.0 | Amp 100 μg/mL + | BW25113 | wild type | pKK223-aroH*447 | A | 35 | <0.1 | ≧3 | 75 |
| | 1 mM IPTG | | | | | | | | |
| BX_00359.0 | Amp 100 μg/mL + | BW25113 | wild type | pKK223-aroH*457 | A | 35 | <0.1 | ≧3 | 75 |
| | 1 mM IPTG | | | | | | | | |
| BX_00118.0 | Kan(20 μg/mL) | BW25113 | wild type | pJ251 | None | 25 | <0.1 | ≧3 | — |
| BX_00322.0 | Kan 20 μg/mL | BW25113 | wild type | pJ61-speFED | C | 35 | <0.1 | ≧3 | 40 |
| BX_00323.0 | Kan 20 μg/mL | BW25113 | wild type | pJ61-aroG | A | 35 | <0.1 | ≧3 | 40 |
| BX_00324.0 | Kan 20 μg/mL | BW25113 | wild type | pJ61-thrA | B | 35 | <0.1 | ≧3 | 40 |
| BX_00325.0 | Kan 20 μg/mL | BW25113 | wild type | pJ61-asd | B | 35 | <0.1 | ≧3 | 40 |
| BX_00326.0 | Kan 20 μg/mL | BW25113 | wild type | pJ61-ilvA | B | 35 | <0.1 | ≧3 | 40 |
| BX_00327.0 | Kan 20 μg/mL | BW25113 | wild type | pJ61-cysM | B | 35 | <0.1 | ≧3 | 40 |
| BX_00361.0 | Kan 20 μg/mL | BW25113 | wild type | pACYC177 (Kan | C | 35 | <0.1 | ≧3 | 40 |
| | | | | only)-cynTS | | | | | |
| BX_00362.0 | Kan 20 μg/mL + 1 mM | BW25113 | wild type | pACYC177 (Kan | A | 30 | <0.1 | ≧3 | 20 |
| | IPTG | | | only)-aroH | | | | | |
| BX_00363.0 | Kan 20 μg/mL | BW25113 | wild type | pACYC177 (kan | C | 35 | <0.1 | ≧3 | 40 |
| | | | | only)-speB | | | | | |
| BX_00364.0 | Kan 20 μg/mL + 1 mM | BW25113 | wild type | pACYC177 (Kan | B | 35 | <0.1 | ≧3 | 40 |
| | IPTG | | | only)-metE | | | | | |
| | | | | (Version1) | | | | | |
| | | | | (SS090608_13) | | | | | |
| BX_00365.0 | Kan 20 μg/mL | BW25113 | wild type | pACYC177 (Kan | B | 35 | <0.1 | ≧3 | 40 |
| | | | | only)-metC | | | | | |
| | | | | (Version1) | | | | | |
| | | | | (SS090608_17) | | | | | |
| BX_00144.0 | Amp (100 μg/mL) | BW25113 | wild type | pSmart-HC-Amp | None | 25 | <0.1 | ≧3 | — |
| BX_00334.0 | Amp 100 μg/mL | BW25113 | wild type | pSmart-HC-Amp- | D | 40 | <0.1 | ≧3 | 60 |
| | | | | cadA | | | | | |
| BX_00335.0 | Amp 100 μg/mL | BW25113 | wild type | pSmart-HC-Amp-prs | E | 35 | <0.1 | ≧3 | 40 |
| BX_00336.0 | Amp 100 μg/mL | BW25113 | wild type | pSmart-HC-Amp- | E | 35 | <0.1 | ≧3 | 40 |
| | | | | nrdAB | | | | | |
| BX_00337.0 | Amp 100 μg/mL | BW25113 | wild type | pSmart-HC-Amp- | E | 35 | <0.1 | ≧3 | 40 |
| | | | | nrdEF | | | | | |
| BX_00353.0 | Amp 100 μg/mL | BW25113 | wild type | pSmart-HC-Amp- | B | 45 | <0.1 | ≧3 | 80 |
| | | | | metC | | | | | |
| BX_00354.0 | Amp 100 μg/mL | BW25113 | wild type | pSmart-HC-Amp- | C | 45 | <0.1 | ≧3 | 80 |
| | | | | cynTS | | | | | |
| BX_00356.0 | Amp 100 μg/mL | BW25113 | wild type | pSmart-HC-Amp- | D | 30 | <0.1 | ≧3 | 20 |
| | | | | LysA | | | | | |
| BX_00419.0 | Amp (100 μg/mL) | BW25113 | ΔlysR::frt | pSmart-HC-Amp-prs | D, E | 30 | <0.1 | ≧3 | 20 |
| BX_00420.0 | Amp (100 μg/mL) | BW25113 | ΔlysR::frt | pSmart-HC-Amp- | D, E | 45 | <0.1 | ≧3 | 80 |
| | | | | nrdAB | | | | | |
| BX_00421.0 | Amp (100 μg/mL) | BW25113 | ΔlysR::frt | pSmart-HC-Amp- | D, E | 30 | <0.1 | ≧3 | 20 |
| | | | | nrdEF | | | | | |
| BX_00425.0 | Amp (100 μg/mL) | BW25113 | ΔnrdR::frt | pSmart-HC-Amp- | D, E | 35 | <0.1 | ≧3 | 40 |
| | | | | dapA | | | | | |
| BX_00426.0 | Amp (100 μg/mL) | BW25113 | ΔnrdR::frt | pSmart-HC-Amp- | D, E | 45 | <0.1 | ≧3 | 80 |
| | | | | cadA | | | | | |
| BX_00437.0 | Amp (100 μg/mL) | BW25113 | ΔlysR::frt | pSmart-HC-Amp- | B, D | 30 | <0.1 | ≧3 | 20 |
| | | | | metC | | | | | |
| BX_00438.0 | Amp (100 μg/mL) | BW25113 | ΔnrdR::frt | pSmart-HC-amp- | B, D | 35 | <0.1 | ≧3 | 40 |
| | | | | metC | | | | | |
| BW25113 | M9 | none | none | none | None | 27.5 | <0.1 | ≧3 | — |
| BX_00341.0 | none | BW25113 | ΔtyrR::frt | none | A | 40 | <0.1 | ≧3 | 45 |
| BX_00342.0 | none | BW25113 | ΔtrpR::frt | none | A | 35 | <0.1 | ≧3 | 27 |
| BX_00345.0 | none | BW25113 | ΔmetJ::frt | none | B | 35 | <0.1 | ≧3 | 27 |
| BX_00347.0 | none | BW25113 | ΔpurR::frt | none | C | 35 | <0.1 | ≧3 | 27 |
| BX_00348.0 | none | BW25113 | ΔlysR::frt | none | D | 35 | <0.1 | ≧3 | 27 |
| BX_00349.0 | none | BW25113 | ΔnrdR::frt | none | E | 35 | <0.1 | ≧3 | 27 |
| BX_00003.0 | Cm(20 μg/mL) | BW25113 | wild type | pBT-3 | None | 25 | <0.1 | ≧3 | — |
| BX_00368.0 | Cm (20 μg/mL) | BW25113 | wild type | pBT-3-cynTS | C | 30 | <0.1 | ≧3 | 20 |
| BX_00370.0 | Cm (20 μg/mL) | BW25113 | wild type | pBT-3-speB | C | 30 | <0.1 | ≧3 | 20 |
| BX_00142.0 | Kan(20 μg/mL), | BW25113 | wild type | pSmart-LC-kan, | None | 20 | <0.1 | ≧3 | — |
| | Cm(20 μg/mL) | | | pBT-3 | | | | | |
| BX_00463.0 | Cm (20 μg/mL)/ | BW25113 | ΔnrdR::frt | pBT-3-aroH*, | A, C, E | 30 | <0.1 | ≧3 | 50 |
| | Kan(20 μg/mL) + | | | pSmart-LC-Kan | | | | | |
| | 1 mM IPTG | | | cynTS | | | | | |
| BX_00468.0 | Cm (20 μg/mL)/ | BW25113 | ΔnrdR::frt | pSmart-LC-Kan- | B, C, E | 30 | <0.1 | ≧3 | 50 |
| | Kan(20 μg/mL) | | | metC, pBT3-cynTS | | | | | |

TABLE 7
E. coli Genetic Modification Results under Anaerobic Conditions

| | | | | | | | | | % |
|---|---|---|---|---|---|---|---|---|---|
| | | | Chromosomal | Vector based | | MIC Assay | | | Increase |
| Strain | | | Genetic | Genetic | Tolerance | Result | P- | MIC Assay | Over |
| Name | Media (M9+) | Parent | Modifications | Modifications | Group | (g/L 3-HP) | value | Number | Control |
| BX_00138.0 | Kan (20 μg/mLI) | BW25113 | wild type | pSmart-LC-Kan | None | 25 | <0.1 | ≧3 | — |
| BX_00311.0 | Kan 20 μg/mL | BW25113 | wild type | pSmart-LC-Kan- | B | 30 | <0.1 | ≧3 | 20 |
| | | | | glyA-ORF | | | | | |
| BX_00002.0 | Amp (100 μg/mL) | BW25113 | wild type | pKK223-mcs1 | None | 15 | <0.1 | ≧3 | — |
| BX_00319.0 | Amp 100 μg/mL + | BW25113 | wild type | pK223-aroH | A | 20 | <0.1 | ≧3 | 33 |
| | 1 mM IPTG | | | | | | | | |
| BX_00320.0 | Amp 100 μg/mL + | BW25113 | wild type | pK223-metE | B | 20 | <0.1 | ≧3 | 33 |
| | 1 mM IPTG | | | C645A | | | | | |
| BX_00321.0 | Amp 100 μg/mL + | BW25113 | wild type | pK223-ct-his-thrA | B | 20 | <0.1 | ≧3 | 33 |
| | 1 mM IPTG | | | | | | | | |
| BX_00357.0 | Amp 100 μg/mL + | BW25113 | wild type | pKK223-aroH*445 | B | 20 | <0.1 | ≧3 | 33 |
| | 1 mM IPTG | | | | | | | | |
| BX_00358.0 | Amp 100 μg/mL + | BW25113 | wild type | pKK223-aroH*447 | A | 20 | <0.1 | ≧3 | 33 |
| | 1 mM IPTG | | | | | | | | |
| BX_00359.0 | Amp 100 μg/mL + | BW25113 | wild type | pKK223-aroH*457 | A | 20 | <0.1 | ≧3 | 33 |
| | 1 mM IPTG | | | | | | | | |
| BX_00118.0 | Kan(20 μg/mL) | BW25113 | wild type | pJ251 | None | 15 | <0.1 | ≧3 | — |
| BX_00322.0 | Kan 20 μg/mL | BW25113 | wild type | pJ61-speFED | C | 25 | <0.1 | ≧3 | 67 |
| BX_00323.0 | Kan 20 μg/mL | BW25113 | wild type | pJ61-aroG | A | 20 | <0.1 | ≧3 | 33 |
| BX_00324.0 | Kan 20 μg/mL | BW25113 | wild type | pJ61-thrA | B | 20 | <0.1 | ≧3 | 33 |
| BX_00325.0 | Kan 20 μg/mL | BW25113 | wild type | pJ61-asd | B | 20 | <0.1 | ≧3 | 33 |
| BX_00326.0 | Kan 20 μg/mL | BW25113 | wild type | pJ61-ilvA | B | 20 | <0.1 | ≧3 | 33 |
| BX_00327.0 | Kan 20 μg/mL | BW25113 | wild type | pJ61-cysM | B | 20 | <0.1 | ≧3 | 33 |
| BX_00360.0 | Kan 20 μg/mL | BW25113 | wild type | pACYC177(Kan | C | 20 | <0.1 | ≧3 | 33 |
| | | | | only)-cynTS | | | | | |
| BX_00362.0 | Kan 20 μg/mL + 1 mM | BW25113 | wild type | pACYC177(Kan | A | 20 | <0.1 | ≧3 | 33 |
| | IPTG | | | only)-aroH | | | | | |
| BX_00363.0 | Kan 20 μg/mL | BW25113 | wild type | pACYC177(Kan | C | 20 | <0.1 | ≧3 | 33 |
| | | | | only)-speB | | | | | |
| BX_00364.0 | Kan 20 μg/mL + 1 mM | BW25113 | wild type | pACYC177(Kan | B | 20 | <0.1 | ≧3 | 33 |
| | IPTG | | | only)-metE | | | | | |
| BX_00365.0 | Kan 20 μg/mL | BW25113 | wild type | pACYC177(Kan | B | 20 | <0.1 | ≧3 | 33 |
| | | | | only)-metC | | | | | |
| BX_00144.0 | Amp (100 μg/mL) | BW25113 | wild type | pSmart-HC-Amp | None | 25 | <0.1 | ≧3 | — |
| BX_00426.0 | Amp (100 μg/mL) | BW25113 | ΔnrdR::frt | pSmart-HC-Amp- | D, E | 26.7 | <0.1 | ≧3 | 7 |
| | | | | cadA | | | | | |
| BX_00003.0 | Cm(20 μg/mL) | BW25113 | wild type | pBT-3 | None | 15 | <0.1 | ≧3 | — |
| BX_00368.0 | Cm (20 μg/mL) | BW25113 | wild type | pBT-3-cynTS | C | 20 | <0.1 | ≧3 | 33 |

TABLE 8
E. coli Supplement Results under Aerobic Conditions

| | | | | average | | | % |
|---|---|---|---|---|---|---|---|
| | | | | MIC Assay | | MIC | Increase |
| | Strain | | | Result | | Assay | Over |
| | Name | Media | Supplements (Group) | (g/L 3-HP) | P-value | Number | Control |
| CONTROLS | BW25113 | M9 | none | 28 | <0.1 | ≧3 | — |
| | BW25113 | EZ Rich | none | 75 | <0.1 | ≧3 | 173 |
| | BW25113 | M9 | Phenylalanine (A) | 32 | <0.1 | ≧3 | 17 |
| | BW25113 | M9 | Shikimate (A) | 28 | <0.1 | ≧3 | 3 |
| | BW25113 | M9 | p-aminobenzoate (A) | 35 | <0.1 | ≧3 | 27 |
| | BW25113 | M9 | Dihydroxybenzoate (A) | 35 | <0.1 | ≧3 | 27 |
| | BW25113 | M9 | Tetrahydrofolate (A) | 30 | <0.1 | ≧3 | 9 |
| | BW25113 | M9 | Chorismate Group Mix (A) | 30 | <0.1 | ≧3 | 9 |
| | BW25113 | M9 | Homocysteine (B) | 30 | <0.1 | ≧3 | 9 |
| | BW25113 | M9 | Isoleucine (B) | 32 | <0.1 | ≧3 | 17 |
| | BW25113 | M9 | Serine (B) | 32 | <0.1 | ≧3 | 17 |
| | BW25113 | M9 | Glycine (B) | 28 | <0.1 | ≧3 | 3 |
| | BW25113 | M9 | Methionine (B) | 38 | <0.1 | ≧3 | 36 |
| | BW25113 | M9 | Threonine (B) | 32 | <0.1 | ≧3 | 17 |
| | BW25113 | M9 | Homoserine (B) | 35 | <0.1 | ≧3 | 27 |
| | BW25113 | M9 | Homocysteine Group Mix (B) | 40 | <0.1 | ≧3 | 45 |
| | BW25113 | M9 | Putrescine (C) | 30 | <0.1 | ≧3 | 9 |
| | BW25113 | M9 | Cadaverine (C) | 35 | <0.1 | ≧3 | 27 |
| | BW25113 | M9 | Spermidine (C) | 40 | <0.1 | ≧3 | 45 |
| | BW25113 | M9 | Ornithine (C) | 30 | <0.1 | ≧3 | 9 |
| | BW25113 | M9 | Citrulline (C) | 30 | <0.1 | ≧3 | 9 |
| | BW25113 | M9 | Bicarbonate (C) | 44 | <0.1 | ≧3 | 59 |
| | BW25113 | M9 | Glutamine (C) | 30 | <0.1 | ≧3 | 9 |
| | BW25113 | M9 | Polyamine Group Mix (C) | 57 | <0.1 | ≧3 | 106 |
| | BW25113 | M9 | Lysine (D) | 37 | <0.1 | ≧3 | 33 |
| Double | BW25113 | M9 | Tyrosine (A), Homocysteine (B) | 35 | <0.1 | ≧3 | 27 |
| Supplements | BW25113 | M9 | Tyrosine (A), Methionine (B) | 30 | <0.1 | ≧3 | 9 |
| | BW25113 | M9 | Tyrosine (A), Isoleucine (B) | 30 | <0.1 | ≧3 | 9 |
| | BW25113 | M9 | Tyrosine (A), Putrescine (C) | 40 | <0.1 | ≧3 | 45 |
| | BW25113 | M9 | Tyrosine (A), Spermidine (C) | 40 | <0.1 | ≧3 | 45 |
| | BW25113 | M9 | Tyrosine (A), Ornithine (C) | 30 | <0.1 | ≧3 | 9 |
| | BW25113 | M9 | Tyrosine (A), Bicarbonate (C) | 35 | <0.1 | ≧3 | 27 |
| | BW25113 | M9 | Tyrosine (A), Lysine (D) | 30 | <0.1 | ≧3 | 9 |
| | BW25113 | M9 | Tyrosine (A), Citrate (F) | 35 | <0.1 | ≧3 | 27 |
| | BW25113 | M9 | Shikimate (A), Methionine (B) | 30 | <0.1 | ≧3 | 9 |
| | BW25113 | M9 | Shikimate (A), Bicarbonate (C) | 30 | <0.1 | ≧3 | 9 |
| | BW25113 | M9 | Shikimate (A), Uracil (E) | 30 | <0.1 | ≧3 | 9 |
| | BW25113 | M9 | Tetrahydrofolate (A), Methionine | 30 | <0.1 | ≧3 | 9 |
| | | | (B) | | | | |
| | BW25113 | M9 | Tetrahydrofolate (A), | 30 | <0.1 | ≧3 | 9 |
| | | | Homocysteine (B) | | | | |
| | BW25113 | M9 | Tetrahydrofolate (A), Putrescine | 35 | <0.1 | ≧3 | 27 |
| | | | (C) | | | | |
| | BW25113 | M9 | Tetrahydrofolate (A), Spermidine | 40 | <0.1 | ≧3 | 45 |
| | | | (C) | | | | |
| | BW25113 | M9 | Tetrahydrofolate (A), Ornithine | 35 | <0.1 | ≧3 | 27 |
| | | | (C) | | | | |
| | BW25113 | M9 | Tetrahydrofolate (A), Bicarbonate | 30 | <0.1 | ≧3 | 9 |
| | | | (C) | | | | |
| | BW25113 | M9 | Tetrahydrofolate (A), Uracil (E) | 30 | <0.1 | ≧3 | 9 |
| | BW25113 | M9 | Tetrahydrofolate (A), Citrate (F) | 30 | <0.1 | ≧3 | 9 |
| | BW25113 | M9 | Methionine (B), Putrescine (C) | 47 | <0.1 | ≧3 | 70 |
| | BW25113 | M9 | Methionine (B), Spermidine (C) | 40 | <0.1 | ≧3 | 45 |
| | BW25113 | M9 | Methionine (B), Ornithine (C) | 45 | <0.1 | ≧3 | 64 |
| | BW25113 | M9 | Methionine (B), Bicarbonate (C) | 35 | <0.1 | ≧3 | 27 |
| | BW25113 | M9 | Methionine (B), Lysine (D) | 30 | <0.1 | ≧3 | 9 |
| | BW25113 | M9 | Methionine (B), Uracil (E) | 35 | <0.1 | ≧3 | 27 |
| | BW25113 | M9 | Methionine (B), Citrate (F) | 30 | <0.1 | ≧3 | 9 |
| | BW25113 | M9 | Homocysteine (B), Putrescine (C) | 40 | <0.1 | ≧3 | 45 |
| | BW25113 | M9 | Homocysteine (B), Spermidine | 45 | <0.1 | ≧3 | 64 |
| | | | (C) | | | | |
| | BW25113 | M9 | Homocysteine (B), Ornithine (C) | 30 | <0.1 | ≧3 | 9 |
| | BW25113 | M9 | Homocysteine (B), Bicarbonate | 42 | <0.1 | ≧3 | 52 |
| | | | (C) | | | | |
| | BW25113 | M9 | Homocysteine (B), Lysine (D) | 35 | <0.1 | ≧3 | 27 |
| | BW25113 | M9 | Homocysteine (B), Uracil (E) | 30 | <0.1 | ≧3 | 9 |
| | BW25113 | M9 | Homocysteine (B), Citrate (F) | 30 | <0.1 | ≧3 | 9 |
| | BW25113 | M9 | Isoleucine (B), Putrescine (C) | 35 | <0.1 | ≧3 | 27 |
| | BW25113 | M9 | Isoleucine (B), Spermidine (C) | 35 | <0.1 | ≧3 | 27 |
| | BW25113 | M9 | Isoleucine (B), Bicarbonate (C) | 35 | <0.1 | ≧3 | 27 |
| | BW25113 | M9 | Isoleucine (B), Lysine (D) | 30 | <0.1 | ≧3 | 9 |
| | BW25113 | M9 | Isoleucine (B), Uracil (E) | 35 | <0.1 | ≧3 | 27 |
| | BW25113 | M9 | Isoleucine (B), Citrate (F) | 35 | <0.1 | ≧3 | 27 |
| | BW25113 | M9 | Putrescine (C), Lysine (D) | 42 | <0.1 | ≧3 | 52 |
| | BW25113 | M9 | Putrescine (C), Uracil (E) | 30 | <0.1 | ≧3 | 9 |
| | BW25113 | M9 | Putrescine (C), Citrate (F) | 30 | <0.1 | ≧3 | 9 |
| | BW25113 | M9 | Spermidine (C), Lysine (D) | 40 | <0.1 | ≧3 | 45 |
| | BW25113 | M9 | Spermidine (C), Uracil (E) | 30 | <0.1 | ≧3 | 9 |
| | BW25113 | M9 | Spermidine (C), Citrate (F) | 38 | <0.1 | ≧3 | 39 |
| | BW25113 | M9 | Ornithine (C), Lysine (D) | 32 | <0.1 | ≧3 | 15 |
| | BW25113 | M9 | Ornithine (C), Uracil (E) | 30 | <0.1 | ≧3 | 9 |
| | BW25113 | M9 | Ornithine (C), Citrate (F) | 30 | <0.1 | ≧3 | 9 |
| | BW25113 | M9 | Bicarbonate (C), Lysine (D) | 35 | <0.1 | ≧3 | 27 |
| | BW25113 | M9 | Bicarbonate (C), Uracil (E) | 35 | <0.1 | ≧3 | 27 |
| | BW25113 | M9 | Bicarbonate (C), Citrate (F) | 40 | <0.1 | ≧3 | 45 |
| | BW25113 | M9 | Lysine (D), Uracil (E) | 30 | <0.1 | ≧3 | 9 |
| | BW25113 | M9 | Lysine (D), Citrate (F) | 30 | <0.1 | ≧3 | 9 |
| Triple | BW25113 | M9 | Tyrosine (A), Methionine (B), | 35 | <0.1 | ≧3 | 27 |
| Supplements | | | Putrescine (C) | | | | |
| | BW25113 | M9 | Tyrosine (A), Methionine (B), | 35 | <0.1 | ≧3 | 27 |
| | | | Spermidine (C) | | | | |
| | BW25113 | M9 | Tyrosine (A), Methionine (B), | 30 | <0.1 | ≧3 | 9 |
| | | | Bicarbonate (C) | | | | |
| | BW25113 | M9 | Tyrosine (A), Methionine (B), | 30 | <0.1 | ≧3 | 9 |
| | | | Lysine (D) | | | | |
| | BW25113 | M9 | Tyrosine (A), Methionine (B), | 40 | <0.1 | ≧3 | 45 |
| | | | Uracil (E) | | | | |
| | BW25113 | M9 | Tyrosine (A), Methionine (B), | 30 | <0.1 | ≧3 | 9 |
| | | | Citrate (F) | | | | |
| | BW25113 | M9 | Tyrosine (A), Putrescine (C), | 30 | <0.1 | ≧3 | 9 |
| | | | Homocysteine (B) | | | | |
| | BW25113 | M9 | Tyrosine (A), Putrescine (C), | 28 | <0.1 | ≧3 | 3 |
| | | | Isoleucine (B) | | | | |
| | BW25113 | M9 | Tyrosine (A), Putrescine (C), | 35 | <0.1 | ≧3 | 27 |
| | | | Lysine (D) | | | | |
| | BW25113 | M9 | Tyrosine (A), Putrescine (C), | 30 | <0.1 | ≧3 | 9 |
| | | | Uracil (E) | | | | |
| | BW25113 | M9 | Tyrosine (A), Spermidine (C), | 30 | <0.1 | ≧3 | 9 |
| | | | Homocysteine (B) | | | | |
| | BW25113 | M9 | Tyrosine (A), Spermidine (C), | 30 | <0.1 | ≧3 | 9 |
| | | | Isoleucine (B) | | | | |
| | BW25113 | M9 | Tyrosine (A), Spermidine (C), | 30 | <0.1 | ≧3 | 9 |
| | | | Lysine (D) | | | | |
| | BW25113 | M9 | Tyrosine (A), Spermidine (C), | 35 | <0.1 | ≧3 | 27 |
| | | | Uracil (E) | | | | |
| | BW25113 | M9 | Tyrosine (A), Spermidine (C), | 30 | <0.1 | ≧3 | 9 |
| | | | Citrate (F) | | | | |
| | BW25113 | M9 | Tyrosine (A), Bicarbonate (C), | 35 | <0.1 | ≧3 | 27 |
| | | | Homocysteine (B) | | | | |
| | BW25113 | M9 | Tyrosine (A), Bicarbonate (C), | 35 | <0.1 | ≧3 | 27 |
| | | | Isoleucine (B) | | | | |
| | BW25113 | M9 | Tyrosine (A), Bicarbonate (C), | 45 | <0.1 | ≧3 | 64 |
| | | | Lysine (D) | | | | |
| | BW25113 | M9 | Tyrosine (A), Bicarbonate (C), | 45 | <0.1 | ≧3 | 64 |
| | | | Uracil (E) | | | | |
| | BW25113 | M9 | Tyrosine (A), Bicarbonate (C), | 40 | <0.1 | ≧3 | 45 |
| | | | Citrate (F) | | | | |
| | BW25113 | M9 | Shikimate (A), Putrescine (C), | 30 | <0.1 | ≧3 | 9 |
| | | | Homocysteine (B) | | | | |
| | BW25113 | M9 | Shikimate (A), Putrescine (C), | 30 | <0.1 | ≧3 | 9 |
| | | | Uracil (E) | | | | |
| | BW25113 | M9 | Shikimate (A), Putrescine (C), | 30 | <0.1 | ≧3 | 9 |
| | | | Methionine (B) | | | | |
| | BW25113 | M9 | Shikimate (A), Spermidine (C), | 30 | <0.1 | ≧3 | 9 |
| | | | Methionine (B) | | | | |
| | BW25113 | M9 | Shikimate (A), Uracil (C), | 30 | <0.1 | ≧3 | 9 |
| | | | Homocysteine (B) | | | | |
| | BW25113 | M9 | Shikimate (A), Uracil (C), | 30 | <0.1 | ≧3 | 9 |
| | | | Isoleucine (B) | | | | |
| | BW25113 | M9 | Shikimate (A), Uracil (C), | 35 | <0.1 | ≧3 | 27 |
| | | | Methionine (B) | | | | |
| | BW25113 | M9 | Shikimate (A), Uracil (C), Lysine | 30 | <0.1 | ≧3 | 9 |
| | | | (D) | | | | |
| | BW25113 | M9 | Shikimate (A), Uracil (C), Citrate | 30 | <0.1 | ≧3 | 9 |
| | | | (F) | | | | |
| | BW25113 | M9 | Methionine (B), Putrescine (C), | 35 | <0.1 | ≧3 | 27 |
| | | | Lysine (D) | | | | |
| | BW25113 | M9 | Methionine (B), Putrescine (C), | 35 | <0.1 | ≧3 | 27 |
| | | | Uracil (E) | | | | |
| | BW25113 | M9 | Methionine (B), Putrescine (C), | 35 | <0.1 | ≧3 | 27 |
| | | | Citrate (F) | | | | |
| | BW25113 | M9 | Methionine (B), Spermidine (C), | 45 | <0.1 | ≧3 | 64 |
| | | | Lysine (D) | | | | |
| | BW25113 | M9 | Methionine (B), Spermidine (C), | 35 | <0.1 | ≧3 | 27 |
| | | | Uracil (E) | | | | |
| | BW25113 | M9 | Methionine (B), Spermidine (C), | 40 | <0.1 | ≧3 | 45 |
| | | | Citrate (F) | | | | |
| | BW25113 | M9 | Methionine (B), Bicarbonate (C), | 45 | <0.1 | ≧3 | 64 |
| | | | Lysine (D) | | | | |
| | BW25113 | M9 | Methionine (B), Bicarbonate (C), | 45 | <0.1 | ≧3 | 64 |
| | | | Uracil (E) | | | | |
| | BW25113 | M9 | Methionine (B), Bicarbonate (C), | 45 | <0.1 | ≧3 | 64 |
| | | | Citrate (F) | | | | |
| | BW25113 | M9 | Methionine (B), Lysine (D), Uracil | 35 | <0.1 | ≧3 | 27 |
| | | | (E) | | | | |
| | BW25113 | M9 | Homocysteine (B), Bicarbonate | 50 | <0.1 | ≧3 | 82 |
| | | | (C), Lysine (D) | | | | |
| | BW25113 | M9 | Homocysteine (B), Bicarbonate | 40 | <0.1 | ≧3 | 45 |
| | | | (C), Uracil (E) | | | | |
| | BW25113 | M9 | Isoleucine (B), Putrescine (C), | 35 | <0.1 | ≧3 | 27 |
| | | | Lysine (D) | | | | |
| | BW25113 | M9 | Isoleucine (B), Putrescine (C), | 30 | <0.1 | ≧3 | 9 |
| | | | Uracil (E) | | | | |
| | BW25113 | M9 | Isoleucine (B), Putrescine (C), | 35 | <0.1 | ≧3 | 27 |
| | | | Citrate (F) | | | | |
| | BW25113 | M9 | Isoleucine (B), Bicarbonate (C), | 55 | <0.1 | ≧3 | 100 |
| | | | Lysine (D) | | | | |
| | BW25113 | M9 | Isoleucine (B), Bicarbonate (C), | 40 | <0.1 | ≧3 | 45 |
| | | | Uracil (E) | | | | |
| | BW25113 | M9 | Isoleucine (B), Bicarbonate (C), | 35 | <0.1 | ≧3 | 27 |
| | | | Citrate (F) | | | | |
| | BW25113 | M9 | Lysine (B), Bicarbonate (C), | 35 | <0.1 | ≧3 | 27 |
| | | | Uracil (E) | | | | |
| | BW25113 | M9 | Lysine (B), Bicarbonate (C), | 35 | <0.1 | ≧3 | 27 |
| | | | Citrate (F) | | | | |
| | BW25113 | M9 | Methionine (B), Putrescine (C), | 30 | <0.1 | ≧3 | 9 |
| | | | Lysine (D) | | | | |
| | BW25113 | M9 | Methionine (B), Bicarbonate (C), | 30 | <0.1 | ≧3 | 9 |
| | | | Lysine (D) | | | | |
| 4 | BW25113 | M9 | Tyrosine (A), Methionine (B), | 50 | <0.1 | ≧3 | 82 |
| Supplements | | | Putrescine (C), Lysine (D) | | | | |
| | BW25113 | M9 | Tyrosine (A), Methionine (B), | 40 | <0.1 | ≧3 | 45 |
| | | | Putrescine (C), Uracil (E) | | | | |
| | BW25113 | M9 | Tyrosine (A), Methionine (B), | 35 | <0.1 | ≧3 | 27 |
| | | | Putrescine (C), Citrate (F) | | | | |
| | BW25113 | M9 | Tyrosine (A), Methionine (B), | 40 | <0.1 | ≧3 | 45 |
| | | | Bicarbonate (C), Lysine (D) | | | | |
| | BW25113 | M9 | Tyrosine (A), Methionine (B), | 40 | <0.1 | ≧3 | 45 |
| | | | Bicarbonate (C), Uracil (E) | | | | |
| | BW25113 | M9 | Tyrosine (A), Methionine (B), | 45 | <0.1 | ≧3 | 64 |
| | | | Bicarbonate (C), Citrate (F) | | | | |
| | BW25113 | M9 | Tyrosine (A), Putrescine (C), | 40 | <0.1 | ≧3 | 45 |
| | | | Homocysteine (B), Lysine (D) | | | | |
| | BW25113 | M9 | Tyrosine (A), Putrescine (C), | 30 | <0.1 | ≧3 | 9 |
| | | | Homocysteine (B), Uracil (E) | | | | |
| | BW25113 | M9 | Tyrosine (A), Putrescine (C), | 35 | <0.1 | ≧3 | 27 |
| | | | Homocysteine (B), Citrate (F) | | | | |
| | BW25113 | M9 | Tyrosine (A), Bicarbonate (C), | 30 | <0.1 | ≧3 | 9 |
| | | | Homocysteine (B), Uracil (E) | | | | |
| | BW25113 | M9 | Tyrosine (A), Bicarbonate (C), | 35 | <0.1 | ≧3 | 27 |
| | | | Homocysteine (B), Citrate (F) | | | | |
| | BW25113 | M9 | Shikimate (A), Putrescine (C), | 30 | <0.1 | ≧3 | 9 |
| | | | Methionine (B), Lysine (D) | | | | |
| | BW25113 | M9 | Shikimate (A), Putrescine (C), | 35 | <0.1 | ≧3 | 27 |
| | | | Methionine (B), Uracil (E) | | | | |
| | BW25113 | M9 | Shikimate (A), Putrescine (C), | 30 | <0.1 | ≧3 | 9 |
| | | | Methionine (B), Citrate (F) | | | | |
| | BW25113 | M9 | Shikimate (A), Uracil (E), | 35 | <0.1 | ≧3 | 27 |
| | | | Methionine (B), Lysine (D) | | | | |
| | BW25113 | M9 | Shikimate (A), Uracil (E), | 35 | <0.1 | ≧3 | 27 |
| | | | Methionine (B), Bicarbonate (C) | | | | |
| | BW25113 | M9 | Shikimate (A), Uracil (E), | 30 | <0.1 | ≧3 | 9 |
| | | | Methionine (B), Citrate (F) | | | | |
| | BW25113 | M9 | Methionine (B), Putrescine (C), | 30 | <0.1 | ≧3 | 9 |
| | | | Lysine (D), Uracil (E) | | | | |
| | BW25113 | M9 | Methionine (B), Bicarbonate (C), | 30 | <0.1 | ≧3 | 9 |
| | | | Lysine (D), Uracil (E) | | | | |
| | BW25113 | M9 | Methionine (B), Bicarbonate (C), | 35 | <0.1 | ≧3 | 27 |
| | | | Lysine (D), Citrate (F) | | | | |
| | BW25113 | M9 | Bicarbonate (C), Lysine (D), | 30 | <0.1 | ≧3 | 9 |
| | | | Uracil (E), Citrate (F) | | | | |
| | BW25113 | M9 | Methionine (B), Lysine (D), Uracil | 35 | <0.1 | ≧3 | 27 |
| | | | (E), Citrate (F) | | | | |
| 5 | BW25113 | M9 | Shikimate (A), Methionine (B), | 40 | <0.1 | ≧3 | 45 |
| supplements | | | Bicarbonate (C), Lysine (D), | | | | |
| | | | Uracil (E) | | | | |
| | BW25113 | M9 | Shikimate (A), Homocsyteine (B), | 40 | <0.1 | ≧3 | 45 |
| | | | Bicarbonate (C), Lysine (D), | | | | |
| | | | Uracil (E) | | | | |
| | BW25113 | M9 | Tyrosine (A), Methionine (B), | 40 | <0.1 | ≧3 | 45 |
| | | | Bicarbonate (C), Lysine (D), | | | | |
| | | | Citrate (F) | | | | |
| | BW25113 | M9 | Shikimate (A), Methionine (B), | 40 | <0.1 | ≧3 | 45 |
| | | | Bicarbonate (C), Lysine (D), | | | | |
| | | | Citrate (F) | | | | |
| | BW25113 | M9 | Shikimate (A), Homocsyteine (B), | 40 | <0.1 | ≧3 | 45 |
| | | | Bicarbonate (C), Lysine (D), | | | | |
| | | | Citrate (F) | | | | |
| | BW25113 | M9 | Methionine (B), Bicarbonate (C), | 40 | <0.1 | ≧3 | 45 |
| | | | Lysine (D), Uracil (E), Citric (F) | | | | |
| | BW25113 | M9 | Tyrosine (A), Methionine (B), | 37 | <0.1 | ≧3 | 33 |
| | | | Bicarbonate (C), Lysine (D), | | | | |
| | | | Uracil (E) | | | | |
| | BW25113 | M9 | Tyrosine (A), Methionine (B), | 35 | <0.1 | ≧3 | 27 |
| | | | Putrescine (C), Lysine (D), Uracil | | | | |
| | | | (E) | | | | |
| | BW25113 | M9 | Shikimate (A), Methionine (B), | 35 | <0.1 | ≧3 | 27 |
| | | | Putrescine (C), Lysine (D), Uracil | | | | |
| | | | (E) | | | | |
| | BW25113 | M9 | Tyrosine (A), Homocysteine (B), | 35 | <0.1 | ≧3 | 27 |
| | | | Putrescine (C), Lysine (D), Uracil | | | | |
| | | | (E) | | | | |
| | BW25113 | M9 | Shikimate (A), Homocsyteine (B), | 35 | <0.1 | ≧3 | 27 |
| | | | Putrescine (C), Lysine (D), Uracil | | | | |
| | | | (E) | | | | |
| | BW25113 | M9 | Tyrosine (A), Methionine (B), | 35 | <0.1 | ≧3 | 27 |
| | | | Putrescine (C), Lysine (D), | | | | |
| | | | Citrate (F) | | | | |
| | BW25113 | M9 | Tyrosine (A), Homocysteine (B), | 35 | <0.1 | ≧3 | 27 |
| | | | Putrescine (C), Lysine (D), | | | | |
| | | | Citrate (F) | | | | |
| | BW25113 | M9 | Shikimate (A), Homocsyteine (B), | 35 | <0.1 | ≧3 | 27 |
| | | | Putrescine (C), Lysine (D), | | | | |
| | | | Citrate (F) | | | | |
| | BW25113 | M9 | Tyrosine (A), Homocysteine (B), | 35 | <0.1 | ≧3 | 27 |
| | | | Bicarbonate (C), Lysine (D), | | | | |
| | | | Citrate (F) | | | | |
| | BW25113 | M9 | Methionine (B), Spermidine (C), | 35 | <0.1 | ≧3 | 27 |
| | | | Lysine (D), Uracil (E), Citric (F) | | | | |
| | BW25113 | M9 | Methionine (B), Putrescine (C), | 35 | <0.1 | ≧3 | 27 |
| | | | Lysine (D), Uracil (E), Citric (F) | | | | |
| | BW25113 | M9 | Tyrosine (A), Bicarbonate (C), | 35 | <0.1 | ≧3 | 27 |
| | | | Lysine (D), Uracil (E), Citrate (F) | | | | |
| | BW25113 | M9 | Tyrosine (A), Methionine (B), | 35 | <0.1 | ≧3 | 27 |
| | | | Lysine (D), Uracil (E), Citrate (F) | | | | |
| | BW25113 | M9 | Shikimate (A), Methionine (B), | 35 | <0.1 | ≧3 | 27 |
| | | | Lysine (D), Uracil (E), Citrate (F) | | | | |
| | BW25113 | M9 | Shikimate (A), Putrescine (C), | 30 | <0.1 | ≧3 | 9 |
| | | | Lysine (D), Uracil (E), Citrate (F) | | | | |
| | BW25113 | M9 | Tyrosine (A), Homocysteine (B), | 38 | <0.1 | ≧3 | 39 |
| | | | Bicarbonate (C), Lysine (D), | | | | |
| | | | Uracil (E) | | | | |
| | BW25113 | M9 | Shikimate (A), Methionine (B), | 30 | <0.1 | ≧3 | 9 |
| | | | Putrescine (C), Lysine (D), | | | | |
| | | | Citrate (F) | | | | |
| 6 | BW25113 | M9 | Tyrosine (A), Methionine (B), | 42 | <0.1 | ≧3 | 52 |
| supplements | | | Putrescine (C), Lysine (D), Uracil | | | | |
| | | | (E), Citrate (F) | | | | |
| | BW25113 | M9 | Shikimate (A), Methionine (B), | 40 | <0.1 | ≧3 | 45 |
| | | | Bicarbonate (C), Lysine (D), | | | | |
| | | | Uracil (E), Citrate (F) | | | | |
| | BW25113 | M9 | Shikimate (A), Methionine (B), | 35 | <0.1 | ≧3 | 27 |
| | | | Putrescine (C), Lysine (D), Uracil | | | | |
| | | | (E), Citrate (F) | | | | |
| | BW25113 | M9 | Tyrosine (A), Methionine (B), | 37 | <0.1 | ≧3 | 33 |
| | | | Bicarbonate (C), Lysine (D), | | | | |
| | | | Uracil (E), Citrate (F) | | | | |

TABLE 9
E. coli Supplement Results under Anaerobic Conditions

| | | | | | | | % |
|---|---|---|---|---|---|---|---|
| | | | | MIC Assay | | MIC | Increase |
| | Strain | | | Result | | Assay | Over |
| | Name | Media | Supplements (Group) | (g/L 3-HP) | P-value | Number | Control |
| CONTROLS | BW25113 | M9 | none | 30.0 | <0.1 | ≧3 | — |
| | BW25113 | EZ Rich | none | 75.0 | <0.1 | ≧3 | 150 |
| Single | BW25113 | M9 | Phenylalanine (A) | 32.1 | <0.1 | ≧3 | 7 |
| Supplements | BW25113 | M9 | p-aminobenzoate (A) | 40.0 | <0.1 | ≧3 | 33 |
| | BW25113 | M9 | Dihydroxybenzoate (A) | 40.0 | <0.1 | ≧3 | 33 |
| | BW25113 | M9 | Tetrahydrofolate (A) | 40.0 | <0.1 | ≧3 | 33 |
| | BW25113 | M9 | Serine (B) | 32.1 | <0.1 | ≧3 | 7 |
| | BW25113 | M9 | Methionine (B) | 42.8 | <0.1 | ≧3 | 43 |
| | BW25113 | M9 | Homoserine (B) | 30.0 | <0.1 | ≧3 | 0 |
| | BW25113 | M9 | Homocysteine Group Mix (B) | 45.0 | <0.1 | ≧3 | 50 |
| | BW25113 | M9 | Putrescine(C) | 35.0 | <0.1 | ≧3 | 17 |
| | BW25113 | M9 | Spermidine (C) | 35.0 | <0.1 | ≧3 | 17 |
| | BW25113 | M9 | Polyamine Group Mix (C) | 60.0 | <0.1 | ≧3 | 100 |
| | BW25113 | M9 | Lysine (D) | 41.7 | <0.1 | ≧3 | 39 |
| Double | BW25113 | M9 | Tetrahydrofolate (A), Putrescine | 35.0 | <0.1 | ≧3 | 17 |
| Supplements | | | (C) | | | | |
| | BW25113 | M9 | Tetrahydrofolate (A), Spermidine | 30.0 | <0.1 | ≧3 | 0 |
| | | | (C) | | | | |
| | BW25113 | M9 | Tetrahydrofolate (A), Bicarbonate | 35.0 | <0.1 | ≧3 | 17 |
| | | | (C) | | | | |
| | BW25113 | M9 | Tetrahydrofolate (A), Lysine (D) | 35.0 | <0.1 | ≧3 | 17 |
| | BW25113 | M9 | Homocysteine (B), Bicarbonate (C) | 35.0 | <0.1 | ≧3 | 17 |
| | BW25113 | M9 | Putrescine (C), Lysine (D) | 30.0 | <0.1 | ≧3 | 0 |
| | BW25113 | M9 | Putrescine (C), Citrate (F) | 36.7 | <0.1 | ≧3 | 22 |
| Triple | BW25113 | M9 | Methionine (B), Spermidine (C), | 35.0 | <0.1 | ≧3 | 17 |
| Supplements | | | Lysine (D) | | | | |
| | BW25113 | M9 | Isolucine (B), Putrescine (C), | 35.0 | <0.1 | ≧3 | 17 |
| | | | Lysine (D) | | | | |
| 4 Supplements | | | | | <0.1 | ≧3 | |
| | BW25113 | M9 | Tyrosine (A), Methionine (B), | 40.0 | <0.1 | ≧3 | 33 |
| | | | Putrescine (C), Lysine (D) | | | | |
| | BW25113 | M9 | Tyrosine (A), Methionine (B), | 35.0 | <0.1 | ≧3 | 17 |
| | | | Bicarbonate (C), Lysine (D) | | | | |
| | BW25113 | M9 | Tyrosine (A), Methionine (B), | 35.0 | <0.1 | ≧3 | 17 |
| | | | Bicarbonate (C), Citrate (F) | | | | |

TABLE 10
B. subtilis Tolerance Plasmid Construction

| | | | | | PCR Sequence or | |
|---|---|---|---|---|---|---|
| Gene(s) or | | Cloning | | | Codon Optimized | |
| Region Name | Vector | Method | Primer A | Primer B | Sequence (Region) | Plasmid Name |
| speB | pWH1520 | A | SEQID. 0142 | SEQID. 0143 | SEQID. 0144 | pWH1520-Pxyl:speB |
| metE | pWH1520 | A | SEQID 0145 | SEQID 0146 | SEQID 0147 | pWH1520-Pxyl:metE |

TABLE 11
B. subtilis Supplement and Genetic Modification Results under Aerobic Conditions

| | | | | | | | | | % |
|---|---|---|---|---|---|---|---|---|---|
| | | | | | Chromosomal | Vector Based | | | Increase |
| | | | Group Re- | | Genetic | Genetic | Avg 24 hr | Standard | Over |
| Strain Name | Media | Supplements | presented | Parent | Modifications | Modifications | ΔOD600 | Error | Control |
| B. subtilis 168 | M9 + glu + | none | none | NA | none | none | 0.04 | 0.004 | 0 |
| | trp* | | | | | | | | |
| B. subtilis 168 | M9 + glu + | Chorismate | A | NA | none | none | 0.26 | 0.043 | 577 |
| | trp | Group | | | | | | | |
| B. subtilis 168 | M9 + glu + | Homocysteine | B | NA | none | none | 0.08 | 0.005 | 104 |
| | trp | Group Mix | | | | | | | |
| B. subtilis 168 | M9 + glu + | Methionine | B | NA | none | none | 0.15 | 0.007 | 282 |
| | trp | | | | | | | | |
| B. subtilis 168 | M9 + glu + | Bicarbonate | C | NA | none | none | 0.06 | 0.002 | 56 |
| | trp | | | | | | | | |
| B. subtilis 168 | M9 + glu + | p- | A | NA | none | none | 0.07 | 0.015 | 89 |
| | trp | aminobenzoate | | | | | | | |
| B. subtilis 168 | M9 + glu + | spermidine | C | NA | none | none | 0.09 | 0.024 | 140 |
| | trp | | | | | | | | |
| B. subtilis 168 | M9 + glu + | Isoleucine, | B, C, D | NA | none | none | 0.05 | 0.006 | 29 |
| | trp | Bicarbonate, | | | | | | | |
| | | Lysine | | | | | | | |
| B. subtilis 168 | M9 + glu + | Citrate | F | NA | none | none | 0.30 | 0.046 | 674 |
| | trp | | | | | | | | |
| BSX_0003.0 | M9 + glu + | none | none | B. subtilis | none | pWH1520 | 0.00 | 0.000 | 0 |
| | trp + | | | 168 | | | | | |
| | 1 mM | | | | | | | | |
| | Xylose | | | | | | | | |
| BSX_0011.0 | M9 + glu + | none | C | B. subtilis | none | pWH1520- | 0.07 | 0.060 | ** |
| | trp + | | | 168 | | Pxyl:speB | | | |
| | 1 mM | | | | | region | | | |
| | Xylose | | | | | | | | |
| BSX_0015.0 | M9 + glu + | none | B | B. subtilis | none | pWH1520- | 0.06 | 0.063 | ** |
| | trp + | | | 168 | | Pxyl:metE | | | |
| | 1 mM | | | | | region | | | |
| | Xylose | | | | | | | | |

*M9 + glu + trp means M9 minimal + glutamate (1.47 g/L) and tryptophan (0.021 g/L)
**Genetically modified strains had a positive change in growth after 24 hours, compared to control BSX_0003.0 which had a decrease in OD600 after 34 hours resulting in a reading of 0.

TABLE 12
Yeast Tolerance Primers

| Gene | Primer A | Primer B |
|---|---|---|
| spe3 | SEQID 0155 | SEQID 0156 |
| hom2 | SEQID 0157 | SEQID 0158 |
| MET6 | SEQID 0159 | SEQID 0160 |
| ILV2 | SEQID 0161 | SEQID 0162 |
| ILV6 | SEQID 0163 | SEQID 0164 |
| THR1 | SEQID 0165 | SEQID 0166 |
| SER2 | SEQID 0167 | SEQID 0168 |
| SER3 | SEQID 0169 | SEQID 0170 |
| ARG2 | SEQID 0171 | SEQID 0172 |
| RNR1 | SEQID 0173 | SEQID 0174 |
| aro3 | SEQID 0175 | SEQID 0176 |
| ARO7 | SEQID 0177 | SEQID 0178 |
| TYR1 | SEQID 0179 | SEQID 0180 |
| TRP1 | SEQID 0181 | SEQID 0182 |

TABLE 13
Yeast Supplement Results Under Aerobic Conditions

| | | | | | | | % |
|---|---|---|---|---|---|---|---|
| | | | | Average MIC | | MIC | Increase |
| | Strain | | | Assay Result | | Assay | Over |
| | Name | Media | Supplements (Group) | (g/L 3-HP) | S.D. | Number | Control |
| CONTROLS | S288C | SD | none | 45 | 2.5 | ≧3 | — |
| | S288C | SC | none | 60 | <2.5 | ≧3 | 33 |
| | S288C | SD | Tryptophan (A) | 54 | 17.4 | ≧3 | 20 |
| | S288C | SD | Shikimate (A) | 80 | <2.5 | ≧3 | 78 |
| | S288C | SD | Chorismate Group Mix (A) | 80 | <2.5 | ≧3 | 78 |
| | S288C | SD | Glycine (B) | 50 | 11.0 | ≧3 | 11 |
| | S288C | SD | Methionine (B) | 72 | 16.9 | ≧3 | 59 |
| | S288C | SD | 2-oxobutyrate (B) | 50 | <2.5 | ≧3 | 11 |
| | S288C | SD | Aspartate | 57 | 2.9 | ≧3 | 26 |
| | S288C | SD | Homocysteine Group Mix (B) | 87 | 5.8 | ≧3 | 93 |
| | S288C | SD | Putrescine(C) | 55 | 16.4 | ≧3 | 22 |
| | S288C | SD | Citrulline (C) | 58 | 21.4 | ≧3 | 28 |

Supplement Combinations

| | | | | | | | |
|---|---|---|---|---|---|---|---|
| Control | S288C | SD | none | 45 | 2.5 | ≧3 | — |
| | S288C | SD | Tyrosine (A), Methionine (B), | 77 | 4.7 | ≧3 | 70 |
| | | | Putrescine (C), Lysine (D) | | | | |
| | S288C | SD | Methionine (B), Ornithine (C) | 80 | 0.0 | ≧3 | 78 |
| | S288C | SD | Homocysteine (B), Spermidine (C) | 77 | 4.7 | ≧3 | 70 |
| | S288C | SD | Tyrosine (A), Bicarbonate (C), Lysine (D) | 70 | <2.5 | ≧3 | 56 |
| | S288C | SD | Tyrosine (A), Bicarbonate (C), Uracil (E) | 67 | 4.7 | ≧3 | 48 |
| | S288C | SD | Methionine (B), Spermidine (C), | 77 | 4.7 | ≧3 | 70 |
| | | | Lysine (D) | | | | |
| | S288C | SD | Methionine (B), Bicarbonate (C), | 70 | <2.5 | ≧3 | 56 |
| | | | Lysine (D) | | | | |
| | S288C | SD | Methionine (B), Bicarbonate (C), | 77 | 4.7 | ≧3 | 70 |
| | | | Uracil (E) | | | | |
| | S288C | SD | Methionine (B), Bicarbonate (C), | 50 | <2.5 | ≧3 | 11 |
| | | | Citrate (F) | | | | |
| | S288C | SD | Putrescine (C), Lysine (D) | 57 | 4.7 | ≧3 | 26 |
| | S288C | SD | Tyrosine (A), Methionine (B), | 77 | 4.7 | ≧3 | 70 |
| | | | Putrescine (C), Lysine (D), Uracil (E), | | | | |
| | | | Citrate (F) | | | | |
| | S288C | SD | Tyrosine (A), Putrescine (C) | 77 | 4.7 | ≧3 | 70 |
| | S288C | SD | Tetrahydrofolate (A), Spermidine (C) | 70 | <2.5 | ≧3 | 56 |
| | S288C | SD | Homocysteine (B), Putrescine (C) | 80 | <2.5 | ≧3 | 78 |
| | S288C | SD | Spermidine (C), Lysine (D) | 70 | <2.5 | ≧3 | 56 |
| | S288C | SD | Bicarbonate (C), Citrate (F) | 50 | <2.5 | ≧3 | 11 |
| | S288C | SD | Tyrosine (A), Bicarbonate (C), Citrate | 50 | <2.5 | ≧3 | 11 |
| | | | (F) | | | | |
| | S288C | SD | Methionine (B), Spermidine (C), | 67 | 4.7 | ≧3 | 48 |
| | | | Citrate (F) | | | | |
| | S288C | SD | Homocysteine (B), Bicarbonate (C), | 60 | <2.5 | ≧3 | 33 |
| | | | Uracil (E) | | | | |

TABLE 14
Yeast Supplement Results Under Anaerobic Conditions

| | | | | | | | % |
|---|---|---|---|---|---|---|---|
| | | | | Average MIC | | MIC | Increase |
| | Strain | | | Assay Result | | Assay | Over |
| | Name | Media | Supplements (Group) | (g/L 3-HP) | S.D. | Number | Control |
| CONTROLS | S288C | SD | none | 38 | 2.7 | ≧3 | — |
| | S288C | SD | Phenylalanine (A) | 38 | 2.9 | ≧3 | 2 |
| | S288C | SD | Tryptophan (A) | 55 | 5.5 | ≧3 | 47 |
| | S288C | SD | Shikimate (A) | 60 | <2.5 | ≧3 | 60 |
| | S288C | SD | Chorismate Group Mix (A) | 48 | 4.1 | ≧3 | 29 |
| | S288C | SD | Homocysteine (B) | 40 | <2.5 | ≧3 | 7 |
| | S288C | SD | Isoleucine (B) | 38 | 2.9 | ≧3 | 2 |
| | S288C | SD | Serine (B) | 45 | <2.5 | ≧3 | 20 |
| | S288C | SD | Glycine (B) | 60 | <2.5 | ≧3 | 60 |
| | S288C | SD | Methionine (B) | 100 | <2.5 | ≧3 | 167 |
| | S288C | SD | Threonine (B) | 38 | 2.9 | ≧3 | 2 |
| | S288C | SD | 2-oxobutyrate (B) | 38 | 2.9 | ≧3 | 2 |
| | S288C | SD | Homocysteine Group Mix (B) | 100 | <2.5 | ≧3 | 167 |
| | S288C | SD | Putrescine(C) | 58 | 4.1 | ≧3 | 56 |
| | S288C | SD | Cadaverine (C) | 60 | 4.1 | ≧3 | 60 |
| | S288C | SD | Spermidine (C) | 60 | <2.5 | ≧3 | 60 |
| | S288C | SD | Citrulline (C) | 97 | 5.8 | ≧3 | 158 |
| | S288C | SD | Bicarbonate (C) | 90 | <2.5 | ≧3 | 140 |
| | S288C | SD | Polyamine Group Mix (C) | 42 | 2.9 | ≧3 | 11 |
| | S288C | SD | Lysine (D) | 45 | <2.5 | ≧3 | 20 |

Supplement Combinations

| | | | | | | | |
|---|---|---|---|---|---|---|---|
| Control | S288C | SD | none | 38 | 2.7 | ≧3 | 0 |
| | S288C | SD | Isoleucine (B), Bicarbonate | 67 | <2.5 | ≧3 | 78 |
| | | | (C), Lysine (D) | | | | |
| | S288C | SD | Homocysteine (B), | 80 | <2.5 | ≧3 | 113 |
| | | | Bicarbonate (C), Lysine (D) | | | | |
| | S288C | SD | Tyrosine (A), Methionine (B), | 55 | 4.7 | ≧3 | 47 |
| | | | Putrescine (C), Lysine (D) | | | | |
| | S288C | SD | Methionine (B), Putrescine | 55 | <2.5 | ≧3 | 47 |
| | | | (C) | | | | |
| | S288C | SD | Methionine (B), Ornithine (C) | 50 | <2.5 | ≧3 | 33 |
| | S288C | SD | Homocysteine (B), | 40 | 4.7 | ≧3 | 7 |
| | | | Spermidine (C) | | | | |
| | S288C | SD | Tyrosine (A), Bicarbonate (C), | 70 | <2.5 | ≧3 | 87 |
| | | | Lysine (D) | | | | |
| | S288C | SD | Tyrosine (A), Bicarbonate (C), | 50 | 4.7 | ≧3 | 33 |
| | | | Uracil (E) | | | | |
| | S288C | SD | Methionine (B), Spermidine | 100 | 4.7 | ≧3 | 167 |
| | | | (C), Lysine (D) | | | | |
| | S288C | SD | Methionine (B), Bicarbonate | 80 | <2.5 | ≧3 | 113 |
| | | | (C), Lysine (D) | | | | |
| | S288C | SD | Methionine (B), Bicarbonate | 78 | 4.7 | ≧3 | 107 |
| | | | (C), Uracil (E) | | | | |
| | S288C | SD | Methionine (B), Bicarbonate | 73 | <2.5 | ≧3 | 93 |
| | | | (C), Citrate (F) | | | | |
| | S288C | SD | Homocysteine (B), | 77 | <2.5 | ≧3 | 104 |
| | | | Bicarbonate (C) | | | | |
| | S288C | SD | Putrescine (C), Lysine (D) | 77 | <2.5 | ≧3 | 104 |
| | S288C | SD | Tyrosine (A), Methionine (B), | 68 | 4.7 | ≧3 | 82 |
| | | | Putrescine (C), Lysine (D), | | | | |
| | | | Uracil (E), Citrate (F) | | | | |
| | S288C | SD | Tyrosine (A), Putrescine (C) | 57 | 4.7 | ≧3 | 51 |
| | S288C | SD | Tyrosine (A), Spermidine (C) | 60 | 4.7 | ≧3 | 60 |
| | S288C | SD | Tetrahydrofolate (A), | 50 | <2.5 | ≧3 | 33 |
| | | | Spermidine (C) | | | | |
| | S288C | SD | Methionine (B), Spermidine | 50 | <2.5 | ≧3 | 33 |
| | | | (C) | | | | |
| | S288C | SD | Homocysteine (B), Putrescine | 100 | <2.5 | ≧3 | 167 |
| | | | (C) | | | | |
| | S288C | SD | Spermidine (C), Lysine (D) | 100 | <2.5 | ≧3 | 167 |
| | S288C | SD | Bicarbonate (C), Citrate (F) | 50 | <2.5 | ≧3 | 33 |
| | S288C | SD | Tyrosine (A), Methionine (B), | 40 | <2.5 | ≧3 | 7 |
| | | | Uracil (E) | | | | |
| | S288C | SD | Tyrosine (A), Bicarbonate (C), | 50 | <2.5 | ≧3 | 33 |
| | | | Citrate (F) | | | | |
| | S288C | SD | Methionine (B), Spermidine | 50 | <2.5 | ≧3 | 33 |
| | | | (C), Citrate (F) | | | | |
| | S288C | SD | Homocysteine (B), | 57 | 4.7 | ≧3 | 51 |
| | | | Bicarbonate (C), Uracil (E) | | | | |

TABLE 15
Yeast Genetic Modification Results Under Aerobic Conditions

| | | | | Vector based | MIC Assay | | MIC | % Increase |
|---|---|---|---|---|---|---|---|---|
| | | Group | | Genetic | Result | | Assay | Over |
| Strain Name | Media | Represented | Parent | Modifications | (g/L 3-HP) | S.D. | Number | Control |
| YX-CJR-001 | SD | none | BY4709 | pRS426 EV | 40 | <2.5 | ≧3 | — |
| YX-CJR-002 | SD | C | BY4709 | pYes2.1-spe3 | 50 | <2.5 | ≧3 | 25 |
| YX-CJR-003 | SD | B | BY4709 | pYes2.1-hom2 | 47 | <2.5 | ≧3 | 17 |
| YX-CJR-005 | SD | B | BY4709 | pYes2.1-Met6 | 50 | <2.5 | ≧3 | 25 |
| YX-CJR-006 | SD | B | BY4709 | pYes2.1-Ilv2 | 57 | <2.5 | ≧3 | 42 |
| YX-CJR-010 | SD | B | BY4709 | pyes2.1-Thr1 | 60 | <2.5 | ≧3 | 50 |
| YX-CJR-014 | SD | C | BY4709 | pyes2.1-arg2 | 60 | <2.5 | ≧3 | 50 |
| YX-CJR-017 | SD | A | BY4709 | pyes2.1-Aro7 | 70 | <2.5 | ≧3 | 75 |
| YX-022 | SD | A, B | BY4722 | pyes2.1-Aro3 | 60 | <2.5 | ≧3 | 50 |
| | | | | pRS425-ILV6 | | | | |

TABLE 16
Yeast Genetic Modification Results Under Anaerobic Conditions

| | | | | | MIC Assay | | MIC | % Increase |
|---|---|---|---|---|---|---|---|---|
| | | Group | | Vector based Genetic | Result | | Assay | Over |
| Strain Name | Media | Represented | Parent | Modifications | (g/L 3-HP) | P-value | Number | Control |
| YX-CJR-001 | SD | none | BY4709 | pRS426 EV | 40 | <0.1 | ≧3 | — |
| YX-CJR-005 | SD | B | BY4709 | pYes2.1-Met6 | 60 | <0.1 | ≧3 | 50 |
| YX-CJR-007 | SD | B | BY4709 | pyes2.1-ILV6 | 50 | <0.1 | ≧3 | 25 |
| YX-CJR-008 | SD | B | BY4709 | pyes2.1-ILV1 | 60 | <0.1 | ≧3 | 50 |
| YX-CJR-010 | SD | B | BY4709 | pyes2.1-Thr1 | 50 | <0.1 | ≧3 | 25 |
| YX-CJR-011 | SD | B | BY4709 | pyes2.1-Ser2 | 50 | <0.1 | ≧3 | 25 |
| YX-CJR-013 | SD | B | BY4709 | pyes2.1-ser3 | 50 | <0.1 | ≧3 | 25 |
| YX-CJR-014 | SD | C | BY4709 | pyes2.1-arg2 | 50 | <0.1 | ≧3 | 25 |
| YX-CJR-015 | SD | E | BY4709 | pyes2.1-RNR1 | 50 | <0.1 | ≧3 | 25 |
| YX-CJR-016 | SD | A | BY4709 | pyes2.1-Aro3 | 50 | <0.1 | ≧3 | 25 |
| YX-CJR-018 | SD | A | BY4709 | pyes2.1-Tyr1 | 50 | <0.1 | ≧3 | 25 |
| YX-CJR-021 | SD | A | BY4709 | pYes2.1-Trp1 | 50 | <0.1 | ≧3 | 25 |
| YX-022 | SD | A, B | BY4722 | pyes2.1-Aro3 | 50 | <0.1 | ≧3 | 25 |
| | | | | pRS425-ILV6 | | | | |

TABLE 17
C. necator Supplement Results under Aerobic Conditions

| | | | | average MIC | | MIC | |
|---|---|---|---|---|---|---|---|
| Strain | | | Supplement | Assay Result | | Assay | % Increase |
| Name | Media | Supplements | Codes | (g/L 3-HP) | P-value | Number | Over Control |
| DSM428 | FGN | none | none | 15 | <0.1 | ≧3 | — |
| DSM 542 | EZ Rich | none | none | 60 | <0.1 | ≧3 | 200 |
| DSM 542 | FGN | none | none | 15 | <0.1 | ≧3 | 0 |
| DSM 542 | FGN | Homocysteine Bicarbonate, Lysine | B, C, D | 30 | <0.1 | ≧3 | 100 |
| DSM 542 | FGN | Tyrosine, Methionine, Putrescine, | A, B, C, D | 30 | <0.1 | ≧3 | 100 |
| | | Lysine | | | | | |
| DSM 542 | FGN | Methionine, Putrescine | B, C | 25 | <0.1 | ≧3 | 67 |
| DSM 542 | FGN | Methionine, Ornithine | B, C | 30 | <0.1 | ≧3 | 100 |
| DSM 542 | FGN | Homocysteine, Spermidine | B, C | 25 | <0.1 | ≧3 | 67 |
| DSM 542 | FGN | Methionine, Bicarbonate, Citrate | B, C, F | 25 | <0.1 | ≧3 | 67 |
| DSM 542 | FGN | Homocysteine, Bicarbonate | B, C | 25 | <0.1 | ≧3 | 67 |
| DSM 542 | FGN | Homocysteine Group Mix | B | 20 | <0.1 | ≧3 | 33 |

1-101. (canceled)

102. A genetically modified microorganism comprising:
a) at least one genetic modification to produce 3-hydroxypropionic acid (“3-HP”), wherein the at least one genetic modification to produce 3-HP increases microbial synthesis of 3-HP above a rate or titer of a control microorganism lacking the at least one genetic modification to produce 3-HP; and
b) at least one genetic modification of the 3-HP Toleragenic Complex (“3HPTGC”) effective to increase 3-HP tolerance of the genetically modified microorganism at least about 20 percent, or at least about 50 percent, above the 3-HP tolerance of a control microorganism lacking the at least one genetic modification of the 3HPTGC.

103. The genetically modified microorganism of claim 102, wherein the at least one genetic modification of step b excludes a genetic modification of Group A, or of Group B, or wherein the 3HPTGC excludes arginine decarboxylase.

104. The genetically modified microorganism of claim 102, wherein the at least one genetic modification of step b comprises a genetic modification of Group A and a genetic modification of Group B.

105. The genetically modified microorganism of claim 102, wherein the at least one genetic modification of the 3HPTGC increases enzymatic conversion at one or more enzymatic conversion steps selected from the group consisting of: chorismate to prephenate; prephenate to 4-hydroxyphenylpyruvate; prephenate to phenylpyruvate; D-erythrose-4-phosphate to 3-deoxy-D-arabino-heptulosonate-7-phosphate; 3-deoxy-D-arabino-heptulosonate-7-phosphate to 3-dehydroquinate; 3-dehydroshikimate to shikimate; shikimate to shikimate 3-phosphate; 7,8-dihydrofolate to tetrahydrofolate; 1,4-dihdroxy-2-naphthoate to demethylmenaquinone-8; 3-phosphoglycerate to 3-phospho-hyrdroxypyruvate; serine to glycine; threonine to 2-oxobutanoate; O-acetylserine to cysteine; cysteine and 2-oxobutanoate to cystathionine; homocysteine to methionine; agmatine to putrescine; ornithine to putrescine; putrescine and S-adenosyl-L-methioninamine to spermidine; meso-diaminopimelate to lysine; lysine to cadaverine; cadaverine and S-adenosyl-L-methioninamine to aminopropylcadaverine; aspartate to aspartyl-4-phosphate; aspartyl-4-phosphate to aspartate-semialdehyde; aspartate-semialdehyde to 2,3-dihydrodipicolinate; aspartate-semialdehyde to homoserine; D-ribose-5-phosphate to 5-phosphoribosyl-1-pyrophosphate; GDP to dGDP; ADP to dADP; cyanate to carbamate; CO₂ to HCO₃; and 5,10-methenyltetrahydrofolate to 10-formyl-tetrahydrofolate.

106. The genetically modified microorganism of claim 102, wherein the at least one 3HPTGC genetic modification(s) comprise(s) introducing into at least one microorganism at least one heterologous nucleic acid sequence encoding at least one polypeptide to increase the activity of at least one of the enzymatic conversion steps converting cyanate to carbamate and converting CO₂ to HCO₃.

107. The genetically modified microorganism of claim 102, wherein the at least one 3HPTGC genetic modification(s) comprise(s) at least one genetic modification of one or more 3HPTGC repressor genes selected from tyrR, trpR, metJ, purR, lysR, nrdR, and equivalent repressor genes.

108. The genetically modified microorganism of claim 102, further comprising a genetic modification to increase expression of SEQ ID NO:129.

109. A microorganism culture system comprising:
a) the genetically modified microorganism of claim 1; and
b) a media comprising at least about 50 g/L of 3-HP.

110. The microorganism culture system of claim 109, wherein the media is a minimal media.

111. The microorganism culture system of claim 109, further comprising at least one supplement, wherein the at least one supplement comprises at least one product of an enzymatic conversion step of the 3HPTGC.

112. The microorganism culture system of claim 109, wherein the at least one genetic modification of the 3HPTGC increases enzymatic conversion at one or more enzymatic conversion steps selected from the group consisting of: chorismate to prephenate; prephenate to 4-hydroxyphenylpyruvate; prephenate to phenylpyruvate; D-erythrose-4-phosphate to 3-deoxy-D-arabino-heptulosonate-7-phosphate; 3-deoxy-D-arabino-heptulosonate-7-phosphate to 3-dehydroquinate; 3-dehydroshikimate to shikimate; shikimate to shikimate 3-phosphate; 7,8-dihydrofolate to tetrahydrofolate; 1,4-dihdroxy-2-naphthoate to demethylmenaquinone-8; 3-phosphoglycerate to 3-phospho-hyrdroxypyruvate; serine to glycine; threonine to 2-oxobutanoate; O-acetylserine to cysteine; cysteine and 2-oxobutanoate to cystathionine; homocysteine to methionine; agmatine to putrescine; ornithine to putrescine; putrescine and S-adenosyl-L-methioninamine to spermidine; meso-diaminopimelate to lysine; lysine to cadaverine; cadaverine and S-adenosyl-L-methioninamine to aminopropylcadaverine; aspartate to aspartyl-4-phosphate; aspartyl-4-phosphate to aspartate-semialdehyde; aspartate-semialdehyde to 2,3-dihydrodipicolinate; aspartate-semialdehyde to homoserine; D-ribose-5-phosphate to 5-phosphoribosyl-1-pyrophosphate; GDP to dGDP; ADP to dADP; cyanate to carbamate; CO₂ to HCO₃; and 5,10-methenyltetrahydrofolate to 10-formyl-tetrahydrofolate.

113. A method of making a genetically modified microorganism comprising:
a) introducing to a selected microorganism at least one genetic modification of a 3-hydroxypropionic acid (“3-HP”) production pathway to increase microbial synthesis of 3-HP above the rate of a control microorganism lacking the at least one genetic modification; and
b) introducing to the selected microorganism at least one genetic modification of the 3-HP Toleragenic Complex (“3HPTGC”) that increases 3-HP tolerance of the genetically modified microorganism at least about 20 percent, or at least about 50 percent, above the 3-HP tolerance of a control microorganism lacking the at least one genetic modification of the 3HPTGC.

114. The method of claim 113, wherein the introducing of step b excludes introducing a genetic modification of Group A, or of Group B, or wherein the 3HPTGC excludes arginine decarboxylase.

115. The method of claim 113, wherein the introducing of step b comprises introducing a genetic modification of Group A and a genetic modification of Group B.

116. The method of claim 113, wherein the introducing of step b comprises introducing at least one genetic modification that increases enzymatic conversion of at one or more enzymatic conversion steps of the 3HPTGC selected from the group consisting of: chorismate to prephenate; prephenate to 4-hydroxyphenylpyruvate; prephenate to phenyl pyruvate; D-erythrose-4-phosphate to 3-deoxy-D-arabino-heptulosonate-7-phosphate; 3-deoxy-D-arabino-heptulosonate-7-phosphate to 3-dehydroquinate; 3-dehydroshikimate to shikimate; shikimate to shikimate 3-phosphate; 7,8-dihydrofolate to tetrahydrofolate; 1,4-dihdroxy-2-naphthoate to demethylmenaquinone-8; 3-phosphoglycerate to 3-phospho-hyrdroxypyruvate; serine to glycine; threonine to 2-oxobutanoate; O-acetylserine to cysteine; cysteine and 2-oxobutanoate to cystathionine; homocysteine to methionine; agmatine to putrescine; ornithine to putrescine; putrescine and S-adenosyl-L-methioninamine to spermidine; meso-diaminopimelate to lysine; lysine to cadaverine; cadaverine and S-adenosyl-L-methioninamine to aminopropylcadaverine; aspartate to aspartyl-4-phosphate; aspartyl-4-phosphate to aspartate-semialdehyde; aspartate-semialdehyde to 2,3-dihydrodipicolinate; aspartate-semialdehyde to homoserine; D-ribose-5-phosphate to 5-phosphoribosyl-1-pyrophosphate; GDP to dGDP; ADP to dADP; cyanate to carbamate; CO₂ to HCO₃; and 5,10-methenyltetrahydrofolate to 10-formyl-tetrahydrofolate.

117. The method of claim 113, comprising for step b introducing at least two genetic modifications of the 3HPTGC.

118. The method of claim 113 additionally comprising introducing comprising a genetic modification to increase expression of SEQ ID NO:129.

119. The method of claim 113 comprising introducing at least one heterologous nucleic acid sequence encoding at least one polypeptide to increase the activity of at least one of the enzymatic conversion steps selected from converting cyanate to carbamate and converting CO₂ to HCO₃.

120. The method of claim 119, comprising for step b introducing at least one additional genetic modification of the 3HPTGC.

121. The method of claim 119, comprising for step b introducing at least two additional genetic modifications of the 3HPTGC.
